(19) United States
(12) Patent Application Publication
(10) Pub. No.: US 2024/0174651 A1
(43) Pub. Date: May 30, 2024

(54) COMPOUNDS AND USE THEREOF FOR TREATMENT OF NEURODEGENERATIVE, DEGENERATIVE AND METABOLIC DISORDERS

(71) Applicants: THE SCRIPPS RESEARCH INSTITUTE, La Jolla, CA (US); VOVA IDA THERAPEUTICS, INC., Jupiter, FL (US)

(72) Inventors: Thomas D. BANNISTER, Palm Beach Gardens, FL (US); Corinne LASMÉZAS, Palm Beach Gardens, FL (US); Minghai ZHOU, Jupiter, FL (US); Ronald J. RAHAIM, Jr., Palm Beach Gardens, FL (US)

(21) Appl. No.: 18/262,409

(22) PCT Filed: Jan. 28, 2022
(86) PCT No.: PCT/US22/14312

§ 371 Date: Jul. 21, 2023

Related U.S. Application Data
(60) Provisional application No. 63/142,634, filed on Jan. 28, 2021.

(51) Int. Cl. C07D 405/14; C07D 401/06; C07D 401/14; C07D 413/14; C07D 491/107; C07D 493/08; C07D 498/08

(52) U.S. Cl. CPC C07D 405/14; C07D 401/06; C07D 401/14; C07D 413/14; C07D 491/107; C07D 493/08; C07D 498/08

(57) ABSTRACT

Provided are, inter alia, compounds having a structure of Formula (X), (XI), (XII), or (XIII), or a subordinate structure thereof, composition including the same and methods of use.

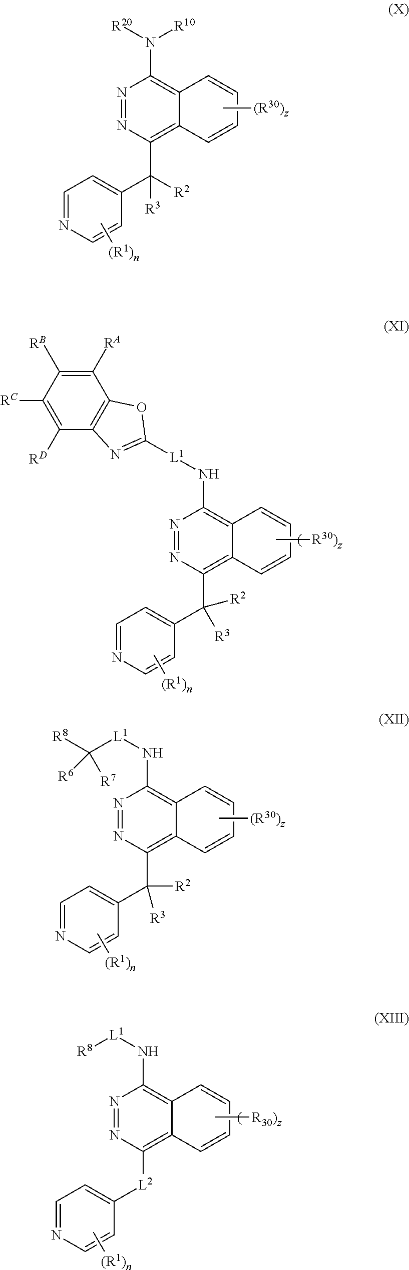

### CROSS-REFERENCES TO RELATED APPLICATIONS

This application claims the benefit of priority of U.S. Provisional Application No. 63/142,634 filed on Jan. 28, 2021, which is incorporated herein by reference in its entirety and for all purposes.

### STATEMENT OF GOVERNMENT SUPPORT

This invention was made with government support under Grant Number 5R01NS103195 awarded by the National Institutes of Health. The government has certain rights in the invention.

### BACKGROUND

A number of fatal neurodegenerative diseases, including prion diseases such as Creutzfeldt-Jakob disease (CJD), Alzheimer's (AD), Parkinson's (PD), frontotemporal dementia (FTD) and amyotrophic lateral sclerosis (ALS), are characterized by toxicity resulting from protein misfolding, and are called protein misfolding neurodegenerative diseases (PMNDs). Proteins involved in these diseases misfold and form aggregates of various sizes. Some of these aggregates are highly toxic for neurons, a phenomenon also referred to as proteotoxicity. Protein aggregates can also exhibit “prion-like” properties, in the sense that they propagate from cell to cell and act as seeds to amplify the misfolding and aggregation process within a cell. Such toxic misfolded proteins include the prion protein PrP in CJD, As and tau in AD; α-synuclein and tau in PD; tau, TDP-43 and C9ORF72 in FTD; SOD1, TDP43, FUS and C9ORF72 in ALS. PD belongs to a broader group of diseases called synucleinopathies, characterized by the accumulation of misfolded α-synuclein aggregates. Lewy body dementia and Multiple System Atrophy are also synucleinopathies. FTD belongs to another group of PMNDs termed tauopathies, a group that also includes chronic traumatic encephalopathy (CTE) and progressive supranuclear palsy (PSP). There are also non-neurological diseases involving protein misfolding, such as diabetes mellitus where the proteins IAPP and proinsulin form protein aggregates that are toxic for pancreatic beta-cells, and cardiomyopathy caused by transthyretin (TTR) amyloidosis (ATTR). TTR amyloid deposits predominantly in peripheral nerves causes a polyneuropathy.

Poor knowledge of the mechanisms of neurotoxicity has hampered the development of effective therapies for PMNDs. To study such mechanisms, a model that uses misfolded and toxic prion protein (TPrP) has been developed, and in particular TPrP reproducibly induces neuronal death in cell culture and after intracerebral injection¹. TPrP induces death of more than 60% of cultured neurons at nanomolar concentration, whereas the natively folded counterpart of the prion protein, NTPrP, does not. Therefore, this model provides a highly efficient system to study mechanisms of neuronal death linked to proteotoxicity that are broadly applicable to protein misfolding diseases. Thus, as demonstrated herein, TPrP-based studies spurred the development of new neuroprotective approaches for treating devastating neurodegenerative diseases and other diseases involving the death of particular cell types.

### SUMMARY

Provided herein, inter alia, are novel compounds that may inhibit NAD consumption and/or increase NAD synthesis.

In an aspect is provided a compound having a structure of Formula (X),

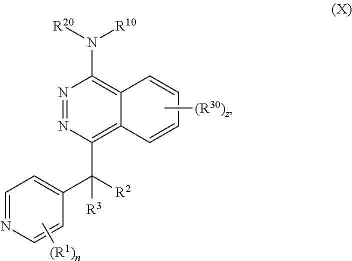

or a pharmaceutically acceptable salt thereof, or an isomer thereof;
wherein:
R¹⁰ is hydrogen, substituted or unsubstituted alkyl, substituted or unsubstituted heteroalkyl;
R²⁰ is -L¹-R²¹;
L¹ is a bond, substituted or unsubstituted alkylene, or substituted or unsubstituted heteroalkylene;
R²¹ is substituted or unsubstituted cycloalkyl, substituted or unsubstituted heterocycloalkyl, substituted or unsubstituted aryl, or substituted or unsubstituted heteroaryl;
R¹⁰ and R²⁰ are optionally joined to form a substituted or unsubstituted bicyclic heterocycloalkyl;
R¹ is independently halogen, —CX¹₃, —CHX¹₂, —CH₂X¹, —OCX¹₃, —OCH₂X¹, —OCHX¹₂, —CN, —OR^{1F}, —SR^{1F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, D, halogen, —CX²₃, —CHX²₂, —CH₂X², —OCX²₃, —OCH₂X², —OCHX²₂, —OR^{2F}, —SR^{2F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, D, halogen, —CX³₃, —CHX³₂, —CH₂X³, —OCX³₃, —OCH₂X³, —OCHX³₂, —OR^{3F}, —SR^{3F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³⁰ is independently halogen, —CX³⁰₃, —CHX³⁰₂, —CH₂X³⁰, —OCX³⁰₃, —OCH₂X³⁰, —OCHX³⁰₂, —CN, —OR^{30F}, —SR^{30F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is an integer of 0 to 4;
z is an integer of 0 to 4;
Each X¹, X², X³ and X³⁰ is independently —F, —Br, —Cl, or —I; and
Each R^{1F}, R^{2F}, R^{3F} and R^{30F} is independently hydrogen, substituted or unsubstituted alkyl, or substituted or unsubstituted heteroalkyl,
provided that when R¹⁰ is hydrogen and L¹ is a bond, then R²¹ is not unsubstituted phenyl nor phenyl substituted with halogen, —C(O)CH₃, —S(O)₂—NH₂, or substituted or unsubstituted alkyl; and when L¹ is a methylene, then R²¹ is not unsubstituted tetrahydro-pyranyl.

In an aspect is provided a compound having a structure of Formula (XII),

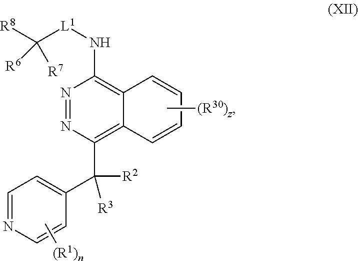

or a pharmaceutically acceptable salt thereof, or an isomer thereof;
wherein:
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is independently halogen, —CX¹₃, —CHX¹₂, —CH₂X¹, —OCX¹₃, —OCH₂X¹, —OCHX¹₂, —CN, —OR^{1F}, —SR^{1F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, D, halogen, —CX²₃, —CHX²₂, —CH₂X², —OCX²₃, —OCH₂X², —OCHX²₂, —OR^{2F}, —SR^{2F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, D, halogen, —CX³₃, —CHX³₂, —CH₂X³, —OCX³₃, —OCH₂X³, —OCHX³₂, —OR^{3F}, —SR^{3F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ are optionally joined together to form a substituted or unsubstituted cycloalkyl, or substituted or unsubstituted heterocycloalkyl;
Each R⁶ and R⁷ is independently hydrogen, substituted or unsubstituted alkyl, or substituted or unsubstituted heteroalkyl; or R⁶ and R⁷ are optionally joined together to form a substituted or unsubstituted cycloalkyl, or substituted or unsubstituted heterocycloalkyl;
R⁸ is a substituted or unsubstituted cycloalkyl, or substituted or unsubstituted heterocycloalkyl, substituted or unsubstituted aryl, or substituted or unsubstituted heteroaryl;
R³⁰ is independently halogen, —CX³⁰₃, —CHX³⁰₂, —CH₂X³⁰, —OCX³⁰₃, —OCH₂X³⁰, —OCHX³⁰₂, —CN, —OR^{30F}, —SR^{30F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is an integer of 0 to 4;
z is an integer of 0 to 4;
Each X¹, X², X³, and X³⁰ is independently —F, —Br, —Cl, or —I; and
Each R^{1F}, R^{2F}, R^{3F}, and R^{30F} is independently hydrogen, substituted or unsubstituted alkyl, or substituted or unsubstituted heteroalkyl,
provided that when n is 0, L¹ is a bond, R⁶ and R⁷ are hydrogen, then R⁸ is not unsubstituted tetrahydro-pyranyl.

In embodiments, in Formula (XII), at least one of R² and R³ is not hydrogen.

In an aspect is provided a compound has a structure of Formula (XIII):

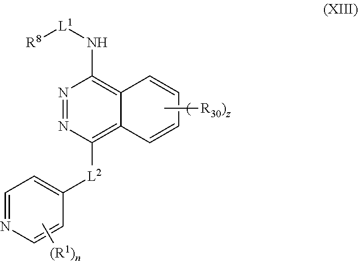

or a pharmaceutically acceptable salt thereof, or an isomer thereof;
wherein:
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is —C(═O)—, —C(═CR^{9A}R^{9B})—, —CR^{10A}R^{10B}—, or —C(═NR¹¹)—;
R¹ is independently halogen, —CX¹₃, —CHX¹₂, —CH₂X¹, —OCX¹₃, —OCH₂X¹, —OCHX¹₂, —CN, —OR^{1F}, —SR^{1F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is a substituted or unsubstituted cycloalkyl, substituted or unsubstituted heterocycloalkyl, substituted or unsubstituted aryl, or substituted or unsubstituted heteroaryl;
Each R^{9A} and R^{9B} is independently hydrogen, —CX⁹₃, —CHX⁹₂, —CH₂X⁹, —OCX⁹₃, —OCH₂X⁹, —OCHX⁹₂, —CN, —OR^{9F}, —SR^{9F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Each R^{10A} and R^{10B} is independently hydrogen, D, halogen, —CX¹⁰₃, —CHX¹⁰₂, —CH₂X¹⁰, —OCX¹⁰₃, —OCH₂X¹⁰, —OCHX¹⁰₂, —CN, —OR^{10F}, —SR^{10F}, —C(O)OR^{10F}, —C(O)NHR^{10F}, —C(O)N(R^{10F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10A} and R^{10B} are optionally joined together to form a substituted or unsubstituted cycloalkyl, or substituted or unsubstituted heterocycloalkyl;
R¹¹ is —R^{11F}, —OR^{11F}, —S(O₂)—R^{11F}, or —C(O)—R^{11F};
R³⁰ is independently halogen, —CX³⁰₃, —CHX³⁰₂, —CH₂X³⁰, —OCX³⁰₃, —OCH₂X³⁰, —OCHX³⁰₂, —CN, —OR^{30F}, —SR^{30F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is an integer of 1 to 4;
z is an integer of 0 to 4;
Each X¹, X⁸, X⁹, X¹⁰, and X³⁰ is independently —F, —Br, —Cl, or —I;
Each R^{1F}, R^{8F}, R^{9F}, R^{10F} and R^{30F} is independently hydrogen, substituted or unsubstituted alkyl, or substituted or unsubstituted heteroalkyl; and
R^{11F} is a hydrogen, substituted or unsubstituted alkyl, substituted or unsubstituted aryl, or substituted or unsubstituted heteroaryl.

In embodiments, in Formula (XIII), at least one of R^{10A} and R^{10B} is not hydrogen.

In an aspect, provided is a pharmaceutical composition including the compound described herein, a pharmaceutically acceptable salt form thereof, an isomer thereof, or a crystal form thereof.

In an aspect, provided is a method of inhibiting NAD consumption and/or increasing NAD synthesis in a patient. The method may include administering to the patient an effective dose of the compound as described herein.

In an aspect, provided is a method of preventing or inhibiting NAD depletion in a patient, or a method of improving a condition linked to alterations of NAD metabolism in a patient. The method may include administering to the patient an effective dose of the compound as described herein.

In an aspect, provided is a method of providing protection from toxicity of misfolded proteins in a patient. The method may include administering to the patient an effective dose of the compound as described herein.

In an aspect, provided is a method of preventing or treating a protein misfolding neurodegenerative disease in a patient. The method may include administering to the patient an effective dose of the compound as described herein.

In an aspect, provided is a method of preventing or treating mitochondrial dysfunction in a patient. The method may include administering to the patient an effective dose of the compound as described herein.

In an aspect, provided is a method of preventing or treating a retinal disease in a patient. The method may include administering to the patient an effective dose of the compound as described herein.

In an aspect, provided is a method of preventing or treating diabetes, non alcoholic fatty liver disease or other metabolic disease in a patient. The method may include administering to the patient an effective dose of the compound as described herein.

In an aspect, provided is a method of preventing or treating a kidney disease or kidney failure in a patient. The method may include administering to the patient an effective dose of the compound as described herein.

In an aspect, provided is a method of mitigating health effects of aging. The method may include administering to the patient an effective dose of the compound as described herein.

In an aspect, provided is a method of preventing or treating neuronal degeneration associated with multiple sclerosis, an axonopathy, an optic neuropathy, a cardiomyopathy, brain or cardiac ischemia, traumatic brain injury, hearing loss, or retinal damage in a patient. The method may include administering to the patient an effective dose of the compound as described herein.

Other aspects of the inventions are disclosed infra.

### BRIEF DESCRIPTION OF THE DRAWINGS

FIGS. 1A-D show dose-response curves of compounds in the TPrP neuroprotection assay.

FIGS. 2A-F show effects of compounds on the activation rate of the enzyme nicotinamide phosphoribosyltransferase (NAMPT).

### DETAILED DESCRIPTION

The misfolded toxic prion protein TPrP induces a profound depletion of neuronal NAD that is responsible for cell death, since NAD replenishment leads to full recovery of cells exposed to TPrP injury in vitro and in vivo, despite continued exposure to TPrP². Intranasal NAD treatment improved motor function and activity in murine prion disease. Further it was discovered that NAD depletion in neurons exposed to TPrP may be due, at least in part, to overconsumption of cellular NAD during metabolic reactions called mono-ADP ribosylations². Inhibitors of poly-ADP-ribosylations, called PARP inhibitors, have previously been developed as anticancer agents. Available selective PARP inhibitors did not alleviate NAD depletion and neuronal death caused by TPrP, demonstrating the need to identify new compounds capable of interfering with the mechanisms at play in misfolded protein-induced toxicity or capable of preventing NAD depletion irrespective of the mechanism underlying NAD imbalance. Imbalance in NAD metabolism is a pathogenic mechanism of a number of human conditions, as described herein.

NAD, as used here, designates both the oxidized (NAD+) and the reduced (NADH) forms of the cofactor. NAD is critical, inter alia, as a co-enzyme for the regulation of energy metabolism pathways such as glycolysis, TCA cycle and oxidative phosphorylation leading to ATP production. In addition, NAD serves as a substrate for signal transduction and post-translational protein modifications called ADP-ribosylations.

Physiological cellular NAD levels result from the balance of activity of NAD synthesis enzymes and NAD consuming enzymes, which may be reasoned that the NAD imbalance induced by misfolded proteins (and that is assessed in our TPrP assay) could therefore result from either impaired NAD biosynthesis or from increased NAD consumption.

In mammalian cells, NAD is mainly synthesized via the salvage pathway using the precursor nicotinamide (NAM). The rate-limiting enzyme for NAD synthesis in the salvage pathway is nicotinamide phosphoribosyltransferase (NAMPT) converting NAM into nicotinamide mononucleotide (NMN). Nicotinamide riboside (NR) is an alternative NAD precursor converted to NMN by nicotinamide riboside kinase. Other NAD synthesis pathways are the de novo pathway utilizing the precursor tryptophan and the Preiss-Handler pathway utilizing the precursor nicotinic acid (NA).

On the other hand, NAD is consumed during the following cellular reactions: 1) the production of calcium-releasing second messengers cyclic ADP-ribose (cADPR) and ADP-ribose (ADPR) from NAD by enzymes called NAD hydrolases or ADP-ribosyl cyclases (CD38 and CD157); 2) sirtuin-mediated protein deacetylations, and 3) protein ADP-ribosylations, in which one or several ADP-ribose moiety of NAD is transferred unto proteins by mono/oligo-ADP-ribose transferases (mARTs) or poly-ADP ribose transferases (called PARPs).

NAD deficiency is a feature of prion diseases² and other PMNDs such as PD^{3,4}, AD⁵⁻⁸ and ALS^{9,10}.

NAD dysregulation is now also recognized as being involved in aging¹¹⁻¹³, neuronal degeneration associated with multiple sclerosis¹⁴, traumatic brain injury¹⁵, hearing loss¹⁶, axonopathy and axonal degeneration^{17,18}. NAD augmentation such as NAD administration or increased NAD synthesis by enzyme overexpression has been shown to mitigate brain ischemia¹⁹ and cardiac ischemia/reperfusion injury²⁰.

Age-related retinal/macular degeneration (AMD) is associated with the death of photoreceptors and retinal pigment epithelium (RPE) cells of the eye's retina, and causes progressive loss of vision. NAD levels are decreased in RPE cells isolated from patients with AMD²². Healthy NAD levels are required for vision in mice²³. Increasing NAD levels by overexpression of cytoplasmic nicotinamide monomucleotide adenyl-transferase-1 (cytNMNAT1) in mice or NAM supplemented diet in rats showed less Zn²⁺ staining, NAD+ loss and cell death after light-induced retinal damage (LIRD)²⁴. Similarly, treatment with the NAD precursor NR maintained retinal NAD levels and protected retinal morphology and function in a mouse model of LIRD²⁵.

NAD metabolism has also been shown to be altered in murine models of type 2 diabetes (T2D)^{26,27}. Alterations of NAD metabolism in diabetes can be explained, at least in part, by our findings that misfolded proteins induce NAD dysregulation. Indeed, diabetes has been shown to be a protein misfolding disease, characterized by pancreatic beta-cell dysfunction and death, concomitant with the deposition of aggregated islet amyloid polypeptide (IAPP), a protein co-expressed and secreted with insulin by pancreatic beta-cells^{28,29}. Similarly to proteins involved in other protein misfolding diseases, IAPP forms toxic oligomers²⁸. Moreover, proinsulin, the precursor of insulin, is also prone to misfold in beta-cells. Misfolding of proinsulin has been linked to type 2, type 1 and some monogenic forms of diabetes progression^{28,30,31}. NR supplementation mitigates type 2 diabetes in mice²⁷.

NAD repletion protects against mitochondrial dysfunction in metabolic diseases³², in age-related amyloidosis³³, and prevents post-ischemic mitochondrial damage and fragmentation³⁴. Overexpression of the NAD synthetic enzyme NAMPT suppresses mitochondrial fragmentation, loss of mitochondrial DNA content and the reductions in expression of the key regulators of mitochondrial biogenesis PGC-1 and NRF-1 in cultured primary neurons subjected to glutamate-induced excitotoxicity or oxygen-glucose deprivation³⁵.

Substantial decreases in NAD levels are found in degenerative renal conditions and NAD augmentation mitigates acute kidney injury triggered by ischaemia-reperfusion, toxic injury and systemic inflammation³⁶.

Using TPrP as a prototypic amyloidogenic misfolded protein exhibiting high neurotoxicity, a high-throughput screening (HTS) assay has been developed to identify compounds effective at a) preventing cell death; and b) preventing NAD depletion induced by TPrP.

The HTS campaign was performed at Scripps Florida using a subset of the Scripps Drug Discovery Library (SDDL). Several potent, novel and chemically tractable small molecules are identified that can provide complete neuroprotection and preservation of NAD levels when used at doses ranging from low nanomolar to low micromolar levels, which is also detailed in international patent Publication WO 2020/232255. Its entire content is incorporated herein by reference for all purposes.

Members of each series of compounds described herein are highly potent in neuroprotection assays designed to reflect the potential for the successful treatment of several neurodegenerative diseases as described herein. Further, many have favorable drug-like properties (e.g., they are PAINS-free³⁷ and compliant with Lipinski and Veber rules for drug-likeness^{38,39}). Since these compounds prevent depletion of cellular NAD levels or increase NAD levels, they have utility in preventing or treating diseases where there is an imbalance in NAD metabolism, such as protein misfolding neurodegenerative diseases, amyloidoses, mitochondrial diseases, aging, retinal degeneration, ischemic conditions, traumatic brain injury, kidney failure and metabolic diseases including diabetes and non alcoholic fatty liver disease.

### Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl (“Me”), ethyl (“Et”), n-propyl (“Pr”), isopropyl (“iPr”), n-butyl (“Bu”), t-butyl (“t-Bu”), isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R′″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R′″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈alkyl, C₁-C₆alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “EC₅₀” or “half maximal effective concentration” as used herein refers to the concentration of a molecule (e.g., small molecule, drug, antibody, chimeric antigen receptor or bispecific antibody) capable of inducing a response which is halfway between the baseline response and the maximum response after a specified exposure time. In embodiments, the EC₅₀ is the concentration of a molecule (e.g., small molecule, drug, antibody, chimeric antigen receptor or bispecific antibody) that produces 50% of the maximal possible effect of that molecule.

As used herein, the term “neurodegenerative disorder” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Chronic Traumatic Encephalopathy, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, Kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Progressive Supranuclear Palsy, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, Tabes dorsalis or Traumatic Brain Injury.

As used herein, the term “retinal degeneration” refers to a disease or condition in which the vision of a subject becomes impaired due to dysfunction and/or damage of the eye's retina. Examples of retinal degeneration include age-related macular degeneration (AMD). Early stage AMD includes abnormalities of the retinal pigment epithelium and drusen. Late-stage AMD can include dry (non-neovascular, atrophic) macular degeneration, wet (neovascular) macular degeneration, proliferative diabetic retinopathy (PDR), diabetic macular edema (DME).

As used herein, the term “axonopathy” refers to functional or structural damage to a neuron or pheripheral nerve.

As used herein, the term “peripheral” refers to the part of the body anatomy located outside of the central nervous system.

As used herein, the term “amyloidosis” refers to a condition linked to the deposition of protein amyloid. An amyloidosis can occur in the central nervous system and is also referred to as a protein misfolding neurodegenerative disease (e.g. prion diseases, AD, PD and other synucleinopathies, ALS, tauopathies). An amyloidosis can occur outside of the central nervous system and can be widespread, i.e. systemic, or located in different organ systems. When amyloid deposits occurs in several organs, it is referred to as “multisystem”. Examples of amyloidoses are cardiomyopathy or polyneuropathy caused by the deposition of the protein TTR in the heart or peripheral nerves, respectively. Other examples of peripheral amyloidoses are AL (Primary) Amyloidosis or AA (Secondary) Amyloidosis.

As used herein, the term “metabolic disorder” refers to a disease or condition in which body metabolism, i.e. the process in which the body gets, makes and stores energy from food, is disrupted. Some metabolic disorders affect the breakdown of amino acids, carbohydrates, or lipids. Other metabolic disorders are known as mitochondrial diseases and affect mitochondria, the cellular organelles that produce energy. Examples of metabolic disorders are diabetes mellitus (sugar metabolism), hypercholesterolemia, Gaucher disease (lipid metabolism), non alcoholic fatty liver disease (NAFLD), metabolic syndrome (dyslipidemia, abdominal obesity, insulin resistance, proinflammatory state).

As used herein, the term “mitochondrial disease” refers to a group of disorders that affect the cellular organelle mitochondria, which main function is to produce energy. Primary mitochondrial disorders are caused by mutations in mitochondrial DNA or in the nuclear DNA. They can affect various organ systems, causing, i.a., a myopathy, diabetes and deafness, blindness, a neuropathy or an encephalopathy. Alternatively, mitochondrial dysfunction is associated with aging and diseases such as diabetes, cancer, Alzheimer's disease, Parkinson's disease, Huntington's disease, bipolar disorder, ischemic conditions.

As used herein, the terms “kidney disease”, “kidney failure”, “renal disease” or “renal failure” refer to a disease or condition in which a subject loses kidney function. The condition can have various etiologies such as infectious, inflammatory, ischemic or traumatic. Kidney failure can be acute, leading to rapid loss of kidney function, or chronic, leading to gradual loss of kidney function. The condition ultimately leads to the accumulation of dangerous levels of fluid, electrolytes and waste products in the body. End-stage kidney failure is fatal without artificial filtering of the blood (dialysis) or kidney transplant.

As used herein, the term “ischemic condition” or “ischemia” refers to a condition in which the blood flow is restricted or reduced in a part of the body, such as the heart or the brain.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

### Compounds

### Embodiments 1

In an aspect, provided herein are compounds that may provide complete neuroprotection and protection of cell types other than neurons, and preservation of NAD levels. The compounds may be highly potent in a) preventing neuronal and/or cellular death; and b) preventing NAD depletion induced by TPrP, for example, as identified by neuroprotection assays when used at doses ranging from low nanomolar to low micromolar levels.

In an aspect, provided is a compound having a structure of Formula (I):

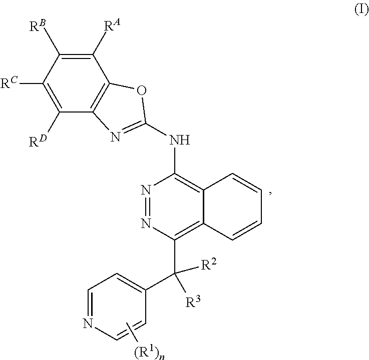

or a pharmaceutically acceptable salt thereof, or an isomer thereof;
wherein:
each R^{A}, R^{B}, R^{C}, and R^{D} is independently hydrogen, substituted or unsubstituted alkyl, substituted or unsubstituted heteroalkyl;
two of R^{A}, R^{B}, R^{C}, and R^{D} are joined to form a substituted or unsubstituted cycloalkyl, substituted or unsubstituted heterocycloalkyl, substituted or unsubstituted aryl, or substituted or unsubstituted heteroaryl;
R¹ is independently halogen, —CX¹₃, —CHX¹₂, —CH₂X¹, —OCX¹₃, —OCH₂X¹, —OCHX¹₂, —CN, —OR^{1F}, —SR^{1F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, D, halogen, —CX²₃, —CHX²₂, —CH₂X², —OCX²₃, —OCH₂X², —OCHX²₂, —CN, —OR^{3F}, —SR^{3F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, D, halogen, —CX³₃, —CHX³₂, —CH₂X³, —OCX³₃, —OCH₂X³, —OCHX³₂, —CN, —OR^{3F}, —SR^{3F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is an integer of 0 to 4,
Each X¹, X² and X³ is independently —F, —Br, —Cl, or —I; and
Each R^{1F}, R^{2F}, and R^{3F} is independently hydrogen, substituted or unsubstituted alkyl, or substituted or unsubstituted heteroalkyl.

In embodiments, R^{A} and R^{B} are joined to form a substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl. In embodiments, R^{A} and R^{B} are joined to form a substituted or unsubstituted C₅-C₆ cycloalkyl. In embodiments, R^{A} and R^{B} are joined to form a substituted or unsubstituted C₅ cycloalkyl. In embodiments, R^{A} and R^{B} are joined to form a substituted or unsubstituted C₆ cycloalkyl. In embodiments, R^{A} and R^{B} are joined to form a substituted or unsubstituted 5 to 6 membered heterocycloalkyl. In embodiments, R^{A} and R^{B} are joined to form a substituted or unsubstituted 5 membered heterocycloalkyl. In embodiments, R^{A} and R^{B} are joined to form a substituted or unsubstituted 6 membered heterocycloalkyl. In embodiments, R^{A} and R^{B} are joined to form a substituted or unsubstituted 5 to 6 membered heterocycloalkyl containing N, O, or S. In embodiments, R^{A} and R^{B} are joined to form a substituted or unsubstituted 5 membered heterocycloalkyl containing N, O, or S. In embodiments, R^{A} and R^{B} are joined to form a substituted or unsubstituted 6 membered heterocycloalkyl containing N, O, or S. In embodiments, R^{A} and R^{B} are joined to form a substituted or unsubstituted phenyl. In embodiments, R^{A} and R^{B} are joined to form substituted or unsubstituted 5 to 6 membered heteroaryl. In embodiments, R^{A} and R^{B} are joined to form substituted or unsubstituted 5 membered heteroaryl. In embodiments, R^{A} and R^{B} are joined to form substituted or unsubstituted 6 membered heteroaryl. In embodiments, R^{A} and R^{B} are joined to form substituted or unsubstituted 5 to 6 membered heteroaryl containing N, O, or S. In embodiments, R^{A} and R^{B} are joined to form substituted or unsubstituted 5 membered heteroaryl containing N, O, or S. In embodiments, R^{A} and R^{B} are joined to form substituted or unsubstituted 6 membered heteroaryl containing N, O, or S.

In embodiments, R^{B} and R^{C} are joined to form a substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl. In embodiments, R^{B} and R^{C} are joined to form a substituted or unsubstituted C₅-C₆ cycloalkyl. In embodiments, R^{B} and R^{C} are joined to form a substituted or unsubstituted C₅ cycloalkyl. In embodiments, R^{B} and R^{C} are joined to form a substituted or unsubstituted C₆ cycloalkyl. In embodiments, R^{B} and R^{C} are joined to form a substituted or unsubstituted 5 to 6 membered heterocycloalkyl. In embodiments, R^{B} and R^{C} are joined to form a substituted or unsubstituted 5 membered heterocycloalkyl. In embodiments, R^{B} and R^{C} are joined to form a substituted or unsubstituted 6 membered heterocycloalkyl. In embodiments, R^{B} and R^{C} are joined to form a substituted or unsubstituted 5 to 6 membered heterocycloalkyl containing N, O, or S. In embodiments, R^{B} and R^{C} are joined to form a substituted or unsubstituted 5 membered heterocycloalkyl containing N, O, or S. In embodiments, R^{B} and R^{C} are joined to form a substituted or unsubstituted 6 membered heterocycloalkyl containing N, O, or S. In embodiments, R^{B} and R^{C} are joined to form a substituted or unsubstituted phenyl. In embodiments, R^{B} and R^{C} are joined to form substituted or unsubstituted 5 to 6 membered heteroaryl. In embodiments, R^{B} and R^{C} are joined to form substituted or unsubstituted 5 membered heteroaryl. In embodiments, R^{B} and R^{C} are joined to form substituted or unsubstituted 6 membered heteroaryl. In embodiments, R^{B} and R^{C} are joined to form substituted or unsubstituted 5 to 6 membered heteroaryl containing N, O, or S. In embodiments, R^{B} and R^{C} are joined to form substituted or unsubstituted 5 membered heteroaryl containing N, O, or S. In embodiments, R^{B} and R^{C} are joined to form substituted or unsubstituted 6 membered heteroaryl containing N, O, or S.

In embodiments, R^{C} and R^{D} are joined to form a substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl. In embodiments, R^{C} and R^{D} are joined to form a substituted or unsubstituted C₅-C₆ cycloalkyl. In embodiments, R^{C} and R^{D} are joined to form a substituted or unsubstituted C₅ cycloalkyl. In embodiments, R^{C} and R^{D} are joined to form a substituted or unsubstituted C₆ cycloalkyl. In embodiments, R^{C} and R^{D} are joined to form a substituted or unsubstituted 5 to 6 membered heterocycloalkyl. In embodiments, R^{C} and R^{D} are joined to form a substituted or unsubstituted 5 membered heterocycloalkyl. In embodiments, R^{C} and R^{D} are joined to form a substituted or unsubstituted 6 membered heterocycloalkyl. In embodiments, R^{C} and R^{D} are joined to form a substituted or unsubstituted 5 to 6 membered heterocycloalkyl containing N, O, or S. In embodiments, R^{C} and R^{D} are joined to form a substituted or unsubstituted 5 membered heterocycloalkyl containing N, O, or S. In embodiments, R^{C} and R^{D} are joined to form a substituted or unsubstituted 6 membered heterocycloalkyl containing N, O, or S. In embodiments, R^{C} and R^{D} are joined to form a substituted or unsubstituted phenyl. In embodiments, R^{C} and R^{D} are joined to form substituted or unsubstituted 5 to 6 membered heteroaryl. In embodiments, R^{C} and R^{D} are joined to form substituted or unsubstituted 5 membered heteroaryl. In embodiments, R^{C} and R^{D} are joined to form substituted or unsubstituted 6 membered heteroaryl. In embodiments, R^{C} and R^{D} are joined to form substituted or unsubstituted 5 to 6 membered heteroaryl containing N, O, or S. In embodiments, R^{C} and R^{D} are joined to form substituted or unsubstituted 5 membered heteroaryl containing N, O, or S. In embodiments, R^{C} and R^{D} are joined to form substituted or unsubstituted 6 membered heteroaryl containing N, O, or S.

In embodiments, the compound has a structure of Formula (I-a), (I-b), or (I-c),

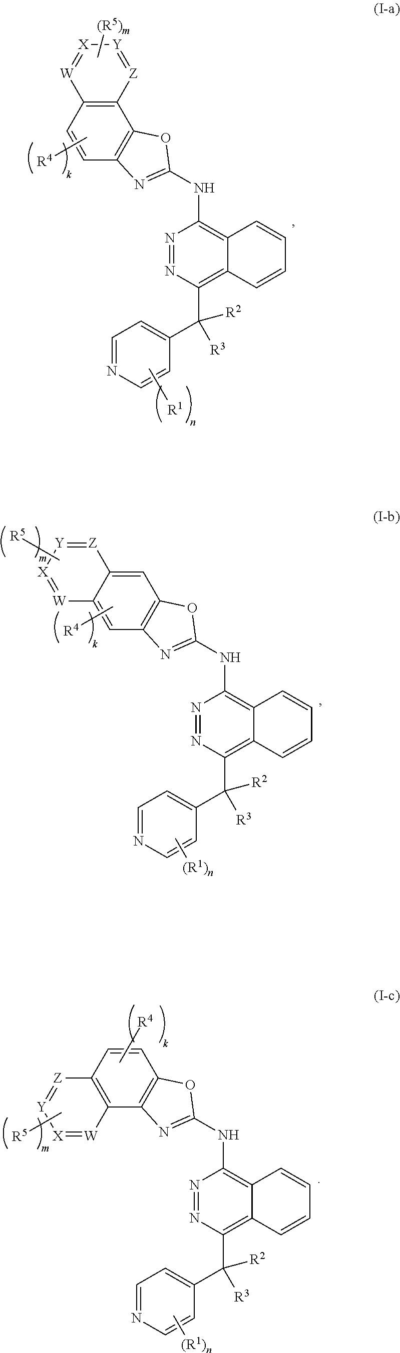

In the Formula (I-a), (I-b), or (I-c),
each W, X, Y and Z is independently ═N— or —CH═;
each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)OR^{4F}, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
each R⁵ is independently halogen, —CX⁵₃, —CHX⁵₂, —CH₂X⁵, —OCX⁵₃, —OCH₂X⁵, —OCHX⁵₂, —CN, —OR^{5F}, —SR^{5F}, —C(O)OR^{5F}, —C(O)NHR^{5F}, —C(O)N(R^{5F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
k is an integer of 0 to 2;
m is an integer of 0 to 4;
Each X⁴ and X⁵ is independently —F, —Br, —Cl, or —I; and
Each R^{4F} and R^{5F} is independently hydrogen, substituted or unsubstituted alkyl, or substituted or unsubstituted heteroalkyl.

In embodiments, W, X, Y and Z are —CH═.

In embodiments, the compound has the structure of (I-a-1), (I-b-1), or (I-c-1),

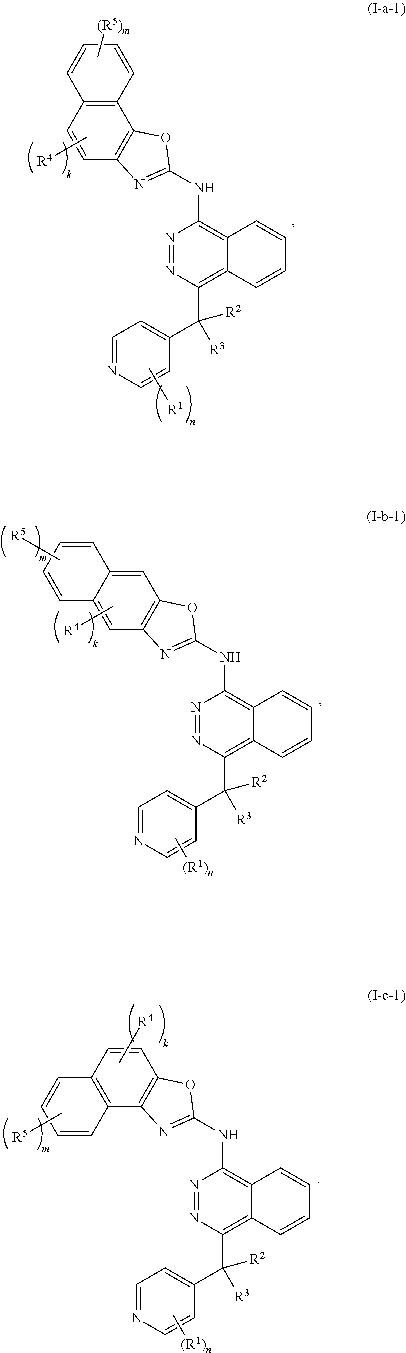

R¹, R², R³, R⁴, R⁵, k, m, and n are described above.

In embodiments, the compound has a structure of Formula (I-d), (I-e), (I-f), (I-d′), (I-e′), or (I-f′),

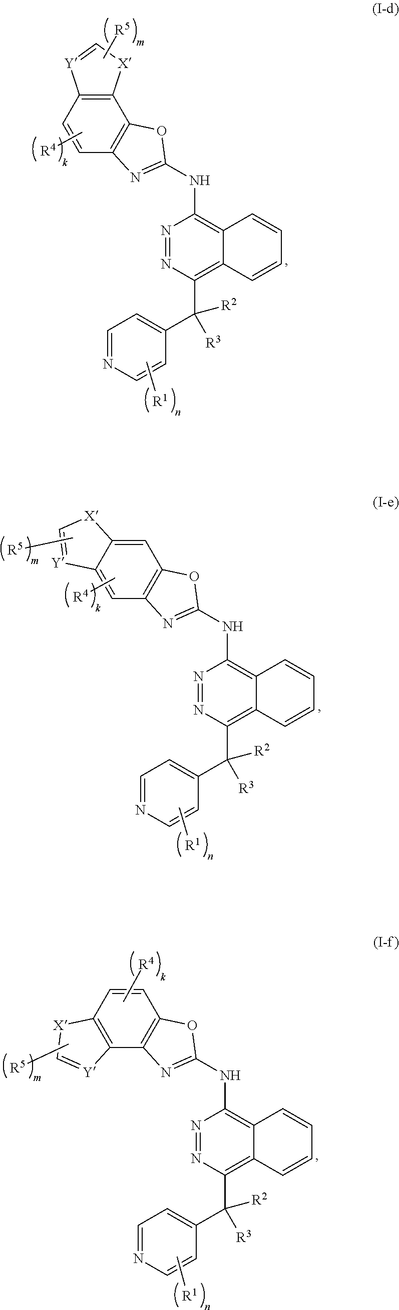

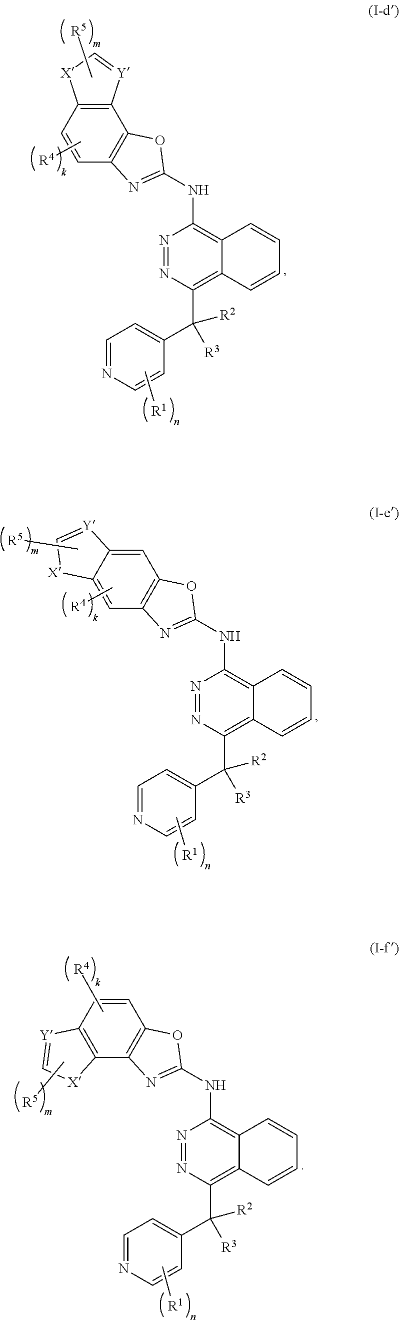

R¹, R², R³, R⁴, R⁵, k, m, and n are described above.

In the Formula (I-d), (I-e), (I-f), (I-d′), (I-e′), or (I-f′),
X′ is —O—, —NH—, or —CH₂—;
Y′ is N—H═, or —CH═;
k is an integer of 0 to 2;
m is an integer of 0 to 3;
Each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)OR^{4F}, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, halogen, —CX⁵₃, —CHX⁵₂, —CH₂X⁵, —OCX⁵₃, —OCH₂X⁵, —OCHX⁵₂, —CN, —OR^{5F}, —SR^{5F}, —C(O)OR^{5F}, —C(O)NHR^{5F}, —C(O)N(R^{5F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
k is an integer of 0 to 2;
m is an integer of 0 to 3;
Each X⁴ and X⁵ is independently —F, —Br, —Cl, or —I; and
Each R^{4F} and R^{5F} is independently hydrogen, substituted or unsubstituted alkyl, or substituted or unsubstituted heteroalkyl.

In embodiments, R² is H, D, halogen, substituted or unsubstituted C₁-C₄ alkyl, —CN, —CX²₃, or OR^{2F}, and R³ is H, D, halogen, substituted or unsubstituted C₁-C₄ alkyl, —CN, —CX³₃, or OR^{3F} In embodiments, each R^{2F} and R^{3F} is independently hydrogen, or unsubstituted C₁-C₄ alkyl. In embodiments, each R^{2F} and R^{3F} is independently hydrogen, or unsubstituted methyl. In embodiments, R^{2F} is hydrogen, or unsubstituted methyl. In embodiments, R^{3F} is hydrogen, or unsubstituted methyl.

In embodiments, R² is —H, -D, halogen, substituted or unsubstituted C₁-C₄ alkyl, —CN, —CX²³, or OR^{2F}. In embodiments, R² is —H. In embodiments, R² is -D. In embodiments, R² is —F. In embodiments, R² is —Cl. In embodiments, R² is —Br. In embodiments, R² is —I. In embodiments, R² is substituted C₁-C₄ alkyl. In embodiments, R² is unsubstituted C₁-C₄ alkyl. In embodiments, R² is methyl. In embodiments, R² is ethyl. In embodiments, R² is —CN. In embodiments, R² is —CF₃. In embodiments, R² is —OH. In embodiments, R² is —OCH₃.

In embodiments, R³ is —H, -D, halogen, substituted or unsubstituted C₁-C₄ alkyl, —CN, —CX³³, or OR^{3F}. In embodiments, R³ is —H. In embodiments, R³ is -D. In embodiments, R³ is —F. In embodiments, R³ is —Cl. In embodiments, R³ is —Br. In embodiments, R³ is —I. In embodiments, R³ is substituted C₁-C₄ alkyl. In embodiments, R³ is unsubstituted C₁-C₄ alkyl. In embodiments, R³ is methyl. In embodiments, R³ is ethyl. In embodiments, R³ is —CN. In embodiments, R³ is —CF₃. In embodiments, R³ is —OH. In embodiments, R³ is —OCH₃.

In embodiments, k is 0. In embodiments, k is 1. In embodiments, k is 2. In embodiments, m is 0. In embodiments, m is 1. In embodiments, m is 2.

In embodiments, each R⁴ is independently halogen, —CX⁴₃, —OCX⁴₃, —CN, —OR^{4F}, —C(O)OR^{4F}, —C(O)NHR^{4F}, —C(O)N(R^{4F})₂, or substituted or unsubstituted C₁-C₄ alkyl. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is —CX⁴₃. In embodiments, R⁴ is —CF₃. In embodiments, R⁴ is —OCX⁴₃. In embodiments, R⁴ is —OCF₃. In embodiments, R⁴ is —CN. In embodiments, R⁴ is —OR^{4F}. In embodiments, R⁴ is —OH. In embodiments, R⁴ is —OCH₃. In embodiments, R⁴ is substituted or unsubstituted C₁-C₄ alkyl. In embodiments, R⁴ is unsubstituted C₁-C₄ alkyl. In embodiments, R⁴ is methyl. In embodiments, R⁴ is ethyl. In embodiments, R⁴ is —C(O)OR^{4F}. In embodiments, R⁴ is —C(O)NHR^{4F} In embodiments, R⁴ is —C(O)N(R^{4F})₂. In embodiments, R⁴ is —C(O)NH₂.

In embodiments, R^{4F} is a hydrogen, substituted or unsubstituted alkyl, substituted or unsubstituted aryl, or substituted or unsubstituted heteroalkyl. In embodiments, R^{4F} is a hydrogen. In embodiments, R^{4F} is a substituted or unsubstituted alkyl. In embodiments, R^{4F} is a substituted or unsubstituted C₁-C₄ alkyl. In embodiments, R^{4F} is an unsubstituted C₁-C₄ alkyl. In embodiments, R^{4F} is methyl. In embodiments, R^{4F} is ethyl. In embodiments, R^{4F} is a substituted or unsubstituted 2 to 6 membered heteroalkyl. In embodiments, R^{4F} is a substituted or unsubstituted 2 membered heteroalkyl. In embodiments, R^{4F} is a substituted or unsubstituted 3 membered heteroalkyl. In embodiments, R^{4F} is a substituted or unsubstituted 4 membered heteroalkyl. In embodiments, R^{4F} is a substituted or unsubstituted 5 membered heteroalkyl. In embodiments, R^{4F} is a substituted or unsubstituted 6 membered heteroalkyl.

In embodiments, each R⁵ is independently halogen, —CX⁵₃, —OCX⁵₃, —CN, —OR^{5F}, —C(O)OR^{5F}, —C(O)NHR^{5F}, —C(O)N(R^{5F})₂, or substituted or unsubstituted C₁-C₄ alkyl. In embodiments, R⁵ is halogen. In embodiments, R⁵ is —F. In embodiments, R⁵ is —Cl. In embodiments, R⁵ is —Br. In embodiments, R⁵ is —I. In embodiments, R⁵ is —CX⁵₃. In embodiments, R⁵ is —CF₃. In embodiments, R⁵ is —OCX⁵₃. In embodiments, R⁵ is —OCF₃. In embodiments, R⁵ is —CN. In embodiments, R⁵ is —OR^{5F}. In embodiments, R⁵ is —OH. In embodiments, R⁵ is —OCH₃. In embodiments, R⁵ is substituted or unsubstituted C₁-C₅ alkyl. In embodiments, R⁵ is unsubstituted C₁-C₅ alkyl. In embodiments, R⁵ is methyl. In embodiments, R⁵ is ethyl. In embodiments, R⁵ is —C(O)OR^{5F}. In embodiments, R⁵ is —C(O)NHR^{5F} In embodiments, R⁵ is —C(O)N(R^{5F})₂. In embodiments, R⁵ is —C(O)NH₂.

In embodiments, R^{5F} is a hydrogen, substituted or unsubstituted alkyl, substituted or unsubstituted aryl, or substituted or unsubstituted heteroalkyl. In embodiments, R^{5F} is a hydrogen. In embodiments, R^{5F} is a substituted or unsubstituted alkyl. In embodiments, R^{5F} is a substituted or unsubstituted C₁-C₄ alkyl. In embodiments, R^{5F} is an unsubstituted C₁-C₄ alkyl. In embodiments, R^{5F} is methyl. In embodiments, R^{5F} is ethyl. In embodiments, R^{5F} is a substituted or unsubstituted 2 to 6 membered heteroalkyl. In embodiments, R^{5F} is a substituted or unsubstituted 2 membered heteroalkyl. In embodiments, R^{5F} is a substituted or unsubstituted 3 membered heteroalkyl. In embodiments, R^{5F} is a substituted or unsubstituted 4 membered heteroalkyl. In embodiments, R^{5F} is a substituted or unsubstituted 5 membered heteroalkyl. In embodiments, R^{5F} is a substituted or unsubstituted 6 membered heteroalkyl.

In embodiments, n is 0. In embodiments, n is 1. In embodiments, n is 2.

In embodiments, R¹ is halogen, substituted or unsubstituted C₁-C₄ alkyl, —CN, —CX¹₃, or OR^{1F}. In embodiments, each R^{1F} is independently hydrogen or unsubstituted C₁-C₄ alkyl. In embodiments, each R^{1F} is independently hydrogen, or unsubstituted methyl.

In embodiments, R¹ is halogen, substituted or unsubstituted C₁-C₄ alkyl, —CN, —CX¹₃, or OR^{1F}. In embodiments, R¹ is —F. In embodiments, R¹ is —Cl. In embodiments, R¹ is —Br. In embodiments, R¹ is —I. In embodiments, R¹ is substituted C₁-C₄ alkyl. In embodiments, R¹ is unsubstituted C₁-C₄ alkyl. In embodiments, R¹ is methyl. In embodiments, R¹ is ethyl. In embodiments, R¹ is —CN. In embodiments, R¹ is —CF₃. In embodiments, R¹ is —OH. In embodiments, R¹ is —OCH₃.

In an aspect, provided is a compound having a structure of Formula (X),

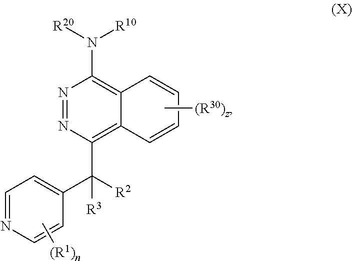

or a pharmaceutically acceptable salt thereof, or an isomer thereof;
wherein:
R¹⁰ is hydrogen, substituted or unsubstituted alkyl, substituted or unsubstituted heteroalkyl;
R²⁰ is -L¹-R²¹;
L¹ is a bond, substituted or unsubstituted alkylene, or substituted or unsubstituted heteroalkylene;
R²¹ is substituted or unsubstituted cycloalkyl, substituted or unsubstituted heterocycloalkyl, substituted or unsubstituted aryl, or substituted or unsubstituted heteroaryl;
R¹⁰ and R²⁰ are optionally joined to form a substituted or unsubstituted bicyclic heterocycloalkyl;
R¹ is independently halogen, —CX¹₃, —CHX¹₂, —CH₂X¹, —OCX¹₃, —OCH₂X¹, —OCHX¹₂, —CN, —OR^{1F}, —SR^{1F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, D, halogen, —CX²₃, —CHX²₂, —CH₂X², —OCX²₃, —OCH₂X², —OCHX²₂, —OR^{2F}, —SR^{2F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, D, halogen, —CX³₃, —CHX³₂, —CH₂X³, —OCX³₃, —OCH₂X³, —OCHX³₂, —OR^{3F}, —SR^{3F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³⁰ is independently halogen, —CX³⁰₃, —CHX³⁰₂, —CH₂X³⁰, —OCX³⁰₃, —OCH₂X³⁰, —OCHX³⁰₂, —CN, —OR^{30F}, —SR^{30F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is an integer of 0 to 4;
z is an integer of 0 to 4;
Each X¹, X², X³ and X³⁰ is independently —F, —Br, —Cl, or —I; and
Each R^{1F}, R^{2F}, R^{3F} and R^{30F} is independently hydrogen, substituted or unsubstituted alkyl, or substituted or unsubstituted heteroalkyl,
provided that when R¹⁰ is hydrogen and L¹ is a bond, then R²¹ is not unsubstituted phenyl nor phenyl substituted with halogen, —C(O)CH₃, —S(O)₂—NH₂, or substituted or unsubstituted alkyl; and when L¹ is a methylene, and R² and R³ are hydrogen, then R²¹ is not unsubstituted tetrahydro-pyranyl.

In embodiments, R¹⁰ is hydrogen and L¹ is a bond, then R²¹ is not unsubstituted phenyl. In embodiments, R¹⁰ is hydrogen and L¹ is a bond, then R²¹ is not phenyl substituted with halogen. In embodiments, R¹⁰ is hydrogen and L¹ is a bond, then R²¹ is not phenyl substituted with —C(O)CH₃. In embodiments, R¹⁰ is hydrogen and L¹ is a bond, then R²¹ is not phenyl substituted with —S(O)₂—NH₂. In embodiments, R¹⁰ is hydrogen and L¹ is a bond, then R²¹ is not phenyl substituted with substituted or unsubstituted alkyl.

In embodiments, L¹ is a bond, substituted or unsubstituted C₁-C₄ alkylene, or substituted or unsubstituted 2 to 4 membered heteroalkylene. In embodiments, L¹ is a bond. In embodiments, L¹ is a substituted or unsubstituted C₁-C₄ alkylene. In embodiments, L¹ is a unsubstituted C₁-C₄ alkylene. In embodiments, L¹ is unsubstituted methylene. In embodiments, L¹ is a substituted methylene. In embodiments, L¹ is substituted or unsubstituted 2 to 4 membered heteroalkylene. In embodiments, L¹ is unsubstituted 2 to 4 membered heteroalkylene. In embodiments, L¹ is —O—CH₂—. In embodiments, L¹ is —O—CH₂—CH₂—.

In embodiments, R²¹ is

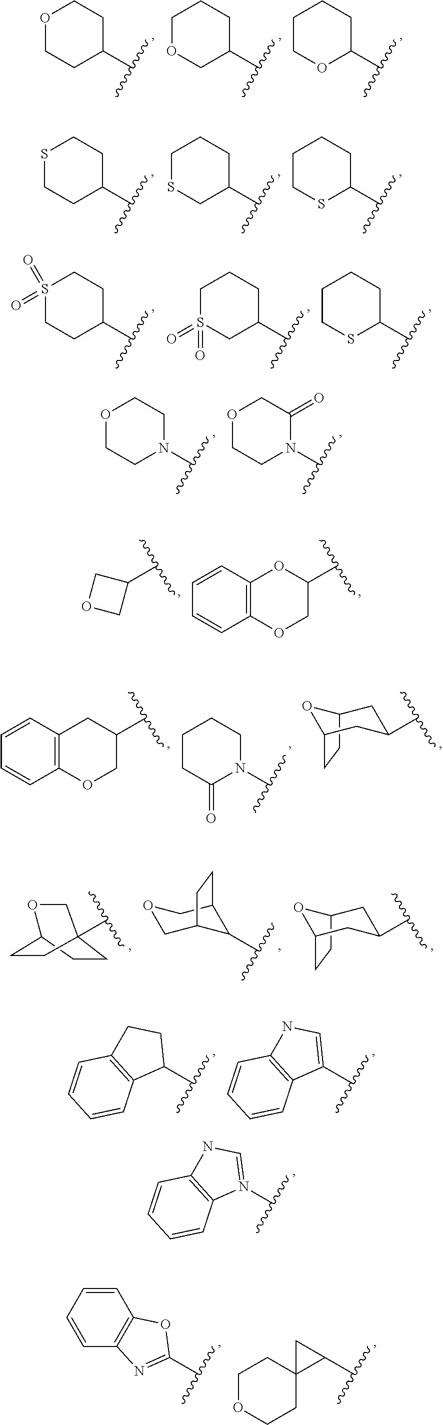

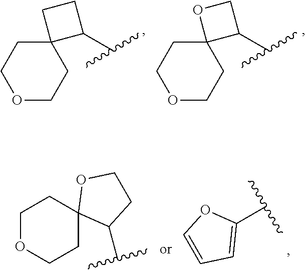

which is substituted or unsubstituted.

In embodiments, R²¹ is

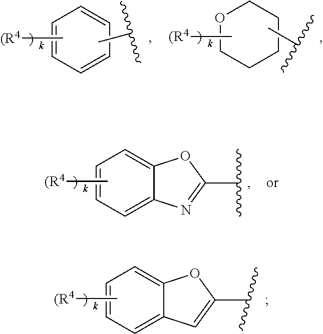

Each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)R^{4F}, —C(O)OR^{4F}, —S(O)₂R^{4F}, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
k is an integer of 0 to 5;
X⁴ is independently —F, —Br, —Cl, or —I; and
Each R^{4F} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, k is 0. In embodiments, k is 1. In embodiments, k is 2. In embodiments, n is 0. In embodiments, n is 1. In embodiments, n is 2.

In embodiments, each R⁴ is independently halogen, —CX⁴₃, —OCX⁴₃, —CN, —OR^{4F}, —C(O)R^{4F}, —C(O)OR^{4F}, —S(O)₂R^{4F}, —C(O)NHR^{4F}, —C(O)N(R^{4F})₂, or substituted or unsubstituted C₁-C₄ alkyl. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is —CX⁴₃. In embodiments, R⁴ is —CF₃. In embodiments, R⁴ is —OCX⁴₃. In embodiments, R⁴ is —OCF₃. In embodiments, R⁴ is —CN. In embodiments, R⁴ is —OR^{4F}. In embodiments, R⁴ is —OH. In embodiments, R⁴ is —OCH₃. In embodiments, R⁴ is substituted or unsubstituted C₁-C₄ alkyl. In embodiments, R⁴ is unsubstituted C₁-C₄ alkyl. In embodiments, R⁴ is methyl. In embodiments, R⁴ is ethyl. In embodiments, R⁴ is —C(O)CH₃ In embodiments, R⁴ is

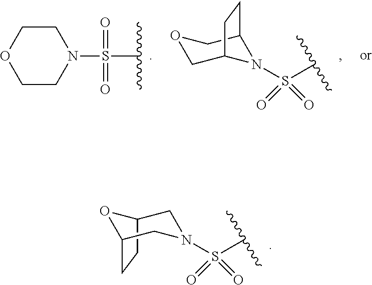

In embodiments, R⁴ is 4 to 6 membered heterocycloalkyl.

In embodiments, n is 0, R² and R³ are hydrogen, and R²¹ is

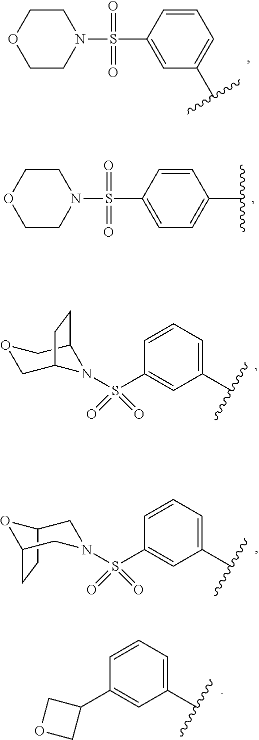 [SMILES: *c1cccc(S(=O)(=O)N2CCOCC2)c1 ; *c1ccc(S(=O)(=O)N2CCOCC2)cc1 ; *c1cccc(S(=O)(=O)N2C3CCC2COC3)c1 ; *c1cccc(S(=O)(=O)N2CC3CCC(C2)O3)c1 ; *c1cccc(C2COC2)c1]

In embodiments, n is 0, and at least one of R² and R³ is not hydrogen. In embodiments, n is 0, and at least one of R² and R³ is methyl. In embodiments, n is 0, at least one of R² and R³ is methyl, and R⁴ is —C(O)CH₃.

In embodiments, when L¹ is a methylene, R² and R³ are hydrogen, and R¹⁰ is hydrogen, and then R²¹ is not unsubstituted tetrahydro-pyranyl.

In embodiments, R¹⁰ and R²⁰ are joined to form a substituted or unsubstituted

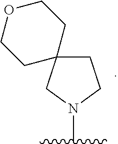 [SMILES: *N1CCC2(CCOCC2)C1]

In embodiments, the compound has a structure of Formula (XI):

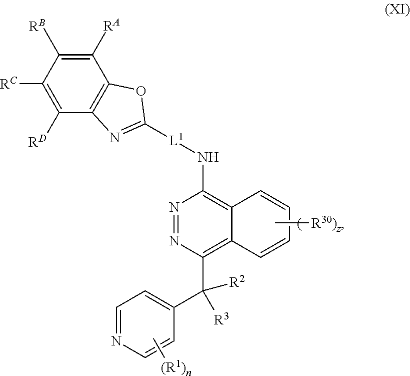

or a pharmaceutically acceptable salt thereof, or an isomer thereof;
wherein:
each R^{A}, R^{B}, R^{C}, and R^{D} is independently hydrogen, substituted or unsubstituted alkyl, substituted or unsubstituted heteroalkyl; or two of R^{A}, R^{B}, R^{C}, and R^{D} are joined to form a substituted or unsubstituted cycloalkyl, substituted or unsubstituted heterocycloalkyl, substituted or unsubstituted aryl, or substituted or unsubstituted heteroaryl.
L¹, R¹, R², R³, R³⁰, n and z as described in Formula (X).

In embodiments, R^{A} and R^{B} are joined to form a substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl. In embodiments, R^{A} and R^{B} are joined to form a substituted or unsubstituted C₅-C₆ cycloalkyl. In embodiments, R^{A} and R^{B} are joined to form a substituted or unsubstituted C₅ cycloalkyl. In embodiments, R^{A} and R^{B} are joined to form a substituted or unsubstituted C₆ cycloalkyl. In embodiments, R^{A} and R^{B} are joined to form a substituted or unsubstituted 5 to 6 membered heterocycloalkyl. In embodiments, R^{A} and R^{B} are joined to form a substituted or unsubstituted 5 membered heterocycloalkyl. In embodiments, R^{A} and R^{B} are joined to form a substituted or unsubstituted 6 membered heterocycloalkyl. In embodiments, R^{A} and R^{B} are joined to form a substituted or unsubstituted 5 to 6 membered heterocycloalkyl containing N, O, or S. In embodiments, R^{A} and R^{B} are joined to form a substituted or unsubstituted 5 membered heterocycloalkyl containing N, O, or S. In embodiments, R^{A} and R^{B} are joined to form a substituted or unsubstituted 6 membered heterocycloalkyl containing N, O, or S. In embodiments, R^{A} and R^{B} are joined to form a substituted or unsubstituted phenyl. In embodiments, R^{A} and R^{B} are joined to form substituted or unsubstituted 5 to 6 membered heteroaryl. In embodiments, R^{A} and R^{B} are joined to form substituted or unsubstituted 5 membered heteroaryl. In embodiments, R^{A} and R^{B} are joined to form substituted or unsubstituted 6 membered heteroaryl. In embodiments, R^{A} and R^{B} are joined to form substituted or unsubstituted 5 to 6 membered heteroaryl containing N, O, or S. In embodiments, R^{A} and R^{B} are joined to form substituted or unsubstituted 5 membered heteroaryl containing N, O, or S. In embodiments, R^{A} and R^{B} are joined to form substituted or unsubstituted 6 membered heteroaryl containing N, O, or S.

In embodiments, R^{B} and R^{C} are joined to form a substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl. In embodiments, R^{B} and R^{C} are joined to form a substituted or unsubstituted C₅-C₆ cycloalkyl. In embodiments, R^{B} and R^{C} are joined to form a substituted or unsubstituted C₅ cycloalkyl. In embodiments, R^{B} and R^{C} are joined to form a substituted or unsubstituted C₆ cycloalkyl. In embodiments, R^{B} and R^{C} are joined to form a substituted or unsubstituted 5 to 6 membered heterocycloalkyl. In embodiments, R^{B} and R^{C} are joined to form a substituted or unsubstituted 5 membered heterocycloalkyl. In embodiments, R^{B} and R^{C} are joined to form a substituted or unsubstituted 6 membered heterocycloalkyl. In embodiments, R^{B} and R^{C} are joined to form a substituted or unsubstituted 5 to 6 membered heterocycloalkyl containing N, O, or S. In embodiments, R^{B} and R^{C} are joined to form a substituted or unsubstituted 5 membered heterocycloalkyl containing N, O, or S. In embodiments, R^{B} and R^{C} are joined to form a substituted or unsubstituted 6 membered heterocycloalkyl containing N, O, or S. In embodiments, R^{B} and R^{C} are joined to form a substituted or unsubstituted phenyl. In embodiments, R^{B} and R^{C} are joined to form substituted or unsubstituted 5 to 6 membered heteroaryl. In embodiments, R^{B} and R^{C} are joined to form substituted or unsubstituted 5 membered heteroaryl. In embodiments, R^{B} and R^{C} are joined to form substituted or unsubstituted 6 membered heteroaryl. In embodiments, R^{B} and R^{C} are joined to form substituted or unsubstituted 5 to 6 membered heteroaryl containing N, O, or S. In embodiments, R^{B} and R^{C} are joined to form substituted or unsubstituted 5 membered heteroaryl containing N, O, or S. In embodiments, R^{B} and R^{C} are joined to form substituted or unsubstituted 6 membered heteroaryl containing N, O, or S.

In embodiments, R^{C} and R^{D} are joined to form a substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl. In embodiments, R^{C} and R^{D} are joined to form a substituted or unsubstituted C₅-C₆ cycloalkyl. In embodiments, R^{C} and R^{D} are joined to form a substituted or unsubstituted C₅ cycloalkyl. In embodiments, R^{C} and R^{D} are joined to form a substituted or unsubstituted C₆ cycloalkyl. In embodiments, R^{C} and R^{D} are joined to form a substituted or unsubstituted 5 to 6 membered heterocycloalkyl. In embodiments, R^{C} and R^{D} are joined to form a substituted or unsubstituted 5 membered heterocycloalkyl. In embodiments, R^{C} and R^{D} are joined to form a substituted or unsubstituted 6 membered heterocycloalkyl. In embodiments, R^{C} and R^{D} are joined to form a substituted or unsubstituted 5 to 6 membered heterocycloalkyl containing N, O, or S. In embodiments, R^{C} and R^{D} are joined to form a substituted or unsubstituted 5 membered heterocycloalkyl containing N, O, or S. In embodiments, R^{C} and R^{D} are joined to form a substituted or unsubstituted 6 membered heterocycloalkyl containing N, O, or S. In embodiments, R^{C} and R^{D} are joined to form a substituted or unsubstituted phenyl. In embodiments, R^{C} and R^{D} are joined to form substituted or unsubstituted 5 to 6 membered heteroaryl. In embodiments, R^{C} and R^{D} are joined to form substituted or unsubstituted 5 membered heteroaryl. In embodiments, R^{C} and R^{D} are joined to form substituted or unsubstituted 6 membered heteroaryl. In embodiments, R^{C} and R^{D} are joined to form substituted or unsubstituted 5 to 6 membered heteroaryl containing N, O, or S. In embodiments, R^{C} and R^{D} are joined to form substituted or unsubstituted 5 membered heteroaryl containing N, O, or S. In embodiments, R^{C} and R^{D} are joined to form substituted or unsubstituted 6 membered heteroaryl containing N, O, or S.

In embodiments, the compound has a structure of Formula (XI-a), (XI-b), or (XI-c),

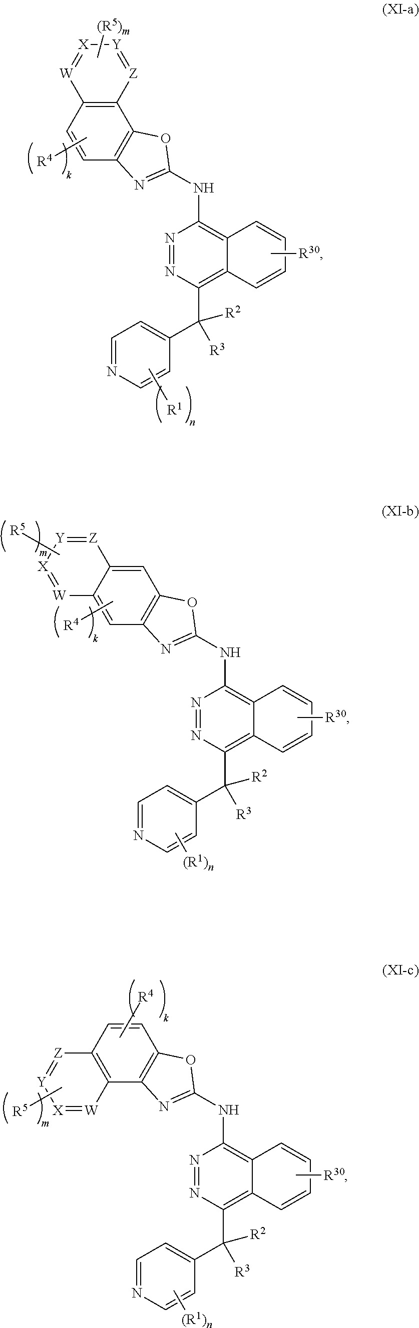

or a pharmaceutically acceptable salt thereof, or an isomer thereof;
wherein
Each W, X, Y and Z is independently ═N— or —CH═;
Each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)R^{4F}, —C(O)OR^{4F}, —S(O)₂RF, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Each R⁵ is independently halogen, —CX⁵₃, —CHX⁵₂, —CH₂X⁵, —OCX⁵₃, —OCH₂X⁵, —OCHX⁵₂, —CN, —OR^{5F}, —SR^{5F}, —C(O)OR^{5F}, —S(O)₂R^{5F}, —C(O)NHR^{5F}, —C(O)N(R^{5F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
k is an integer of 0 to 2;
m is an integer of 0 to 4;
Each X⁴ and X⁵ is independently —F, —Br, —Cl, or —I; and
Each R^{4F} and R^{5F} is independently hydrogen, substituted or unsubstituted alkyl, or substituted or unsubstituted heteroalkyl.
R¹, R², R³, R³⁰, and n are as described in Formula (X).

In embodiments, the compound has the structure of (XI-a-1), (XI-b-a), or (XI-c-1),

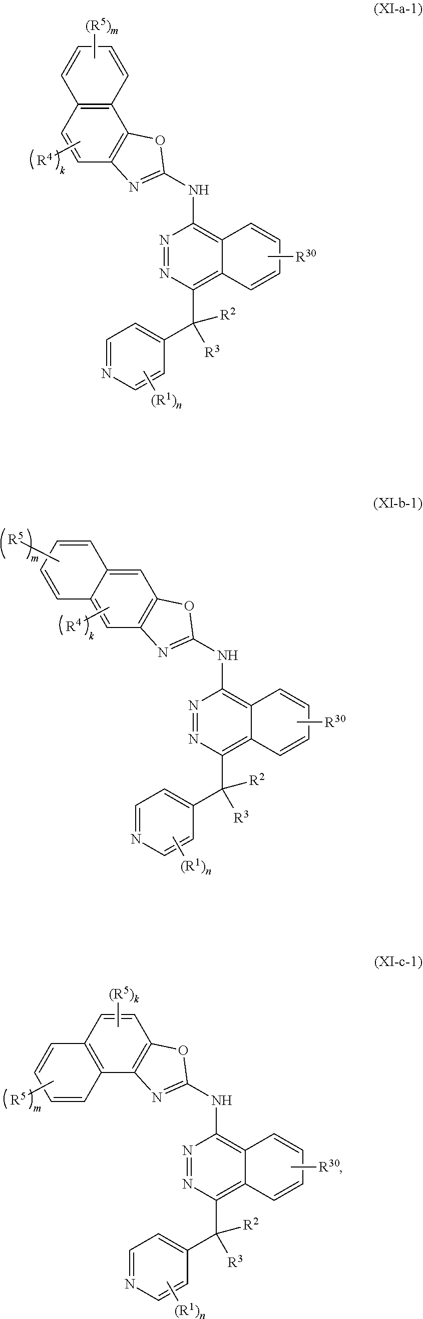

or a pharmaceutically acceptable salt thereof, or an isomer thereof.
R¹, R², R³, R⁴, R⁵, R³⁰, n, k and m are as described in (XI-a), (XI-b) or in (XI-c).

In embodiments, the compound has a structure of Formula (XI-d), (XI-e), (XI-f), (XI-d′), (XI-e′), or (XI-f′),

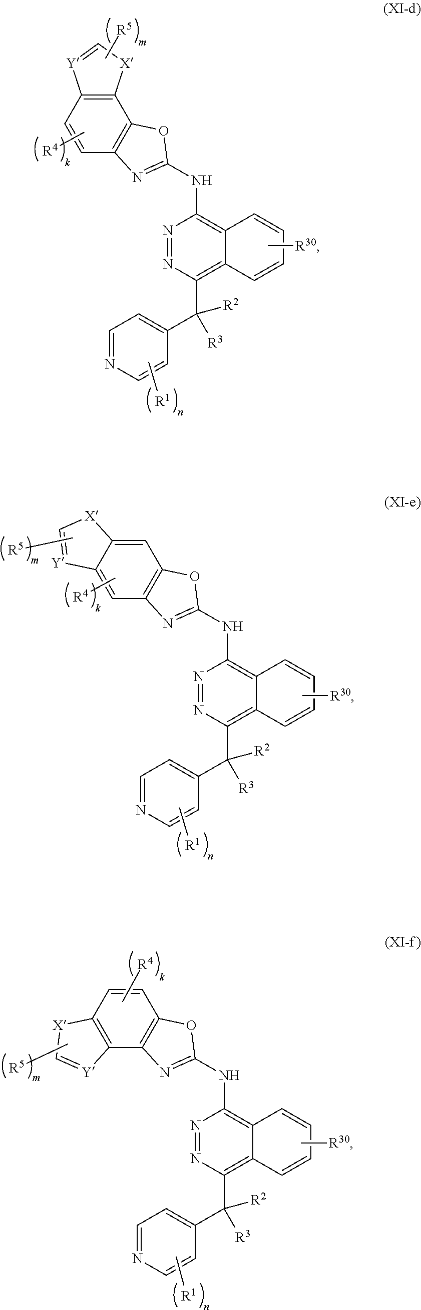

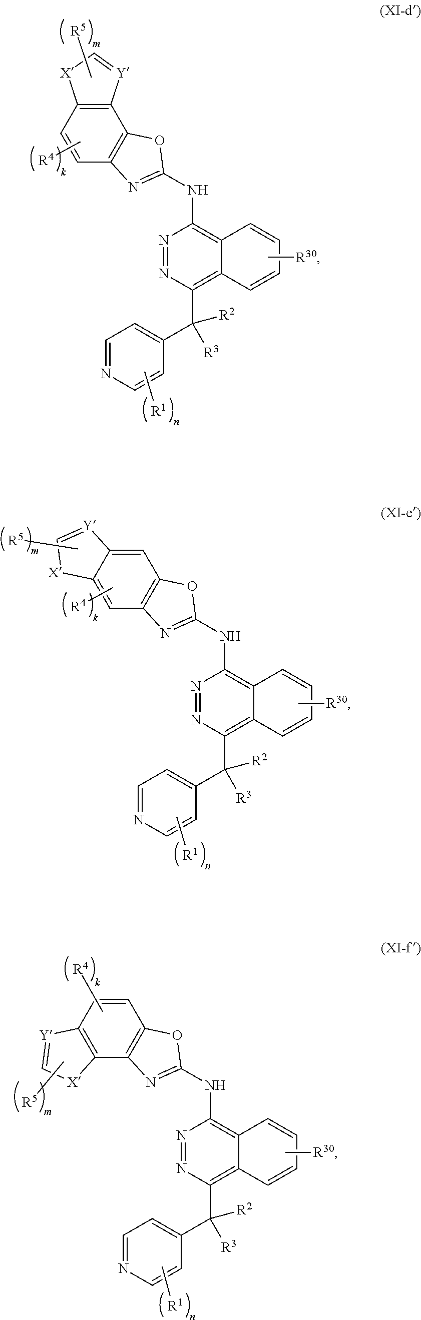

or a pharmaceutically acceptable salt thereof, or an isomer thereof;
wherein:
X′ is —O—, —NH—, or —CH₂—;
Y′ is —NH═, or —CH═;
k is an integer of 0 to 2;
m is an integer of 0 to 3;
Each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)R^{4F}, —C(O)OR^{4F}, —S(O)₂R^{4F}, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Each R⁵ is independently halogen, —CX⁵₃, —CHX⁵₂, —CH₂X⁵, —OCX⁵₃, —OCH₂X⁵, —OCHX⁵₂, —CN, —OR^{5F}, —SR^{5F}, —C(O)OR^{5F}, —S(O)₂R^{5F}, —C(O)NHR^{5F}, —C(O)N(R^{5F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Each X⁴ and X⁵ is independently —F, —Br, —Cl, or —I; and
Each R^{4F} and R^{5F} is independently hydrogen, substituted or unsubstituted alkyl, or substituted or unsubstituted heteroalkyl.
R¹, R², R³, R⁴, R⁵, R³⁰, n, k and m are as described in (XI-a), (XI-b) or in (XI-c).

In embodiments, R² is H, D, halogen, substituted or unsubstituted C₁-C₄ alkyl, —CX²₃, or OR^{2F}, and R³ is H, D, halogen, substituted or unsubstituted C₁-C₄ alkyl, —CX³₃, or OR^{3F} In embodiments, each R^{2F} and R^{3F} is independently hydrogen, or unsubstituted C₁-C₄ alkyl. In embodiments, each R^{2F} and R^{3F} is independently hydrogen, or unsubstituted methyl. In embodiments, R^{2F} is hydrogen, or unsubstituted methyl. In embodiments, R^{3F} is hydrogen, or unsubstituted methyl.

In embodiments, R² is —H, -D, halogen, substituted or unsubstituted C₁-C₄ alkyl, —CX²₃, or OR^{2F}. In embodiments, R² is —H. In embodiments, R² is -D. In embodiments, R² is —F. In embodiments, R² is —Cl. In embodiments, R² is —Br. In embodiments, R² is —I. In embodiments, R² is substituted C₁-C₄ alkyl. In embodiments, R² is unsubstituted C₁-C₄ alkyl. In embodiments, R² is methyl. In embodiments, R² is ethyl. In embodiments, R² is —CF₃. In embodiments, R² is —OH. In embodiments, R² is —OCH₃.

In embodiments, R³ is —H, -D, halogen, substituted or unsubstituted C₁-C₄ alkyl, —CX³₃, or OR^{3F}. In embodiments, R³ is —H. In embodiments, R³ is -D. In embodiments, R³ is —F. In embodiments, R³ is —Cl. In embodiments, R³ is —Br. In embodiments, R³ is —I. In embodiments, R³ is substituted C₁-C₄ alkyl. In embodiments, R³ is unsubstituted C₁-C₄ alkyl. In embodiments, R³ is methyl. In embodiments, R³ is ethyl. In embodiments, R³ is —CF₃. In embodiments, R³ is —OH. In embodiments, R³ is —OCH₃.

In embodiments, k is 0. In embodiments, k is 1. In embodiments, k is 2. In embodiments, m is 0. In embodiments, m is 1. In embodiments, m is 2.

In embodiments, each R⁴ is independently halogen, —CX⁴₃, —OCX⁴₃, —CN, —OR^{4F}, —C(O)R^{4F}, —C(O)OR^{4F}, —S(O)₂R^{4F}, C(O)NHR^{4F}, —C(O)N(R^{4F})₂, or substituted or unsubstituted C₁-C₄ alkyl. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is —CX⁴₃. In embodiments, R⁴ is —CF₃. In embodiments, R⁴ is —OCX⁴₃. In embodiments, R⁴ is —OCF₃. In embodiments, R⁴ is —CN. In embodiments, R⁴ is —OR^{4F}. In embodiments, R⁴ is —OH. In embodiments, R⁴ is —OCH₃. In embodiments, R⁴ is substituted or unsubstituted C₁-C₄ alkyl. In embodiments, R⁴ is unsubstituted C₁-C₄ alkyl. In embodiments, R⁴ is methyl. In embodiments, R⁴ is ethyl. In embodiments, R⁴ is —C(O)OR^{4F}. In embodiments, R⁴ is —S(O)₂R^{4F}. In embodiments, R⁴ is —C(O)NHR^{4F}. In embodiments, R⁴ is —C(O)N(R^{4F})₂. In embodiments, R⁴ is —C(O)NH₂.

In embodiments, R^{4F} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{4F} is a hydrogen. In embodiments, R^{4F} is a substituted or unsubstituted alkyl. In embodiments, R^{4F} is a substituted or unsubstituted C₁-C₄ alkyl. In embodiments, R^{4F} is an unsubstituted C₁-C₄ alkyl. In embodiments, R^{4F} is methyl. In embodiments, R^{4F} is ethyl. In embodiments, R^{4F} is a substituted or unsubstituted 2 to 6 membered heteroalkyl. In embodiments, R^{4F} is a substituted or unsubstituted 2 membered heteroalkyl. In embodiments, R^{4F} is a substituted or unsubstituted 3 membered heteroalkyl. In embodiments, R^{4F} is a substituted or unsubstituted 4 membered heteroalkyl. In embodiments, R^{4F} is a substituted or unsubstituted 5 membered heteroalkyl. In embodiments, R^{4F} is a substituted or unsubstituted 6 membered heteroalkyl. In embodiments, R^{4F} is substituted or unsubstituted cycloalkyl. In embodiments, R^{4F} is substituted or unsubstituted C₃-C₆ cycloalkyl. In embodiments, R^{4F} is substituted or unsubstituted 5 to 12 membered mono-cyclic or bi-cyclic heterocycloalkyl. In embodiments, R^{4F} is unsubstituted 5 to 12 membered mono-cyclic or bi-cyclic heterocycloalkyl. In embodiments, R^{4F} is substituted 5 to 12 membered mono-cyclic or bi-cyclic heterocycloalkyl. In embodiments, R^{4F} is substituted or unsubstituted phenyl. In embodiments, R^{4F} is unsubstituted phenyl. In embodiments, R^{4F} is substituted phenyl. In embodiments, R^{4F} is substituted or unsubstituted 5 to 6 membered heteroaryl. In embodiments, R^{4F} is substituted 5 to 6 membered heteroaryl. In embodiments, R^{4F} is unsubstituted 5 to 6 membered heteroaryl.

In embodiments, each R⁵ is independently halogen, —CX⁵₃, —OCX⁵₃, —CN, —OR^{5F}, —C(O)OR^{5F}, —S(O)₂R^{5F}, —C(O)NHR^{5F}, —C(O)N(R^{5F})₂, or substituted or unsubstituted C₁-C₄ alkyl. In embodiments, R⁵ is halogen. In embodiments, R⁵ is —F. In embodiments, R⁵ is —Cl. In embodiments, R⁵ is —Br. In embodiments, R⁵ is —I. In embodiments, R⁵ is —CX⁵₃. In embodiments, R⁵ is —CF₃. In embodiments, R⁵ is —OCX⁵₃. In embodiments, R⁵ is —OCF₃. In embodiments, R⁵ is —CN. In embodiments, R⁵ is —OR^{5F}. In embodiments, R⁵ is —OH. In embodiments, R⁵ is —OCH₃. In embodiments, R⁵ is substituted or unsubstituted C₁-C₅ alkyl. In embodiments, R⁵ is unsubstituted C₁-C₅ alkyl. In embodiments, R⁵ is methyl. In embodiments, R⁵ is ethyl. In embodiments, R⁵ is —C(O)OR^{5F}. In embodiments, R⁵ is —S(O)₂R^{5F} In embodiments, R⁵ is —C(O)NHR^{5F}. In embodiments, R⁵ is —C(O)N(R^{5F})₂. In embodiments, R⁵ is —C(O)NH₂.

In embodiments, R^{5F} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{5F} is a hydrogen. In embodiments, R^{5F} is a substituted or unsubstituted alkyl. In embodiments, R^{5F} is a substituted or unsubstituted C₁-C₄ alkyl. In embodiments, R^{5F} is an unsubstituted C₁-C₄ alkyl. In embodiments, R^{5F} is methyl. In embodiments, R^{5F} is ethyl. In embodiments, R^{5F} is a substituted or unsubstituted 2 to 6 membered heteroalkyl. In embodiments, R^{5F} is a substituted or unsubstituted 2 membered heteroalkyl. In embodiments, R^{5F} is a substituted or unsubstituted 3 membered heteroalkyl. In embodiments, R^{5F} is a substituted or unsubstituted 4 membered heteroalkyl. In embodiments, R^{5F} is a substituted or unsubstituted 5 membered heteroalkyl. In embodiments, R^{5F} is a substituted or unsubstituted 6 membered heteroalkyl. In embodiments, R^{5F} is substituted or unsubstituted cycloalkyl. In embodiments, R^{5F} is substituted or unsubstituted C₃-C₆ cycloalkyl. In embodiments, R^{5F} is substituted or unsubstituted 5 to 12 membered mono-cyclic or bi-cyclic heterocycloalkyl. In embodiments, R^{5F} is unsubstituted 5 to 12 membered mono-cyclic or bi-cyclic heterocycloalkyl. In embodiments, R^{5F} is substituted 5 to 12 membered mono-cyclic or bi-cyclic heterocycloalkyl. In embodiments, R^{5F} is substituted or unsubstituted phenyl. In embodiments, R^{5F} is unsubstituted phenyl. In embodiments, R^{5F} is substituted phenyl. In embodiments, R^{5F} is substituted or unsubstituted 5 to 6 membered heteroaryl. In embodiments, R^{5F} is substituted 5 to 6 membered heteroaryl. In embodiments, R^{5F} is unsubstituted 5 to 6 membered heteroaryl

In embodiments, n is 0. In embodiments, n is 1. In embodiments, n is 2.

In embodiments, R¹ is halogen, substituted or unsubstituted C₁-C₄ alkyl, —CN, —CX¹₃, or OR^{1F}. In embodiments, each R^{1F} is independently hydrogen or unsubstituted C₁-C₄ alkyl. In embodiments, each R^{1F} is independently hydrogen, or unsubstituted methyl.

In embodiments, R¹ is halogen, substituted or unsubstituted C₁-C₄ alkyl, —CN, —CX¹₃, or OR^{1F}. In embodiments, R¹ is —F. In embodiments, R¹ is —Cl. In embodiments, R¹ is —Br. In embodiments, R¹ is —I. In embodiments, R¹ is substituted C₁-C₄ alkyl. In embodiments, R¹ is unsubstituted C₁-C₄ alkyl. In embodiments, R¹ is methyl. In embodiments, R¹ is ethyl. In embodiments, R¹ is —CN. In embodiments, R¹ is —CF₃. In embodiments, R¹ is —OH. In embodiments, R¹ is —OCH₃.

In embodiments, R³⁰ is halogen, substituted or unsubstituted C₁-C₄ alkyl, —CN, —CX³⁰₃, or OR^{1F}. In embodiments, each R^{30F} is independently hydrogen or unsubstituted C₁-C₄ alkyl. In embodiments, each R^{30F} is independently hydrogen, or unsubstituted methyl.

In embodiments, R³⁰ is halogen, substituted or unsubstituted C₁-C₄ alkyl, —CN, —CX¹₃, or OR^{1F}. In embodiments, R³⁰ is —F. In embodiments, R³⁰ is —Cl. In embodiments, R³⁰ is —Br. In embodiments, R³⁰ is —I. In embodiments, R³⁰ is substituted C₁-C₄ alkyl. In embodiments, R³⁰ is unsubstituted C₁-C₄ alkyl. In embodiments, R³⁰ is methyl. In embodiments, R³⁰ is ethyl. In embodiments, R³⁰ is —CN. In embodiments, R³⁰ is —CF₃. In embodiments, R³⁰ is —OH. In embodiments, R³⁰ is —OCH₃.

Compounds of Formulae (X) and (XI) are shown in Table 1 below.

TABLE 1

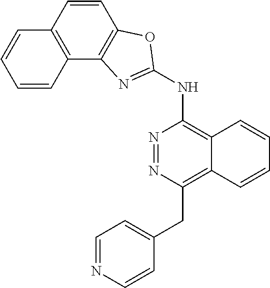 [SMILES: c1ccc2c(c1)ccc1oc(Nc3nnc(Cc4ccncc4)c4ccccc34)nc12]

SR-32077

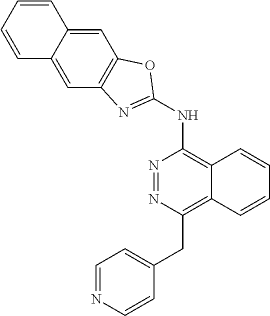 [SMILES: c1ccc2cc3oc(Nc4nnc(Cc5ccncc5)c5ccccc45)nc3cc2c1]

SR-32073

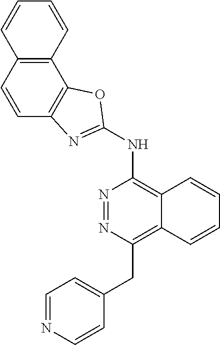 [SMILES: c1ccc2c(c1)ccc1nc(Nc3nnc(Cc4ccncc4)c4ccccc34)oc12]

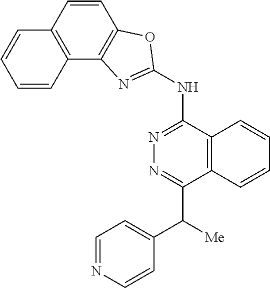 [SMILES: CC(c1ccncc1)c1nnc(Nc2nc3c(ccc4ccccc43)o2)c2ccccc12]

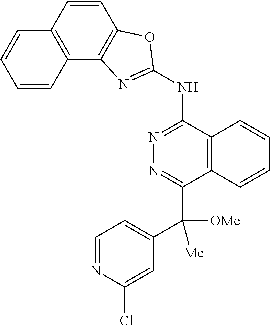 [SMILES: COC(C)(c1ccnc(Cl)c1)c1nnc(Nc2nc3c(ccc4ccccc43)o2)c2ccccc12]

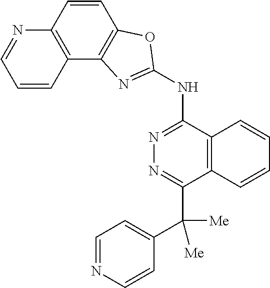 [SMILES: CC(C)(c1ccncc1)c1nnc(Nc2nc3c(ccc4ncccc43)o2)c2ccccc12]

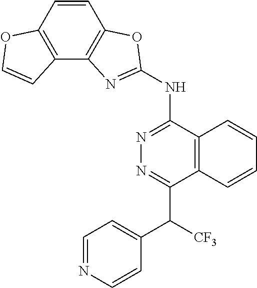 [SMILES: FC(F)(F)C(c1ccncc1)c1nnc(Nc2nc3c(ccc4occc43)o2)c2ccccc12]

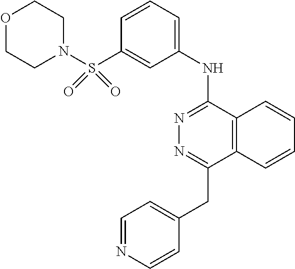 [SMILES: O=S(=O)(c1cccc(Nc2nnc(Cc3ccncc3)c3ccccc23)c1)N1CCOCC1]

SR-35424

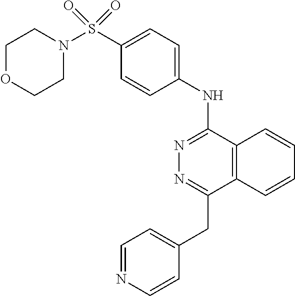 [SMILES: O=S(=O)(c1ccc(Nc2nnc(Cc3ccncc3)c3ccccc23)cc1)N1CCOCC1]

SR-35425

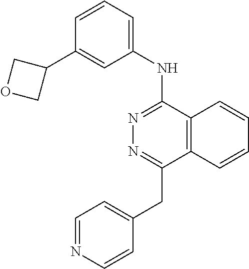 [SMILES: c1cc(Nc2nnc(Cc3ccncc3)c3ccccc23)cc(C2COC2)c1]

SR-35426

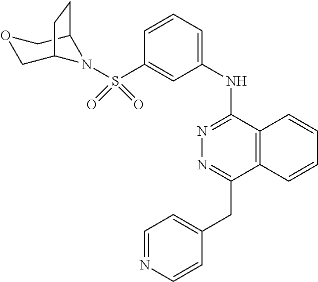 [SMILES: O=S(=O)(c1cccc(Nc2nnc(Cc3ccncc3)c3ccccc23)c1)N1C2CCC1COC2]

SR-35427

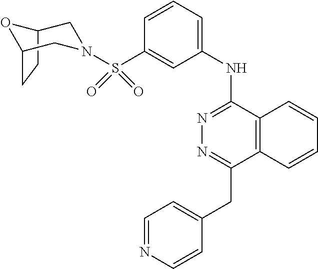 [SMILES: O=S(=O)(c1cccc(Nc2nnc(Cc3ccncc3)c3ccccc23)c1)N1CC2CCC(C1)O2]

SR-35428

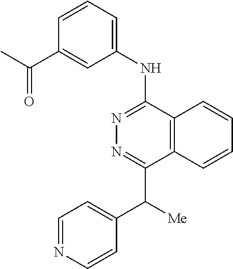 [SMILES: CC(=O)c1cccc(Nc2nnc(C(C)c3ccncc3)c3ccccc23)c1]

SR-34644

In an aspect, a compound has a structure of Formula (II),

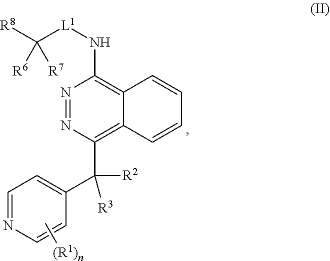

or a pharmaceutically acceptable salt thereof, or an isomer thereof;
wherein:
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is independently halogen, —CX¹₃, —CHX¹₂, —CH₂X¹, —OCX¹₃, —OCH₂X¹, —OCHX¹₂, —CN, —OR^{1F}, —SR^{1F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, D, halogen, —CX²₃, —CHX²₂, —CH₂X², —OCX²₃, —OCH₂X², —OCHX²₂, —CN, —OR^{2F}, —SR^{2F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, D, halogen, —CX³₃, —CHX³₂, —CH₂X³, —OCX³₃, —OCH₂X³, —OCHX³₂, —CN, —OR^{3F}, —SR^{3F} substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶ and R⁷ are joined together to form a substituted or unsubstituted cycloalkyl, or substituted or unsubstituted heterocycloalkyl;
R⁸ is a substituted or unsubstituted aryl, or substituted or unsubstituted heteroaryl;
n is an integer of 0 to 4,
Each X¹, X², and X³ is independently —F, —Br, —Cl, or —I; and
Each R^{1F}, R^{2F}, and R^{3F} is independently hydrogen, substituted or unsubstituted alkyl, or substituted or unsubstituted heteroalkyl.

In embodiments, R⁸ is

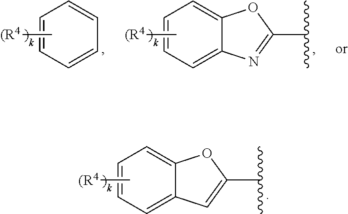

In embodiments, each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)OR^{4F}, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, X⁴ is independently —F, —Br, —Cl, or —I; and each R^{4F} is independently hydrogen, substituted or unsubstituted alkyl, or substituted or unsubstituted heteroalkyl. In embodiments, k is an integer of 0 to 5.

In embodiments, k is an integer of 0 to 5. In embodiments, k is 0. In embodiments, k is 1. In embodiments, k is 2.

In embodiments, each R⁴ is independently halogen, —CX⁴₃, —OCX⁴₃, —CN, —OR^{4F}, —C(O)OR^{4F}, —C(O)NHR^{4F}, —C(O)N(R^{4F})₂, or substituted or unsubstituted C₁-C₄ alkyl. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is —CX⁴₃. In embodiments, R⁴ is —CF₃. In embodiments, R⁴ is —OCX⁴₃. In embodiments, R⁴ is —OCF₃. In embodiments, R⁴ is —CN. In embodiments, R⁴ is —OR^{4F}. In embodiments, R⁴ is —OH. In embodiments, R⁴ is —OCH₃. In embodiments, R⁴ is substituted or unsubstituted C₁-C₄ alkyl. In embodiments, R⁴ is unsubstituted C₁-C₄ alkyl. In embodiments, R⁴ is methyl. In embodiments, R⁴ is ethyl. In embodiments, R⁴ is —C(O)OR^{4F}. In embodiments, R⁴ is —C(O)NHR^{4F} In embodiments, R⁴ is —C(O)N(R^{4F})₂. In embodiments, R⁴ is —C(O)NH₂.

In embodiments, R^{4F} is a hydrogen, substituted or unsubstituted alkyl, substituted or unsubstituted aryl, or substituted or unsubstituted heteroalkyl. In embodiments, R^{4F} is a hydrogen. In embodiments, R^{4F} is a substituted or unsubstituted alkyl. In embodiments, R^{4F} is a substituted or unsubstituted C₁-C₄ alkyl. In embodiments, R^{4F} is an unsubstituted C₁-C₄ alkyl. In embodiments, R^{4F} is methyl. In embodiments, R^{4F} is ethyl. In embodiments, R^{4F} is a substituted or unsubstituted 2 to 6 membered heteroalkyl. In embodiments, R^{4F} is a substituted or unsubstituted 2 membered heteroalkyl. In embodiments, R^{4F} is a substituted or unsubstituted 3 membered heteroalkyl. In embodiments, R^{4F} is a substituted or unsubstituted 4 membered heteroalkyl. In embodiments, R^{4F} is a substituted or unsubstituted 5 membered heteroalkyl. In embodiments, R^{4F} is a substituted or unsubstituted 6 membered heteroalkyl.

In embodiments, R⁶ and R⁷ are joined together to form a substituted or unsubstituted C₃-C₆ cycloalkyl, or substituted or unsubstituted 4 to 6 membered heterocycloalkyl. In embodiments, R⁶ and R⁷ are joined together to form a substituted or unsubstituted C₃-C₆ cycloalkyl. In embodiments, R⁶ and R⁷ are joined together to form a substituted or unsubstituted cyclopropyl. In embodiments, R⁶ and R⁷ are joined together to form a substituted or unsubstituted cyclobutyl. In embodiments, R⁶ and R⁷ are joined together to form a substituted or unsubstituted cyclopentyl. In embodiments, R⁶ and R⁷ are joined together to form a substituted or unsubstituted cyclohexyl. In embodiments, R⁶ and R⁷ are joined together to form a substituted or unsubstituted 4 to 6 membered heterocycloalkyl. In embodiments, R⁶ and R⁷ are joined together to form a substituted or unsubstituted 4 membered heterocycloalkyl. In embodiments, R⁶ and R⁷ are joined together to form a substituted or unsubstituted 5 membered heterocycloalkyl. In embodiments, R⁶ and R⁷ are joined together to form a substituted or unsubstituted 6 membered heterocycloalkyl.

In embodiments, R⁶ and R⁷ are joined together to form a substituted or unsubstituted 4 to 6 membered heterocycloalkyl containing N, O, or S. In embodiments, R⁶ and R⁷ are joined together to form a substituted or unsubstituted 4 membered heterocycloalkyl containing N, O, or S. In embodiments, R⁶ and R⁷ are joined together to form a substituted or unsubstituted 5 membered heterocycloalkyl containing N, O, or S. In embodiments, R⁶ and R⁷ are joined together to form a substituted or unsubstituted 6 membered heterocycloalkyl containing N, O, or S. In embodiments, R⁶ and R⁷ are joined together to form a substituted or unsubstituted 4 to 6 membered heterocycloalkyl containing O. In embodiments, R⁶ and R⁷ are joined together to form a substituted or unsubstituted 4 membered heterocycloalkyl containing O. In embodiments, R⁶ and R⁷ are joined together to form a substituted or unsubstituted 5 membered heterocycloalkyl containing O. In embodiments, R⁶ and R⁷ are joined together to form a substituted or unsubstituted 6 membered heterocycloalkyl containing O.

In embodiments, the compound has a structure of Formula (II-a) or (II-a′):

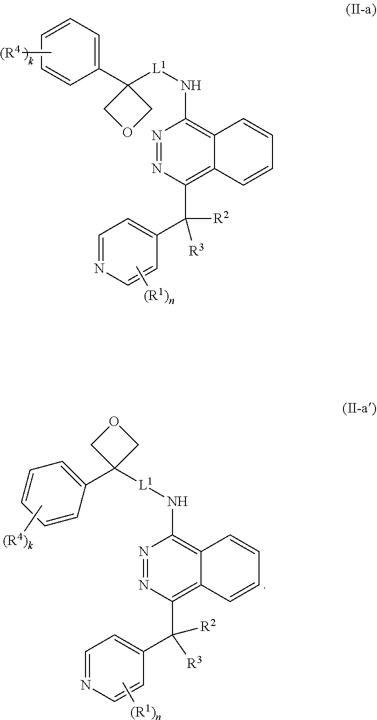

R¹, L¹, R⁴, n and k are described above.

In embodiments, L¹ is a bond or C₁-C₄ alkylene.

In embodiments, each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)OR^{4F}, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; X⁴ is independently —F, —Br, —Cl, or —I; and each R^{4F} is independently hydrogen, substituted or unsubstituted alkyl, or substituted or unsubstituted heteroalkyl.

In embodiments, R² is hydrogen. In embodiments, R³ is hydrogen.

In embodiments, k is an integer of 0 to 5. In embodiments, k is 0. In embodiments, k is 1. In embodiments, k is 2.

In embodiments, each R⁴ is independently halogen, —CX⁴₃, —OCX⁴₃, —CN, —OR^{4F}, —C(O)OR^{4F}, —C(O)NHR^{4F}, —C(O)N(R^{4F})₂, or substituted or unsubstituted C₁-C₄ alkyl. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is —CX⁴₃. In embodiments, R⁴ is —CF₃. In embodiments, R⁴ is —OCX⁴₃. In embodiments, R⁴ is —OCF₃. In embodiments, R⁴ is —CN. In embodiments, R⁴ is —OR^{4F}. In embodiments, R⁴ is —OH. In embodiments, R⁴ is —OCH₃. In embodiments, R⁴ is substituted or unsubstituted C₁-C₄ alkyl. In embodiments, R⁴ is unsubstituted C₁-C₄ alkyl. In embodiments, R⁴ is methyl. In embodiments, R⁴ is ethyl. In embodiments, R⁴ is —C(O)OR^{4F}. In embodiments, R⁴ is —C(O)NHR^{4F} In embodiments, R⁴ is —C(O)N(R^{4F})₂. In embodiments, R⁴ is —C(O)NH₂.

In embodiments, R^{4F} is a hydrogen, substituted or unsubstituted alkyl, substituted or unsubstituted aryl, or substituted or unsubstituted heteroalkyl. In embodiments, R^{4F} is a hydrogen. In embodiments, R^{4F} is a substituted or unsubstituted alkyl. In embodiments, R^{4F} is a substituted or unsubstituted C₁-C₄ alkyl. In embodiments, R^{4F} is an unsubstituted C₁-C₄ alkyl. In embodiments, R^{4F} is methyl. In embodiments, R^{4F} is ethyl. In embodiments, R^{4F} is a substituted or unsubstituted 2 to 6 membered heteroalkyl. In embodiments, R^{4F} is a substituted or unsubstituted 2 membered heteroalkyl. In embodiments, R^{4F} is a substituted or unsubstituted 3 membered heteroalkyl. In embodiments, R^{4F} is a substituted or unsubstituted 4 membered heteroalkyl. In embodiments, R^{4F} is a substituted or unsubstituted 5 membered heteroalkyl. In embodiments, R^{4F} is a substituted or unsubstituted 6 membered heteroalkyl.

In an aspect, provided is compound having a structure of Formula (XII),

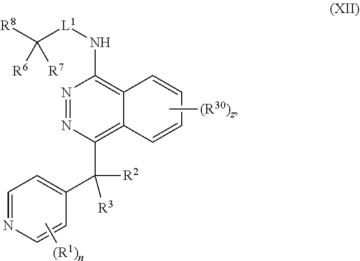

or a pharmaceutically acceptable salt thereof, or an isomer thereof; wherein:
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is independently halogen, —CX¹₃, —CHX¹₂, —CH₂X¹, —OCX¹₃, —OCH₂X¹, —OCHX¹₂, —CN, —OR^{1F}, —SR^{1F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, D, halogen, —CX²₃, —CHX²₂, —CH₂X², —OCX²₃, —OCH₂X², —OCHX²₂, —OR^{2F}, —SR^{2F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, D, halogen, —CX³₃, —CHX³₂, —CH₂X³, —OCX³₃, —OCH₂X³, —OCHX³₂, —OR^{3F}, —SR^{3F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ are optionally joined together to form a substituted or unsubstituted cycloalkyl, or substituted or unsubstituted heterocycloalkyl;
Each R⁶ and R⁷ is independently hydrogen, substituted or unsubstituted alkyl, or substituted or unsubstituted heteroalkyl; or R⁶ and R⁷ are optionally joined together to form a substituted or unsubstituted cycloalkyl, or substituted or unsubstituted heterocycloalkyl;
R⁸ is a substituted or unsubstituted cycloalkyl, or substituted or unsubstituted heterocycloalkyl, substituted or unsubstituted aryl, or substituted or unsubstituted heteroaryl;
R³⁰ is independently halogen, —CX³, —CHX³⁰₂, —CH₂X⁰, —OCX³⁰, —OCH₂X⁰, —OCHX³⁰₂, —CN, —OR^{30F}, —SR^{30F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is an integer of 0 to 4;
z is an integer of 0 to 4;
Each X¹, X², X³, and X³⁰ is independently —F, —Br, —Cl, or —I; and
Each R^{1F}, R^{2F}, R^{3F}, and R^{30F} is independently hydrogen, substituted or unsubstituted alkyl, or substituted or unsubstituted heteroalkyl,
provided that when n is 0, L¹ is a bond, R⁶ and R⁷ are hydrogen, then R⁸ is not unsubstituted tetrahydro-pyranyl.

In embodiments, at least one of R² or R³ is not hydrogen. In embodiments, R² is not hydrogen. In embodiments, R³ is not hydrogen. In embodiments, R¹ is not hydrogen and R² is not hydrogen. In embodiments, R¹ is not hydrogen and R³ is not hydrogen.

In embodiments, R⁸ is

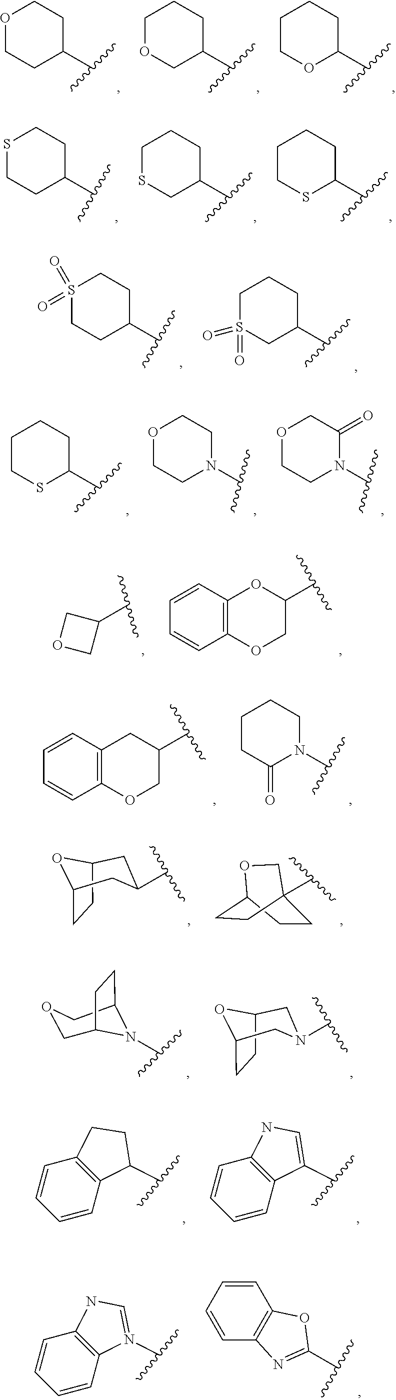

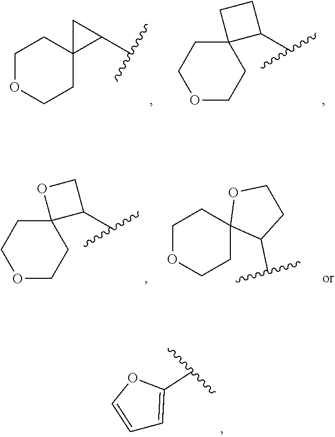 [SMILES: *C1CC12CCOCC2 ; *C1CCC12CCOCC2 ; *C1COC12CCOCC2 ; *C1CCOC12CCOCC2 ; *c1ccco1]

which is substituted or unsubstituted.

In embodiments, R⁸ is

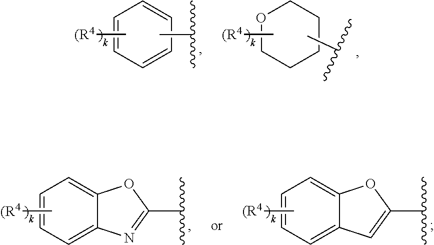

Each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)R^{4F}, —C(O)OR^{4F}, —S(O)₂R^{4F}, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
k is an integer of 0 to 5;
X⁴ is independently —F, —Br, —Cl, or —I; and
Each R^{4F} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the compound has a structure of Formula (XII-a):

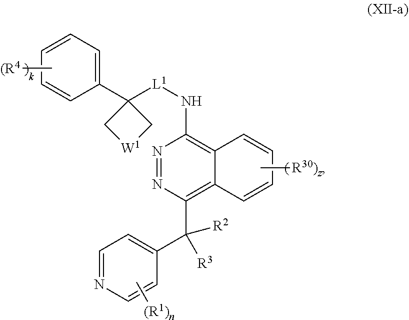

wherein:
W¹ is —O— or CH₂—;
L¹ is a bond or C₁-C₄ alkylene,
Each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)R^{4F}, —C(O)OR^{4F}, —S(O)₂RF, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
k is an integer of 0 to 5;
X⁴ is independently —F, —Br, —Cl, or —I; and
Each R^{4F} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.
L¹, R¹, R³⁰, n and z are as described in (XII).

In embodiments, R⁸ is

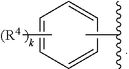

In embodiments, R⁸ is

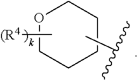

In embodiments, R⁸ is

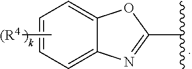

In embodiments, R⁸ is

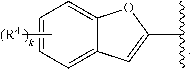

In embodiments, each R⁴ is independently halogen, —CX⁴₃, —OCX⁴₃, —CN, —OR^{4F}, —C(O)R^{4F}, —C(O)OR^{4F}, —S(O)₂R^{4F}, —C(O)NHR^{4F}, —C(O)N(R^{4F})₂, or substituted or unsubstituted C₁-C₄ alkyl. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is —CX⁴₃. In embodiments, R⁴ is —CF₃. In embodiments, R⁴ is —OCX⁴₃. In embodiments, R⁴ is —OCF₃. In embodiments, R⁴ is —CN. In embodiments, R⁴ is —OR^{4F}. In embodiments, R⁴ is —OH. In embodiments, R⁴ is —OCH₃. In embodiments, R⁴ is substituted or unsubstituted C₁-C₄ alkyl. In embodiments, R⁴ is unsubstituted C₁-C₄ alkyl. In embodiments, R⁴ is methyl. In embodiments, R⁴ is ethyl. In embodiments, R⁴ is —C(O)R^{4F}. In embodiments, R⁴ is —C(O)OR^{4F} In embodiments, R⁴ is —S(O)₂R^{4F}. In embodiments, R⁴ is —C(O)NHR^{4F}. In embodiments, R⁴ is —C(O)N(R^{4F})₂. In embodiments, R⁴ is —C(O)NH₂.

In embodiments, R^{4F} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{4F} is a hydrogen. In embodiments, R^{4F} is a substituted or unsubstituted alkyl. In embodiments, R^{4F} is a substituted or unsubstituted C₁-C₄ alkyl. In embodiments, R^{4F} is an unsubstituted C₁-C₄ alkyl. In embodiments, R^{4F} is methyl. In embodiments, R^{4F} is ethyl. In embodiments, R^{4F} is a substituted or unsubstituted 2 to 6 membered heteroalkyl. In embodiments, R^{4F} is a substituted or unsubstituted 2 membered heteroalkyl. In embodiments, R^{4F} is a substituted or unsubstituted 3 membered heteroalkyl. In embodiments, R^{4F} is a substituted or unsubstituted 4 membered heteroalkyl. In embodiments, R^{4F} is a substituted or unsubstituted 5 membered heteroalkyl. In embodiments, R^{4F} is a substituted or unsubstituted 6 membered heteroalkyl. In embodiments, R^{4F} is substituted or unsubstituted cycloalkyl. In embodiments, R^{4F} is substituted or unsubstituted C₃-C₆ cycloalkyl. In embodiments, R^{4F} is substituted or unsubstituted 5 to 12 membered mono-cyclic or bi-cyclic heterocycloalkyl. In embodiments, R^{4F} is unsubstituted 5 to 12 membered mono-cyclic or bi-cyclic heterocycloalkyl. In embodiments, R^{4F} is substituted 5 to 12 membered mono-cyclic or bi-cyclic heterocycloalkyl. In embodiments, R^{4F} is substituted or unsubstituted phenyl. In embodiments, R^{4F} is unsubstituted phenyl. In embodiments, R^{4F} is substituted phenyl. In embodiments, R^{4F} is substituted or unsubstituted 5 to 6 membered heteroaryl. In embodiments, R^{4F} is substituted 5 to 6 membered heteroaryl. In embodiments, R^{4F} is unsubstituted 5 to 6 membered heteroaryl.

In embodiments, n is 0. In embodiments, n is 1. In embodiments, n is 2.

In embodiments, R¹ is halogen, substituted or unsubstituted C₁-C₄ alkyl, —CN, —CX¹₃, or OR^{1F}. In embodiments, each R^{1F} is independently hydrogen or unsubstituted C₁-C₄ alkyl. In embodiments, each R^{1F} is independently hydrogen, or unsubstituted methyl.

In embodiments, R¹ is halogen, substituted or unsubstituted C₁-C₄ alkyl, —CN, —CX¹₃, or OR^{1F}. In embodiments, R¹ is —F. In embodiments, R¹ is —Cl. In embodiments, R¹ is —Br. In embodiments, R¹ is —I. In embodiments, R¹ is substituted C₁-C₄ alkyl. In embodiments, R¹ is unsubstituted C₁-C₄ alkyl. In embodiments, R¹ is methyl. In embodiments, R¹ is ethyl. In embodiments, R¹ is —CN. In embodiments, R¹ is —CF₃. In embodiments, R¹ is —OH. In embodiments, R¹ is —OCH₃.

In embodiments, R³⁰ is halogen, substituted or unsubstituted C₁-C₄ alkyl, —CN, —CX³, or OR^{30F}. In embodiments, each R^{30F} is independently hydrogen or unsubstituted C₁-C₄ alkyl. In embodiments, each R^{30F} is independently hydrogen, or unsubstituted methyl.

In embodiments, R³⁰ is halogen, substituted or unsubstituted C₁-C₄ alkyl, —CN, —CX¹₃, or OR^{1F}. In embodiments, R³⁰ is —F. In embodiments, R³⁰ is —Cl. In embodiments, R³⁰ is —Br. In embodiments, R³⁰ is —I. In embodiments, R³⁰ is substituted C₁-C₄ alkyl. In embodiments, R³⁰ is unsubstituted C₁-C₄ alkyl. In embodiments, R³⁰ is methyl. In embodiments, R³⁰ is ethyl. In embodiments, R³⁰ is —CN. In embodiments, R³⁰ is —CF₃. In embodiments, R³⁰ is —OH. In embodiments, R³⁰ is —OCH₃.

In embodiments, n is not 0. In embodiments, n is 0. In embodiments, R¹ is substituted or unsubstituted C₁-C₄ alkyl and R² is substituted or unsubstituted C₁-C₄ alkyl. In embodiments, R¹ is methyl and R² is methyl. In embodiments, R¹ is substituted or unsubstituted C₁-C₄ alkyl and R³ is substituted or unsubstituted C₁-C₄ alkyl. In embodiments, R¹ is methyl and R³ is methyl.

Compounds of Formula (XII) are shown in Table 2 below.

TABLE 2

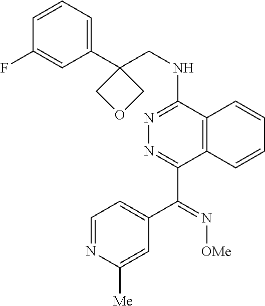 [SMILES: CO/N=C(\c1ccnc(C)c1)c1nnc(NCC2(c3cccc(F)c3)COC2)c2ccccc12]

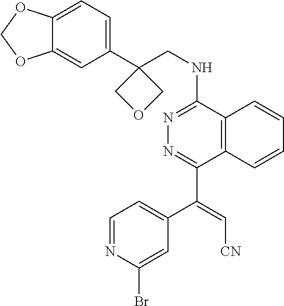 [SMILES: N#C/C=C(\c1ccnc(Br)c1)c1nnc(NCC2(c3ccc4c(c3)OCO4)COC2)c2ccccc12]

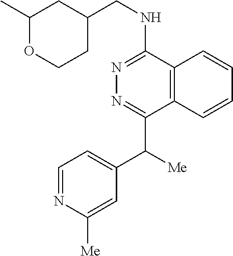 [SMILES: Cc1cc(C(C)c2nnc(NCC3CCOC(C)C3)c3ccccc23)ccn1]

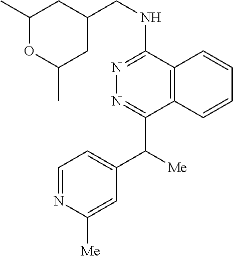 [SMILES: Cc1cc(C(C)c2nnc(NCC3CC(C)OC(C)C3)c3ccccc23)ccn1]

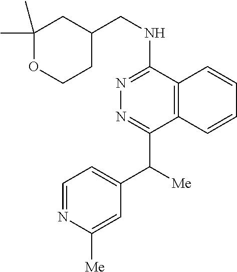 [SMILES: Cc1cc(C(C)c2nnc(NCC3CCOC(C)(C)C3)c3ccccc23)ccn1]

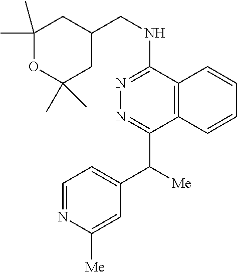 [SMILES: Cc1cc(C(C)c2nnc(NCC3CC(C)(C)OC(C)(C)C3)c3ccccc23)ccn1]

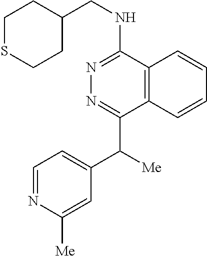 [SMILES: Cc1cc(C(C)c2nnc(NCC3CCSCC3)c3ccccc23)ccn1]

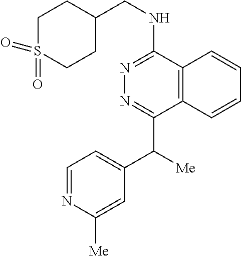 [SMILES: Cc1cc(C(C)c2nnc(NCC3CCS(=O)(=O)CC3)c3ccccc23)ccn1]

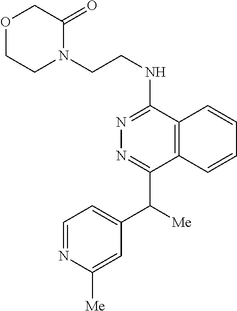 [SMILES: Cc1cc(C(C)c2nnc(NCCN3CCOCC3=O)c3ccccc23)ccn1]

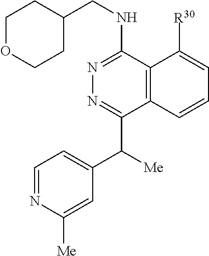

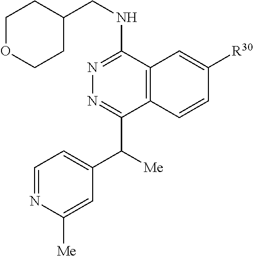

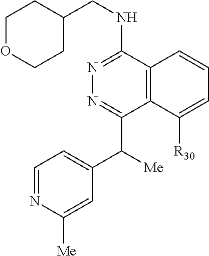

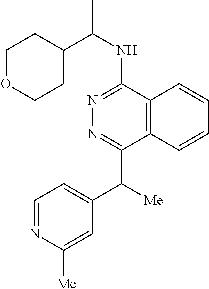 [SMILES: Cc1cc(C(C)c2nnc(NC(C)C3CCOCC3)c3ccccc23)ccn1]

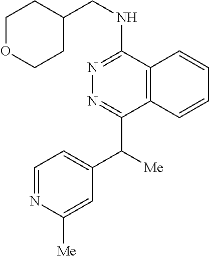 [SMILES: Cc1cc(C(C)c2nnc(NCC3CCOCC3)c3ccccc23)ccn1]

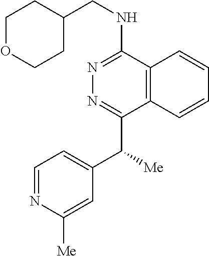 [SMILES: Cc1cc([C@@H](C)c2nnc(NCC3CCOCC3)c3ccccc23)ccn1]

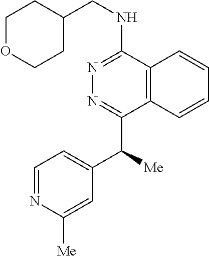 [SMILES: Cc1cc([C@H](C)c2nnc(NCC3CCOCC3)c3ccccc23)ccn1]

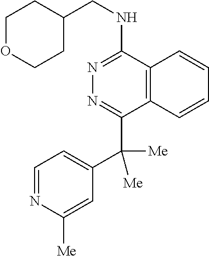 [SMILES: Cc1cc(C(C)(C)c2nnc(NCC3CCOCC3)c3ccccc23)ccn1]

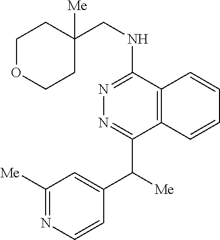 [SMILES: Cc1cc(C(C)c2nnc(NCC3(C)CCOCC3)c3ccccc23)ccn1]

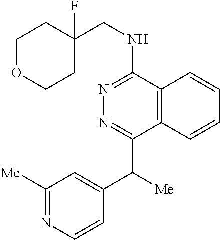 [SMILES: Cc1cc(C(C)c2nnc(NCC3(F)CCOCC3)c3ccccc23)ccn1]

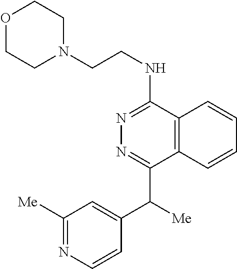 [SMILES: Cc1cc(C(C)c2nnc(NCCN3CCOCC3)c3ccccc23)ccn1]

In an aspect, a compound has a structure o Formula (III):

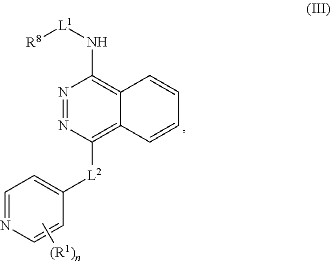

or a pharmaceutically acceptable salt thereof, or an isomer thereof;
   wherein:
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is —C(═O)—, —C(═CR^{9A}R^{9B})—, —CR^{10A}R^{10B}—, or —C(═NR¹¹)—;
R¹ is independently halogen, —CX¹₃, —CHX¹₂, —CH₂X¹, —OCX¹₃, —OCH₂X¹, —OCHX¹₂, —CN, —OR^{1F}, —SR^{1F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is a substituted or unsubstituted aryl, or substituted or unsubstituted heteroaryl;
Each R^{9A} and R^{9B} is independently hydrogen, —CX⁹₃, —CHX⁹₂, —CH₂X⁹, —OCX⁹₃, —OCH₂X⁹, —OCHX⁹₂, —CN, —OR^{9F}, —SR^{9F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Each R^{10A} and R^{10B} is independently hydrogen, D, halogen, —CX¹⁰₃, —CHX¹⁰₂, —CH₂X¹⁰, —OCX¹⁰₃, —OCH₂X¹⁰, —OCHX¹⁰₂, —CN, —OR^{10F}, —SR^{10F}, —C(O)OR^{10F}, —C(O)NHR^{10F}, —C(O)N(R^{10F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10A} and R^{10B} are optionally joined together to form a substituted or unsubstituted cycloalkyl, or substituted or unsubstituted heterocycloalkyl;
R¹¹ is —R^{11F}, —OR^{11F}, —S(O₂)—R^{11F}, or —C(O)—R^{11F};
n is an integer of 0 to 4,
Each X¹, X⁸, X⁹, and X¹⁰ is independently —F, —Br, —Cl, or —I;
Each R^{1F}, R^{8F}, R^{9F}, and R^{10F} is independently hydrogen, substituted or unsubstituted alkyl, or substituted or unsubstituted heteroalkyl; and
R^{11F} is a hydrogen, substituted or unsubstituted alkyl, substituted or unsubstituted aryl, or substituted or unsubstituted heteroaryl.

In embodiments, L² is —C(═O)—.

In embodiments, the compound has a structure of Formula (III-a),

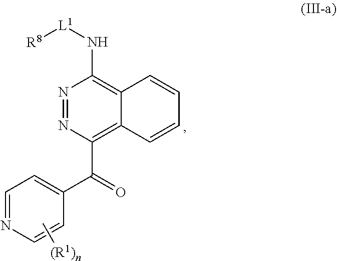

or a pharmaceutically acceptable salt thereof, or an isomer thereof. R¹, L¹, R⁸ and n are described above.

In embodiments, L² is —C(═CR^{9A}R^{9B})—.

In embodiments, the compound has a structure of Formula (III-b),

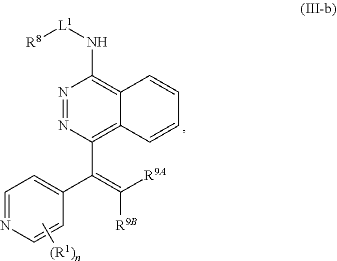

or a pharmaceutically acceptable salt thereof, or an isomer thereof. R¹, L¹, R⁸, R^{9A}, R^{9B}, and n are described above.

In embodiments, L² is —CR^{10A}R^{10B}—.

In embodiments, the compound has a structure of Formula (III-c),

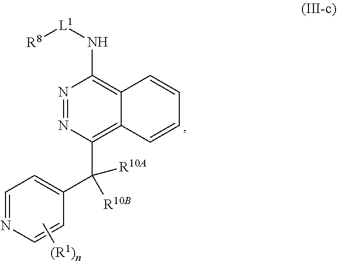

or a pharmaceutically acceptable salt thereof, or an isomer thereof. R¹, L¹, R⁸, R^{10A}, R^{10B}, and n are described above.

In embodiments, n is 1 to 4. In embodiments, n is not 0.

In embodiments, R^{10A} and R^{10B} are joined to form a substituted or unsubstituted C₃-C₆ cycloalkyl, or substituted or unsubstituted 4 to 6 membered heterocycloalkyl.

In embodiments, R^{10A} and R^{10B} are joined to form a substituted or unsubstituted C₃-C₆ cycloalkyl. In embodiments, R^{10A} and R^{10B} are joined to form a substituted or unsubstituted C₃-C₆ cycloalkyl.

In embodiments, R^{10A} and R^{10B} are joined together to form a substituted or unsubstituted C₃-C₆ cycloalkyl, or substituted or unsubstituted 4 to 6 membered heterocycloalkyl. In embodiments, R^{10A} and R^{10B} are joined together to form a substituted or unsubstituted C₃-C₆ cycloalkyl. In embodiments, R^{10A} and R^{10B} are joined together to form a substituted or unsubstituted cyclopropyl. In embodiments, R^{10A} and R^{10B} are joined together to form a substituted or unsubstituted cyclobutyl. In embodiments, R^{10A} and R^{10B} are joined together to form a substituted or unsubstituted cyclopentyl. In embodiments, R^{10A} and R^{10B} are joined together to form a substituted or unsubstituted cyclohexyl. In embodiments, R^{10A} and R^{10B} are joined together to form a substituted or unsubstituted 4 to 6 membered heterocycloalkyl. In embodiments, R^{10A} and R^{10B} are joined together to form a substituted or unsubstituted 4 membered heterocycloalkyl. In embodiments, R^{10A} and R^{10B} are joined together to form a substituted or unsubstituted 5 membered heterocycloalkyl. In embodiments, R^{10A} and R^{10B} are joined together to form a substituted or unsubstituted 6 membered heterocycloalkyl.

In embodiments, R^{10A} and R^{10B} are joined together to form a substituted or unsubstituted 4 to 6 membered heterocycloalkyl containing N, O, or S. In embodiments, R^{10A} and R^{10B} a joined together to form a substituted or unsubstituted 4 membered heterocycloalkyl containing N, O, or S. In embodiments, R^{10A} and R^{10B} are joined together to form a substituted or unsubstituted 5 membered heterocycloalkyl containing N, O, or S. In embodiments, R^{10A} and R^{10B} are joined together to form a substituted or unsubstituted 6 membered heterocycloalkyl containing N, O, or S. In embodiments, R^{10A} and R^{10B} are joined together to form a substituted or unsubstituted 4 to 6 membered heterocycloalkyl containing O. In embodiments, R^{10A} and R^{10B} are joined together to form a substituted or unsubstituted 4 membered heterocycloalkyl containing O. In embodiments, R^{10A} and R^{10B} are joined together to form a substituted or unsubstituted 5 membered heterocycloalkyl containing O. In embodiments, R^{10A} and R^{10B} are joined together to form a substituted or unsubstituted 6 membered heterocycloalkyl containing O.

In embodiments, the compound has a structure of Formula (III-c-1) or (III-c-2),

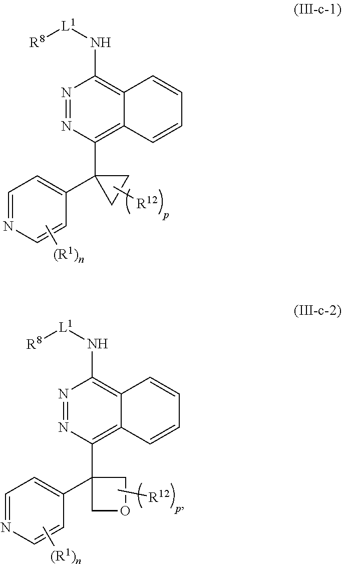

or a pharmaceutically acceptable salt thereof, or an isomer thereof. R¹, L¹, R⁸ and n are described above.

In embodiments, n is an integer of 0 to 4. In embodiments, n is 0. In embodiments, n is 1. In embodiments, n is 2.

In embodiments, each R¹² is independently halogen, —CX¹²₃, —CHX¹²₂, —CH₂X¹², —OCX¹²₃, —OCH₂X¹², —OCHX¹²₂, —CN, —OR^{12F}, —SR^{12F}, —C(O)OR^{12F}, —C(O)NHR^{12F}, —C(O)N(R^{12F})₂ substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; X¹² is independently —F, —Br, —Cl, or —I; and R^{12F} is independently hydrogen, substituted or unsubstituted alkyl, or substituted or unsubstituted heteroalkyl.

In embodiments, p is an integer from 0 to 4. In embodiments, p is 0. In embodiments, p is 1. In embodiments, p is 2.

In embodiments, each R¹² is independently halogen, —CX¹²₃, —OCX¹²₃, —CN, —OR^{1F}, —C(O)OR¹², —C(O)NHR¹², —C(O)N(R^{12F})₂, or substituted or unsubstituted C₁-C₄ alkyl. In embodiments, R¹² is halogen. In embodiments, R¹² is —F. In embodiments, R¹² is —Cl. In embodiments, R¹² is —Br. In embodiments, R¹² is —I. In embodiments, R¹² is —CX¹²₃. In embodiments, R¹² is —CF₃. In embodiments, R¹² is —OCX¹²₃. In embodiments, R¹² is —OCF₃. In embodiments, R¹² is —CN. In embodiments, R¹² is —OR¹². In embodiments, R¹² is —OH. In embodiments, R¹² is —OCH₃. In embodiments, R¹² is substituted or unsubstituted C₁-C₁₂ alkyl. In embodiments, R¹² is unsubstituted C₁-C₁₂ alkyl. In embodiments, R¹² is methyl. In embodiments, R¹² is ethyl. In embodiments, R¹² is —C(O)OR¹². In embodiments, R¹² is —C(O)NHR¹². In embodiments, R¹² is —C(O)N(R^{12F})₂. In embodiments, R^{12F} is —C(O)NH₂. In embodiments, R¹² is substituted or unsubstituted phenyl. In embodiments, R¹² is unsubstituted phenyl. At each occurrence, R¹² may be the same or different.

In embodiments, R¹² is a hydrogen, substituted or unsubstituted alkyl, substituted or unsubstituted aryl, or substituted or unsubstituted heteroalkyl. In embodiments, R¹² is a hydrogen. In embodiments, R¹² is a substituted or unsubstituted alkyl. In embodiments, R¹² is a substituted or unsubstituted C₁-C₄ alkyl. In embodiments, R¹² is an unsubstituted C₁-C₄ alkyl. In embodiments, R¹² is methyl. In embodiments, R¹² is ethyl. In embodiments, R¹² is a substituted or unsubstituted 2 to 6 membered heteroalkyl. In embodiments, R¹² is a substituted or unsubstituted 2 membered heteroalkyl. In embodiments, R¹² is a substituted or unsubstituted 3 membered heteroalkyl. In embodiments, R¹² is a substituted or unsubstituted 4 membered heteroalkyl. In embodiments, R¹² is a substituted or unsubstituted 5 membered heteroalkyl. In embodiments, R¹² is a substituted or unsubstituted 6 membered heteroalkyl.

In embodiments, the compound may have a structure of Formula (III-c-3), (III-c-4), or (III-c-5),

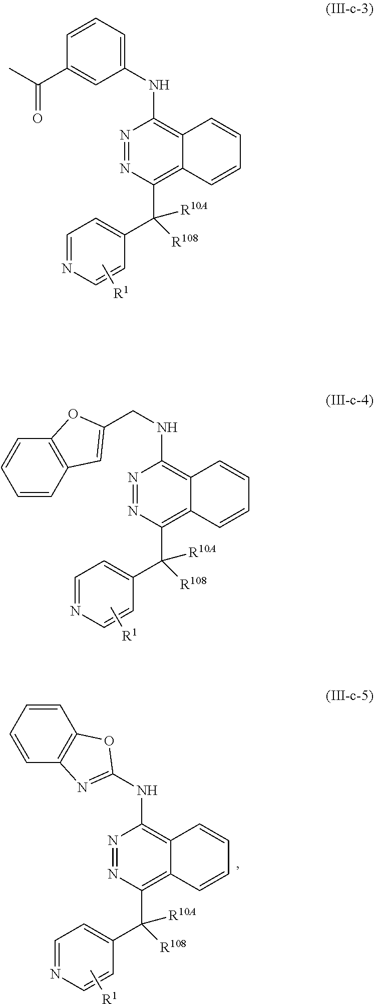

or a pharmaceutically acceptable salt thereof, or an isomer thereof. R¹, R^{10A}, R^{10B} and n are described above.

In embodiments, R^{10A} is H, D, halogen, substituted or unsubstituted C₁-C₄ alkyl, —CN, —CX¹⁰₃, or OR^{10F}, and R^{10B} is H, D, halogen, substituted or unsubstituted C₁-C₄ alkyl, —CN, —CX¹⁰₃, or OR^{10F}. In embodiments, each R^{10F} is independently hydrogen, or unsubstituted C₁-C₄ alkyl. In embodiments, each R^{10F} is independently hydrogen, or unsubstituted methyl. In embodiments, R^{10F} is hydrogen, or unsubstituted methyl.

In embodiments, R^{10A} is —H, -D, halogen, substituted or unsubstituted C₁-C₄ alkyl, —CN, —CX¹⁰₃, or OR^{10F}. In embodiments, R^{10A} is —H. In embodiments, R^{10A} is -D. In embodiments, R^{10A} is —F. In embodiments, R^{10A} is —Cl. In embodiments, R^{10A} is —Br. In embodiments, R^{10A} is —I. In embodiments, R^{10A} is substituted C₁-C₄ alkyl. In embodiments, R^{10A} is unsubstituted C₁-C₄ alkyl. In embodiments, R^{10A} is methyl. In embodiments, R^{10A} is ethyl. In embodiments, R^{10A} is —CN. In embodiments, R^{10A} is —CF₃. In embodiments, R^{10A} is —OH. In embodiments, R^{10A} is —OCH₃.

In embodiments, R^{10B} is —H, -D, halogen, substituted or unsubstituted C₁-C₄ alkyl, —CN, —CX^{10B}_{10B}, or OR^{10BF}. In embodiments, R^{10B} is —H. In embodiments, R^{10B} is -D. In embodiments, R^{10B} is —F. In embodiments, R^{10B} is —Cl. In embodiments, R^{10B} is —Br. In embodiments, R^{10B} is —I. In embodiments, R^{10B} is substituted C₁-C₄ alkyl. In embodiments, R^{10B} is unsubstituted C₁-C₄ alkyl. In embodiments, R^{10B} is methyl. In embodiments, R^{10B} is ethyl. In embodiments, R^{10B} is —CN. In embodiments, R^{10B} is —CF_{10B}. In embodiments, R^{10B} is —OH. In embodiments, R^{10B} is —OCH₃.

In embodiments, k is 0. In embodiments, k is 1. In embodiments, k is 2. In embodiments, m is 0. In embodiments, m is 1. In embodiments, m is 2.

In embodiments, L² is —C(═NR¹¹)—. In embodiments, R¹¹ is —R^{11F}, —OR^{11F}, —S(O₂)—R^{11F}, or —C(O)—R^{11F}. In embodiments, R^{11F} is a hydrogen, substituted or unsubstituted alkyl, substituted or unsubstituted aryl, or substituted or unsubstituted heteroaryl.

In embodiments, the compound has a structure of Formula (III-d-1), (III-d-2), (III-d-3), or (III-d-4),

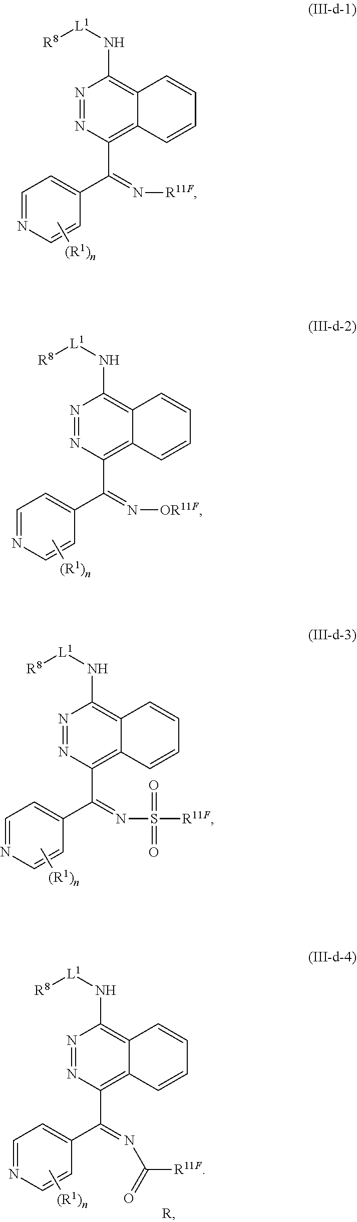

or a pharmaceutically acceptable salt thereof, or an isomer thereof. L¹, R⁸, R^{11F} and n are described above.

In embodiments, R^{11F} is a hydrogen, substituted or unsubstituted alkyl, substituted or unsubstituted aryl, or substituted or unsubstituted heteroaryl. In embodiments, R^{11F} is a hydrogen. In embodiments, R^{11F} is a substituted or unsubstituted alkyl. In embodiments, R^{11F} is a substituted or unsubstituted C₁-C₄ alkyl. In embodiments, R^{11F} is an unsubstituted C₁-C₄ alkyl. In embodiments, R^{11F} is methyl. In embodiments, R^{11F} is ethyl. In embodiments, R^{11F} is a substituted or unsubstituted phenyl. In embodiments, R^{11F} is an unsubstituted phenyl. In embodiments, R^{11F} is a substituted or unsubstituted 5 to 8 membered heteroaryl. In embodiments, R^{11F} is a substituted or unsubstituted 5 to 6 membered heteroaryl. In embodiments, R^{11F} is a substituted or unsubstituted 5 membered heteroaryl. In embodiments, R^{11F} is a substituted or unsubstituted 6 membered heteroaryl.

In embodiments, L¹ is a bond or unsubstituted C₁-C₄ alkylene. In embodiments, L¹ is a bond. In embodiments, L¹ is unsubstituted C₁-C₄ alkylene. In embodiments, L¹ is unsubstituted C₁-C₄ alkylene.

In embodiments, R⁸ is

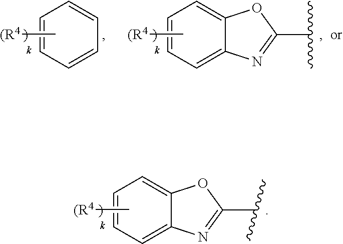

In embodiments, each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)OR^{4F}, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, X⁴ is independently —F, —Br, —Cl, or —I; and each R^{4F} is independently hydrogen, substituted or unsubstituted alkyl, or substituted or unsubstituted heteroalkyl. In embodiments, k is an integer of 0 to 5.

In embodiments, each R⁴ is independently halogen, —CX⁴₃, —OCX⁴₃, —CN, —OR^{2F}, —C(O)OR^{4F}, —C(O)NHR^{4F}, —C(O)N(R^{4F})₂, or substituted or unsubstituted C₁-C₄ alkyl. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is —CX⁴₃. In embodiments, R⁴ is —CF₃. In embodiments, R⁴ is —OCX⁴₃. In embodiments, R⁴ is —OCF₃. In embodiments, R⁴ is —CN. In embodiments, R⁴ is —OR^{4F}. In embodiments, R⁴ is —OH. In embodiments, R⁴ is —OCH₃. In embodiments, R⁴ is substituted or unsubstituted C₁-C₄ alkyl. In embodiments, R⁴ is unsubstituted C₁-C₄ alkyl. In embodiments, R⁴ is methyl. In embodiments, R⁴ is ethyl. In embodiments, R⁴ is —C(O)OR^{4F}. In embodiments, R⁴ is —C(O)NHR^{4F}. In embodiments, R⁴ is —C(O)N(R^{4F})₂. In embodiments, R⁴ is —C(O)NH₂.

In embodiments, R^{4F} is a hydrogen, substituted or unsubstituted alkyl, substituted or unsubstituted aryl, or substituted or unsubstituted heteroalkyl. In embodiments, R^{4F} is a hydrogen. In embodiments, R^{4F} is a substituted or unsubstituted alkyl. In embodiments, R^{4F} is a substituted or unsubstituted C₁-C₄ alkyl. In embodiments, R^{4F} is an unsubstituted C₁-C₄ alkyl. In embodiments, R^{4F} is methyl. In embodiments, R^{4F} is ethyl. In embodiments, R^{4F} is a substituted or unsubstituted 2 to 6 membered heteroalkyl. In embodiments, R^{4F} is a substituted or unsubstituted 2 membered heteroalkyl. In embodiments, R^{4F} is a substituted or unsubstituted 3 membered heteroalkyl. In embodiments, R^{4F} is a substituted or unsubstituted 4 membered heteroalkyl. In embodiments, R^{4F} is a substituted or unsubstituted 5 membered heteroalkyl. In embodiments, R^{4F} is a substituted or unsubstituted 6 membered heteroalkyl.

In embodiments, n is 0 or 1; R¹ is halogen, substituted or unsubstituted C₁-C₄ alkyl, —CN, —CX¹₃, or OR^{1F}; and R^{1F} is hydrogen or unsubstituted C₁-C₄ alkyl.

In embodiments, R¹ is halogen, substituted or unsubstituted C₁-C₄ alkyl, —CN, —CX¹₃, or OR^{1F}. In embodiments, R^{1F} is a hydrogen, substituted or unsubstituted alkyl, substituted or unsubstituted aryl, or substituted or unsubstituted heteroalkyl. In embodiments, R^{1F} is a hydrogen. In embodiments, R^{1F} is a substituted or unsubstituted alkyl. In embodiments, R^{1F} is a substituted or unsubstituted C₁-C₄ alkyl. In embodiments, R^{1F} is an unsubstituted C₁-C₄ alkyl. In embodiments, R^{1F} is methyl. In embodiments, R^{1F} is ethyl.

In embodiments, R¹ is —F. In embodiments, R¹ is —Cl. In embodiments, R¹ is —Br. In embodiments, R¹ is —I. In embodiments, R¹ is substituted C₁-C₄ alkyl. In embodiments, R¹ is unsubstituted C₁-C₄ alkyl. In embodiments, R¹ is methyl. In embodiments, R¹ is ethyl. In embodiments, R¹ is —CN. In embodiments, R¹ is —CF₃. In embodiments, R¹ is —OH. In embodiments, R¹ is —OCH₃.

In embodiments, exemplary compounds of Formula (III) include

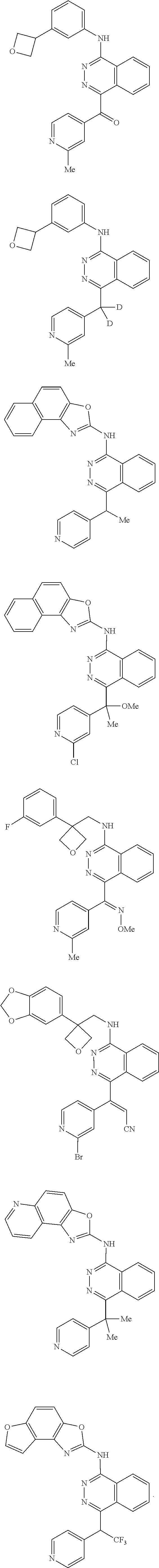

In an aspect provided is a compound has a structure of Formula (XIII):

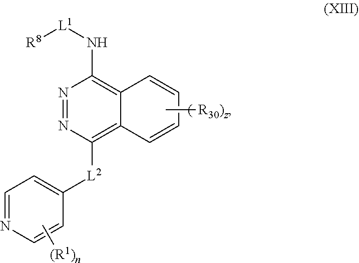

or a pharmaceutically acceptable salt thereof, or an isomer thereof;
wherein:
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is —C(═O)—, —C(═CR^{9A}R^{9B})—, —CR^{10A}R^{10B}—, or —C(═NR¹¹)—;
R¹ is independently halogen, —CX¹₃, —CHX¹₂, —CH₂X¹, —OCX¹₃, —OCH₂X¹, —OCHX¹₂, —CN, —OR^{1F}, —SR^{1F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is a substituted or unsubstituted cycloalkyl, substituted or unsubstituted heterocycloalkyl, substituted or unsubstituted aryl, or substituted or unsubstituted heteroaryl;
Each R^{9A} and R^{9B} is independently hydrogen, —CX⁹₃, —CHX⁹₂, —CH₂X⁹, —OCX⁹₃, —OCH₂X⁹, —OCHX⁹₂, —CN, —OR^{9F}, —SR^{9F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Each R^{10A} and R^{10B} is independently hydrogen, D, halogen, —CX¹⁰₃, —CHX¹⁰₂, —CH₂X¹⁰, —OCX¹⁰₃, —OCH₂X¹⁰, —OCHX¹⁰₂, —CN, —OR^{10F}, —SR^{10F}, —C(O)OR^{10F}, —C(O)NHR^{10F}, —C(O)N(R^{10F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10A} and R^{10B} are optionally joined together to form a substituted or unsubstituted cycloalkyl, or substituted or unsubstituted heterocycloalkyl;
R¹¹ is —R^{11F}, —OR^{11F}, —S(O₂)—R^{11F}, or —C(O)—R^{11F};
R³⁰ is independently halogen, —CX³⁰₃, —CHX³⁰₂, —CH₂X³⁰, —OCX³⁰₃, —OCH₂X³⁰, —OCHX³⁰₂, —CN, —OR^{30F}, —SR^{30F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is an integer of 1 to 4;
z is an integer of 0 to 4;
Each X¹, X⁸, X⁹, X¹⁰, and X³⁰ is independently —F, —Br, —Cl, or —I;
Each R^{1F}, R^{8F}, R^{9F}, R^{10F} and R^{30F} is independently hydrogen, substituted or unsubstituted alkyl, or substituted or unsubstituted heteroalkyl; and
R^{11F} is a hydrogen, substituted or unsubstituted alkyl, substituted or unsubstituted aryl, or substituted or unsubstituted heteroaryl.

In embodiments, at least one of R^{10A} or R^{10B} is not hydrogen. In embodiments, R^{10A} is not hydrogen. In embodiments, R^{10B} is not hydrogen. In embodiments, R¹ is not hydrogen and R^{10A} is not hydrogen. In embodiments, R¹ is not hydrogen and R^{10B} is not hydrogen.

In embodiments, R⁸ is

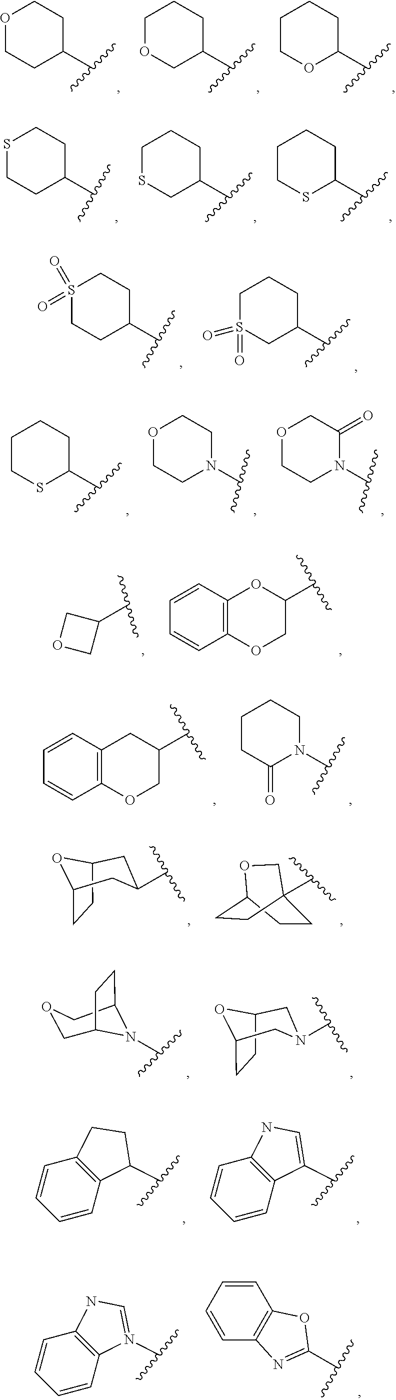

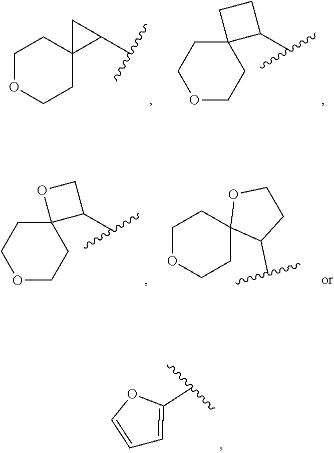 [SMILES: *C1CC12CCOCC2 ; *C1CCC12CCOCC2 ; *C1COC12CCOCC2 ; *C1CCOC12CCOCC2 ; *c1ccco1]

which is substituted or unsubstituted.

In embodiments, R⁸ is

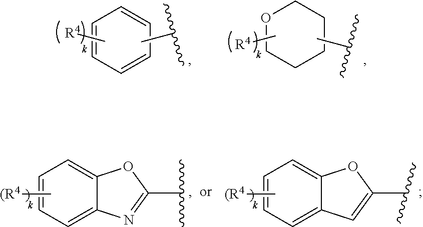

Each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)R^{4F}, —C(O)OR^{4F}, —S(O)₂R^{4F}, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
k is an integer of 0 to 5;
X⁴ is independently —F, —Br, —Cl, or —I; and
Each R^{4F} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments,

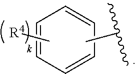

In embodiments, R⁸ is

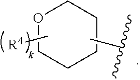

In embodiments, R⁸ is

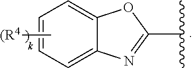

In embodiments, R⁸ is

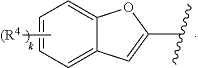

In embodiments, each R⁴ is independently halogen, —CX⁴₃, —OCX⁴₃, —CN, —OR^{4F}, —C(O)R^{4F}, —C(O)OR^{4F}, —S(O)₂R^{4F}, —C(O)NHR^{4F}, —C(O)N(R^{4F})₂, or substituted or unsubstituted C₁-C₄ alkyl. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is —CX⁴₃. In embodiments, R⁴ is —CF₃. In embodiments, R⁴ is —OCX⁴₃. In embodiments, R⁴ is —OCF₃. In embodiments, R⁴ is —CN. In embodiments, R⁴ is —OR^{4F}. In embodiments, R⁴ is —OH. In embodiments, R⁴ is —OCH₃. In embodiments, R⁴ is substituted or unsubstituted C₁-C₄ alkyl. In embodiments, R⁴ is unsubstituted C₁-C₄ alkyl. In embodiments, R⁴ is methyl. In embodiments, R⁴ is ethyl. In embodiments, R⁴ is —C(O)OR^{4F}. In embodiments, R⁴ is —S(O)₂R^{4F}. In embodiments, R⁴ is —C(O)NHR^{4F}. In embodiments, R⁴ is —C(O)N(R^{4F})₂. In embodiments, R⁴ is —C(O)NH₂.

In embodiments, R^{4F} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In embodiments, R^{4F} is a hydrogen. In embodiments, R^{4F} is a substituted or unsubstituted alkyl. In embodiments, R^{4F} is a substituted or unsubstituted C₁-C₄ alkyl. In embodiments, R^{4F} is an unsubstituted C₁-C₄ alkyl. In embodiments, R^{4F} is methyl. In embodiments, R^{4F} is ethyl. In embodiments, R^{4F} is a substituted or unsubstituted 2 to 6 membered heteroalkyl. In embodiments, R^{4F} is a substituted or unsubstituted 2 membered heteroalkyl. In embodiments, R^{4F} is a substituted or unsubstituted 3 membered heteroalkyl. In embodiments, R^{4F} is a substituted or unsubstituted 4 membered heteroalkyl. In embodiments, R^{4F} is a substituted or unsubstituted 5 membered heteroalkyl. In embodiments, R^{4F} is a substituted or unsubstituted 6 membered heteroalkyl. In embodiments, R^{4F} is substituted or unsubstituted cycloalkyl. In embodiments, R^{4F} is substituted or unsubstituted C₃-C₆ cycloalkyl. In embodiments, R^{4F} is substituted or unsubstituted 5 to 12 membered mono-cyclic or bi-cyclic heterocycloalkyl. In embodiments, R^{4F} is unsubstituted 5 to 12 membered mono-cyclic or bi-cyclic heterocycloalkyl. In embodiments, R^{4F} is substituted 5 to 12 membered mono-cyclic or bi-cyclic heterocycloalkyl. In embodiments, R^{4F} is substituted or unsubstituted phenyl. In embodiments, R^{4F} is unsubstituted phenyl. In embodiments, R^{4F} is substituted phenyl. In embodiments, R^{4F} is substituted or unsubstituted 5 to 6 membered heteroaryl. In embodiments, R^{4F} is substituted 5 to 6 membered heteroaryl. In embodiments, R^{4F} is unsubstituted 5 to 6 membered heteroaryl.

In embodiments, the compound has a structure of Formula (XIII-a),

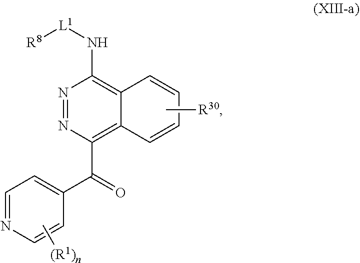

or a pharmaceutically acceptable salt thereof, or an isomer thereof;
wherein n is an integer of 1 to 4.
R¹, L¹, R⁸, R³⁰, and n are defined in Formula (XIII) and described above.

In embodiments, the compound has a structure of Formula (XIII-b),

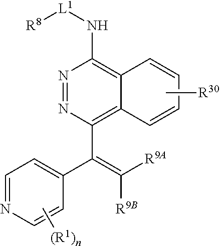

or a pharmaceutically acceptable salt thereof, or an isomer thereof;
wherein n is an integer of 1 to 4.
R¹, L¹, R⁸, R^{9A}, R^{9B}, R³⁰, and n are defined in Formula (XIII) and described above.

In embodiments, the compound has a structure of Formula (XIII-c),

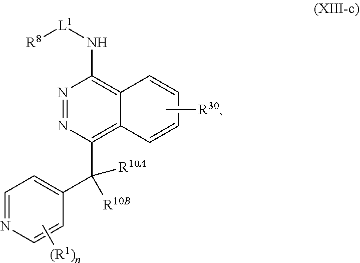

or a pharmaceutically acceptable salt thereof, or an isomer thereof;
wherein n is an integer of 1 to 4.
R¹, L¹, R⁸, R^{10A}, R^{10B}, R³⁰, and n are defined in Formula (XIII) and described above.

In embodiments, L¹ is a bond or unsubstituted C₁-C₄ alkylene. In embodiments, L¹ is a bond. In embodiments, L¹ is unsubstituted C₁-C₄ alkylene. In embodiments, L¹ is unsubstituted C₁-C₄ alkylene.

In embodiments, n is 1 to 4. In embodiments, n is not 0.

In embodiments, R^{10A} and R^{10B} are joined to form a substituted or unsubstituted C₃-C₆ cycloalkyl, or substituted or unsubstituted 4 to 6 membered heterocycloalkyl.

In embodiments, R^{10A} and R^{10B} are joined to form a substituted or unsubstituted C₃-C₆ cycloalkyl. In embodiments, R^{10A} and R^{10B} are joined to form a substituted or unsubstituted C₃-C₆ cycloalkyl.

In embodiments, R^{10A} and R^{10B} are joined together to form a substituted or unsubstituted C₃-C₆ cycloalkyl, or substituted or unsubstituted 4 to 6 membered heterocycloalkyl. In embodiments, R^{10A} and R^{10B} are joined together to form a substituted or unsubstituted C₃-C₆ cycloalkyl. In embodiments, R^{10A} and R^{10B} are joined together to form a substituted or unsubstituted cyclopropyl. In embodiments, R^{10A} and R^{10B} are joined together to form a substituted or unsubstituted cyclobutyl. In embodiments, R^{10A} and R^{10B} are joined together to form a substituted or unsubstituted cyclopentyl. In embodiments, R^{10A} and R^{10B} are joined together to form a substituted or unsubstituted cyclohexyl. In embodiments, R^{10A} and R^{10B} are joined together to form a substituted or unsubstituted 4 to 6 membered heterocycloalkyl. In embodiments, R^{10A} and R^{10B} are joined together to form a substituted or unsubstituted 4 membered heterocycloalkyl. In embodiments, R^{10A} and R^{10B} are joined together to form a substituted or unsubstituted 5 membered heterocycloalkyl. In embodiments, R^{10A} and R^{10B} are joined together to form a substituted or unsubstituted 6 membered heterocycloalkyl.

In embodiments, R^{10A} and R^{10B} are joined together to form a substituted or unsubstituted 4 to 6 membered heterocycloalkyl containing N, O, or S. In embodiments, R^{10A} and R^{10B} a joined together to form a substituted or unsubstituted 4 membered heterocycloalkyl containing N, O, or S. In embodiments, R^{10A} and R^{10B} are joined together to form a substituted or unsubstituted 5 membered heterocycloalkyl containing N, O, or S. In embodiments, R^{10A} and R^{10B} are joined together to form a substituted or unsubstituted 6 membered heterocycloalkyl containing N, O, or S. In embodiments, R^{10A} and R^{10B} are joined together to form a substituted or unsubstituted 4 to 6 membered heterocycloalkyl containing O. In embodiments, R^{10A} and R^{10B} are joined together to form a substituted or unsubstituted 4 membered heterocycloalkyl containing O. In embodiments, R^{10A} and R^{10B} are joined together to form a substituted or unsubstituted 5 membered heterocycloalkyl containing O. In embodiments, R^{10A} and R^{10B} are joined together to form a substituted or unsubstituted 6 membered heterocycloalkyl containing O.

In embodiments, the compound has a structure of Formula (XIII-c-1) or (XIII-c-2),

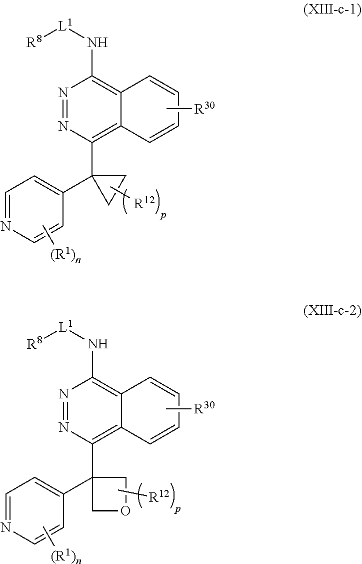

or a pharmaceutically acceptable salt thereof, or an isomer thereof;
wherein:
Each R¹² is independently halogen, —CX¹²₃, —CHX¹²₂, —CH₂X¹², —OCX¹²₃, —OCH₂X¹², —OCHX¹²₂, —CN, —OR^{12F}, —SR^{12F}, —C(O)OR^{12F}, —C(O)NHR^{12F}, —C(O)N(R^{12F})₂ substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X¹² is independently —F, —Br, —Cl, or —I;
Each R¹² is independently hydrogen, substituted or unsubstituted alkyl, or substituted or unsubstituted heteroalkyl; and
p is an integer from 0 to 4.
R¹, L¹, R⁸, R³⁰, and n are defined in Formula (XIII) and described above.

In embodiments, p is an integer from 0 to 4. In embodiments, p is 0. In embodiments, p is 1. In embodiments, p is 2.

In embodiments, each R¹² is independently halogen, —CX¹²₃, —OCX¹²₃, —CN, —OR^{12F}, —C(O)OR^{12F}, —C(O)NHR^{12F}, —C(O)N(R^{12F})₂, or substituted or unsubstituted C₁-C₄ alkyl. In embodiments, R¹² is halogen. In embodiments, R¹² is —F. In embodiments, R¹² is —Cl. In embodiments, R¹² is —Br. In embodiments, R¹² is —I. In embodiments, R¹² is —CX¹²₃. In embodiments, R¹² is —CF₃. In embodiments, R¹² is —OCX¹²₃. In embodiments, R¹² is —OCF₃. In embodiments, R¹² is —CN. In embodiments, R¹² is —OR¹². In embodiments, R¹² is —OH. In embodiments, R¹² is —OCH₃. In embodiments, R¹² is substituted or unsubstituted C₁-C₁₂ alkyl. In embodiments, R¹² is unsubstituted C₁-C₁₂ alkyl. In embodiments, R¹² is methyl. In embodiments, R¹² is ethyl. In embodiments, R¹² is —C(O)OR¹². In embodiments, R¹² is —C(O)NHR¹². In embodiments, R¹² is —C(O)N(R^{12F})₂. In embodiments, R¹² is —C(O)NH₂. In embodiments, R¹² is substituted or unsubstituted phenyl. In embodiments, R¹² is unsubstituted phenyl. At each occurrence, R¹² may be the same or different.

In embodiments, R^{12F} is a hydrogen, substituted or unsubstituted alkyl, substituted or unsubstituted aryl, or substituted or unsubstituted heteroalkyl. In embodiments, R^{12F} is a hydrogen. In embodiments, R^{12F} is a substituted or unsubstituted alkyl. In embodiments, R^{12F} is a substituted or unsubstituted C₁-C₄ alkyl. In embodiments, R^{12F} is an unsubstituted C₁-C₄ alkyl. In embodiments, R^{12F} is methyl. In embodiments, R^{12F} is ethyl. In embodiments, R^{12F} is a substituted or unsubstituted 2 to 6 membered heteroalkyl. In embodiments, R^{12F} is a substituted or unsubstituted 2 membered heteroalkyl. In embodiments, R^{12F} is a substituted or unsubstituted 3 membered heteroalkyl. In embodiments, R^{12F} is a substituted or unsubstituted 4 membered heteroalkyl. In embodiments, R^{12F} is a substituted or unsubstituted 5 membered heteroalkyl. In embodiments, R^{12F} is a substituted or unsubstituted 6 membered heteroalkyl.

In embodiments, the compound has a structure of Formula (XIII-c-3), (XIII-c-4), or (XIII-c-5),

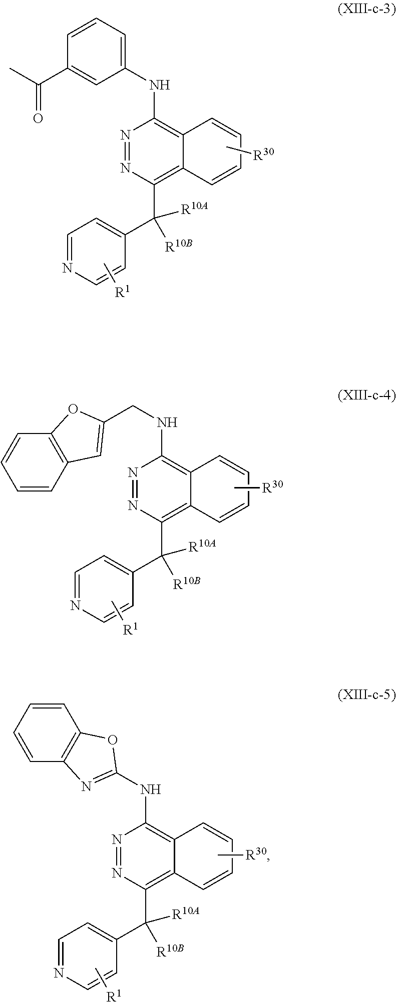

or a pharmaceutically acceptable salt thereof, or an isomer thereof.

In embodiments, R^{10A} is H, D, halogen, substituted or unsubstituted C₁-C₄ alkyl, —CN, —CX¹⁰₃, or OR^{10F}, and R^{10B} is H, D, halogen, substituted or unsubstituted C₁-C₄ alkyl, —CN, —CX¹⁰₃, or OR^{10F}. In embodiments, each R^{10F} is independently hydrogen, or unsubstituted C₁-C₄ alkyl. In embodiments, each R^{10F} is independently hydrogen, or unsubstituted methyl. In embodiments, R^{10F} is hydrogen, or unsubstituted methyl.

In embodiments, R^{10A} is —H, -D, halogen, substituted or unsubstituted C₁-C₄ alkyl, —CN, —CX¹⁰₃, or OR^{10F}. In embodiments, R^{10A} is —H. In embodiments, R^{10A} is -D. In embodiments, R^{10A} is —F. In embodiments, R^{10A} is —Cl. In embodiments, R^{10A} is —Br. In embodiments, R^{10A} is —I. In embodiments, R^{10A} is substituted C₁-C₄ alkyl. In embodiments, R^{10A} is unsubstituted C₁-C₄ alkyl. In embodiments, R^{10A} is methyl. In embodiments, R^{10A} is ethyl. In embodiments, R^{10A} is —CN. In embodiments, R^{10A} is —CF₃. In embodiments, R^{10A} is —OH. In embodiments, R^{10A} is —OCH₃.

In embodiments, R^{10B} is —H, -D, halogen, substituted or unsubstituted C₁-C₄ alkyl, —CN, —CX^{10B}_{10B}, or OR^{10BF}. In embodiments, R^{10B} is —H. In embodiments, R^{10B} is -D. In embodiments, R^{10B} is —F. In embodiments, R^{10B} is —Cl. In embodiments, R^{10B} is —Br. In embodiments, R^{10B} is —I. In embodiments, R^{10B} is substituted C₁-C₄ alkyl. In embodiments, R^{10B} is unsubstituted C₁-C₄ alkyl. In embodiments, R^{10B} is methyl. In embodiments, R^{10B} is ethyl. In embodiments, R^{10B} is —CN. In embodiments, R^{10B} is —CF_{10B}. In embodiments, R^{10B} is —OH. In embodiments, R^{10B} is —OCH₃.

In embodiments, k is 0. In embodiments, k is 1. In embodiments, k is 2. In embodiments, m is 0. In embodiments, m is 1. In embodiments, m is 2.

In embodiments, L² is —C(═NR¹¹)—. In embodiments, R¹¹ is —R^{11F}, —OR^{11F}, —S(O₂)—R^{11F}, or —C(O)—R^{11F}. In embodiments, R^{11F} is a hydrogen, substituted or unsubstituted alkyl, substituted or unsubstituted aryl, or substituted or unsubstituted heteroaryl.

In embodiments, the compound has a structure of Formula (XIII-d-1), (XIII-d-2), (XIII-d-3), or (XIII-d-4),

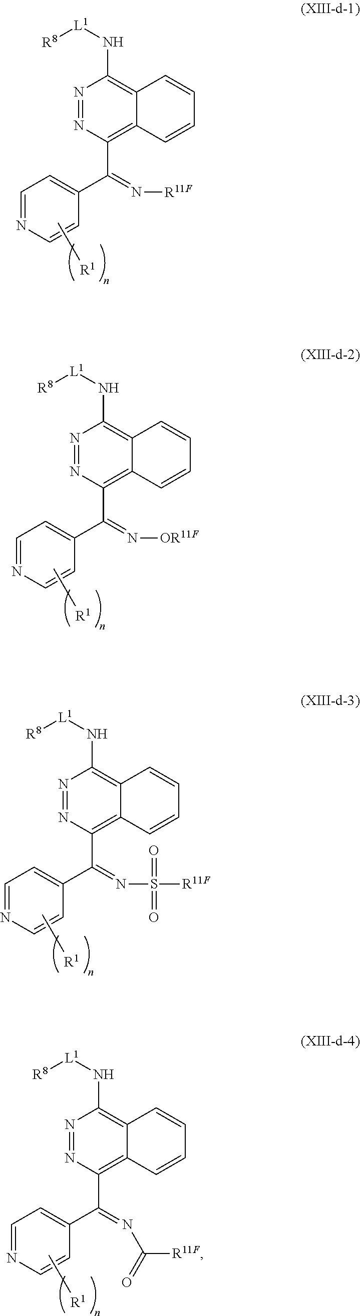

or a pharmaceutically acceptable salt thereof, or an isomer thereof.

In embodiments, R^{11F} is a hydrogen, substituted or unsubstituted alkyl, substituted or unsubstituted aryl, or substituted or unsubstituted heteroaryl. In embodiments, R^{11F} is a hydrogen. In embodiments, R^{11F} is a substituted or unsubstituted alkyl. In embodiments, R^{11F} is a substituted or unsubstituted C₁-C₄ alkyl. In embodiments, R^{11F} is an unsubstituted C₁-C₄ alkyl. In embodiments, R^{11F} is methyl. In embodiments, R^{11F} is ethyl. In embodiments, R^{11F} is a substituted or unsubstituted phenyl. In embodiments, R^{11F} is an unsubstituted phenyl. In embodiments, R^{11F} is a substituted or unsubstituted 5 to 8 membered heteroaryl. In embodiments, R^{11F} is a substituted or unsubstituted 5 to 6 membered heteroaryl. In embodiments, R^{11F} is a substituted or unsubstituted 5 membered heteroaryl. In embodiments, R^{11F} is a substituted or unsubstituted 6 membered heteroaryl.

In embodiments, R¹ is halogen, substituted or unsubstituted C₁-C₄ alkyl, —CN, —CX¹₃, or OR^{1F}. In embodiments, each R^{1F} is independently hydrogen or unsubstituted C₁-C₄ alkyl. In embodiments, each R^{1F} is independently hydrogen, or unsubstituted methyl.

In embodiments, R¹ is halogen, substituted or unsubstituted C₁-C₄ alkyl, —CN, —CX¹₃, or OR^{1F}. In embodiments, R¹ is —F. In embodiments, R¹ is —Cl. In embodiments, R¹ is —Br. In embodiments, R¹ is —I. In embodiments, R¹ is substituted C₁-C₄ alkyl. In embodiments, R¹ is unsubstituted C₁-C₄ alkyl. In embodiments, R¹ is methyl. In embodiments, R¹ is ethyl. In embodiments, R¹ is —CN. In embodiments, R¹ is —CF₃. In embodiments, R¹ is —OH. In embodiments, R¹ is —OCH₃.

In embodiments, R³⁰ is halogen, substituted or unsubstituted C₁-C₄ alkyl, —CN, —CX³⁰₃, or OR^{30F}. In embodiments, each R^{30F} is independently hydrogen or unsubstituted C₁-C₄ alkyl. In embodiments, each R^{30F} is independently hydrogen, or unsubstituted methyl.

In embodiments, R³⁰ is halogen, substituted or unsubstituted C₁-C₄ alkyl, —CN, —CX¹₃, or OR^{1F}. In embodiments, R³⁰ is —F. In embodiments, R³⁰ is —Cl. In embodiments, R³⁰ is —Br. In embodiments, R³⁰ is —I. In embodiments, R³⁰ is substituted C₁-C₄ alkyl. In embodiments, R³⁰ is unsubstituted C₁-C₄ alkyl. In embodiments, R³⁰ is methyl. In embodiments, R³⁰ is ethyl. In embodiments, R³⁰ is —CN. In embodiments, R³⁰ is —CF₃. In embodiments, R³⁰ is —OH. In embodiments, R³⁰ is —OCH₃.

In embodiments, the compound has a structure of Formula (XIII-e),

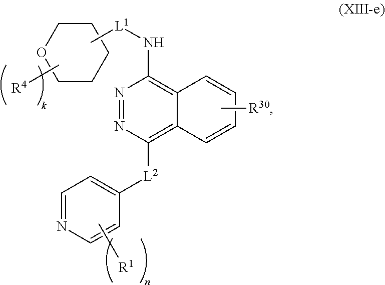

or a pharmaceutically acceptable salt thereof, or an isomer thereof.

In embodiments, the compound has a structure of Formula (XIII-f),

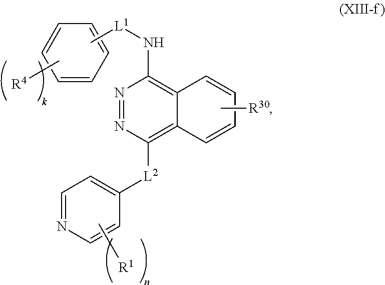

or a pharmaceutically acceptable salt thereof, or an isomer thereof.

In embodiments, the compound has a structure of Formula (XIII-g),

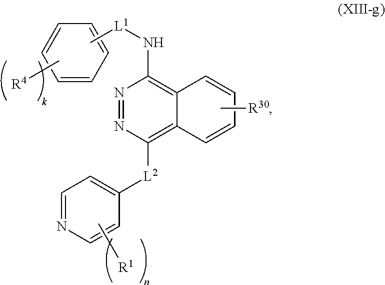

or a pharmaceutically acceptable salt thereof, or an isomer thereof.

In embodiments, k is 0. In embodiments, k is 1. In embodiments, k is 2. In embodiments, n is 1. In embodiments, n is 2.

In embodiments, each R⁴ is independently halogen, —CX⁴₃, —OCX⁴₃, —CN, —OR^{4F}, —C(O)R^{4F}, —C(O)OR^{4F}, —S(O)₂R^{4F}, —C(O)NHR^{4F}, —C(O)N(R^{4F})₂, or substituted or unsubstituted C₁-C₄ alkyl. In embodiments, R⁴ is halogen. In embodiments, R⁴ is —F. In embodiments, R⁴ is —Cl. In embodiments, R⁴ is —Br. In embodiments, R⁴ is —I. In embodiments, R⁴ is —CX⁴₃. In embodiments, R⁴ is —CF₃. In embodiments, R⁴ is —OCX⁴₃. In embodiments, R⁴ is —OCF₃. In embodiments, R⁴ is —CN. In embodiments, R⁴ is —OR^{4F}. In embodiments, R⁴ is —OH. In embodiments, R⁴ is —OCH₃. In embodiments, R⁴ is substituted or unsubstituted C₁-C₄ alkyl. In embodiments, R⁴ is unsubstituted C₁-C₄ alkyl. In embodiments, R⁴ is methyl. In embodiments, R⁴ is ethyl. In embodiments, R⁴ is —C(O)CH₃ In embodiments, R⁴ is

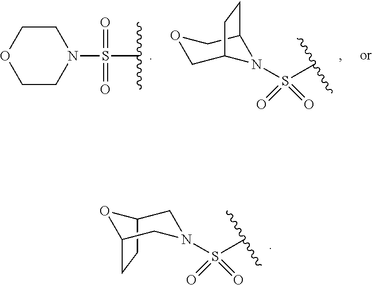

In embodiments, R¹ is halogen, substituted or unsubstituted C₁-C₄ alkyl, —CN, —CX¹₃, or OR^{1F}. In embodiments, each R^{1F} is independently hydrogen or unsubstituted C₁-C₄ alkyl. In embodiments, each R^{1F} is independently hydrogen, or unsubstituted methyl.

In embodiments, R¹ is halogen, substituted or unsubstituted C₁-C₄ alkyl, —CN, —CX¹₃, or OR^{1F}. In embodiments, R¹ is —F. In embodiments, R¹ is —Cl. In embodiments, R¹ is —Br. In embodiments, R¹ is —I. In embodiments, R¹ is substituted C₁-C₄ alkyl. In embodiments, R¹ is unsubstituted C₁-C₄ alkyl. In embodiments, R¹ is methyl. In embodiments, R¹ is ethyl. In embodiments, R¹ is —CN. In embodiments, R¹ is —CF₃. In embodiments, R¹ is —OH. In embodiments, R¹ is —OCH₃.

In embodiments, R³⁰ is halogen, substituted or unsubstituted C₁-C₄ alkyl, —CN, —CX³⁰₃, or OR^{30F}. In embodiments, each R^{30F} is independently hydrogen or unsubstituted C₁-C₄ alkyl. In embodiments, each R^{30F} is independently hydrogen, or unsubstituted methyl.

In embodiments, R³⁰ is halogen, substituted or unsubstituted C₁-C₄ alkyl, —CN, —CX¹₃, or OR^{1F}. In embodiments, R³⁰ is —F. In embodiments, R³⁰ is —Cl. In embodiments, R³⁰ is —Br. In embodiments, R³⁰ is —I. In embodiments, R³⁰ is substituted C₁-C₄ alkyl. In embodiments, R³⁰ is unsubstituted C₁-C₄ alkyl. In embodiments, R³⁰ is methyl. In embodiments, R³⁰ is ethyl. In embodiments, R³⁰ is —CN. In embodiments, R³⁰ is —CF₃. In embodiments, R³⁰ is —OH. In embodiments, R³⁰ is —OCH₃.

In embodiments, R¹ is substituted or unsubstituted C₁-C₄ alkyl and R^{10A} is substituted or unsubstituted C₁-C₄ alkyl. In embodiments, R¹ is methyl and R^{10A} is methyl. In embodiments, R¹ is substituted or unsubstituted C₁-C₄ alkyl and R^{10B} is substituted or unsubstituted C₁-C₄ alkyl. In embodiments, R¹ is methyl and R^{10B} is methyl.

Exemplary compounds of Formula (XIII) are shown in Table 3 below.

TABLE 3

| | |
|---|---|
| 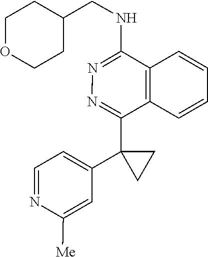 | |
| 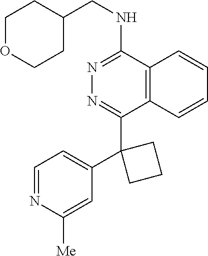 [SMILES: Cc1cc(C2(c3nnc(NCC4CCOCC4)c4ccccc34)CCC2)ccn1] | |
| 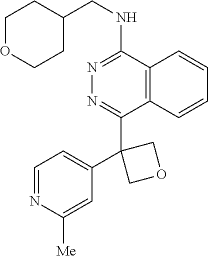 [SMILES: Cc1cc(C2(c3nnc(NCC4CCOCC4)c4ccccc34)COC2)ccn1] | |
| 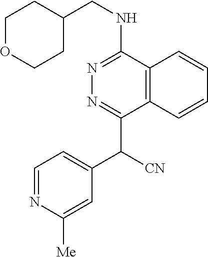 [SMILES: Cc1cc(C(C#N)c2nnc(NCC3CCOCC3)c3ccccc23)ccn1] | |
| 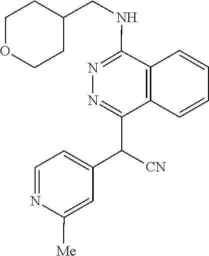 | |
| 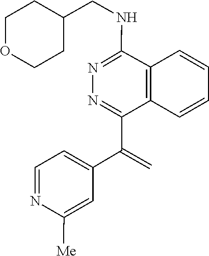 | |
| 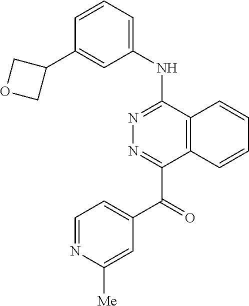 [SMILES: Cc1cc(C(=O)c2nnc(Nc3cccc(C4COC4)c3)c3ccccc23)ccn1] | |
| 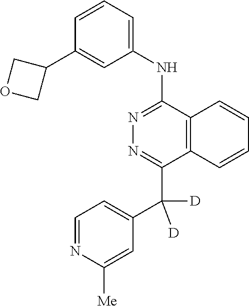 [SMILES: [2H]C([2H])(c1ccnc(C)c1)c1nnc(Nc2cccc(C3COC3)c2)c2ccccc12] | |
| 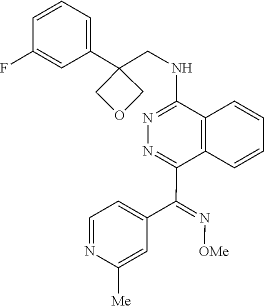 [SMILES: CO/N=C(\c1ccnc(C)c1)c1nnc(NCC2(c3cccc(F)c3)COC2)c2ccccc12] | |
| 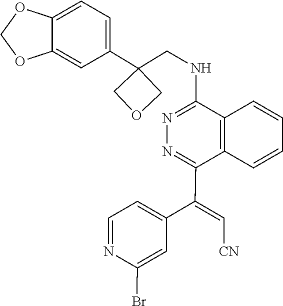 [SMILES: N#C/C=C(\c1ccnc(Br)c1)c1nnc(NCC2(c3ccc4c(c3)OCO4)COC2)c2ccccc12] | |
| 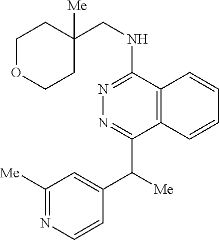 [SMILES: Cc1cc(C(C)c2nnc(NCC3(C)CCOCC3)c3ccccc23)ccn1] | SR-36584 |
| 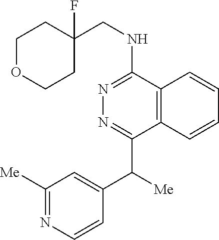 [SMILES: Cc1cc(C(C)c2nnc(NCC3(F)CCOCC3)c3ccccc23)ccn1] | SR-36585 |
| 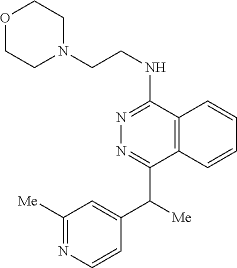 [SMILES: Cc1cc(C(C)c2nnc(NCCN3CCOCC3)c3ccccc23)ccn1] | SR-36586 |
| 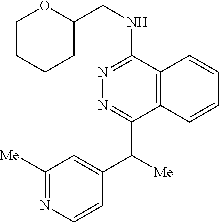 [SMILES: Cc1cc(C(C)c2nnc(NCC3CCCCO3)c3ccccc23)ccn1] | SR-36587 |
| 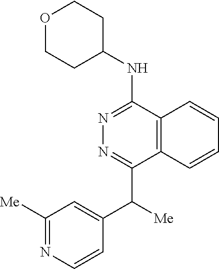 [SMILES: Cc1cc(C(C)c2nnc(NC3CCOCC3)c3ccccc23)ccn1] | SR-36588 |
| 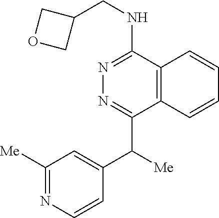 [SMILES: Cc1cc(C(C)c2nnc(NCC3COC3)c3ccccc23)ccn1] | SR-36589 |
| 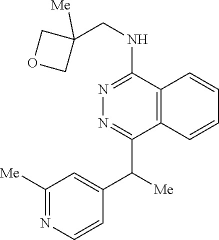 [SMILES: Cc1cc(C(C)c2nnc(NCC3(C)COC3)c3ccccc23)ccn1] | SR-36590 |
| 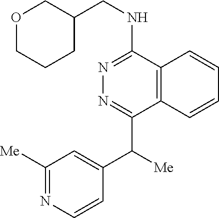 [SMILES: Cc1cc(C(C)c2nnc(NCC3CCCOC3)c3ccccc23)ccn1] | SR-36591 |
| 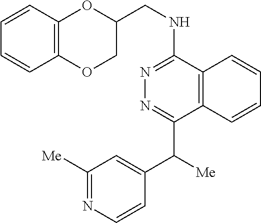 [SMILES: Cc1cc(C(C)c2nnc(NCC3COc4ccccc4O3)c3ccccc23)ccn1] | SR-36592 |
| 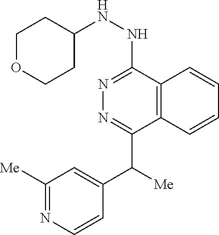 [SMILES: Cc1cc(C(C)c2nnc(NNC3CCOCC3)c3ccccc23)ccn1] | SR-36593 |
| 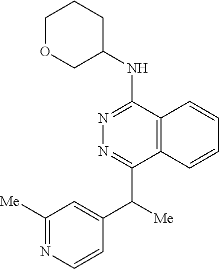 [SMILES: Cc1cc(C(C)c2nnc(NC3CCCOC3)c3ccccc23)ccn1] | SR-36595 |
| 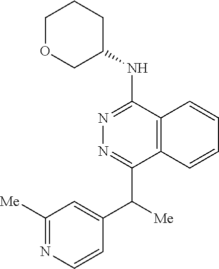 | SR-36595-1 |
| 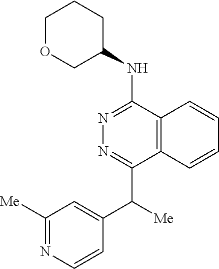 | SR-36595-2 |
| 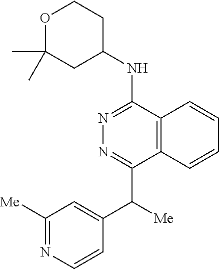 [SMILES: Cc1cc(C(C)c2nnc(NC3CCOC(C)(C)C3)c3ccccc23)ccn1] | SR-36596 |
| 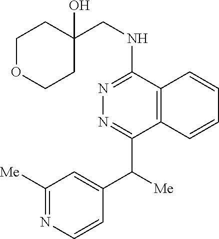 [SMILES: Cc1cc(C(C)c2nnc(NCC3(O)CCOCC3)c3ccccc23)ccn1] | SR-36597 |
| 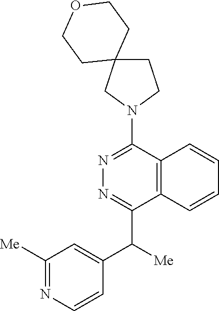 [SMILES: Cc1cc(C(C)c2nnc(N3CCC4(CCOCC4)C3)c3ccccc23)ccn1] | SR-36598 |
| 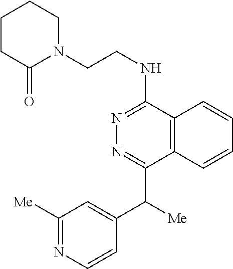 [SMILES: Cc1cc(C(C)c2nnc(NCCN3CCCCC3=O)c3ccccc23)ccn1] | SR-36599 |
| 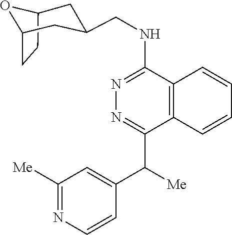 [SMILES: Cc1cc(C(C)c2nnc(NCC3CC4CCC(C3)O4)c3ccccc23)ccn1] | SR-36600 |
| 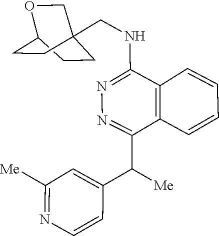 [SMILES: Cc1cc(C(C)c2nnc(NCC34CCC(CC3)OC4)c3ccccc23)ccn1] | SR-36601 |
| 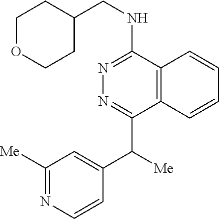 [SMILES: Cc1cc(C(C)c2nnc(NCC3CCOCC3)c3ccccc23)ccn1] | SR-35429 |
| 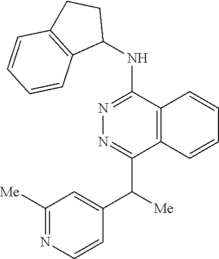 [SMILES: Cc1cc(C(C)c2nnc(NC3CCc4ccccc43)c3ccccc23)ccn1] | SR-35430 |
| 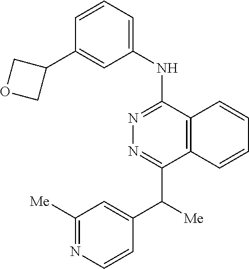 [SMILES: Cc1cc(C(C)c2nnc(Nc3cccc(C4COC4)c3)c3ccccc23)ccn1] | SR-35431 |
| 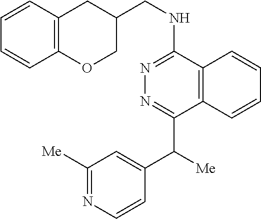 [SMILES: Cc1cc(C(C)c2nnc(NCC3COc4ccccc4C3)c3ccccc23)ccn1] | SR-35432 |
| 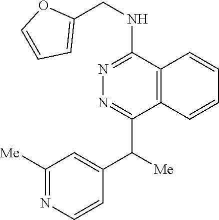 [SMILES: Cc1cc(C(C)c2nnc(NCc3ccco3)c3ccccc23)ccn1] | SR-35433 |
| 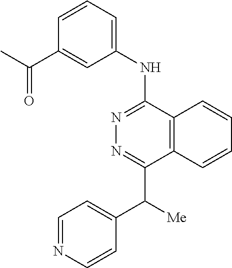 [SMILES: CC(=O)c1cccc(Nc2nnc(C(C)c3ccncc3)c3ccccc23)c1] | SR-34644 |
| 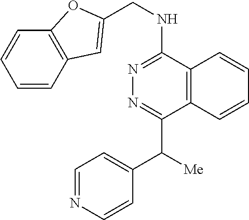 [SMILES: CC(c1ccncc1)c1nnc(NCc2cc3ccccc3o2)c2ccccc12] | SR-34645 |
| 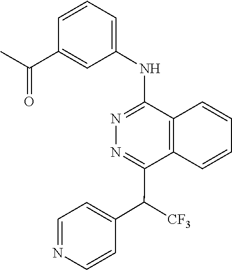 [SMILES: CC(=O)c1cccc(Nc2nnc(C(c3ccncc3)C(F)(F)F)c3ccccc23)c1] | SR-34646 |

### Pharmaceutical Compositions

In an aspect, provided is a pharmaceutical composition including the compound described herein, a pharmaceutically acceptable salt form thereof, an isomer thereof, or a crystal form thereof. Also provided herein are pharmaceutical formulations. In embodiments, the pharmaceutical formulation includes a compound (e.g. formulae (X), (XI), (XII), and (XIII) including all embodiments and subordinates thereof, or compounds in Tables 1-3 described above) and a pharmaceutically acceptable excipient.

The pharmaceutical composition may contain a dosage of the compound in a therapeutically effective amount.

In embodiments, the pharmaceutical composition includes any compound described above.

1. Formulations

The pharmaceutical composition may be prepared and administered in a wide variety of dosage formulations. Compounds described may be administered orally, rectally, or by injection (e.g. intravenously, intramuscularly, intracutaneously, subcutaneously, intraduodenally, or intraperitoneally).

For preparing pharmaceutical compositions from compounds described herein, pharmaceutically acceptable carriers can be either solid or liquid. Solid form preparations include powders, tablets, pills, capsules, cachets, suppositories, and dispersible granules. A solid carrier may be one or more substance that may also act as diluents, flavoring agents, binders, preservatives, tablet disintegrating agents, or an encapsulating material.

In powders, the carrier may be a finely divided solid in a mixture with the finely divided active component. In tablets, the active component may be mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight. Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The pharmaceutical compositions may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The pharmaceutical composition may be intended for intravenous use. The pharmaceutically acceptable excipient can include buffers to adjust the pH to a desirable range for intravenous use. Many buffers including salts of inorganic acids such as phosphate, borate, and sulfate are known.

2. Effective Dosages

The pharmaceutical composition may include compositions wherein the active ingredient is contained in a therapeutically effective amount, i.e., in an amount effective to achieve its intended purpose. The actual amount effective for a particular application will depend, inter alia, on the condition being treated.

The dosage and frequency (single or multiple doses) of compounds administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Therapeutically effective amounts for use in humans may be determined from animal models. For example, a dose for humans can be formulated to achieve a concentration that has been found to be effective in animals. The dosage in humans can be adjusted by monitoring response of the constipation or dry eye to the treatment and adjusting the dosage upwards or downwards, as described above.

Dosages may be varied depending upon the requirements of the subject and the compound being employed. The dose administered to a subject, in the context of the pharmaceutical compositions presented herein, should be sufficient to effect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

3. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual physician in view of the patient's condition and the particular method in which the compound is used.

When parenteral application is needed or desired, particularly suitable admixtures for the compounds included in the pharmaceutical composition may be injectable, sterile solutions, oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. Pharmaceutical admixtures suitable for use in the pharmaceutical compositions presented herein may include those described, for example, in Pharmaceutical Sciences (17th Ed., Mack Pub. Co., Easton, PA) and WO 96/05309, the teachings of both of which are hereby incorporated by reference.

### Methods

In an aspect, provided is a method for inhibiting NAD consumption and/or increasing NAD synthesis in a patient, and the method includes administering to the patient an effective dose of a compound (e.g. formulae (X), (XI), (XII), and (XIII) including all embodiments and subordinates thereof, or compounds in Tables 1-3 described above) and a pharmaceutically acceptable excipient.

The compound can inhibit NAD consuming reactions such as protein ADP-ribosylation reactions. The compound can inhibit NAD cleavage by protein deacetylases or NAD hydrolases. The compound can increase NAD synthesis. The compound can activate enzymes of the NAD synthetic pathways such as the rate-limiting enzyme for NAD synthesis in the salvage pathway called NAMPT. The patient is afflicted with, or at risk for, a protein misfolding neurodegenerative disease, another protein misfolding disease, another degenerative or metabolic disease.

The protein misfolding neurodegenerative disease includes a prion disease, Parkinson's disease, dementia with Lewy Bodies, multiple system atrophy or other synucleinopathies, Alzheimer's disease, amyotrophic lateral sclerosis, fronto-temporal dementia or other tauopathy, chronic traumatic encephalopathy, and the protein misfolding disease includes diabetes mellitus and amyloidoses.

In an aspect, provided is a method for preventing or inhibiting NAD depletion in a patient. In another aspect, provided is a method for increasing NAD levels to improve cellular function. In another aspect, provided is a method for improving a condition linked to alterations of NAD metabolism in a patient. The method includes administering to the patient an effective dose of the compound described herein.

The condition includes a metabolic disorder, a liver disorder, aging, a degenerative disease, a neurodegenerative disease, neuronal degeneration associated with multiple sclerosis, hearing loss, retinal damage or multiple sclerosis, macular degeneration, brain or cardiac ischemia, kidney failure, kidney disease, traumatic brain injury, or an axonopathy.

In an aspect, provided is a method for providing protection from toxicity of misfolded proteins in a patient. The method includes administering to the patient an effective dose of the compound described herein. The patient is afflicted with a prion disease, Parkinson's disease or other synucleinopathy, Alzheimer's disease, amyotrophic lateral sclerosis, a tauopathy, an amyloidosis or diabetes mellitus.

In an aspect, provided is a method for preventing or treating a protein misfolding neurodegenerative disease in a patient. The method includes administering to the patient an effective dose of the compound described herein.

In embodiments, the protein misfolding neurodegenerative disease is a disorder associated with protein aggregate-induced neurodegeneration and NAD depletion. In embodiments, the protein misfolding neurodegenerative disease includes a prion disease, Parkinson's disease, dementia with Lewy Bodies, multiple system atrophy or other synucleinopathy, Alzheimer's disease, amyotrophic lateral sclerosis, fronto-temporal dementia or other tauopathy, chronic traumatic encephalopathy. In embodiments, the neurodegenerative disease is multiple sclerosis, brain ischemia or an axonopathy.

In embodiments, the metabolic disorder includes diabetes or a liver disorder.

In embodiments, the condition linked to alterations of NAD metabolism includes aging, a retinal disease, a mitochondrial disease or a kidney disease.

In an aspect, provided is a method of preventing or treating a retinal disease in a patient. The method includes administering to the patient an effective dose of the compound described herein.

In an aspect, provided is a method of preventing or treating diabetes, non alcoholic fatty liver disease or other metabolic disease in a patient, comprising administering to the patient an effective dose of the compound described herein.

In an aspect, provided is a method of preventing or treating a kidney disease in a patient, comprising administering to the patient an effective dose of the compound described herein.

In an aspect, provided is a method of mitigating health effects of aging, comprising administering to the patient an effective dose of the compound described herein.

### EXAMPLES

### Example 1: Cell Viability Assays

The table below show the structures of specific examples of compounds useful for practice of methods of the invention, associated with corresponding data such as compound identifier, and biological results.

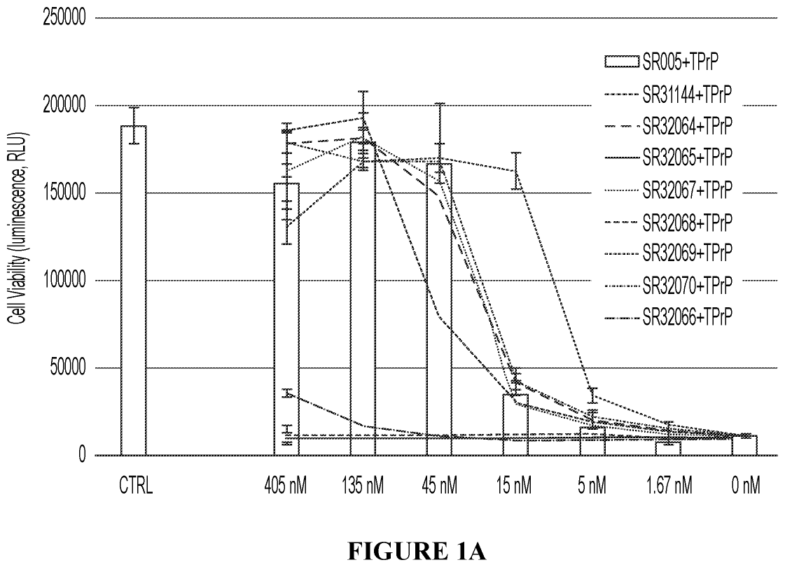

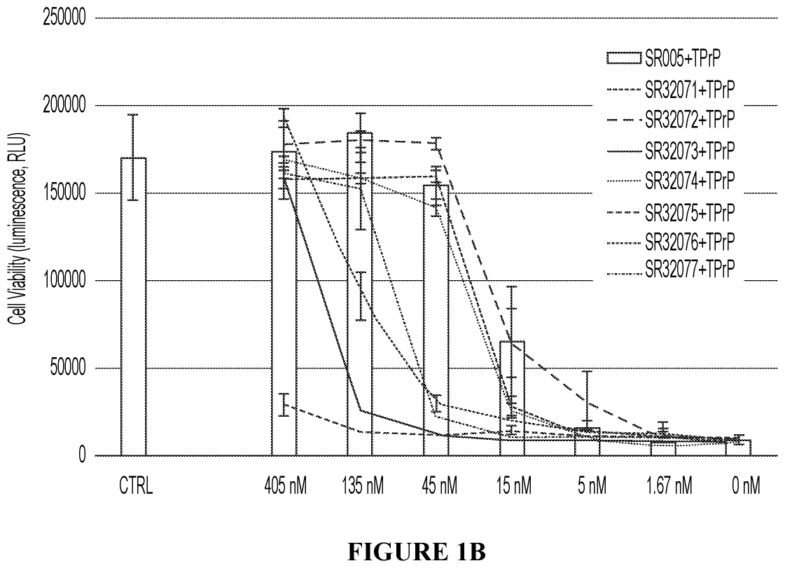

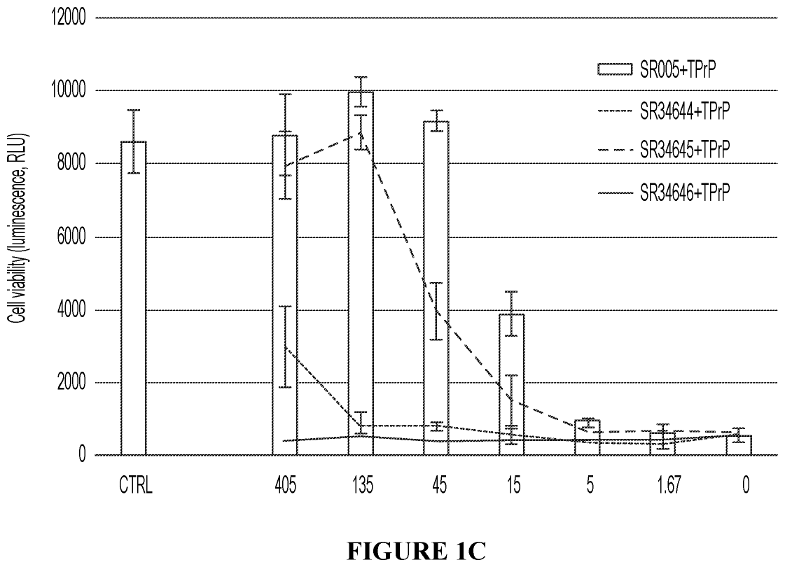

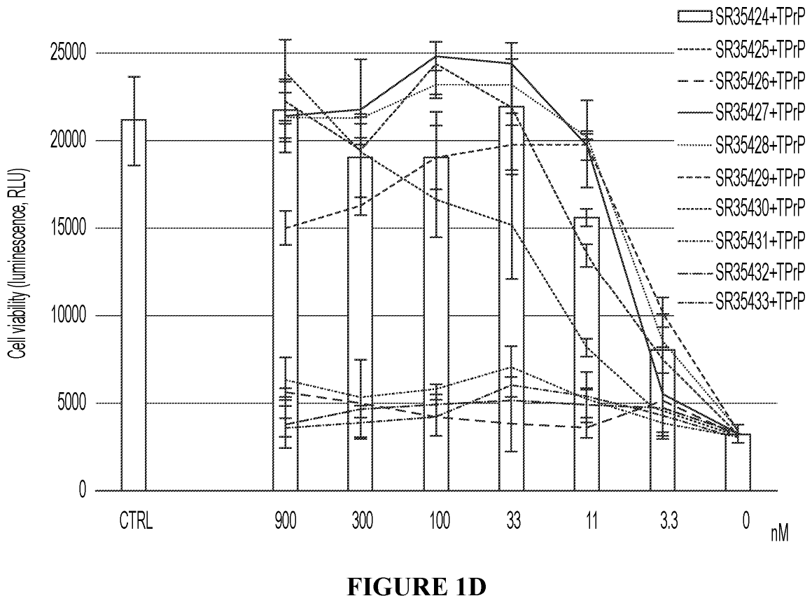

The biological activity of test compounds was quantified in a cell viability assay (CellTiter-Glo®) assessing the ability of compounds to prevent neuronal death due to NAD deprivation induced by the misfolded protein TPrP. Dose-response profiles were established in the TPrP neuroprotection assay for each compound. PK1 neuroblastoma cells (˜1000 cells/well, 96-well plates) were exposed to TPrP at 5 μg/ml and to compounds at doses ranging 1.67 nM to 405 nM or 900 nM for 4 days. TPrP was prepared as described in Zhou, et. al., Proc Natl Acad Sci USA 109, 3113-3118 (2012)¹. Compounds were added at the doses indicated in 0.5% DMSO final concentration. Cell viability was measured using CellTiter-Glo® (Promega). Efficacious concentrations (EC₅₀ values) were determined. TPrP EC₅₀ for the compounds described herein are shown in Table 4. Dose-response activity curves are shown in FIG. 1.

### Example 2: Microsomal Stability Assays

The metabolic stability of some test compounds was determined in hepatic human and mouse microsomes. The compound was incubated with 1 mg/ml human or mouse hepatic microsomes at 37° C. with continuous shaking. Aliquots were removed at various time points between 5 minutes and 2 hours and acetonitrile was added to quench the reactions and precipitate the proteins. Samples were then centrifuged through 0.45 μm filter plates and half-lives were determined by LC-MS/MS. Microsomal stability 215 minutes for tested compounds is shown in Table 4.

### Example 3: NAMPT Activation Assays

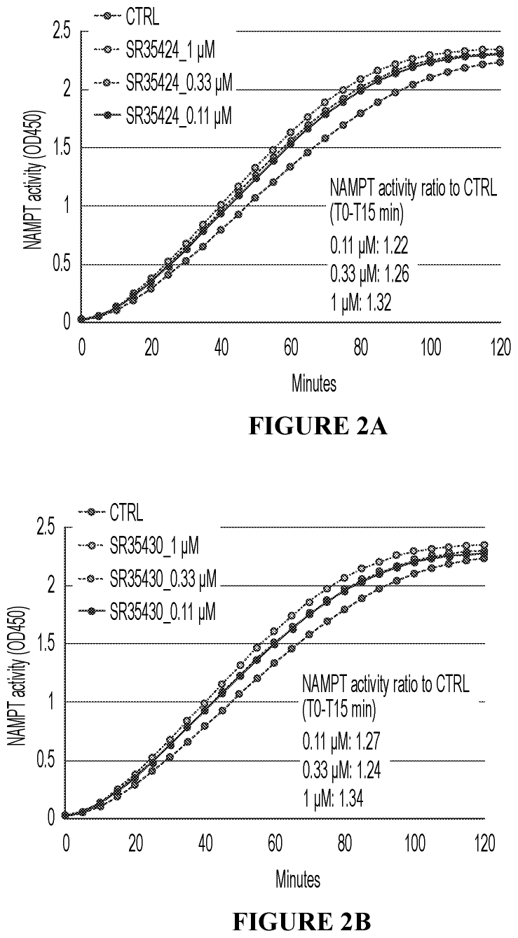

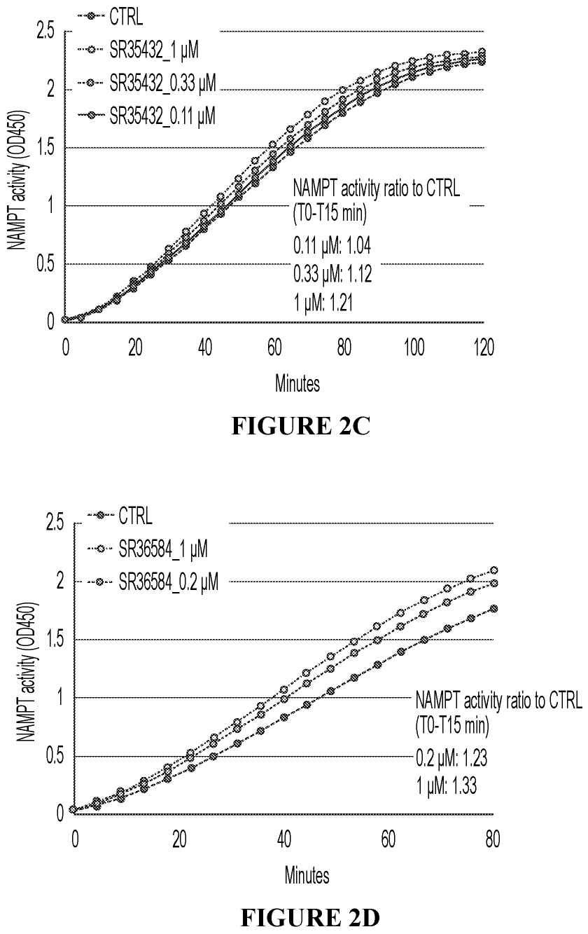

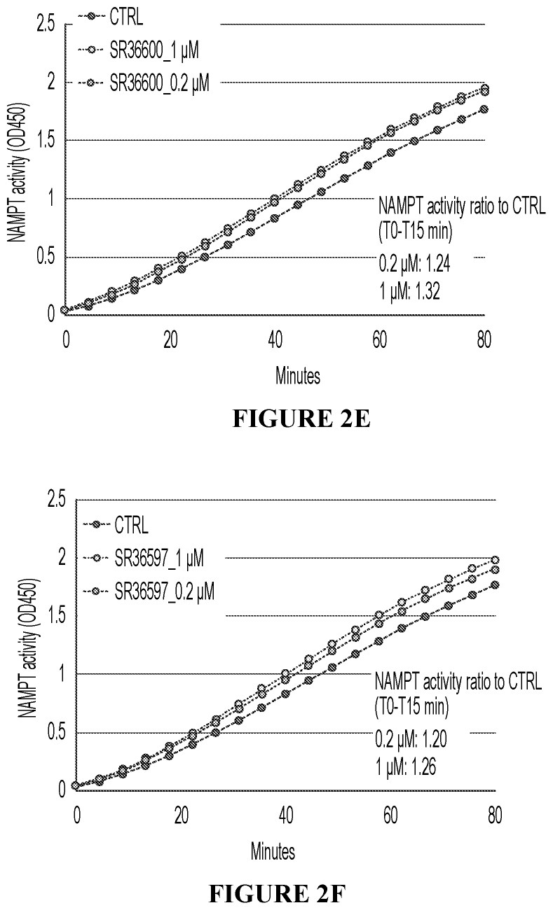

The ability of some test compounds to activate human NAMPT was tested in a colorimetric NAMPT activity assay (AbCam ab221819). The assay was performed according to the manufacturer's instructions. Enzymatic activity rate was calculated by the formula: ((A at T2)−(A at T1))/(T2−T1) where A is the OD450 at each time point T (min). Examples of activation curves are shown in FIG. 2. Activation ratios compared to baseline (CTRL, no compound) are also indicated in FIG. 2. NAMPT activation ≥10% at 1 μM compound for tested compounds is shown in Table 4.

TABLE 4

| | | | Human | |
|---|---|---|---|---|
| | | | NAMPT | |
| | | | activity | Microsomal |
| | | TPrP | ≥10% at | stab human |
| Compound | Structure | EC50 | 1 μM | ≥15 min |
| SR-36584 | 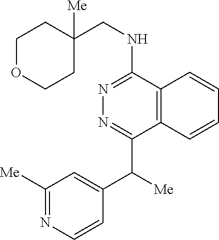 [SMILES: Cc1cc(C(C)c2nnc(NCC3(C)CCOCC3)c3ccccc23)ccn1] | | yes | |
| SR-36585 | 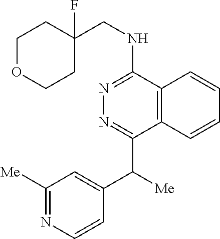 [SMILES: Cc1cc(C(C)c2nnc(NCC3(F)CCOCC3)c3ccccc23)ccn1] | | yes | yes |
| SR-36586 | 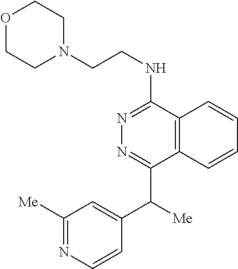 [SMILES: Cc1cc(C(C)c2nnc(NCCN3CCOCC3)c3ccccc23)ccn1] | | | yes |
| SR-36587 | 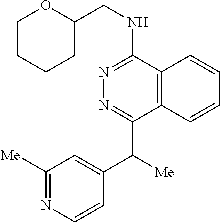 [SMILES: Cc1cc(C(C)c2nnc(NCC3CCCCO3)c3ccccc23)ccn1] | | | |
| SR-36588 | 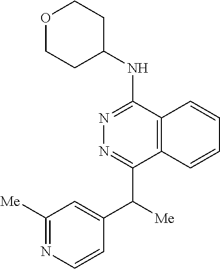 [SMILES: Cc1cc(C(C)c2nnc(NC3CCOCC3)c3ccccc23)ccn1] | | yes | yes |
| SR-36589 | 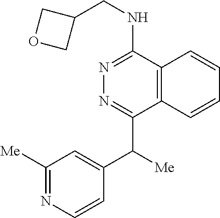 [SMILES: Cc1cc(C(C)c2nnc(NCC3COC3)c3ccccc23)ccn1] | | yes | |
| SR-36590 | 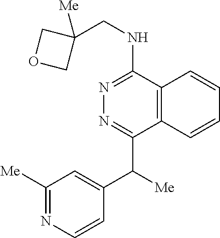 [SMILES: Cc1cc(C(C)c2nnc(NCC3(C)COC3)c3ccccc23)ccn1] | | yes | |
| SR-36591 | 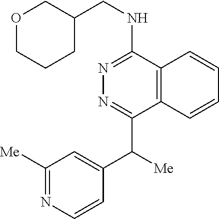 [SMILES: Cc1cc(C(C)c2nnc(NCC3CCCOC3)c3ccccc23)ccn1] | | | yes |
| SR-36592 | 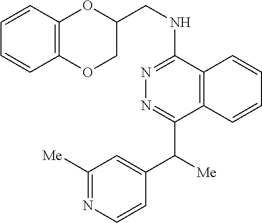 [SMILES: Cc1cc(C(C)c2nnc(NCC3COc4ccccc4O3)c3ccccc23)ccn1] | | | |
| SR-36593 | 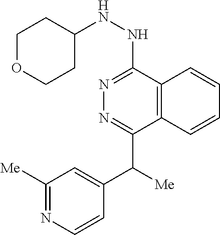 [SMILES: Cc1cc(C(C)c2nnc(NNC3CCOCC3)c3ccccc23)ccn1] | | | |
| SR-36595-1 | 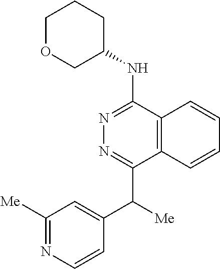 | | | yes |
| SR-36595-2 | 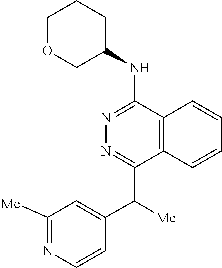 | | | yes |
| SR-36596 | 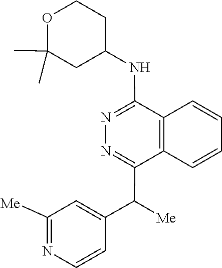 [SMILES: Cc1cc(C(C)c2nnc(NC3CCOC(C)(C)C3)c3ccccc23)ccn1] | | | yes |
| SR-36597 | 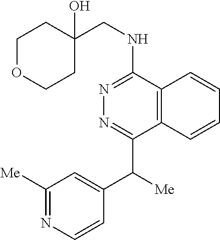 [SMILES: Cc1cc(C(C)c2nnc(NCC3(O)CCOCC3)c3ccccc23)ccn1] | | yes | yes |
| SR-36598 | 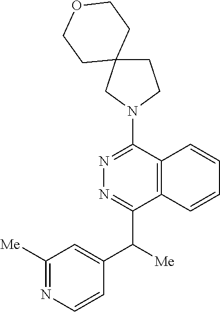 [SMILES: Cc1cc(C(C)c2nnc(N3CCC4(CCOCC4)C3)c3ccccc23)ccn1] | | | yes |
| SR-36599 | 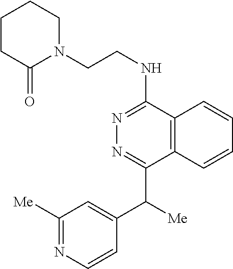 [SMILES: Cc1cc(C(C)c2nnc(NCCN3CCCCC3=O)c3ccccc23)ccn1] | | yes | |
| SR-36600 | 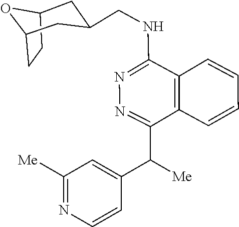 [SMILES: Cc1cc(C(C)c2nnc(NCC3CC4CCC(C3)O4)c3ccccc23)ccn1] | | yes | |
| SR-36601 | 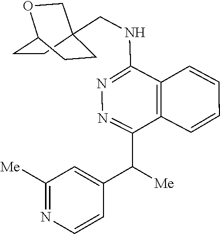 [SMILES: Cc1cc(C(C)c2nnc(NCC34CCC(CC3)OC4)c3ccccc23)ccn1] | | | |
| SR-35424 | 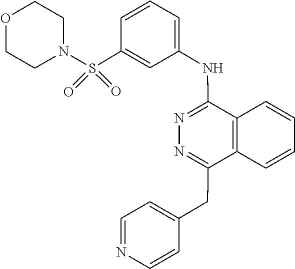 [SMILES: O=S(=O)(c1cccc(Nc2nnc(Cc3ccncc3)c3ccccc23)c1)N1CCOCC1] | 9 nM | yes | |
| SR-35425 | 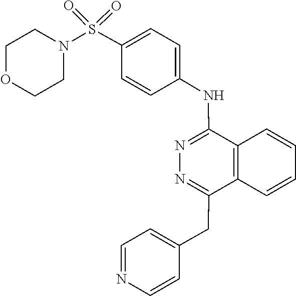 [SMILES: O=S(=O)(c1ccc(Nc2nnc(Cc3ccncc3)c3ccccc23)cc1)N1CCOCC1] | 5 nM | yes | |
| SR-35426 | 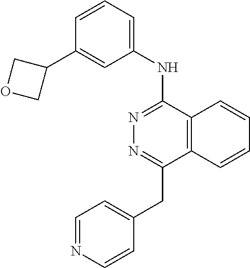 [SMILES: c1cc(Nc2nnc(Cc3ccncc3)c3ccccc23)cc(C2COC2)c1] | 10 nM | yes | |
| SR-35427 | 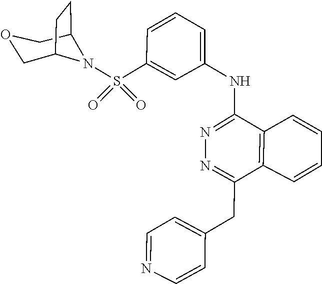 [SMILES: O=S(=O)(c1cccc(Nc2nnc(Cc3ccncc3)c3ccccc23)c1)N1C2CCC1COC2] | 7 nM | yes | |
| SR-35428 | 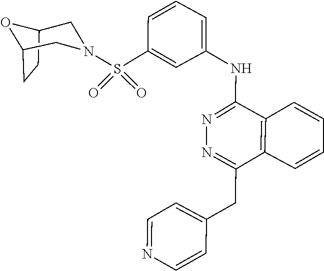 [SMILES: O=S(=O)(c1cccc(Nc2nnc(Cc3ccncc3)c3ccccc23)c1)N1CC2CCC(C1)O2] | 6 nM | yes | |
| SR-35429 | 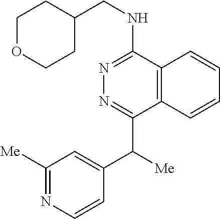 [SMILES: Cc1cc(C(C)c2nnc(NCC3CCOCC3)c3ccccc23)ccn1] | 26 nM | yes | yes |
| SR-35430 | 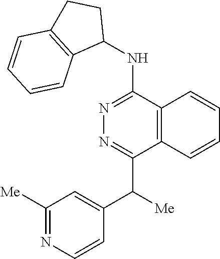 [SMILES: Cc1cc(C(C)c2nnc(NC3CCc4ccccc43)c3ccccc23)ccn1] | >900 nM | yes | yes |
| SR-35431 | 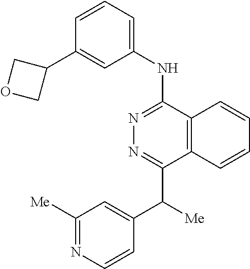 [SMILES: Cc1cc(C(C)c2nnc(Nc3cccc(C4COC4)c3)c3ccccc23)ccn1] | >900 nM | yes | |
| SR-35432 | 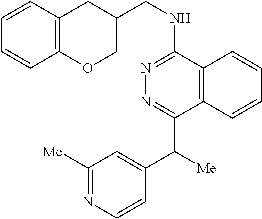 [SMILES: Cc1cc(C(C)c2nnc(NCC3COc4ccccc4C3)c3ccccc23)ccn1] | >900 nM | yes | |
| SR-35433 | 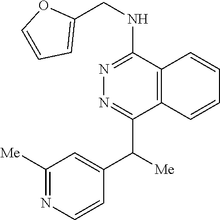 [SMILES: Cc1cc(C(C)c2nnc(NCc3ccco3)c3ccccc23)ccn1] | >900 nM | yes | yes |
| SR005 | 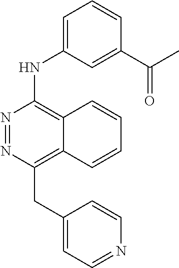 [SMILES: CC(=O)c1cccc(Nc2nnc(Cc3ccncc3)c3ccccc23)c1] | 15 nM, 20 nM 25 nM | | |
| SR-34779 | 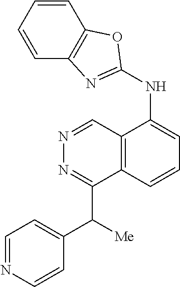 [SMILES: CC(c1ccncc1)c1nncc2c(Nc3nc4ccccc4o3)cccc12] | >500 nM | | |
| SR-34644 | 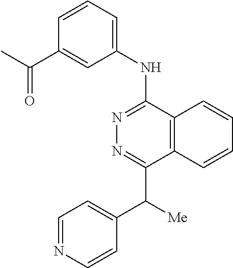 [SMILES: CC(=O)c1cccc(Nc2nnc(C(C)c3ccncc3)c3ccccc23)c1] | 45 nM | | |
| SR-34645 | 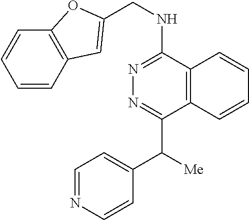 [SMILES: CC(c1ccncc1)c1nnc(NCc2cc3ccccc3o2)c2ccccc12] | ~500 nM | | |
| SR-34646 | 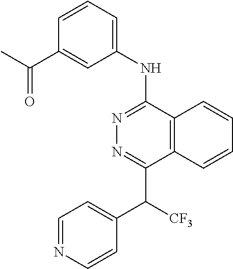 [SMILES: CC(=O)c1cccc(Nc2nnc(C(c3ccncc3)C(F)(F)F)c3ccccc23)c1] | >500 nM | | |
| vatalanib | 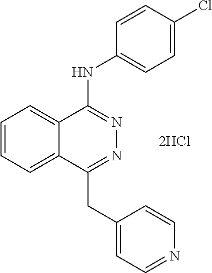 [SMILES: Clc1ccc(Nc2nnc(Cc3ccncc3)c3ccccc23)cc1] | 60 nM | | |
| SR-33871 | 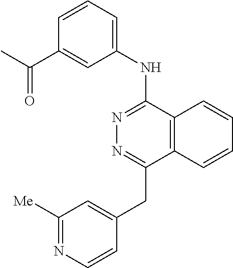 [SMILES: CC(=O)c1cccc(Nc2nnc(Cc3ccnc(C)c3)c3ccccc23)c1] | 70 nM | | yes |
| SR-33872 | 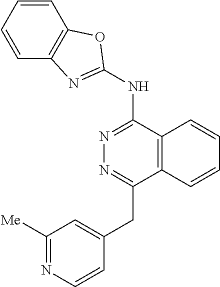 [SMILES: Cc1cc(Cc2nnc(Nc3nc4ccccc4o3)c3ccccc23)ccn1] | >500 nM | | yes |
| SR-33873 | 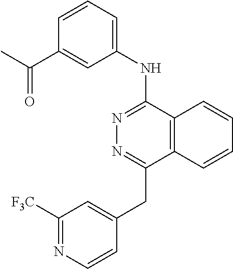 [SMILES: CC(=O)c1cccc(Nc2nnc(Cc3ccnc(C(F)(F)F)c3)c3ccccc23)c1] | >500 nM | | yes |
| SR-33874 | 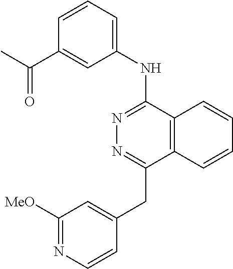 [SMILES: COc1cc(Cc2nnc(Nc3cccc(C(C)=O)c3)c3ccccc23)ccn1] | >500 nM | | yes |
| SR-33875 | 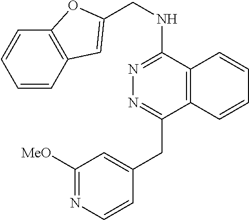 [SMILES: COc1cc(Cc2nnc(NCc3cc4ccccc4o3)c3ccccc23)ccn1] | >500 nM | | |
| SR-33876 | 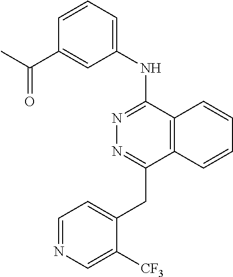 [SMILES: CC(=O)c1cccc(Nc2nnc(Cc3ccncc3C(F)(F)F)c3ccccc23)c1] | >500 nM | | |
| SR-32069 | 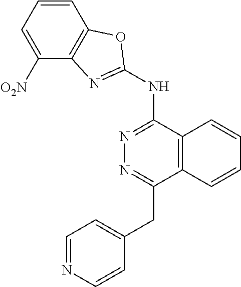 [SMILES: O=[N+]([O-])c1cccc2oc(Nc3nnc(Cc4ccncc4)c4ccccc34)nc12] | 8 nM (tox >135) | | |
| SR-32067 | 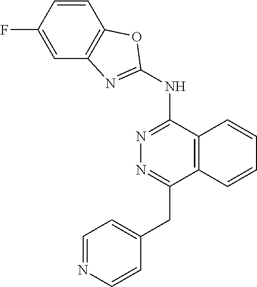 [SMILES: Fc1ccc2oc(Nc3nnc(Cc4ccncc4)c4ccccc34)nc2c1] | 20 nM | | |
| SR-32068 | 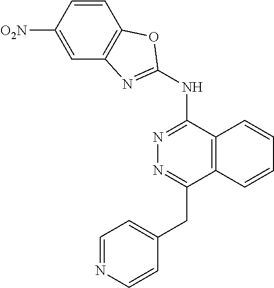 [SMILES: O=[N+]([O-])c1ccc2oc(Nc3nnc(Cc4ccncc4)c4ccccc34)nc2c1] | 20 nM | | |
| SR-32070 | 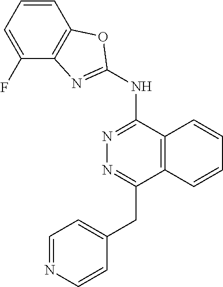 [SMILES: Fc1cccc2oc(Nc3nnc(Cc4ccncc4)c4ccccc34)nc12] | 20 nM | | |
| SR-32076 | 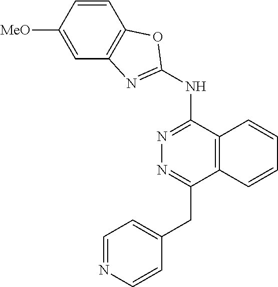 [SMILES: COc1ccc2oc(Nc3nnc(Cc4ccncc4)c4ccccc34)nc2c1] | 20 nM | | |
| SR-32072 | 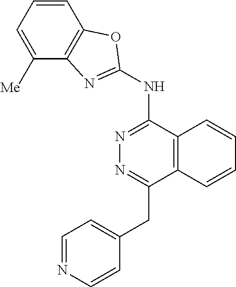 [SMILES: Cc1cccc2oc(Nc3nnc(Cc4ccncc4)c4ccccc34)nc12] | 30 nM | | |
| SR-32074 | 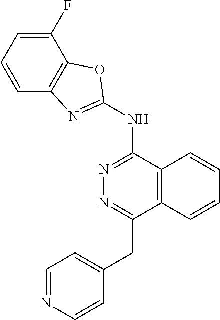 [SMILES: Fc1cccc2nc(Nc3nnc(Cc4ccncc4)c4ccccc34)oc12] | 30 nM | | |
| SR-32066 | 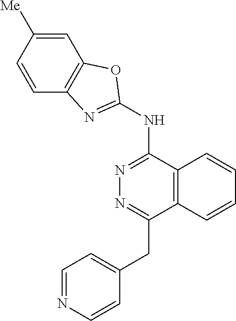 [SMILES: Cc1ccc2nc(Nc3nnc(Cc4ccncc4)c4ccccc34)oc2c1] | 50 nM | | |
| SR-32071 | 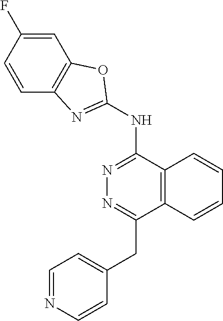 [SMILES: Fc1ccc2nc(Nc3nnc(Cc4ccncc4)c4ccccc34)oc2c1] | 90 nM | | |
| SR-32077 | 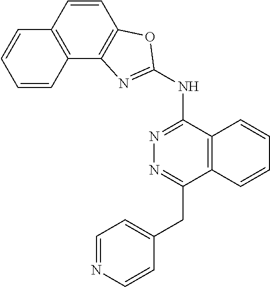 [SMILES: c1ccc2c(c1)ccc1oc(Nc3nnc(Cc4ccncc4)c4ccccc34)nc12] | 135 nM | | |
| SR-32073 | 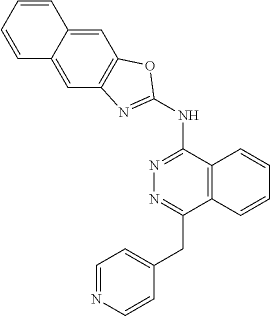 [SMILES: c1ccc2cc3oc(Nc4nnc(Cc5ccncc5)c5ccccc45)nc3cc2c1] | 250 nM | | |
| SR-31144 | 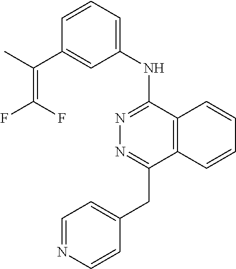 [SMILES: CC(=C(F)F)c1cccc(Nc2nnc(Cc3ccncc3)c3ccccc23)c1] | >400 nM | yes | yes |
| SR-32064 | 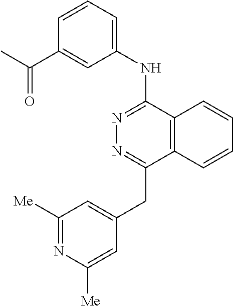 [SMILES: CC(=O)c1cccc(Nc2nnc(Cc3cc(C)nc(C)c3)c3ccccc23)c1] | >400 nM | | |
| SR-32065 | 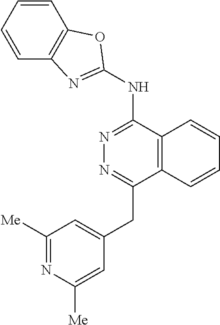 [SMILES: Cc1cc(Cc2nnc(Nc3nc4ccccc4o3)c3ccccc23)cc(C)n1] | >400 nM | | |
| SR-32075 | 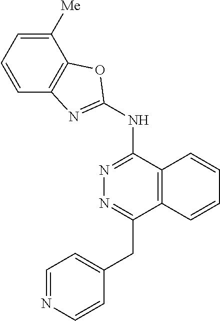 [SMILES: Cc1cccc2nc(Nc3nnc(Cc4ccncc4)c4ccccc34)oc12] | >400 nM | | |
| SR-30010 | 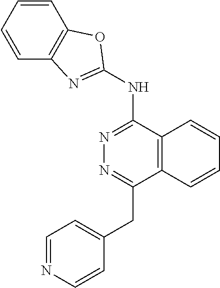 [SMILES: c1ccc2oc(Nc3nnc(Cc4ccncc4)c4ccccc34)nc2c1] | 2 nM | | yes |
| SR-30011 | 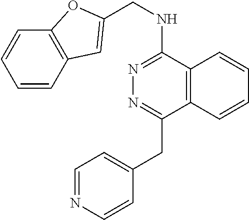 [SMILES: c1ccc2oc(CNc3nnc(Cc4ccncc4)c4ccccc34)cc2c1] | 2 nM | yes | |
| SR-29684 | 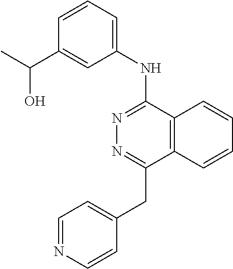 [SMILES: CC(O)c1cccc(Nc2nnc(Cc3ccncc3)c3ccccc23)c1] | 2 nM | | |
| SR-30012 | 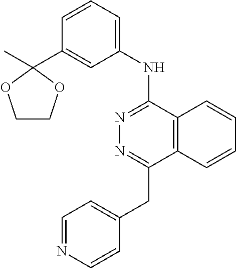 [SMILES: CC1(c2cccc(Nc3nnc(Cc4ccncc4)c4ccccc34)c2)OCCO1] | 2 nM | | |
| SR-30013 | 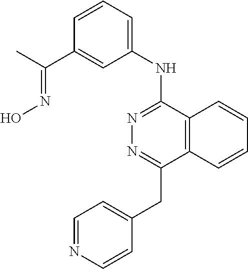 [SMILES: C/C(=N\O)c1cccc(Nc2nnc(Cc3ccncc3)c3ccccc23)c1] | 2 nM | | |
| SR-30014 | 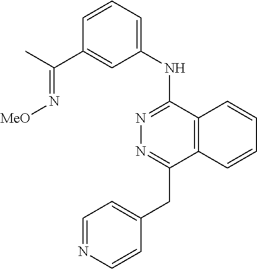 [SMILES: CO/N=C(\C)c1cccc(Nc2nnc(Cc3ccncc3)c3ccccc23)c1] | 2 nM | | |
| SR-30008 | 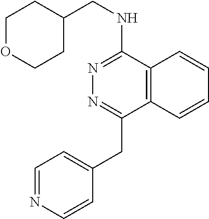 [SMILES: c1ccc2c(NCC3CCOCC3)nnc(Cc3ccncc3)c2c1] | 15 nM | yes | |
| SR-30024 | 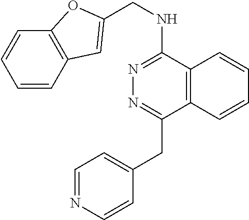 [SMILES: c1ccc2oc(CNc3nnc(Cc4ccncc4)c4ccccc34)cc2c1] | 20 nM | yes | |
| SR-30005 | 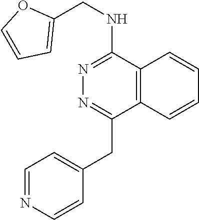 [SMILES: c1coc(CNc2nnc(Cc3ccncc3)c3ccccc23)c1] | 140 nM | yes | |
| SR-30006 | 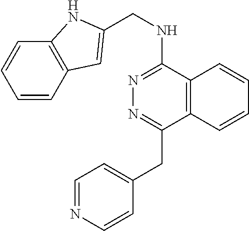 [SMILES: c1ccc2[nH]c(CNc3nnc(Cc4ccncc4)c4ccccc34)cc2c1] | 50 nM | | |
| SR-30004 | 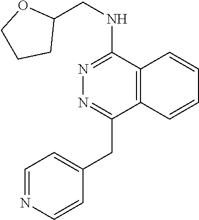 [SMILES: c1ccc2c(NCC3CCCO3)nnc(Cc3ccncc3)c2c1] | 150 nM | yes | |
| SR-30007 | 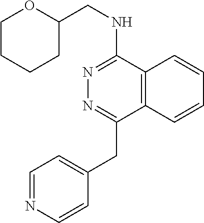 [SMILES: c1ccc2c(NCC3CCCCO3)nnc(Cc3ccncc3)c2c1] | 60 nM | yes | |
| SR-27888 | 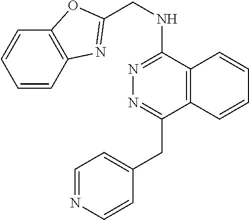 [SMILES: c1ccc2oc(CNc3nnc(Cc4ccncc4)c4ccccc34)nc2c1] | 45 nM | | |
| SR-27886 | 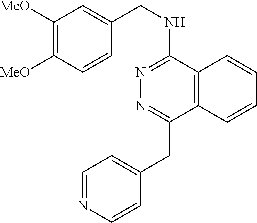 [SMILES: COc1ccc(CNc2nnc(Cc3ccncc3)c3ccccc23)cc1OC] | 150 nM | yes | |
| SR-27890 | 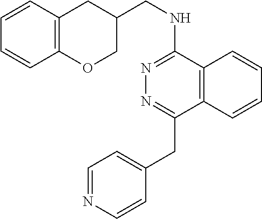 [SMILES: c1ccc2c(c1)CC(CNc1nnc(Cc3ccncc3)c3ccccc13)CO2] | 165 nM | yes | |
| SR-27891 | 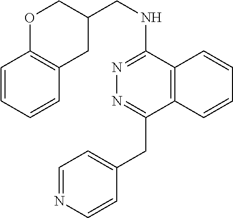 [SMILES: c1ccc2c(c1)CC(CNc1nnc(Cc3ccncc3)c3ccccc13)CO2] | 260 nM | yes | |
| SR-27887 | 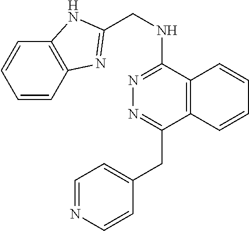 [SMILES: c1ccc2[nH]c(CNc3nnc(Cc4ccncc4)c4ccccc34)nc2c1] | 220 nM | | |
| SR-27889 | 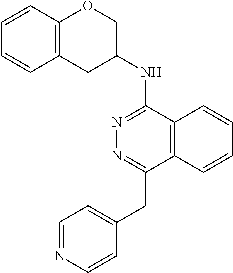 [SMILES: c1ccc2c(c1)CC(Nc1nnc(Cc3ccncc3)c3ccccc13)CO2] | 285 nM | | |
| SR-27885 | 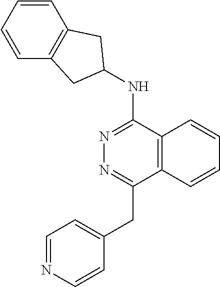 [SMILES: c1ccc2c(c1)CC(Nc1nnc(Cc3ccncc3)c3ccccc13)C2] | 200 nM | | |
| SR-27892 | 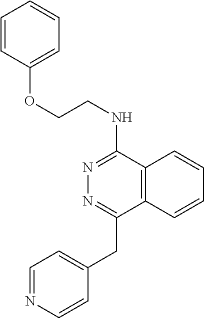 [SMILES: c1ccc(OCCNc2nnc(Cc3ccncc3)c3ccccc23)cc1] | 500 nM | | |
| SR-27884 | 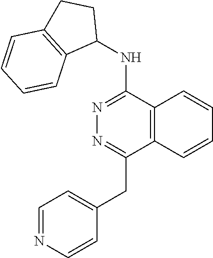 [SMILES: c1ccc2c(c1)CCC2Nc1nnc(Cc2ccncc2)c2ccccc12] | 500 nM | yes | |

### Example 4: Synthesis I

Schemes below show synthesis of examples of compounds useful for practice of the invention. These examples are provided for illustrative purposes only and not to limit the scope of the claims provided herein. The starting materials and reagents used for the synthesis of the compounds described herein may be synthesized or can be obtained from commercial sources.

### Scheme 1: Synthesis of Compound SR-32077

Compound SR-32077 can be synthesized via Buckwald-Hartwig coupling of 1-chloro-4-(pyridin-4-ylmethyl)phthalazines and 2-aminobenzoxazoles according to Scheme 1.

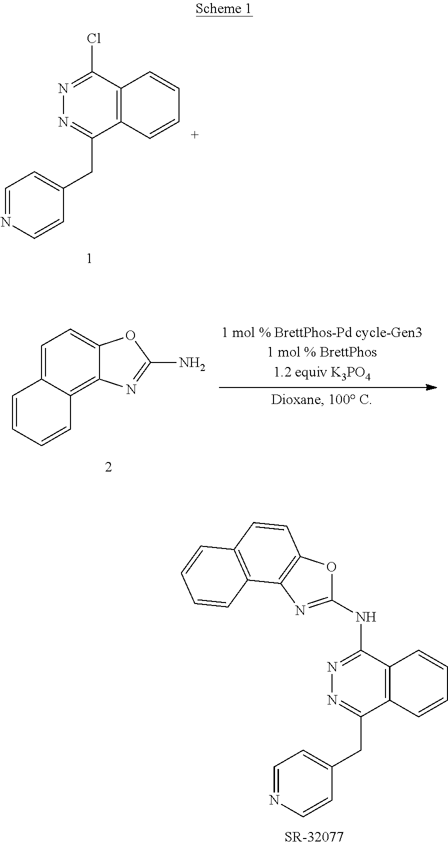 [SMILES: Clc1nnc(Cc2ccncc2)c2ccccc12 ; Nc1nc2c(ccc3ccccc32)o1 ; c1ccc2c(c1)ccc1oc(Nc3nnc(Cc4ccncc4)c4ccccc34)nc12]

Compound 1 in Scheme 1 can be synthesized according to Scheme 1-1 and Scheme 1-2.

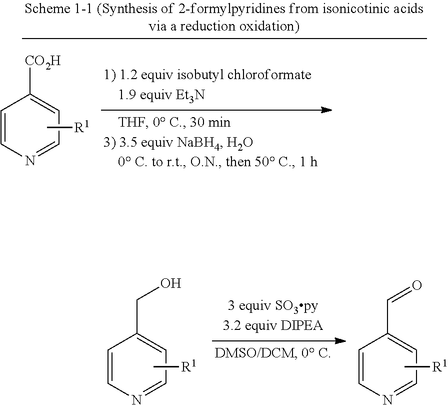

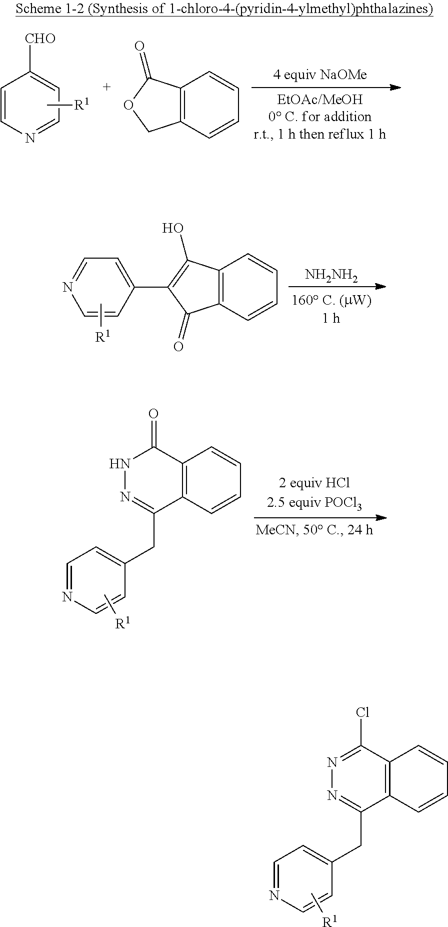

Compound 2 in Scheme 1 can be synthesized according to Scheme 1-3.

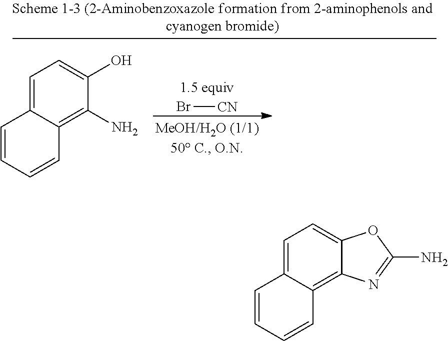

### Scheme 2: Synthesis of Compound SR-32073

Compound SR-32073 can be synthesized via Buckwald-Hartwig coupling of 1-chloro-4-(pyridin-4-ylmethyl)phthalazines and 2-aminobenzoxazoles according to Scheme 2.

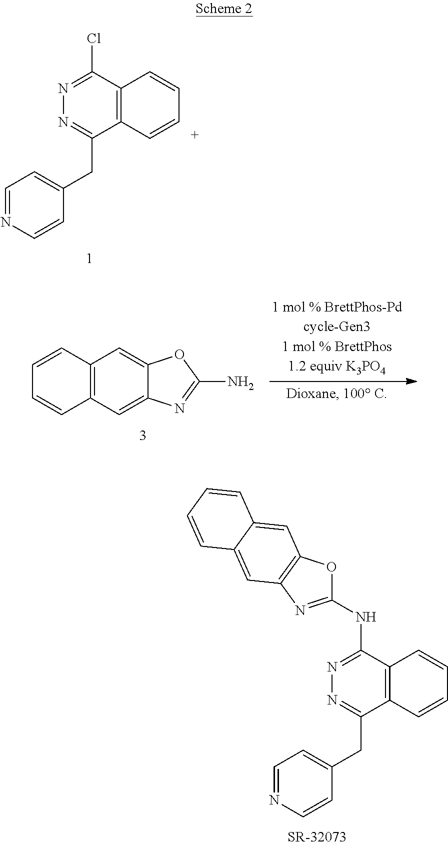 [SMILES: Clc1nnc(Cc2ccncc2)c2ccccc12 ; Nc1nc2cc3ccccc3cc2o1 ; c1ccc2cc3oc(Nc4nnc(Cc5ccncc5)c5ccccc45)nc3cc2c1]

Compound 1 in Scheme 2 can be synthesized according to Scheme 1-1 and Scheme 1-2 described above. Compound 3 can be synthesized according to Scheme 2-1.

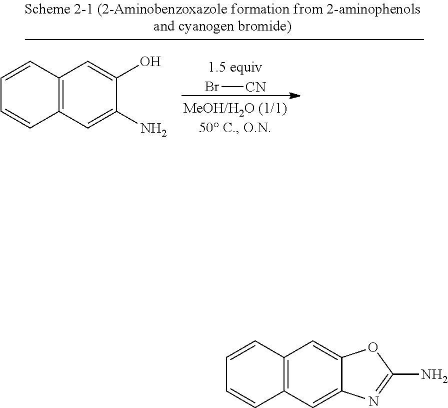

### Scheme 3

Compound of the present invention can be synthesized via Buckwald-Hartwig coupling of 1-chloro-4-(pyridin-4-ylmethyl)phthalazines and (3-aryloxetan-3-yl)methanamines according to Scheme 3.

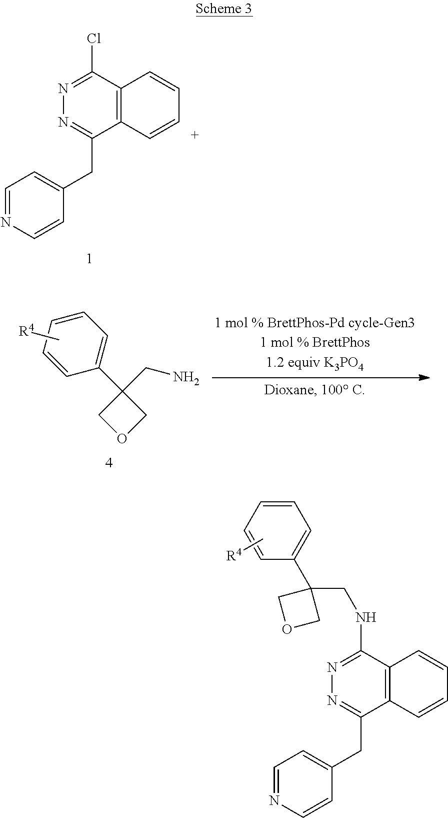

Compound 1 in Scheme 3 can be synthesized according to Scheme 1-1 and Scheme 1-2 described above. Compound 4 can be synthesized according to Scheme 3-1.

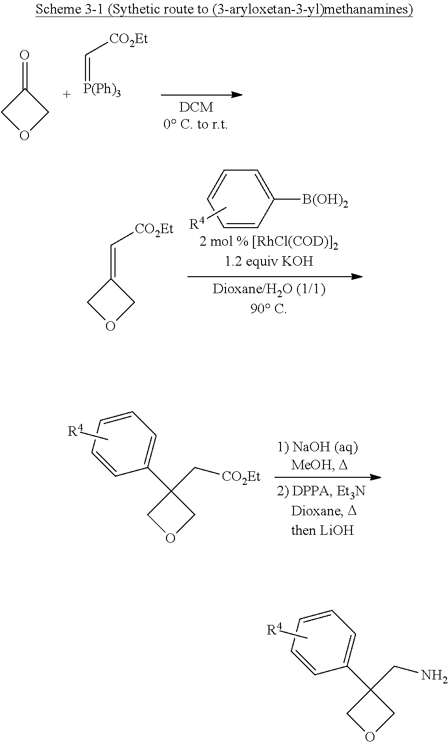

### Scheme 4

Compound of the present invention can be synthesized via Buckwald-Hartwig coupling of 1-chloro-4-(pyridin-4-ylmethyl)phthalazines and 3-aryloxetan-3-amines according to Scheme 4.

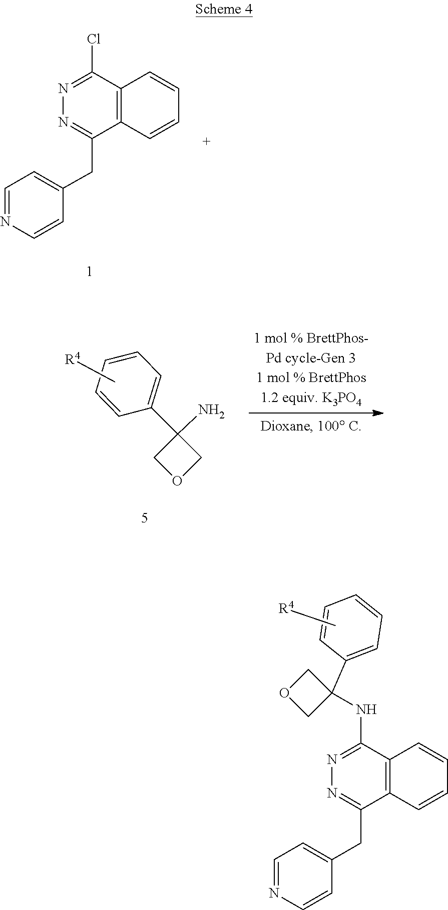

Compound 1 in Scheme 4 can be synthesized according to Scheme 1-1 and Scheme 1-2 described above. Compound 5 can be synthesized according to Scheme 4-1.

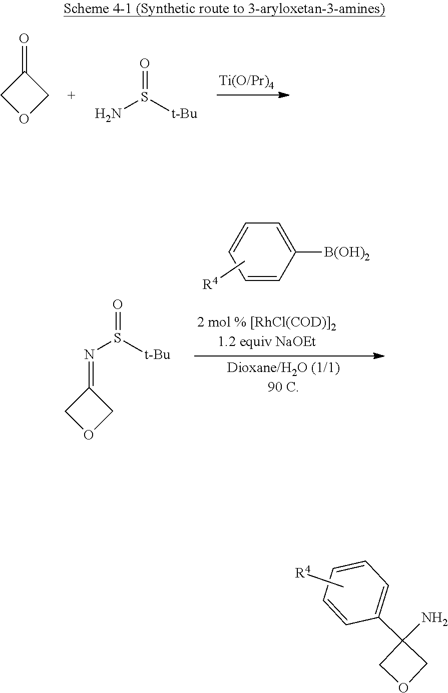

### Scheme 5

Compound of the present invention can be synthesized via Buckwald-Hartwig coupling of N-(3-(oxetan-3-yl)phenyl)-4-(pyridin-4-ylmethyl)phthalazin-1-amine according to Scheme 5.

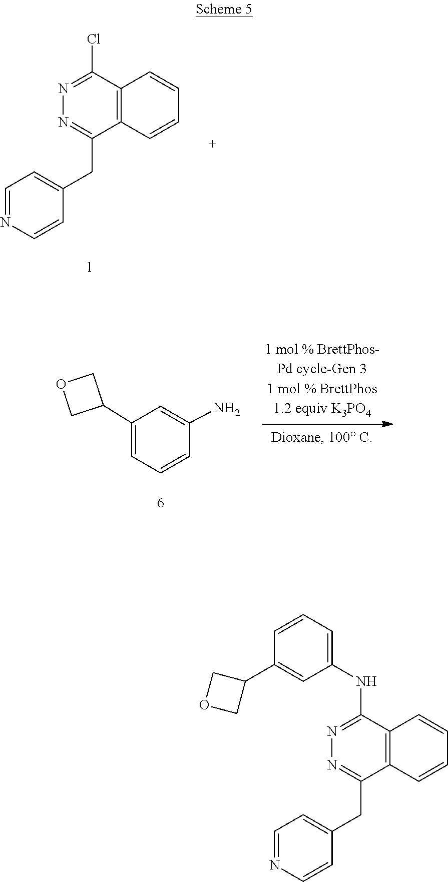 [SMILES: Clc1nnc(Cc2ccncc2)c2ccccc12 ; Nc1cccc(C2COC2)c1 ; c1cc(Nc2nnc(Cc3ccncc3)c3ccccc23)cc(C2COC2)c1]

Compound 1 in Scheme 5 can be synthesized according to Scheme 1-1 and Scheme 1-2 described above. Compound 6 can be synthesized according to Scheme 5-1.

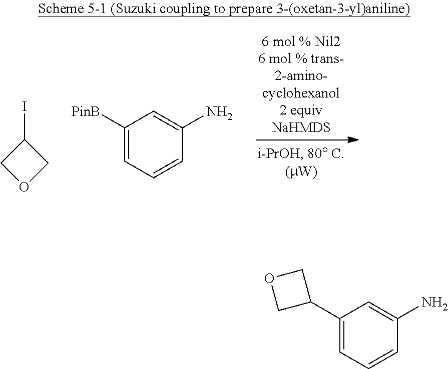

### Scheme 6: Alternative Scheme for Scheme 1-1 and Scheme 1-2

Compound 1 in Schemes 1-5 can be synthesized according to Scheme 6-1, or according Schemes 6-2 and 6-3.

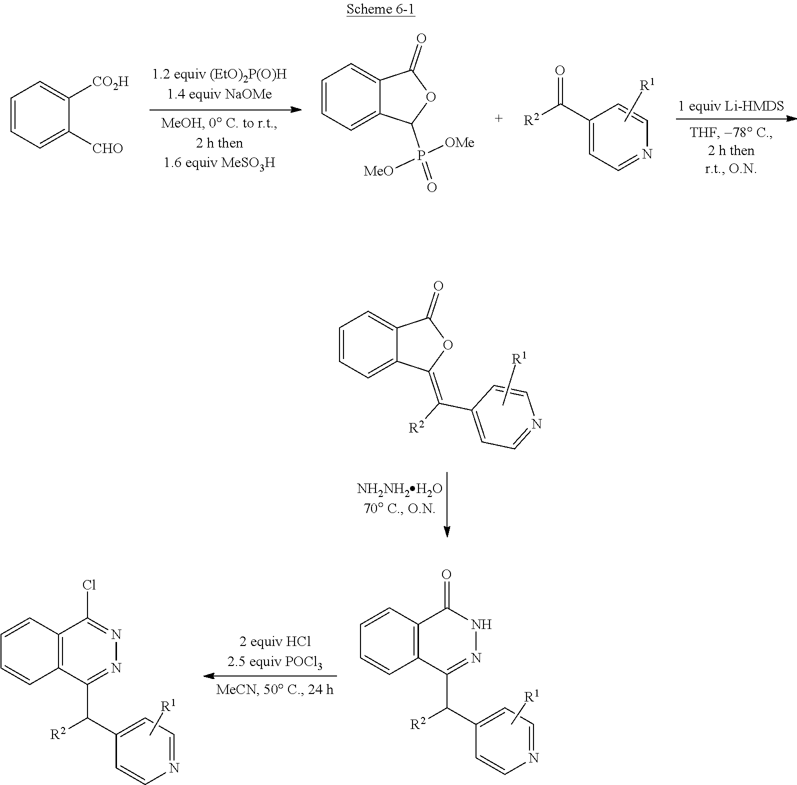

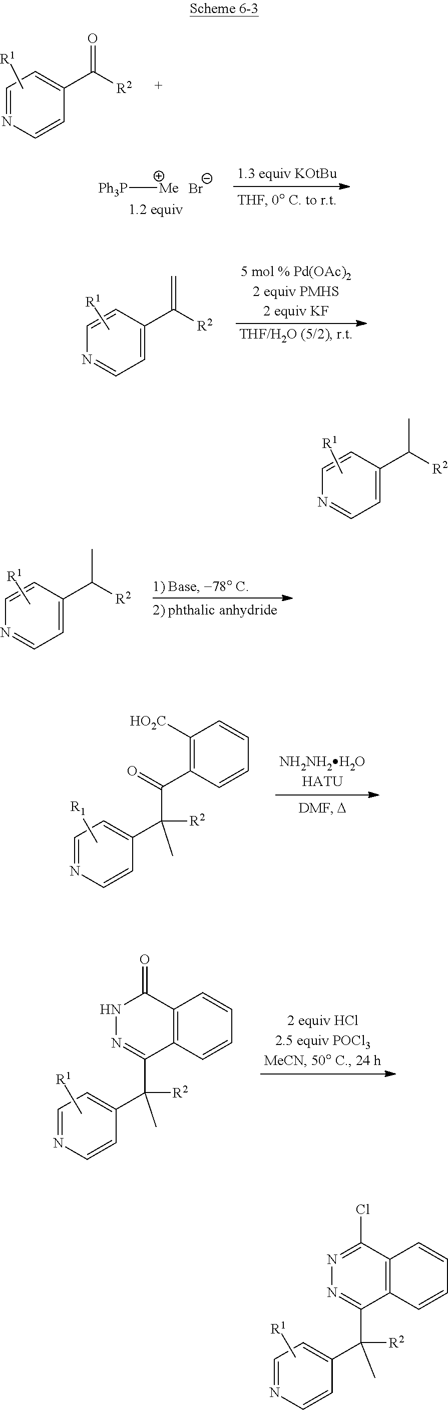

### Example 5: Synthesis H1

### Synthesis of Starting Material

### Starting Material 1

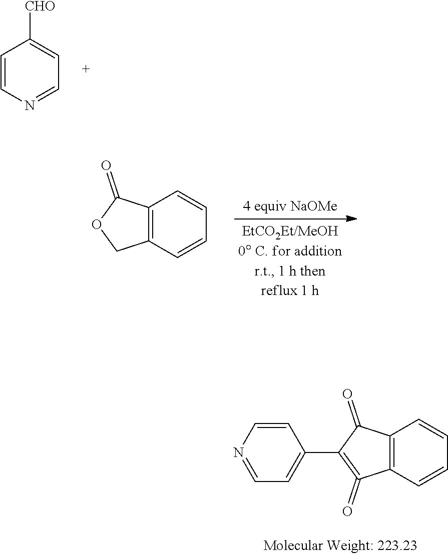

A solution of sodium methoxide (596 mmol, 32.2 g) in methanol (298 mL) in a 1 L R.B. was cooled to 0° C. with an ice-bath. An addition funnel was attached containing a solution of the 1-isobenzofuranone (149 mmol, 20.0 g), 4-pyridinecarboxaldehyde (156 mmol, 14.7 mL), and ethyl propionate (80 mL), which was added dropwise over 20 minutes. After compete addition the ice-bath was removed and the reaction was stirred at r.t for 1 hour. The addition funnel was replaced with a reflux condenser and the reaction was heated to reflux (100° C.) for 1.5 hour. The cooled reaction mixture was concentrated down to a thick slurry, diluted with water (˜700 mL), and transferred to a sep-funnel. The dark red solution was extracted with diethyl ether (3×200 mL). The aqueous layer in a 2 L Erlenmeyer flask was acidified with acetic acid and swirled producing a yellow precipitate. The precipitated solid was filtered, rinsed with water, ethyl acetate, and diethyl ether. The collected solid was placed in a vacuum desiccator and dried over night under high vacuum, affording 22.8 g (69%) of 2-[4(1H)-pyridinylidene]indan-1,3-dione as a bright yellow solid.

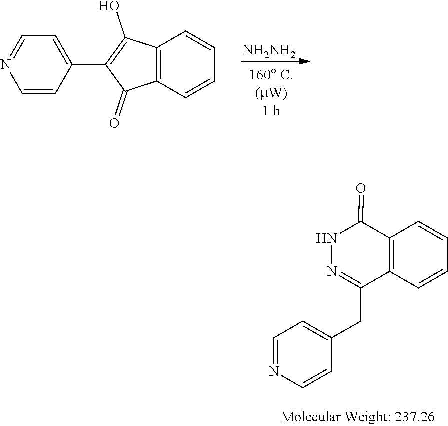

A 10-20 mL microwave vial was charged with 2-[4(1H)-pyridinylidene]indan-1,3-dione (11.2 mmol, 2.5 g) and hydrazine monohydrate (11 mL), sealed with a crimp cap and heated for 1 hour at 160° C. in a Biotage Microwave reactor. The vial was placed in an ice-bath for 10 minutes and the precipitated solid was filtered and rinsed with ethanol (20 mL) then diethyl ether (4×20 mL) affording 2.19 g (83%) of 4-(pyridin-4-ylmethyl)phthalazin-1(2H)-one as a light tan solid. ¹H NMR (400 MHz, d₆-DMSO) δ 12.62 (bs, 1H), 8.47 (dd, J=1.6 & 6.0 Hz, 2H), 8.27 (m, 1H), 7.94-7.82 (m, 3H), 7.32 (dd, J=1.6 & 6.0 Hz, 2H), 4.35 (s, 2H).

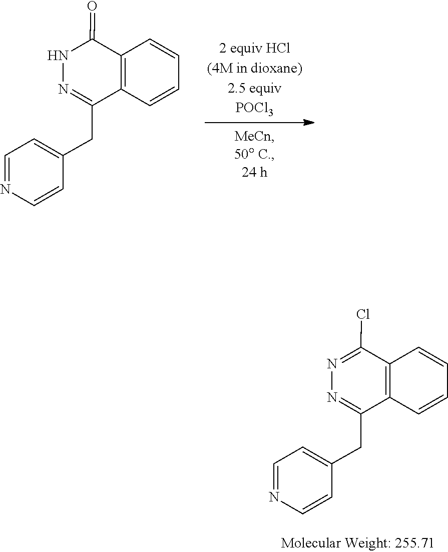 [SMILES: O=c1[nH]nc(Cc2ccncc2)c2ccccc12 ; Clc1nnc(Cc2ccncc2)c2ccccc12]

A flame dried 500 mL R.B. was charged with the phthalazin-1-one (23.2 mmol, 5.53 g) and placed under an atmosphere of argon. Freshly distilled dry acetonitrile (90 mL) and 4M HCl in dioxane (46.6 mmol, 11.65 mL) were injected, and the mixture was stirred for 10 minutes. To the semi-homogenous solution was added phosphoryl chloride (58.3 mmol, 5.43 mL). The R.B. was placed in oil bath and heated to 50° C. for 24 hours. The reaction was cooled to room temperature followed by placing the R.B. in an ice bath. To the cooled pale orange slurry was carefully and slowly added 268 mL of a 1 M NaHCO₃ (aq) solution. The cooled mixture was stirred for 30 minutes then filtered and rinsed with ether. The solid was then dried over night under high vacuum affording 4.95 g (83%) of 1-chloro-4-(pyridin-4-ylmethyl)phthalazine as a burnt orange solid. ¹H NMR (400 MHz, d₆-DMSO) δ 8.46 (d, J=5.0 Hz, 2H), 8.35 (m, 2H), 8.14 (m, 2H), 7.33 (d, J=5.0 Hz, 2H), 4.77 (s, 2H).

### Starting Material 2

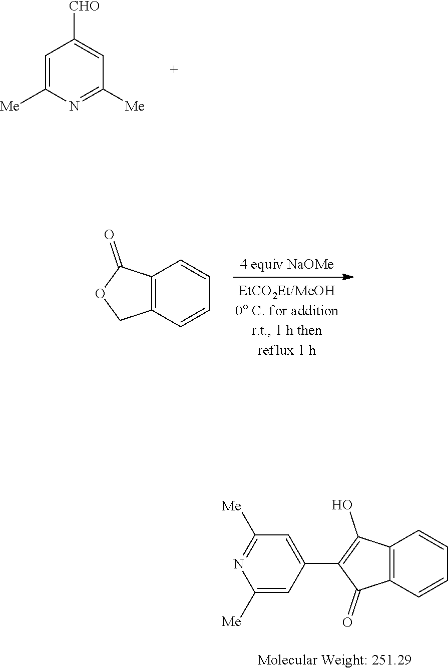 [SMILES: Cc1cc(C=O)cc(C)n1 ; O=C1OCc2ccccc21 ; Cc1cc(C2=C(O)c3ccccc3C2=O)cc(C)n1]

A solution of sodium methoxide (28.16 mmol, 1.52 g) in 14 mL of methanol in a 50 mL R.B. was cooled to 0° C. with an ice-bath. In a separate 20 mL scintillation vial was prepared a solution of 1-isobenzofuranone (7.04 mmol, 0.944 g), and 2,6-dimethylpyridine-4-carboxaldehyde (7.39 mmol, 1.00 g), in 3.8 mL of ethyl propionate, which was taken up into a syringe and added dropwise over 10 minutes to the methoxide solution. After compete addition the reaction was stirred at r.t for 1 hour. A reflux condenser was attached and the reaction was heated to reflux (100° C.) for 1.5 hour. The cooled reaction mixture was concentrated down to a thick slurry, diluted with water (30 mL), and transferred to a sep-funnel. The dark red solution was extracted with diethyl ether (3×15 mL). The aqueous layer in a 250 mL Erlenmeyer flask was acidified with acetic acid producing a yellow precipitate. The precipitated solid was filtered, rinsed with water, ethyl acetate, and diethyl ether. The collected solid was dried overnight under high vacuum, affording 1.149 g (65%) of 2-(2,6-dimethylpyridin-4-yl)-3-hydroxy-1H-inden-1-one as a bright yellow solid. ¹H NMR (400 MHz, d₆-DMSO) δ 12.94 (bs, 1H), 8.45 (s, 2H), 7.51 (m, 2H), 7.45 (m, 2H), 2.46 (s, 6H)

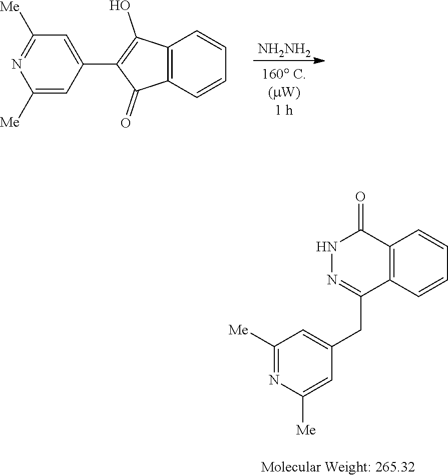

A 5 mL microwave vial was charged with 2-(2,6-dimethylpyridin-4-yl)-3-hydroxy-1H-inden-1-one (4.57 mmol, 1.149 g) and 4 mL of hydrazine monohydrate. The vial was sealed with a crimp cap and heated in a microwave reactor to 160° C. for 1 hour. The cooled reaction mixture was placed in an ice-bah and chilled for 1 hour. The precipitated solid was filtered and rinsed with ethanol then ether (×4), affording 0.751 g (61%) of 4-((2,6-dimethylpyridin-4-yl)methyl)phthalazin-1(2H)-one as a tan solid. Extraction of the filtrate with EtOAc (×2) afforded an additional 0.219 g (18%, 90% purity) of 4-((2,6-dimethylpyridin-4-yl)methyl)phthalazin-1(2H)-one as a yellow solid. ¹H NMR (400 MHz, d₆-DMSO) δ 12.60 (bs, 1H), 8.27 (d, 1H), 7.89 (m, 2H), 7.87-7.81 (m, 1H), 6.97 (s, 2H), 4.24 (s, 2H), 2.36 (s, 6H)

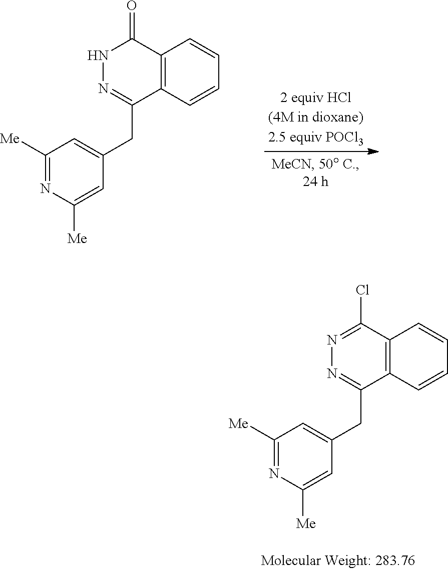 [SMILES: Cc1cc(Cc2n[nH]c(=O)c3ccccc23)cc(C)n1 ; Cc1cc(Cc2nnc(Cl)c3ccccc23)cc(C)n1]

A flame dried 50 mL R.B. was charged with the 4-((2,6-dimethylpyridin-4-yl)methyl)phthalazin-1(2H)-one (3.66 mmol, 0.97 g) and placed under an atmosphere of argon. Freshly distilled dry acetonitrile (14.6 mL) and HCl in dioxane (4M, 7.32 mmol, 1.93 mL) was injected. To the homogenous solution was added phosphoryl chloride (9.14 mmol, 0.852 mL). The R.B. was placed in oil bath and heated to 50° C. for 24 hours. The reaction was cooled to room temperature followed by placing the R.B. in an ice bath. To the cooled pale orange slurry was added 45 mL of a 1 M NaHCO₃ (aq) solution. The cooled mixture was stirred for 30 minutes. The precipitated solid was filtered and rinsed with water. The solid was dried under vacuum, affording 1.03 g (99%) of 1-chloro-4-((2,6-dimethylpyridin-4-yl)methyl)phthalazine as a tan solid. ¹H NMR (400 MHz, d₆-DMSO) δ 8.33 (m, 2H), 8.14 (m, 2H), 6.99 (s, 2H), 4.66 (s, 2H), 2.34 (s, 6H)

### Starting Material 3

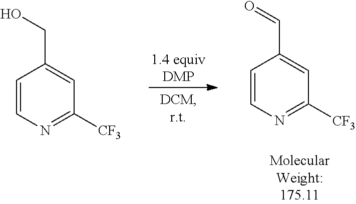 [SMILES: OCc1ccnc(C(F)(F)F)c1 ; O=Cc1ccnc(C(F)(F)F)c1]

A flame dried 200 mL flask was charged with DMP (12.6 mmol, 5.34 g) and placed under an atmosphere of argon. To the R.B. was injected 45 mL of dry DCM. Once the DMP was dissolved (2-(trifluoromethyl)pyridine-4-yl)methanol (9.0 mmol, 1.60 g) was injected dropwise. The reaction was stirred at r.t. until complete. The reaction mixture was diluted with DCM and quenched with sat. NaHCO₃ (aq). The layers were separated and the organic layer was washed with brine, dried over sodium sulfate, filtered, and concentrated down. The crude material was loaded onto a 100 g silica-gel cartridge and purified on a Biotage flash system eluting with DCM/MeOH, affording 0.636 g (40%) of 2-(trifluoromethyl)isonicotinaldehyde as a clear oil. ¹H NMR (400 MHz, d₆-DMSO) δ 10.16 (s, 1H), 9.08 (d, J=4.8 Hz, 1H), 8.29 (s, 1H), 8.14 (d, J=4.8 Hz, 1H)

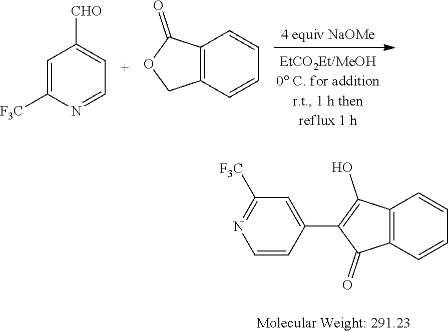

A solution of sodium methoxide (13.84 mmol, 0.747 g) in 7 mL of methanol, in a 25 mL R.B. was cooled to 0° C. with an ice-bath. In a separate vial was prepared a solution of the 1-isobenzofuranone (3.46 mmol, 0.464 g), 2-(trifluoromethyl)isonicotinaldehyde (3.63 mmol, 0.635 g), and 5 mL of ethyl propionate, which was taken up into a syringe and added dropwise over 10 minutes to the methoxide solution, down the reflux condenser that was attached. After compete addition the reaction was stirred at r.t for 1 hour. The reaction was heated to reflux (100° C.) for 1.5 hour. The cooled reaction mixture was concentrated down to a thick slurry, diluted with water (100 mL), and transferred to a sep-funnel. The dark red solution was extracted with diethyl ether (3×50 mL). The aqueous layer in a 100 mL Erlenmeyer flask was acidified with acetic acid. A precipitate formed which was filtered and rinsed with water and a small amount of ether, affording 0.241 g (24%) of 3-hydroxy-2-(2-(trifluoromethyl)pyridin-4-yl)-1H-inden-1-one as an orange solid. The aqueous filtrate was then extracted with EtOAc. The organic layer was washed with brine twice, dried over sodium sulfate, filtered, and concentrated down affording 0.358 g (35%) of 3-hydroxy-2-(2-(trifluoromethyl)pyridin-4-yl)-1H-inden-1-one as a yellow solid. ¹H NMR (400 MHz, d₆-DMSO) δ 13.22 (bs, 1H), 9.04 (d, J=1.8 Hz, 1H), 8.69 (dd, J=1.9 & 6.5 Hz, 1H), 8.30 (d, J=6.7 Hz, 1H), 7.57-7.48 (m, 4H)

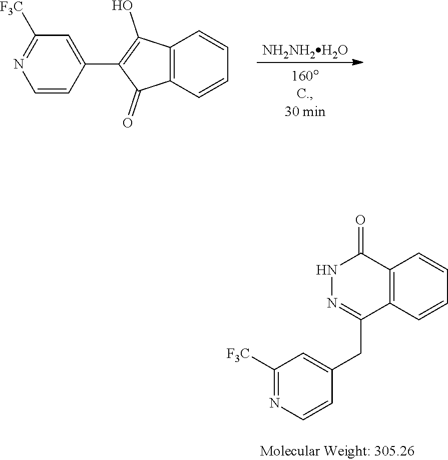 [SMILES: O=C1C(c2ccnc(C(F)(F)F)c2)=C(O)c2ccccc21 ; "NH2NH2·H2O" ; O=c1[nH]nc(Cc2ccnc(C(F)(F)F)c2)c2ccccc12]

A 2 mL vial was charged with the 3-hydroxy-2-(2-(trifluoromethyl)pyridin-4-yl)-1H-inden-1-one (0.83 mmol, 0.241 g) and 1.66 mL of hydrazine monohydrate. The vial was sealed with a crimp cap and heated in a microwave reactor at 160° C. for 30 minutes. The cooled reaction mixture was filtered and rinsed with water. The collected solid was dried under air flow in the Buchner funnel for 1 hour, affording 0.055 g (22%) of 4-((2-(trifluoromethyl)pyridin-4-yl)methyl)phthalazin-1(2H)-one as a white solid. ¹H NMR (400 MHz, d₆-DMSO) δ 12.60 (s, 1H), 8.68 (d, J=4.9 Hz, 1H), 8.28 (d, J=7.3 Hz, 1H), 8.01 (m, 1H), 7.94 (m, 2H), 7.87 (m, 1H), 7.64 (d, J=4.9 Hz, 1H), 4.51 (s, 2H)

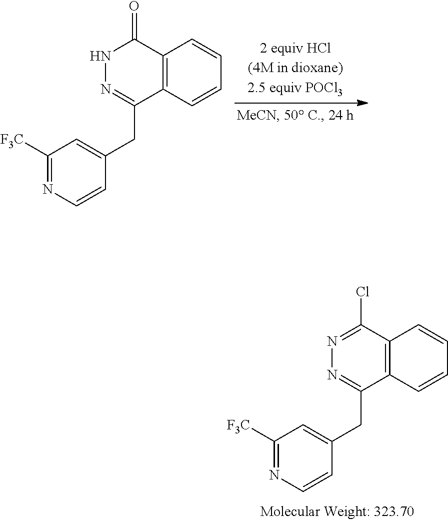 [SMILES: O=c1[nH]nc(Cc2ccnc(C(F)(F)F)c2)c2ccccc12 ; FC(F)(F)c1cc(Cc2nnc(Cl)c3ccccc23)ccn1]

A flame dried 5 mL vial was charged with the 4-((2-(trifluoromethyl)pyridin-4-yl)methyl)phthalazin-1(2H)-one (0.18 mmol, 0.055 g) and placed under an atmosphere of argon. Freshly distilled dry acetonitrile (0.72 mL) and HCl in dioxane (4M, 0.36 mmol, 0.09 mL) was injected. To the homogenous solution was added phosphoryl chloride (0.45 mmol, 0.042 mL). The vial was placed in a reactor plate and heated to 50° C. for 24 hours. The reaction was cooled to room temperature followed by placing the vial in an ice bath. To the cooled pale yellow slurry was added 2.5 mL of a 1 M NaHCO₃ (aq) solution. The cooled mixture was stirred for 30 minutes and the precipitated solid was filtered and rinsed with water. The material was dried under vacuum affording 0.046 g (79%) of 1-chloro-4-((2-(trifluoromethyl)pyridin-4-yl)methyl)phthalazine as a white solid. LC/MS (ESI, M+1): found 324.5

### Starting Material 4

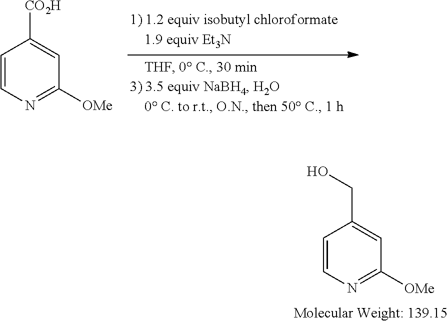 [SMILES: COc1cc(C(=O)O)ccn1 ; COc1cc(CO)ccn1]

A flame dried 500 mL R.B. was charged with 2-methoxyisonicotinic acid (20 mmol, 3.06 g) and placed under an atmosphere of argon. To the flask was injected 80 mL of dry THF and freshly distilled TEA (38 mmol, 5.26 mL). The R.B. was placed in an ice-bath and the mixture was equilibrated to temperature. Once cool, isobutyl chloroformate (24 mmol, 3.12 mL) was added dropwise to the solution. After complete addition the reaction was stirred for 30 minutes. To the slurry was slowly injected a solution of NaBH₄ (70 mmol, 2.65 g) in 40 mL of water. The ice-bath was removed and the reaction was warmed to r.t. and stirred for 14 hours. The R.B. was then placed in an oil bath and heated to 50° C. for 1 h. The reaction mixture was re-cooled in an ice-bath and then quenched with sat. NH₄Cl(aq). The mixture was poured into a separatory-funnel and extracted with EtOAc. The organic layer was washed with water and brine, dried over sodium sulfate, filtered, and concentrated down. The crude material was loaded onto a 100 g silica-gel cartridge and purified on a Biotage flash system eluting with Hex/EtOAc, affording 1.67 g (67%) of (2-methoxypyridin-4-yl)methanol as a clear oil. ¹H NMR (400 MHz, d₆-DMSO) δ 8.08 (d, J=5.3 Hz, 1H), 6.91 (d, J=5.3 Hz, 1H), 6.73 (s, 1H), 5.39 (bs, 1H), 4.49 (s, 2H), 3.83 (s, 3H)

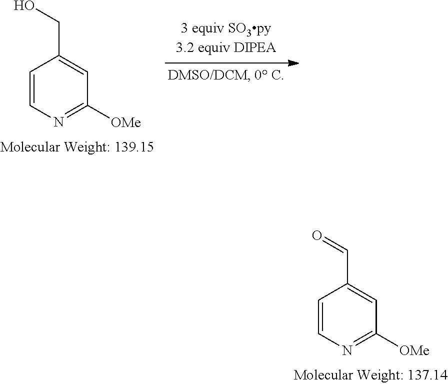 [SMILES: COc1cc(CO)ccn1 ; COc1cc(C=O)ccn1]

To a flame dried 250 mL R.B. under an argon atmosphere was injected the (2-methoxypyridin-4-yl)methanol (12 mmol, 1.67 g), 30 mL of DMSO, 30 mL of DCM, and freshly distilled DIPEA (38.4 mmol, 6.69 mL). The R.B. was placed in an ice bath and the mixture was equilibrated to temperature. To the mixture was injected dropwise a solution of pyridine sulfur trioxide complex (36 mmol, 5.73 g) in 72 mL of DMSO, that also contained pyridine (10 mmol, 0.80 mL). The reaction was stirred at 0° C. gradually warming to r.t. over 14 hours. The reaction mixture was poured into a sep-funnel containing EtOAc, washed with water (×2) and brine. The combined aqueous washes were extracted with EtOAc, which was washed with water (×2) and brine. The combined organic layers were dried over sodium sulfate, filtered, and concentrated down. The crude material was loaded onto a 100 g silica-gel cartridge and purified on a Biotage flash system eluting with Hex/EtOAc, affording 1.30 (79%) of 2-methoxyisonicotinaldehyde as a clear light yellow oil. ¹H NMR (400 MHz, d₆-DMSO) δ 10.03 (s, 1H), 8.41 (d, J=5.2 Hz, 1H), 7.36 (dd, J=0.7 & 5.2 Hz, 1H), 7.28 (m, 1H), 3.92 (s, 3H)

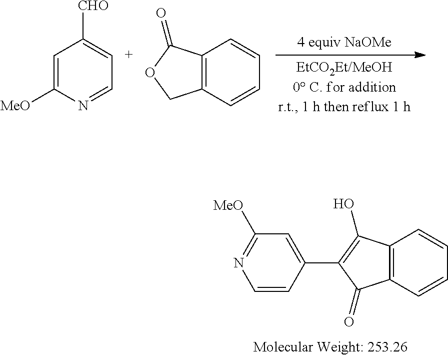

A solution of sodium methoxide (120 mmol, 6.48 g) in 60 mL of methanol in a 250 mL R.B. was cooled to 0° C. with an ice-bath. In a separate pear-shaped R.B. was prepared a solution of 1-isobenzofuranone (30 mmol, 4.02 g), 2-methoxyisonicotinaldehyde (31.5 mmol, 4.32 g), and 30 mL of ethyl propionate, which was taken up into a syringe and added dropwise over 10 minutes to the methoxide solution, down the reflux condenser that was attached. After compete addition the reaction was stirred at r.t for 1 hour. The reaction was heated to reflux (100° C.) for 1.5 hour. The cooled reaction mixture was concentrated down to a thick slurry, diluted with water (100 mL), and transferred to a sep-funnel. The dark red solution was extracted with diethyl ether (3×50 mL). The aqueous layer in a 250 mL Erlenmeyer flask was acidified with acetic acid. A precipitate formed which was filtered and rinsed with water, EtOAc, and Et₂O. The material was dried under vacuum affording 4.90 g (64%) of 3-hydroxy-2-(2-methoxypyridin-4-yl)-1H-inden-1-one as a bright yellow solid. ¹H NMR (400 MHz, d₆-DMSO) δ 13.35 (bs, 1H), 8.37 (dd, J=1.5 & 6.8 Hz, 1H), 8.27 (d, J=1.4 Hz, 1H), 7.83 (d, J=6.7 Hz, 1H), 7.50 (m, 2H), 7.43 (m, 2H), 3.99 (s, 3H)

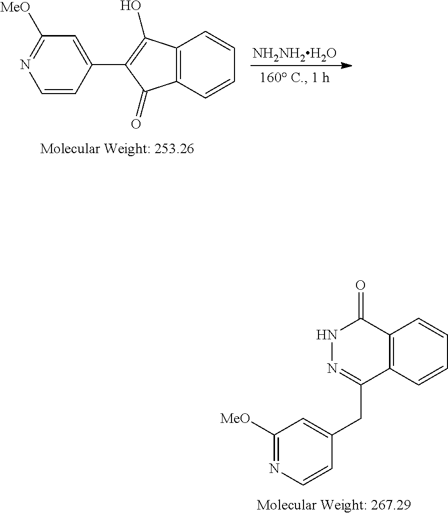

A 5 mL microwave vial was charged with 3-hydroxy-2-(2-methoxypyridin-4-yl)-1H-inden-1-one (4 mmol, 1.01 g) and 4 mL of hydrazine monohydrate. The vial was sealed with a crimp cap. The reaction was heated in a microwave reactor at 160° C. for 1 hour. The precipitated solid was filtered and rinsed with water. The crude material was loaded onto a 50 g silica-gel cartridge and purified on a Biotage flash system eluting with DCM/MeOH, affording 0.300 g (28%) of 4-((2-methoxypyridin-4-yl)methyl)phthalazin-1(2H)-one as a white solid. ¹H NMR (400 MHz, d₆-DMSO) δ 12.63 (bs, 1H), 8.28 (m, 1H), 7.94 (m, 2H), 7.27 (d, J=6.7 Hz, 1H), 6.24 (s, 1H), 6.08 (dd, J=1.7 & 6.8 Hz, 1H), 4.12 (s, 2H), 3.33 (s, 3H)

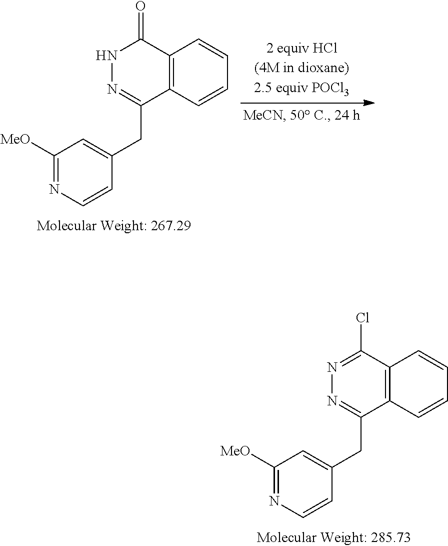 [SMILES: COc1cc(Cc2n[nH]c(=O)c3ccccc23)ccn1 ; COc1cc(Cc2nnc(Cl)c3ccccc23)ccn1]

A flame dried 50 mL R.B. was charged with the 4-((2-methoxypyridin-4-yl)methyl)phthalazin-1(2H)-one (3.6 mmol, 0.963 g) and placed under an atmosphere of argon. Freshly distilled dry acetonitrile (14.4 mL) and HCl in dioxane (4M, 7.2 mmol, 1.8 mL) was injected. To the homogenous solution was added phosphoryl chloride (9 mmol, 0.839 mL). The R.B. was placed in an oil-bath and heated to 50° C. for 24 hours. The reaction was cooled to room temperature followed by placing the R.B. in an ice bath. To the cooled pale pink slurry was added 51 mL of a 1 M NaHCO₃ (aq) solution. The cooled mixture was stirred for 30 minutes. Typically, the product crashes out of solution, but this did not occur. So, the reaction mixture was poured into a separatory-funnel containing 90/10 EtOAc/MeOH. The layers were separated and the aqueous layer was extracted with 90/10 EtOAc/MeOH. The combined organic layers were washed with sat. NaHCO₃, water, and brine, dried over sodium sulfate, filtered, and concentrated down. The crude material was loaded onto a 50 g silica-gel cartridge and purified on a Biotage flash system eluting with DCM/MeOH, affording 0.762 g (74%) of 1-chloro-4-((2-methoxypyridin-4-yl)methyl)phthalazine as a light tan solid. ¹H NMR (400 MHz, d₆-DMSO) δ 8.35 (m, 1H), 8.29 (m, 1H), 8.12 (m, 2H), 8.04 (d, J=5.3 Hz, 1H), 6.90 (d, J=5.0 Hz, 1H), 6.78 (s, 1H), 4.71 (s, 2H), 3.79 (s, 3H)

### Starting Material 5

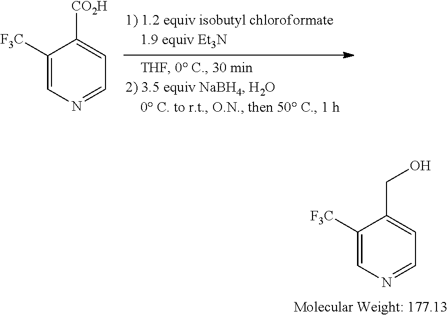 [SMILES: O=C(O)c1ccncc1C(F)(F)F ; OCc1ccncc1C(F)(F)F]

A flame dried 500 mL R.B. was charged with 3-(trifluoromethyl)isonicotinic acid (20 mmol, 3.82 g) and placed under an atmosphere of argon. To the flask was injected 80 mL of THF and freshly distilled TEA (38 mmol, 5.26 mL). The R.B. was placed in an ice-bath and the mixture was equilibrated to temperature. Once cool, isobutyl chloroformate (24 mmol, 3.12 mL) was added dropwise to the solution. After complete addition the reaction was stirred for 30 minutes. To the slurry was slowly injected a solution of NaBH₄ (70 mmol, 2.65 g) in 40 mL of water. The ice-bath was removed and the reaction was warmed to r.t. and stirred for 14 hours. The R.B. was then placed in an oil bath and heated to 50° C. for 1 h. The reaction mixture was re-cooled in an ice-bath and then quenched with sat. NH₄Cl(aq). The mixture was poured into a separatory-funnel and extracted with EtOAc. The organic layer was washed with water and brine, dried over sodium sulfate, filtered, and concentrated down. The crude material was loaded onto a 100 g silica-gel cartridge and purified on a Biotage flash system eluting with Hex/EtOAc, affording 1.032 g (29%) of (3-(trifluoromethyl)pyridin-4-yl)methanol as a light yellow waxy solid. ¹H NMR (400 MHz, d₆-DMSO) δ 8.87 (d, J=5.1 Hz, 1H), 8.84 (s, 1H), 7.81 (d, J=5.1 Hz, 1H), 5.77 (t, J=5.7 Hz, 1H), 4.71 (d, J=5.7 Hz, 1H)

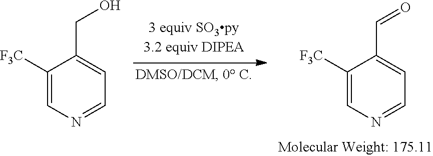 [SMILES: OCc1ccncc1C(F)(F)F ; O=Cc1ccncc1C(F)(F)F]

To a flame dried 250 mL R.B. under an argon atmosphere was injected the (3-(trifluoromethyl)pyridin-4-yl)methanol (5.65 mmol, 1.00 g), 14 mL of DMSO, 14 mL of DCM, and freshly distilled DIPEA (18.1 mmol, 3.15 mL). The R.B. was placed in an ice bath and the mixture was equilibrated to temperature. To the mixture was injected dropwise a solution of pyridine sulfur trioxide complex (16.95 mmol, 2.70 g) in 34 mL of DMSO, that also contained pyridine (10 mmol, 0.80 mL). The reaction was stirred at 0° C. gradually warming to r.t. over 14 hours. The reaction mixture was poured into a sep-funnel containing EtOAc, washed with water (×2) and brine. The combined aqueous washes were extracted with EtOAc, which was washed with water (×2) and brine. The combined organic layers were dried over sodium sulfate, filtered, and concentrated down. The crude material was loaded onto a 50 g silica-gel cartridge and purified on a Biotage flash system eluting with Hex/EtOAc, affording 0.609 g (61%) of 3-(trifluoromethyl)isonicotinaldehyde as a clear oil. ¹H NMR (400 MHz, d₆-DMSO) δ10.30 (q, J=1.8 Hz, 1H), 9.18 (m, 2H), 8.00 (d, J=4.9 Hz, 1H)

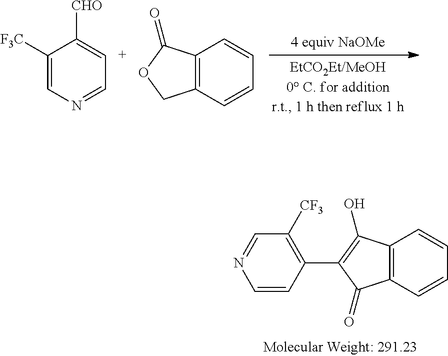

A solution of sodium methoxide (13.25 mmol, 0.716 g) in 6.6 mL of methanol in a 25 mL R.B. was cooled to 0° C. with an ice-bath. In a separate vial was prepared a solution of the 1-isobenzofuranone (3.31 mmol, 0.444 g), 3-(trifluoromethyl)isonicotinaldehyde (3.48 mmol, 0.609 g), and 3.5 mL of ethyl propionate, which was taken up into a syringe and added dropwise over 10 minutes to the methoxide solution, down the reflux condenser that was attached. After compete addition the reaction was stirred at r.t for 1 hour. The reaction was heated to reflux (100° C.) for 1.5 hour. The cooled reaction mixture was concentrated down to a thick slurry, diluted with water (100 mL), and transferred to a sep-funnel. The dark red solution was extracted with diethyl ether (3×50 mL). The aqueous layer in a 250 mL Erlenmeyer flask was acidified with acetic acid. Typically, a precipitate forms but did not happen, so the aqueous solution was extracted with EtOAc (×2). The organic layer was dried over sodium sulfate, filtered, and concentrated down. The crude material was take onto the next step. LC/MS (ESI, M+1): found 292.1

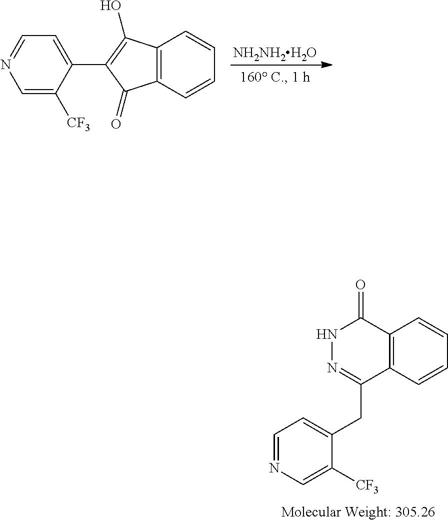

A 5 mL microwave vial was charged with 3-hydroxy-2-(3-(trifluoromethyl)pyridin-4-yl)-1H-inden-1-one (3 mmol, 0.874 g) and 2 mL of hydrazine monohydrate. The vial was sealed with a crimp cap. The reaction was heated in a microwave reactor at 160° C. for 1 hour. The precipitated solid was filtered and rinsed with water. The crude material was loaded onto a 50 g silica-gel cartridge and purified on a Biotage flash system eluting with DCM/MeOH, affording 0.185 g (20%) of 4-((3-(trifluoromethyl)pyridin-4-yl)methyl)phthalazin-1(2H)-one as a yellow solid. ¹H NMR (400 MHz, d₆-DMSO) δ 12.53 (s, 1H), 8.95 (s, 1H), 8.77 (d, J=5.1 Hz, 1H), 8.30 (d, J=7.8 Hz, 1H), 7.96 (m, 2H), 7.94 (m, 1H), 7.47 (d, J=5.1 Hz, 1H), 4.55 (s, 2H)

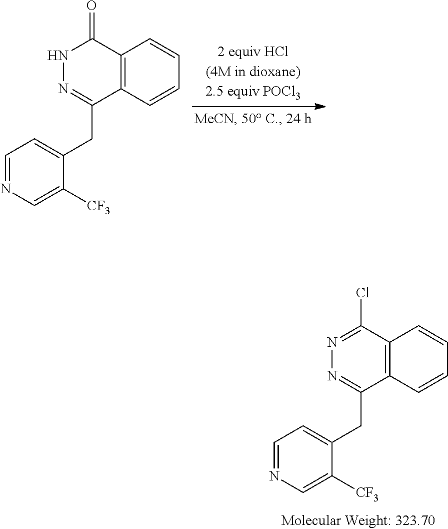 [SMILES: O=c1[nH]nc(Cc2ccncc2C(F)(F)F)c2ccccc12 ; FC(F)(F)c1cnccc1Cc1nnc(Cl)c2ccccc12]

A flame dried 50 mL R.B. was charged with the 4-((3-(trifluoromethyl)pyridin-4-yl)methyl)phthalazin-1(2H)-one (0.589 mmol, 0.180 g) and placed under an atmosphere of argon. Freshly distilled dry acetonitrile (2.35 mL) and HCl in dioxane (4M, 1.178 mmol, 0.294 mL) was injected. To the homogenous solution was added phosphoryl chloride (1.473 mmol, 0.137 mL). The R.B. was placed in an oil-bath and heated to 50° C. for 24 hours. The reaction was cooled to room temperature followed by placing the R.B. in an ice bath. To the cooled pale pink slurry was added 8.4 mL of a 1 M NaHCO₃ (aq) solution. The cooled mixture was stirred for 30 minutes. The precipitated solid was filtered and rinsed with water. The material was dried under a vacuum, affording 0.123 g (65%) of 1-chloro-4-((3-(trifluoromethyl)pyridin-4-yl)methyl)phthalazine as a tan solid. ¹H NMR (400 MHz, d₆-DMSO) δ 8.98 (s, 1H), 8.77 (d, J=5.1 Hz, 1H), 8.37 (m, 2H), 8.21 (m, 2H), 7.39 (d, J=5.1 Hz, 1H), 4.97 (s, 2H)

### Starting Material 6

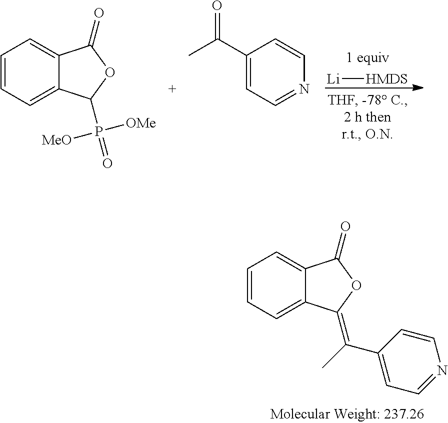

A flame dried 500 mL R.B. was charged with dimethyl (3-oxo-1,3-dihydroisobenzofuran-1-yl)phosphonate (5.78 mmol, 1.40 g) and the system was placed under an atmosphere of argon. To the flask was injected 157 mL of THF. The R.B. was placed in a dry ice/acetone bath and the mixture was equilibrated to temperature. A THF solution of Li-HMDS (1M, 5.78 mmol, 5.78 mL) was injected and the mixture was stirred for 1 hour. The reaction mixture turned a greenish-yellow color. 4-Acetyl-2-methylpyridine (5.78 mmol, 0.64 mL) was injected in and the reaction mixture was stirred for 1 hour at −78° C., then the bath was removed and the reaction mixture was stirred at room temperature overnight. The reaction mixture was quenched with sat. NH₄Cl (aq) and transferred to a separatory-funnel with EtOAc and water. The layers were separated, and the aqueous layer was extracted with EtOAc. The combined organic layers were washed with brine, dried over sodium sulfate, filtered, and concentrated down. The crude material was loaded onto a 100 g silica-gel cartridge and purified on a Biotage flash system eluting with DCM/MeOH, affording 1.3 g (99%) of 3-(1-(pyridin-4-yl)ethylidene)isobenzofuran-1(3H)-one as an off white waxy solid. ¹H NMR (400 MHz, d₆-DMSO) δ 8.81 (m, 1H), 8.74 (m, 2H), 7.93 (m, 1H), 7.82 (m, 1H), 7.51 (m, 2H), 6.70 (m, 1H), 2.31 (s, 3H)

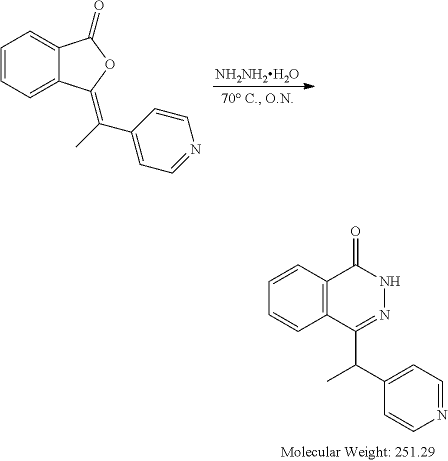

A 10 mL microwave vial was charged with 3-(1-(pyridin-4-yl)ethylidene)isobenzofuran-1(3H)-one (5.3 mmol, 1.259 g) and 5.3 mL of hydrazine monohydrate. The vial was sealed with a crimp cap and placed in an aluminum heat block, and heated at 70° C. for 14 hours. The precipitated solid was filtered and rinsed with EtOH. The filtrate was concentrated down, and the crude material was loaded onto a 50 g silica-gel cartridge and purified on a Biotage flash system eluting with DCM/MeOH, affording 0.4187 g (31%) of 4-(1-(pyridin-4-yl)ethyl)phthalazin-1(2H)-one as a white solid. ¹H NMR (400 MHz, d₆-DMSO) δ 13.30-11.89 (bs, 1H), 8.46 (d, 2H), 8.26 (M, 1H), 7.90-7.75 (m, 3H), 7.33 (d, 2H), 4.85 (q, 1H), 1.58 (d, 3H)

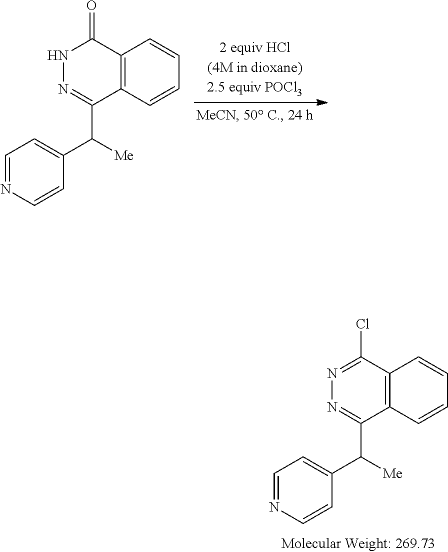 [SMILES: CC(c1ccncc1)c1n[nH]c(=O)c2ccccc12 ; CC(c1ccncc1)c1nnc(Cl)c2ccccc12]

A flame dried 50 mL R.B. was charged with the 4-(1-(pyridin-4-yl)ethyl)phthalazin-1(2H)-one (1.66 mmol, 0.418 g) and placed under an atmosphere of argon. Freshly distilled dry acetonitrile (6.6 mL) and HCl in dioxane (4M, 3.32 mmol, 0.83 mL) was injected. To the homogenous solution was added phosphoryl chloride (4.15 mmol, 0.386 mL). The R.B. was placed in an oil-bath and heated to 50° C. for 24 hours. The reaction was cooled to room temperature followed by placing the R.B. in an ice bath. To the cooled slurry was added 23.6 mL of a 1 M NaHCO₃ (aq) solution. The cooled mixture was stirred for 30 minutes. The precipitated solid was filtered and rinsed with water. The material was dried under a vacuum, affording 0.374 g (83%) of 1-chloro-4-(1-(pyridin-4-yl)ethyl)phthalazine as an off white solid. ¹H NMR (400 MHz, d₆-DMSO) δ 8.46 (d, 2H), 8.37 (m, 2H), 8.11 (m, 2H), 7.38 (d, 2H), 5.31 (q, J=7.0 Hz, 1H), 1.79 (d, J=7.0 Hz, 1H)

### Starting Material 7

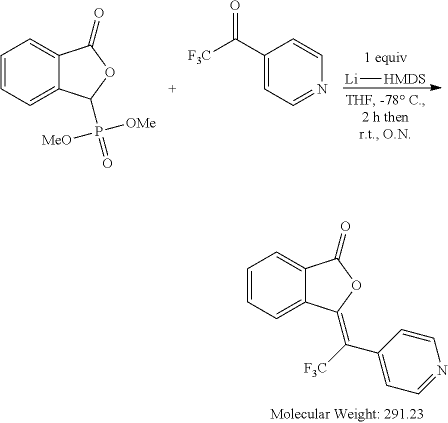

A flame dried 500 mL R.B. was charged with dimethyl (3-oxo-1,3-dihydroisobenzofuran-1-yl)phosphonate (5.0 mmol, 1.21 g) and the system was placed under an atmosphere of argon. To the flask was injected 116 mL of THF. The R.B. was placed in a dry ice/acetone bath and the mixture was equilibrated to temperature. A THF solution of Li-HMDS (1M, 5.0 mmol, 5.0 mL) was injected and the mixture was stirred for 1 hour. The reaction mixture turned a greenish-yellow color. A solution of 2,2,2-trifluoro-1-(pyridin-4-yl)ethan-1-one (5 mmol, 0.875 g) in 20 mL of THF, prepared in a flame dried 100 mL pear shaped flask under argon, was cannulated into the ylide reaction mixture. The reaction mixture was stirred for 1 hour at −78° C., then the bath was removed and the reaction mixture was stirred at r.t. for 14 hours. The reaction mixture was quenched with sat. NH₄Cl (aq) and transferred to a separatory funnel with EtOAc and water. The layers were separated, and the aqueous layer was extracted with EtOAc. The combined organic layers were washed with brine, dried over sodium sulfate, filtered, and concentrated down. The crude material was loaded onto a 50 g silica-gel cartridge and purified on a Biotage flash system eluting with DCM/MeOH, affording 1.395 g (93%) of 3-(2,2,2-trifluoro-1-(pyridin-4-yl)ethylidene)isobenzofuran-1(3H)-one as a white solid. LC/MS (ESI, M+1): found 292.1

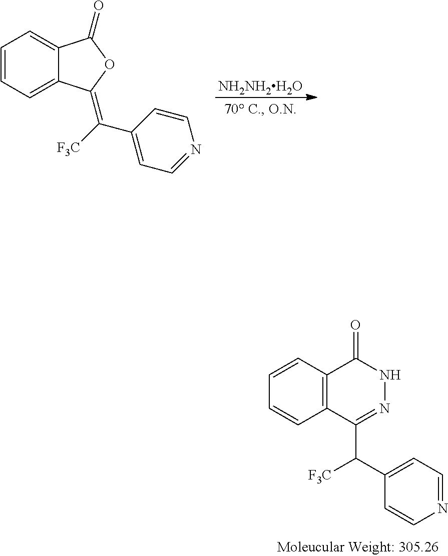

A 10 mL microwave vial was charged with 3-(2,2,2-trifluoro-1-(pyridin-4-yl)ethylidene)isobenzofuran-1(3H)-one (4.77 mmol, 1.39 g) and 4.77 mL of hydrazine monohydrate. The vial was sealed with a crimp cap and placed in an aluminum heat block and heated at 70° C. for 14 hours. The precipitated solid was filtered and rinsed with EtOH. The filtrate was concentrated down, and the crude material was loaded onto a 50 g silica-gel cartridge and purified on a Biotage flash system eluting with DCM/MeOH, affording 0.236 g (16%) of 4-(2,2,2-trifluoro-1-(pyridin-4-yl)ethyl)phthalazin-1(2H)-one as a white solid. LC/MS (ESI, M+1): found 306.1

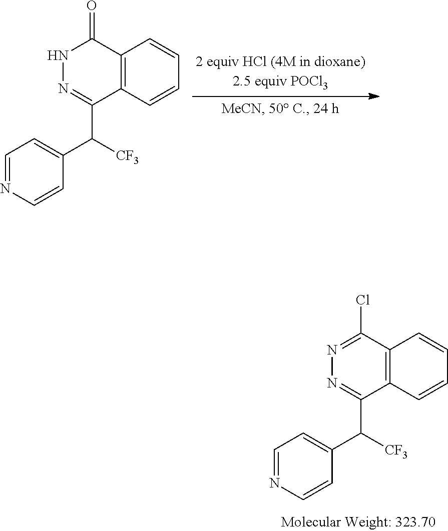 [SMILES: O=c1[nH]nc(C(c2ccncc2)C(F)(F)F)c2ccccc12 ; FC(F)(F)C(c1ccncc1)c1nnc(Cl)c2ccccc12]

A flame dried 50 mL R.B. was charged with 4-(2,2,2-trifluoro-1-(pyridin-4-yl)ethyl)phthalazin-1(2H)-one (0.773 mmol, 0.236 g) and placed under an atmosphere of argon. Freshly distilled dry acetonitrile (3.1 mL) and HCl in dioxane (4M, 1.546 mmol, 0.386 mL) was injected. To the homogenous solution was added phosphoryl chloride (1.932 mmol, 0.180 mL). The R.B. was placed in an oil-bath and heated to 50° C. for 24 hours. The reaction was cooled to room temperature followed by placing the R.B. in an ice bath. To the cooled slurry was added 11 mL of a 1 M NaHCO₃ (aq) solution. The cooled mixture was stirred for 30 minutes. The precipitated solid was filtered and rinsed with water. The material was dried under a vacuum, affording 0.1511 g (60%) of 1-chloro-4-(2,2,2-trifluoro-1-(pyridin-4-yl)ethyl)phthalazine as light orange solid. ¹H NMR (400 MHz, d₆-DMSO) δ 8.38 (m, 3H), 8.29 (m, 3H), 8.25-7.81 (m, 3H) LC/MS (ESI, M+1): found 324.5

### Starting Material 8

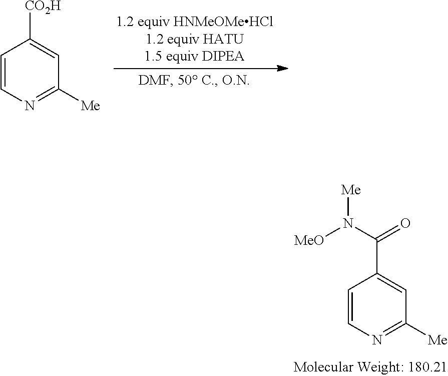 [SMILES: Cc1cc(C(=O)O)ccn1 ; CON(C)C(=O)c1ccnc(C)c1]

A flame dried 250 mL R.B. was charged with 2-methylisonicotinic acid (40 mmol, 5.48 g), N,O-dimethylhydroxylamine hydrochloride (48 mmol, 4.66 g), and HATU (48 mmol, 18.25 g), and flushed under a positive pressure of argon. To the flask was injected 80 mL of DMF then DIPEA (60 mmol, 10.5 mL). The R.B. was placed in oil bath that was heated to 50° C. and the reaction was stirred for 14 hours. The cooled reaction mixture was poured into a separatory funnel containing EtOAc and water. The organic layer was washed with sat NaHCO₃ (aq), then brine. The combined aqueous layers were extracted with EtOAc which was then washed with water then brine. The combined organic layers were dried over sodium sulfate, filtered, and concentrated down. The crude material was loaded onto a 220 g silica-gel cartridge and purified on a Biotage flash system eluting with DCM/MeOH, affording 7.19 g (99%) of N-methoxy-N,2-dimethylisonicotinamide as a yellow oil. ¹H NMR (400 MHz, d₆-DMSO) δ 8.54 (dd, 1H), 7.37 (s, 1H), 7.30 (d, 1H), 3.55 (s, 3H), 3.27 (s, 3H), 2.52 (s, 3H)

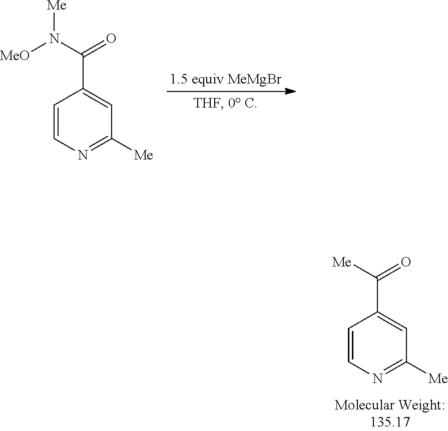 [SMILES: CON(C)C(=O)c1ccnc(C)c1 ; CC(=O)c1ccnc(C)c1]

A flame dried 1 L R.B. was charged with N-methoxy-N,2-dimethylisonicotinamide (40 mmol, 7.2 g) and placed under an argon atmosphere. To the flask was injected 200 mL of dry THF. The R.B. was placed in an ice bath and the solution was equilibrated to temperature. A THF solution of MeMgBr (3M, 60 mmol, 20 mL) was injected in dropwise slowly. The reaction was stirred overnight gradually warming to room temperature. The reaction mixture was re-cooled with an ice bath and quenched with sat NH₄Cl (aq). The reaction mixture was transferred to a separatory-funnel and extracted with EtOAc three times. The combined organic layers were washed water then brine, dried over sodium sulfate, filtered, and concentrated down. The crude material was loaded onto a 220 g silica-gel cartridge and purified on a Biotage flash system eluting with DCM/MeOH, affording 2.63 g (48%) of 1-(2-methylpyridin-4-yl)ethan-1-one as a dark red oil. ¹H NMR (400 MHz, d₆-DMSO) δ 8.66 (dd, 1H), 7.70 (s, 1H), 7.61 (d, 1H), 2.61 (s, 3H), 2.57 (s, 3H)

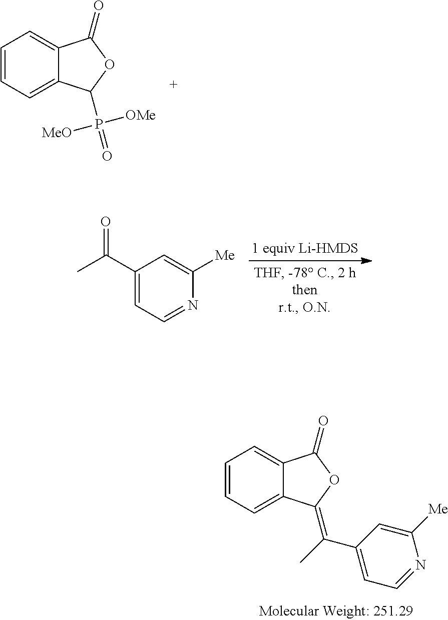

A flame dried 1 L R.B. was connected to the solvent system and ˜486 mL of dry THF was dispensed in. To the flask was added dimethyl (3-oxo-1,3-dihydroisobenzofuran-1-yl)phosphonate (19.45 mmol, 4.71 g) and the system was placed under an atmosphere of argon. The R.B. was placed in a dry ice/acetone bath and the mixture was equilibrated to temperature. A THF solution of Li-HMDS (1M, 19.45 mmol, 19.45 mL) was injected and the mixture was stirred for 1 hour. The reaction mixture turned a greenish-yellow color. 4-Acetyl-2-methylpyridine was injected in and the reaction mixture was stirred for 1 hour at −78° C., then the bath was removed and the reaction mixture was stirred at room temperature overnight. The reaction mixture was quenched with sat. NH₄Cl(aq) and transferred to a separatory-funnel with EtOAc and water. The layers were separated, and the aqueous layer was extracted with EtOAc. The combined organic layers were washed with brine, dried over sodium sulfate, filtered, and concentrated down. The crude material was loaded onto a 220 g silica-gel cartridge and purified on a Biotage flash system eluting with DCM/MeOH, affording 3.9 g (80%) of 3-(1-(2-methylpyridin-4-yl)ethylidene)isobenzofuran-1(3H)-one as a red solid. ¹H NMR (400 MHz, d₆-DMSO) δ 8.58 (d, 1H), 7.91 (m, 1H), 7.57 (m, 2H), 7.36 (s, 1H), 7.28 (d, 1H), 6.72 (m, 1H), 2.52 (s, 3H), 2.27 (s, 3H). LC/MS (ESI, M+1): found 252.2

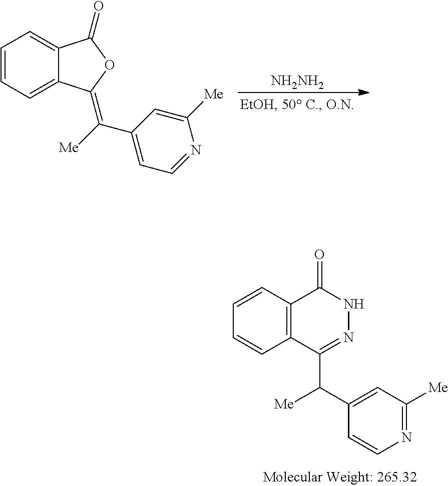 [SMILES: C/C(=C1/OC(=O)c2ccccc21)c1ccnc(C)c1 ; "NH2NH2" ; Cc1cc(C(C)c2n[nH]c(=O)c3ccccc23)ccn1]

A 20 mL vial was charged with 3-(1-(2-methylpyridin-4-yl)ethylidene)isobenzofuran-1(3H)-one (5.13 mmol, 1.29 g) and sealed with a crimp cap. To the vial was injected 10.2 mL of a 1M solution of hydrazine in EtOH. The vial was placed in a reactor plate and the reaction was heated to 50° C. overnight. The next day a bright yellow precipitate formed. A small amount of water was added to help break up the precipitate so it could be filtered, but this dissolved it up. The reaction mixture was loaded onto a 100 g C18 silica-gel cartridge and purified on a Biotage flash system eluting with H₂O/MeOH+0.1% TFA, affording 0.852 g (63%) of 4-(1-(2-methylpyridin-4-yl)ethyl)phthalazin-1(2H)-one as a dark yellow viscous oil. ¹H NMR (400 MHz, d₆-DMSO) δ 12.79 (bs, 1H), 8.70 (d, 1H), 8.29 (m, 1H), 7.96-7.81 (m, 5H), 5.15 (q, 1H), 2.66 (s, 3H), 1.65 (d, 3H). LC/MS (ESI, M+1): found 266.2

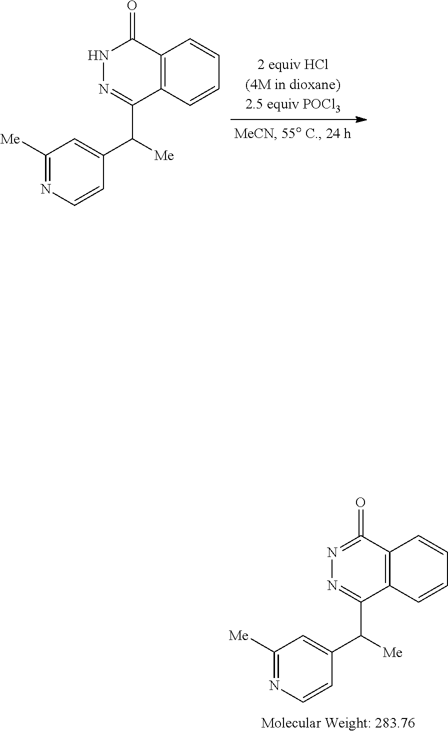

A flame dried 200 mL R.B. was charged with the 4-(1-(2-methylpyridin-4-yl)ethyl)phthalazin-1(2H)-one (9.61 mmol, 2.55 g) and placed under an atmosphere of argon. To the flask was injected 36.6 mL of acetonitrile and 4.8 mL of HCl in dioxane (4M, 19.22 mmol) was injected. To the homogenous solution was added phosphoryl chloride (24.02 mmol, 2.24 mL). The R.B. was placed in an oil-bath and heated to 55° C. for 24 hours. The reaction was cooled to room temperature followed by placing the R.B. in an ice bath. To the cooled solution was slowly added 137 mL of a 1 M NaHCO₃ (aq) solution. The cooled mixture was stirred for 30 minutes. The mixture was transferred to a separatory funnel and extracted with EtOAc. The layers were separated and the aqueous layer was extracted with EtOAc. The combined organic layers were washed with water then brine, dried over sodium sulfate, and concentrated down. The crude material was loaded onto a 120 g silica-gel cartridge and purified on a Biotage flash system eluting with DCM/MeOH, affording 1.71 g (63%) of 1-chloro-4-(1-(2-methylpyridin-4-yl)ethyl)phthalazine as a golden yellow solid. ¹H NMR (400 MHz, d₆-DMSO) δ 8.39-8.28 (m, 3H), 8.11 (m, 2H), 7.24 (s, 1H), 7.16 (d, 1H), 5.25 (q, 1H), 2.39 (s, 3H), 1.77 (d, 3H). LC/MS (ESI, M+1): found 284.6

### Synthesis of SR-35429

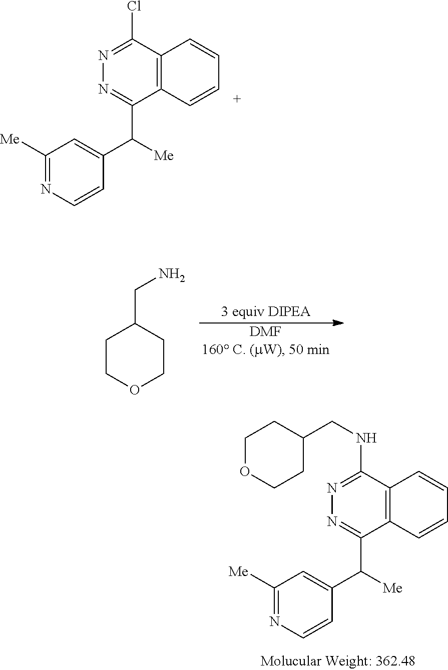 [SMILES: Cc1cc(C(C)c2nnc(Cl)c3ccccc23)ccn1 ; NCC1CCOCC1 ; "DMF160° C. (mW), 50 min" ; Cc1cc(C(C)c2nnc(NCC3CCOCC3)c3ccccc23)ccn1]

SR-35429: A 20 mL vial was sequentially charged with 1-chloro-4-(1-(2-methylpyridin-4-yl)ethyl)phthalazine (3.52 mmol, 1.0 g), 7 mL of DMF, DIPEA (10.56 mmol, 1.84 mL), and tetryhydropyran-4-ylmethylamine (7.04 mmol, 0.764 mL) and sealed with a crimp cap. The vial was placed in a microwave reactor and heated to 160° C. for 55 minutes. The reaction mixture was loaded onto a 120 g silica-gel cartridge and purified on a Biotage flash system eluting with DCM/MeOH. The fractions containing the product were concentrated down, then loaded onto a 120 g C18 silica-gel cartridge and purified on a Biotage flash system eluting with H₂O/MeOH+0.1% TFA, affording 1.09 g (85%) of 4-(1-(2-methylpyridin-4-yl)ethyl)-N-((tetrahydro-2H-pyran-4-yl)methyl)phthalazin-1-amine as a clear oil. ¹H NMR (400 MHz, d₆-DMSO) δ 10.09 (bt, 1H), 8.77 (m, 1H), 8.73 (d, 1H), 8.20 (m, 1H), 8.12 (m, 1H), 7.87 (s, 1H), 7.82 (m, 1H), 5.37 (q, 1H), 3.89 (m, 2H), 3.53 (m, 2H), 3.30 (m, 2H), 2.66 (s, 3H), 2.06 (m, 1H), 1.76 (m, 1H), 1.72 (m, 3H), 1.31 (m, 2H). 13C NMR (101 MHz d₆-DMSO) δ 158.50, 158.15, 154.12, 151.71, 150.44, 142.08, 135.63, 133.48, 126.25, 126.11, 126.02, 125.02, 123.25, 121.00, 66.53, 55.00, 48.54, 47.45, 33.16, 30.00, 19.90, 19.71. LC/MS (ESI, M+1): found 363.3

### Starting Material 9

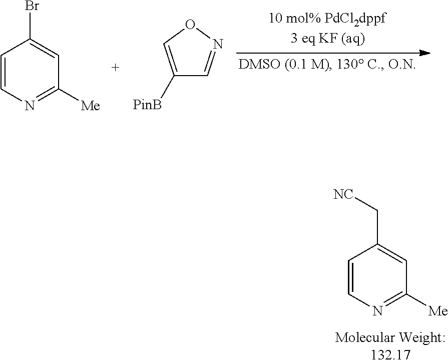

A 500 mL R.B. was charged with 4-(4,4,5,5-tetramethyl-1,3,2-dioxaborolan-2-yl)isoxazole (12 mmol, 2.34 g), KF (30 mmol, 1.74 g), 100 mL of DMSO, and 30 mL of water. The mixture was degassed by bubbling argon through it for 15 minutes. To the R.B. was added PdCl₂dppf and the system was flushed under a positive pressure of argon. While flushing the system with argon 4-bromo-2-methylpyridine (10 mmol, 1.18 mL) was injected in, then a balloon of argon was attached. The R.B. was placed in an oil-bath and heated to 130° C. for 16 hours. The cooled reaction mixture was filtered through a plug of celite that was rinsed with EtOAc. The filtrate was transferred to a sep-funnel containing Brine and EtOAc. The aqueous layer was extracted with EtoAc (×3). The combined organic layers were washed with brine, dried over sodium sulfate, filtered, and concentrated. The crude material was loaded onto a 220 g silica-gel cartridge and purified on a Biotage flash system eluting with DCM/MeOH, affording 1.0 g of 2-(2-methylpyridin-4-yl)acetonitrile as a dark red oil, which was stored under argon in a −20° C. freezer. ¹H NMR (400 MHz, d₆-DMSO) δ 8.45 (d, J=5.1 Hz, 1H), 7.24 (s, 1H), 7.18 (d, J=5.1 Hz, 1H), 4.10 (s, 2H), 2.48 (s, 3H)

### Starting Material 9-1

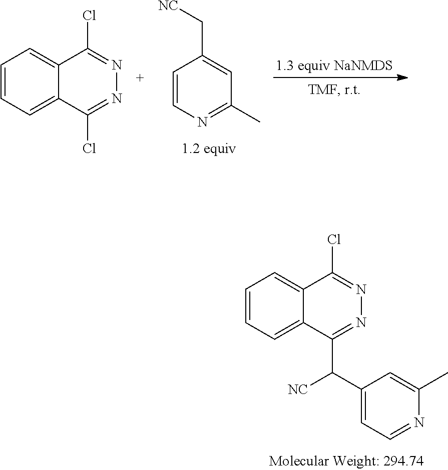

A flame dried 25 mL R.B. was charged with 1,4-dichlorophthalazine (1 mmol, 0.199 g) and placed under an atmosphere of argon. To the flask was injected 2-(2-methylpyridin-4-yl)acetonitrile (1.2 mmol, 0.158 g) and 2 mL of dry THF. A 1M solution of NaHMDS (1.3 mmol, 1.3 mL) in THF was injected dropwise and the reaction was stirred at r.t. The reaction mixture was quenched with 0.2 mL of sat NH₄Cl(aq), then concentrated down. The crude material was loaded onto a 220 g silica-gel cartridge and purified on a Biotage flash system eluting with DCM/MeOH, affording 0.201 g (68%) of 2-(4-chlorophthalazin-1-yl)-2-(2-methylpyridin-4-yl)acetonitrile as a dark red solid. LC/MS (ESI, M+1): found 295.6

### Starting Material 9-2

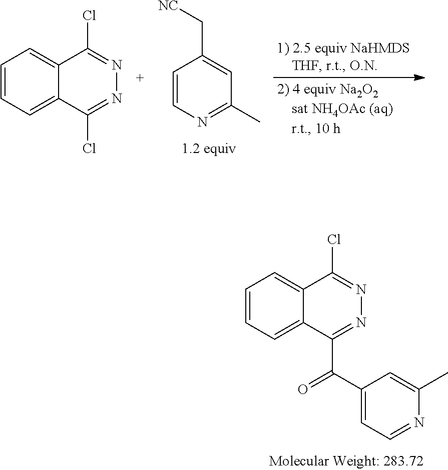

A flame dried 100 mL R.B. was charged with 1,4-dichlorophthalazine (1 mmol, 0.199 g) and placed under a positive pressure of argon. To the R.B. was injected 20 mL of THF and 2-(2-methylpyridin-4-yl)acetonitrile (1.2 mmol, 0.158 g). The argon inlet was removed but the outlet to the bubbler was left attached. A 1M solution of NaHMDS (2.5 mmol, 2.5 mL) in THF was injected dropwise. The reaction mixture turned dark red upon addition. The reaction was stirred at r.t. for 12 hours To the reaction was added 5 mL of a sat NH₄OAc (aq) and sodium peroxide and the reaction was stirred for an additional 10 hours. Insoluble solids were filtered off and rinsed with MeOH. The filtrate was concentrated down and the crude material was loaded onto a 50 g silica-gel cartridge and purified on a Biotage flash system eluting with DCM/MeOH, affording 0.113 g (39%) of (4-chlorophthalazin-1-yl)(2-methylpyridin-4-yl)methanone as a red solid. LC/MS (ESI, M+1): found 284.5

### Starting Material 10

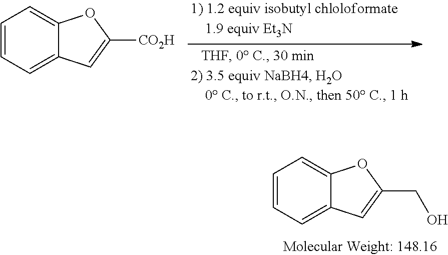 [SMILES: O=C(O)c1cc2ccccc2o1 ; OCc1cc2ccccc2o1]

A flame dried 500 mL R.B. was charged with benzofuran-2-carboxylic acid (20 mmol, 3.24 g) and placed under an atmosphere of argon. To the flask was injected 80 mL of THF and freshly distilled TEA (38 mmol, 5.26 mL). The R.B. was placed in an ice-bath and the mixture was equilibrated to temperature. Once cool, isobutyl chloroformate (24 mmol, 3.12 mL) was added dropwise to the solution. After complete addition the reaction was stirred for 30 minutes. To the slurry was slowly injected a solution of NaBH₄ (70 mmol, 2.65 g) in 40 mL of water. The ice-bath was removed and the reaction was warmed to r.t. and stirred O.N. The R.B. was then placed in an oil bath and heated to 50° C. for 1 h. The reaction mixture was re-cooled in an ice-bath and then quenched with sat. NH₄Cl (aq). The mixture was poured into a separatory-funnel and extracted with EtOAc. The organic layer was washed with water and brine, dried over sodium sulfate, filtered, and concentrated down. The crude material was loaded onto a 120 g silica-gel cartridge and purified on a Biotage flash system eluting with DCM, affording 2.02 g (68%) of benzofuran-2-ylmethanol as a light yellow oil. ¹H NMR (400 MHz, d₆-DMSO) δ 7.57 (m, 1H), 7.49 (m, 1H), 7.28 (m, 2H), 6.66 (s, 1H), 4.78 (s, 2H), 2.38 (bs, 1H)

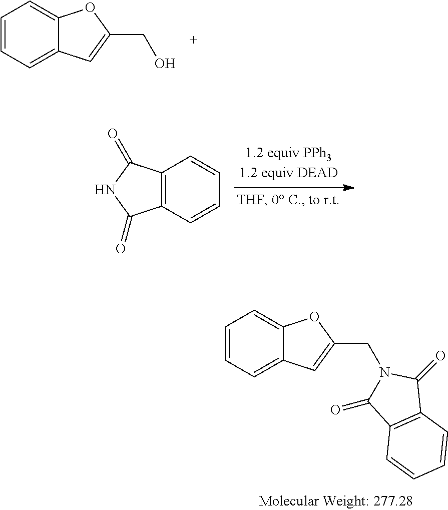 [SMILES: OCc1cc2ccccc2o1 ; O=C1NC(=O)c2ccccc21 ; O=C1c2ccccc2C(=O)N1Cc1cc2ccccc2o1]

A flame dried 200 mL R.B. was charged with phthalimide (16.2 mmol, 2.38 g) and PPh₃ (16.2 mmol, 4.25 g). The R.B. was sealed and placed under an atmosphere of argon. Dry THF (67.5 mL) was injected in followed by benzofuran-2-ylmethanol (13.5 mmol, 2 g), and the R.B. was placed in an ice-bath. Once the mixture equilibrated to temperature DEAD (40 wt % in toluene, 16.2 mmol, 7.37 mL) was injected dropwise over 10 minutes. The reaction was stirred gradually warming to r.t. O.N. The reaction mixture was concentrated down and the crude material was loaded onto a 220 g silica-gel cartridge and purified on a Biotage flash system eluting with Hex/EtOAc, affording 2.22 g (59%) of 2-(benzofuran-2-ylmethyl)isoindoline-1,3-dione as an off white solid. ¹H NMR (400 MHz, d₆-DMSO) δ 7.93 (m, 2H), 7.87 (m, 2H), 7.58 (d, J=8.1 Hz, 1H), 7.52 (d, J=8.1 Hz, 1H), 7.37-7.13 (m, 2H), 6.89 (s, 1H), 4.96 (s, 2H)

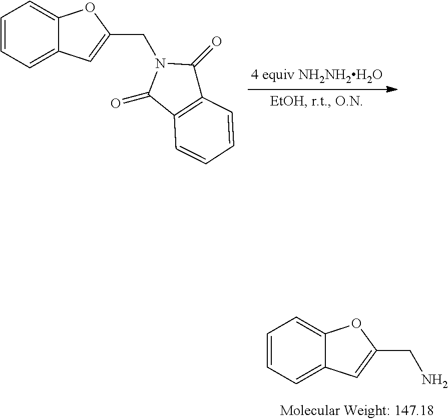 [SMILES: O=C1c2ccccc2C(=O)N1Cc1cc2ccccc2o1 ; NCc1cc2ccccc2o1]

To the 500 mL R.B. flask in which 2-(benzofuran-2-ylmethyl)isoindoline-1,3-dione (8 mmol, 2.22 g) had been concentrated into, under an atmosphere of argon, was added 40 ml of absolute EtOH then 1.56 mL of hydrazine monohydrate. The reaction was stirred O.N. at r.t. Material had precipitated out O.N. The reaction mixture was concentrated down, and the solid material was dissolved in a mixture of EtOAc and 1 M NaOH (aq). The layers were separated and the organic layer was washed with brine, dried over sodium sulfate, filtered, and concentrated down. The crude material was loaded onto a 50 g silica-gel cartridge and purified on a Biotage flash system eluting with DCM/MeOH, affording 0.915 g (77%) of benzofuran-2-ylmethanamine as a clear light yellow oil. ¹H NMR (400 MHz, d₆-DMSO) δ 7.56 (m, 1H), 7.49 (m, 1H), 7.27-7.16 (m, 2H), 6.68 (m, 1H), 5.50 (bs, 2H), 3.84 (d, J=1.1 Hz, 2H)

General Procedure A—Synthesis of 2-amino-benzoxazoles

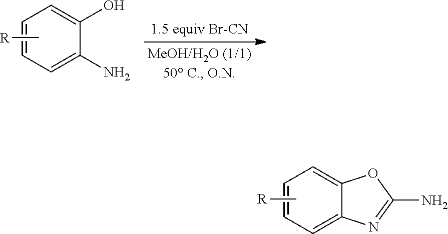

A 20 mL vial was charged with an 2-aminophenol (5 mmol) and 10 mL of a MeOH/H₂O (1/1) solution. The vial was sealed with crimp cap then flushed under a positive pressure of argon. The vial was placed in a reactor plate, and to the solution was injected a 3M solution of cyanogen bromide (7.5 mmol, 2.5 mL) in DCM. The reactor plate was then heated to 50° C. and the reaction was stirred O.N. The cooled reaction mixture was quenched with 1 M NaOH(aq) until a pH of 7-8 was reached. The mixture was poured into a sep-funnel containing EtOAc. The layers were separated and the aqueous layer was extracted with EtOAc. The combined organics were washed with water (×2) then brine, dried over sodium sulfate, filtered, and concentrated down. The crude material was used without further purification.

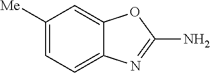 [SMILES: Cc1ccc2nc(N)oc2c1]

6-methylbenzo[d]oxazol-2-amine: Following the general procedure A using 2-amino-5-methylphenol (5 mmol, 0.615 g) afforded 0.741 g (100%) of product as a light brown solid. ¹H NMR (400 MHz, d₆-DMSO) δ 7.23 (bs, 2H), 7.14 (s, 1H), 7.06 (m, 1H), 6.90 (m, 1H), 2.33 (s, 3H)

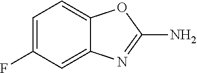 [SMILES: Nc1nc2cc(F)ccc2o1]

5-fluorobenzo[d]oxazol-2-amine: Following the general procedure A using 2-amino-4-fluorophenol (5 mmol, 0.635 g) afforded 0.749 g (98%) of product as a brown solid. ¹H NMR (400 MHz, d₆-DMSO) δ 7.55 (bs, 2H), 7.31 (dd, J=4.5 & 8.7 Hz, 1H), 7.02 (dd, J=2.6 & 9.3 Hz, 1H), 6.75 (ddd, J=2.7, 8.6 & 10.0 Hz, 1H).

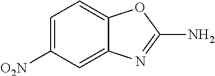 [SMILES: Nc1nc2cc([N+](=O)[O-])ccc2o1]

5-nitorobenzo[d]oxazol-2-amine: Following the general procedure A using 2-amino-4-nitrophenol (5 mmol, 0.77 g) afforded 0.811 g (90%) of product as a yellow-green solid. ¹H NMR (400 MHz, d₆-DMSO) δ 8.00-7.90 (m, 4H), 7.57 (d, J=8.7 Hz, 1H)

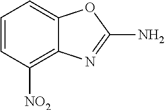 [SMILES: Nc1nc2c([N+](=O)[O-])cccc2o1]

4-nitorobenzo[d]oxazol-2-amine: Following the general procedure A using 2-amino-3-nitrophenol (5 mmol, 0.77 g) afforded 0.631 g (70%) of product as an orange solid. ¹H NMR (400 MHz, d₆-DMSO) δ 8.34 (bs, 2H), 7.91 (dd, 8.5, 1.0 Hz, 1H), 7.74 (dd, J=7.8, 1.0 Hz, 1H), 7.11 (dd, J=8.5, 7.8 Hz, 1H).

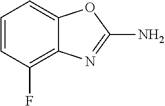 [SMILES: Nc1nc2c(F)cccc2o1]

4-fluorobenzo[d]oxazol-2-amine: Following the general procedure A using 2-amino-3-fluorophenol (5 mmol, 0.635 g) afforded 0.495 g (65%) of product as a burnt red solid. ¹H NMR (400 MHz, d₆-DMSO) δ 7.60 (bs, 2H), 7.23-7.19 (m, 1H), 7.06-6.86 (m, 2H)

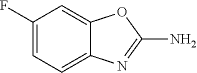 [SMILES: Nc1nc2ccc(F)cc2o1]

6-fluorobenzo[d]oxazol-2-amine: Following the general procedure A using 2-amino-5-fluorophenol (5 mmol, 0.635 g) afforded 0.371 g (49%) of product as a dark purple solid. ¹H NMR (400 MHz, d₆-DMSO) δ 7.41 (bs, 2H), 7.33 (dd, J=8.6, 2.5 Hz, 1H), 7.16 (dd, J=8.5, 4.9 Hz, 1H), 6.94 (ddd, J=10.3, 8.5, 2.6 Hz, 1H).

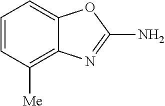 [SMILES: Cc1cccc2oc(N)nc12]

4-methylbenzo[d]oxazol-2-amine: Following the general procedure A using 2-amino-3-methylphenol (5 mmol, 0.615 g) afforded 0.646 g (87%) of product as a red brown solid.

LC/MS (ESI, M+1): found 149.0

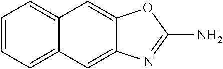 [SMILES: Nc1nc2cc3ccccc3cc2o1]

naphtho[2,3-d]oxazol-2-amine: Following the general procedure A using 3-amino-2-naphthol (5 mmol, 0.796 g) afforded 0.323 g (35%) of product as a red brown solid.

LC/MS (ESI, M+1): found 185.1

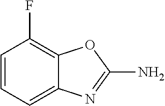 [SMILES: Nc1nc2cccc(F)c2o1]

7-fluorobenzo[d]oxazol-2-amine: Following the general procedure A using 2-amino-6-fluorophenol (5 mmol, 0.635 g) afforded 0.371 g (49%) of product as a dark purple solid.

LC/MS (ESI, M+1): found 153.0

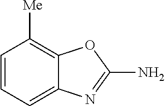 [SMILES: Cc1cccc2nc(N)oc12]

7-methylbenzo[d]oxazol-2-amine: Following the general procedure A using 2-amino-3-methylphenol (5 mmol, 0.615 g) afforded 0.58 g (78%) of product as a orange solid. LC/MS (ESI, M+1): found 149.0

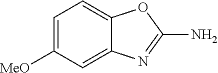 [SMILES: COc1ccc2oc(N)nc2c1]

6-methoxybenzo[d]oxazol-2-amine: Following the general procedure A using 2-amino-4-methoxyphenol (5 mmol, 0.695 g) afforded 0.701 g (85%) of product as a red solid. LC/MS (ESI, M+1): found 165.0

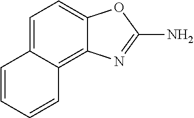 [SMILES: Nc1nc2c(ccc3ccccc32)o1]

naphtho[1,2-d]oxazol-2-amine: Following the general procedure A using 1-aminonaphthalen-2-ol hydrochloride (5 mmol, 0.978 g) and DIPEA (5 mmol, 0.871 mL) afforded 0.412 g (44%) of product as a tan solid. LC/MS (ESI, M+1): found 185.0

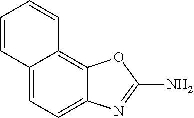 [SMILES: Nc1nc2ccc3ccccc3c2o1]

naphtho[2,1-d]oxazol-2-amine: Following the general procedure A using 2-amino-1-naphthol hydrochloride (5 mmol, 0.978 g) and DIPEA (5 mmol, 0.871 mL) afforded 0.501 g (54%) of product as a tan solid. ¹H NMR (400 MHz, d₆-DMSO) δ 8.02-7.92 (m, 2H), 7.79 (m, 1H), 7.61 (m, 1H, 7.52-7.35 (m, 4H)

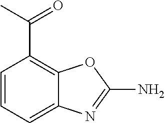

1-(2-aminobenzo[d]oxazol-7-yl)ethan-1-one: Following the general procedure A using 3′-amino-2′-hydroxyacetophenone (5 mmol, 0.756 g) afforded 0.598 g (67%) of product as a tan solid. ¹H NMR (400 MHz, d₆-DMSO) δ 7.54 (dd, J=8.1, 1.4 Hz, 1H), 7.34 (dd, J=7.7, 1.3 Hz, 1H), 7.26 (dd, J=8.4, 7.3 Hz, 1H), 2.65 (s, 3H).

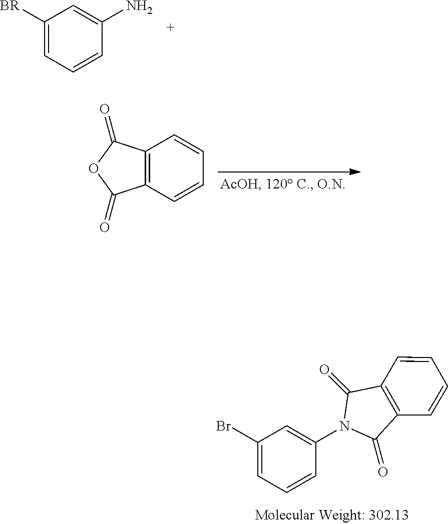

A 200 mL R.B. was charged with 3-bromoaniline (10 mmol, 1.09 mL), phthalic anhydride (10 mmol, 1.48 g), and 60 mL of acetic acid. A reflux condenser was attached and the R.B. was placed in an oil bath. The oil bath was heated to 120° C. and the reaction was refluxed O.N. To the cooled reaction mixture was added 50 mL of water and the resulting slurry was stirred for 15 minutes. The precipitated solid was filtered and rinsed with water, affording 2.7 g (89%) of 2-(3-bromophenyl)isoindoline-1,3-dione as a white solid. ¹H NMR (400 MHz, CDCl₃) δ 7.99 (m, 2H), 7.84 (m, 2H), 7.68 (m, 1H), 7.57 (ddd, J=7.7, 1.9, 1.4 Hz, 1H), 7.50-7.35 (m, 2H).

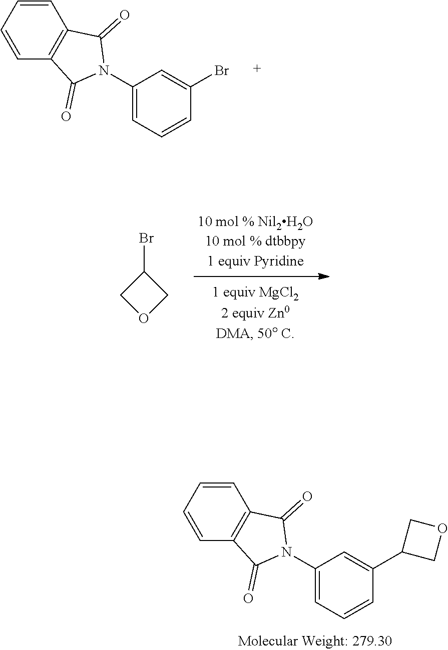 [SMILES: O=C1c2ccccc2C(=O)N1c1cccc(Br)c1 ; BrC1COC1 ; O=C1c2ccccc2C(=O)N1c1cccc(C2COC2)c1]

In an argon filled glove box, MgCl₂ (10 mmol, 0.952 g) and zinc powder (−325 mesh, 20 mmol, 1.31 g) were weighed into a 250 mL R.B. The R.B. was sealed and taken out of the glovebox. The NiI₂ (1 mmol, 0.375 g), dtbbpy (1 mmol, 0.268 g), and 2-(3-bromophenyl)isoindoline-1,3-dione (10 mmol, 3.02 g) were added to the R.B., which was resealed and placed under an atmosphere of argon via three vacuum-purge cycles. While under a positive pressure of argon pyridine (10 mmol, 0.81 mL), 3-bromooxetane (20 mmol, 1.66 mL), and 60 mL degassed DMA were injected in. A balloon of argon was attached and the R.B. was placed in an oil bath and stirred at 50° C. for 14 hours. The cooled reaction mixture was transferred to a sep-funnel containing EtOAc and water. The organic layer was washed with brine (×2). The combined aqueous layers were extracted with EtOAc, which was washed with water then brine (×2). The combined organic layers were dried over sodium sulfate, filtered, and concentrated down. The crude material was loaded onto a 100 g silica-gel cartridge and purified on a Biotage flash system eluting with Hex/EtOAc, affording 1.364 g (49%) of 2-(3-(oxetan-3-yl)phenyl)isoindoline-1,3-dione as an off white solid. ¹H NMR (400 MHz, d₆-DMSO) δ 7.98 (m, 2H), 7.92 (m, 2H), 7.57-7.48 (m, 3H), 7.36 (m, 1H), 4.98 (dd, J=8.4, 5.9 Hz, 2H), 4.65 (dd, J=6.7, 5.9 Hz, 2H), 4.33 (tt, J=8.4, 6.7 Hz, 1H)

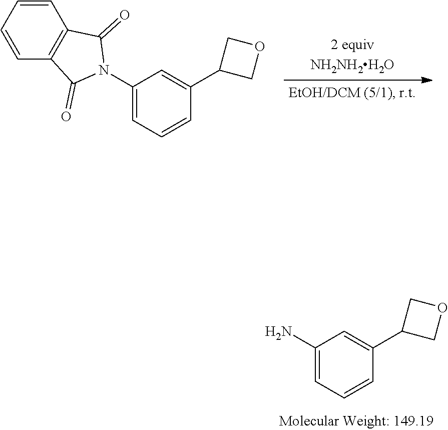 [SMILES: O=C1c2ccccc2C(=O)N1c1cccc(C2COC2)c1 ; Nc1cccc(C2COC2)c1]

To a 5 mL vial was sequentially added 2-(3-(oxetan-3-yl)phenyl)isoindoline-1,3-dione (0.85 mmol, 0.237 g), 2.8 mL of a EtOH/DCM (5/1) solution, and NH₂NH₂·H₂O (1.7 mmol, 0.08 mL). The vial was sealed and stirred at r.t. for 14 hours. A white precipitate had formed. The reaction mixture was filtered and rinsed with diethyl ether. The filtrate was concentrated down and the crude material loaded onto a 50 g silica-gel cartridge and purified on a Biotage flash system eluting with Hex/EtOAc, affording 0.085 g (67%) of 3-(oxetan-3-yl)aniline as a yellow oil. ¹H NMR (400 MHz, d₆-DMSO) δ 7.17 (t, J=7.5 Hz, 1H), 6.89-6.74 (m, 2H), 6.63 (m, 1H), 5.05 (dd, J=8.4, 5.9 Hz, 2H), 4.77 (dd, J=6.8, 5.9 Hz, 2H), 4.15 (m, 1H), 3.66-3.08 (bs, 2H).

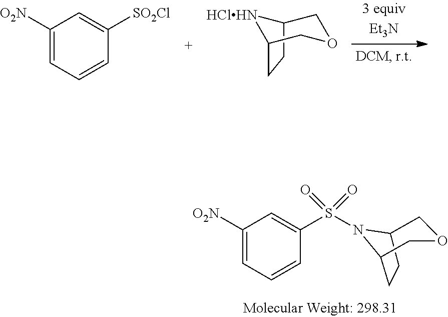

A 20 ml vial was sequentially charged with 3-nitrobenzenesulfonyl chloride (1 mmol, 0.221 g), 3-oxa-8-azabicyclo[3.2.1]octane hydrochloride (1.1 mmol, 0.164 g), 5 ml of DCM, then triethylamine (3 mmol, 0.418 mL). The vial was sealed and stirred at room temperature for 12 hours. The reaction mixture was directly loaded onto a 50 g silica-gel cartridge and purified on a Biotage flash system eluting with DCM, affording 0.211 g (71%) of 8-((3-nitrophenyl)sulfonyl)-3-oxa-8-azabicyclo[3.2.1]octane as a white solid. ¹H NMR (400 MHz, CDCl₃) δ 8.74 (t, J=2.0 Hz, 1H), 8.47 (ddd, J=8.2, 2.3, 1.1 Hz, 1H), 8.23 (ddd, J=7.8, 1.7, 1.1 Hz, 1H), 7.77 (t, J=8.0 Hz, 1H), 4.16 (m, 2H), 3.76 (m, 2H), 3.64 (m, 2H), 1.97 (m, 2H), 1.66 (m, 2H)

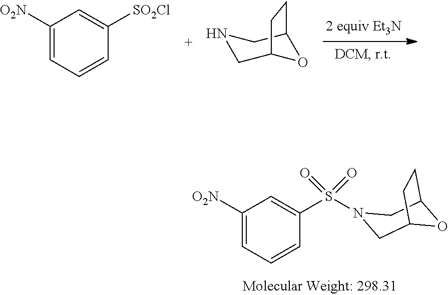 [SMILES: O=[N+]([O-])c1cccc(S(=O)(=O)Cl)c1 ; C1CC2CNCC1O2 ; O=[N+]([O-])c1cccc(S(=O)(=O)N2CC3CCC(C2)O3)c1]

A 20 ml vial was sequentially charged with 3-nitrobenzenesulfonyl chloride (1 mmol, 0.221 g), 8-oxa-3-azabicyclo[3.2.1]octane hydrochloride (1.1 mmol, 0.124 g), 5 ml of DCM, then triethylamine (2 mmol, 0.279 mL). The vial was sealed and stirred at room temperature for 12 hours. The reaction mixture was directly loaded onto a 50 g silica-gel cartridge and purified on a Biotage flash system eluting with DCM, affording 0.211 g (71%) of 8-((3-nitrophenyl)sulfonyl)-3-oxa-8-azabicyclo[3.2.1]octane as a white solid. ¹H NMR (400 MHz, CDCl₃) δ 8.59 (m, 1H), 8.49 (ddd, J=8.2, 2.3, 1.1 Hz, 1H), 8.07 (ddd, J=7.8, 1.7, 1.1 Hz, 1H), 7.80 (t, J=8.0 Hz, 1H), 4.42 (m, 2H), 3.48 (m, 2H), 2.69 (dd, J=11.3, 2.3 Hz, 2H), 2.12-1.95 (m, 4H).

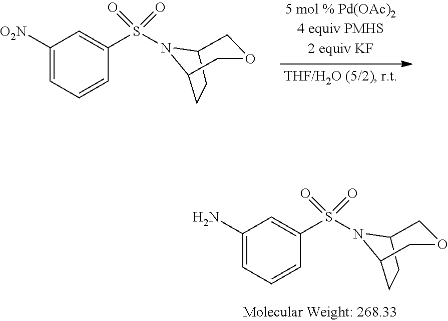 [SMILES: O=[N+]([O-])c1cccc(S(=O)(=O)N2C3CCC2COC3)c1 ; Nc1cccc(S(=O)(=O)N2C3CCC2COC3)c1]

8-((3-nitrophenyl)sulfonyl)-3-oxa-8-azabicyclo[3.2.1]octane (0.707 mmol, 0.211 g) was reduced affording 0.167 g (88%) of 3-((3-oxa-8-azabicyclo[3.2.1]octan-8-yl)sulfonyl)aniline as a light pink solid. ¹H NMR (400 MHz, d₆-DMSO) δ 7.09 (t, J=7.9 Hz, 1H), 6.92 (t, J=2.0 Hz, 1H), 6.81 (ddd, J=7.7, 1.9, 1.0 Hz, 1H), 6.70 (ddd, J=8.1, 2.3, 1.0 Hz, 1H), 5.50 (bs, 2H), 3.87 (m, 2H), 3.44 (m, 4H), 1.63-1.50 (m, 2H), 1.33-1.16 (m, 2H).

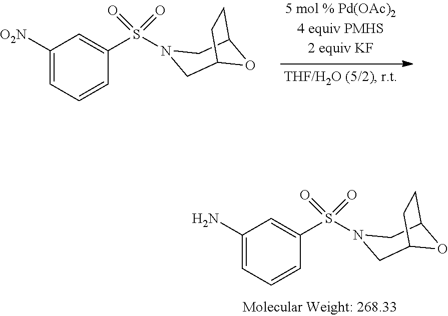 [SMILES: O=[N+]([O-])c1cccc(S(=O)(=O)N2CC3CCC(C2)O3)c1 ; Nc1cccc(S(=O)(=O)N2CC3CCC(C2)O3)c1]

8-((3-nitrophenyl)sulfonyl)-8-oxa-3-azabicyclo[3.2.1]octane (0.69 mmol, 0.206 g) was reduced affording 0.146 g (79%) of 3-((8-oxa-3-azabicyclo[3.2.1]octan-3-yl)sulfonyl)aniline as a pink solid. ¹H NMR (400 MHz, d₆-DMSO) δ 7.14 (m, 1H), 6.77 (m, 1H), 6.72 (m, 1H), 6.65 (m, 1H), 5.55 (bs, 2H), 4.23 (m, 2H), 3.07 (m, 2H), 2.37 (m, 2H), 1.69 (m, 4H)

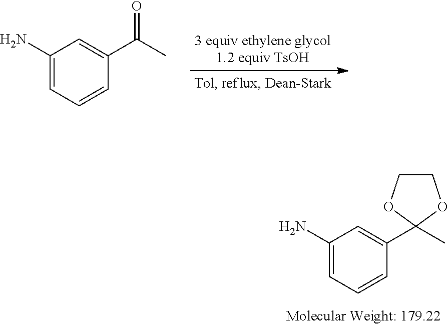

A 250 mL R.B. was charged with 3′-aminoacetophenone (10 mmol, 1.35 g), p-toluenesulfonic acid monohydrate (12 mmol, 2.28 g), ethylene glycol (30 mmol, 1.67 mL), and 130 mL of toluene. A Dean-Stark trap, equipped with a reflux condenser and a drying tube, was attached to the R.B. The reaction was heated to reflux and stirred for 24 hours. The cooled reaction mixture was partially concentrated down to ˜25 mL, transferred to a separatory funnel with EtOAc, and washed with sat NaHCO₃ (aq). The organic layer was dried over sodium sulfate, filtered, and concentrated down affording 0.53 g (29%) of 3-(2-methyl-1,3-dioxolan-2-yl)aniline as dark tan solid. ¹H NMR (400 MHz, d₆-DMSO) δ 6.97 (t, J=7.8 Hz, 1H), 6.64 (t, J=2.0 Hz, 1H), 6.54 (dt, J=7.6, 1.3 Hz, 1H), 6.47 (ddd, J=7.9, 2.4, 1.0 Hz, 1H), 5.07 (bs, 2H), 3.94 (m, 2H), 3.66 (m, 2H), 1.49 (s, 3H)

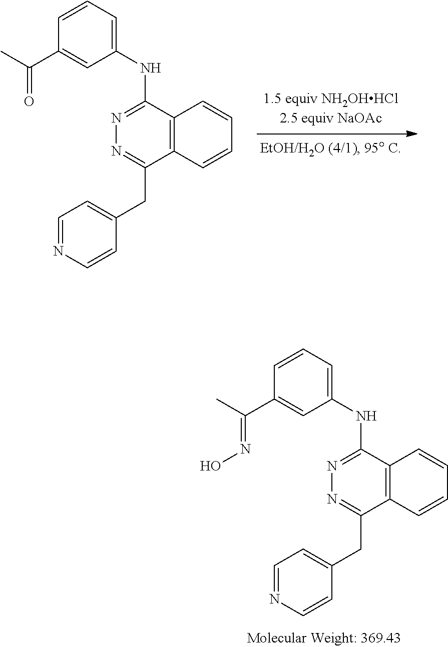 [SMILES: CC(=O)c1cccc(Nc2nnc(Cc3ccncc3)c3ccccc23)c1 ; C/C(=N\O)c1cccc(Nc2nnc(Cc3ccncc3)c3ccccc23)c1]

SR-30013: A 5 mL vial was charged with 1-(3-((4-(pyridin-4-ylmethyl)phthalazin-1-yl)amino)phenyl)ethan-1-one (“SR1-34005”, 0.25 mmol, 0.089 g), hydroxylamine hydrochloride (0.375 mmol, 0.026 g), sodium acetate (0.625 mmol, 0.051 g), and 1 mL of a (4/1) EtOH/H₂O solution. The vial was sealed with a crimp cap, placed in a reactor plate that was preheated to 95° C., and stirred for 14 hours. The reaction mixture was cooled to r.t., and the precipitated solid was filtered and rinsed with water, EtOH, then Et₂O affording 0.047 g (51%) of SR-30013 as a light-yellow solid. ¹H NMR (400 MHz, d₆-DMSO) δ11.23 (s, 1H), 9.23 (bs, 1H), 8.64 (m, 1H), 8.45 (m, 2H), 8.34 (t, J=2.0 Hz, 1H), 8.12 (dd, J=8.3, 1.2 Hz, 1H), 8.03-7.89 (m, 3H), 7.38 (t, J=7.9 Hz, 1H), 7.34-7.27 (m, 3H), 4.60 (s, 2H), 2.20 (s, 3H). LC/MS (ESI, M+1): found 370.3

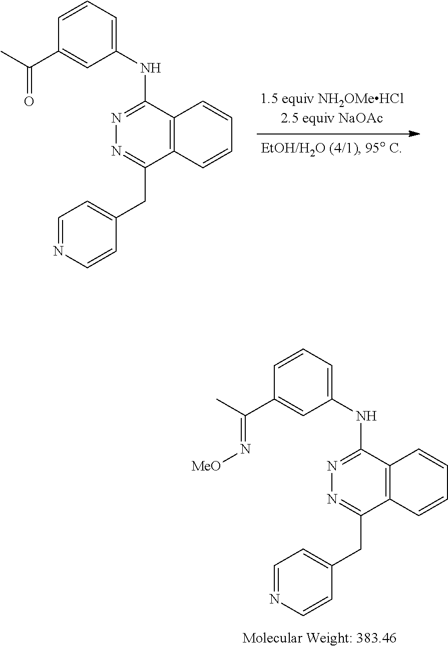 [SMILES: CC(=O)c1cccc(Nc2nnc(Cc3ccncc3)c3ccccc23)c1 ; CO/N=C(\C)c1cccc(Nc2nnc(Cc3ccncc3)c3ccccc23)c1]

SR-30014: A 5 mL vial was charged with SR1-34005 (0.25 mmol, 0.089 g), O-methylhydroxylamine hydrochloride (0.375 mmol, 0.031 g), sodium acetate (0.625 mmol, 0.051 g), and 1 mL of a (4/1) EtOH/H₂O solution. The vial was sealed with a crimp cap, placed in a reactor plate that was preheated to 95° C., and stirred for 14 hours. The reaction mixture was cooled to r.t., and transferred to a separatory funnel with the aid of EtOAc and water. The reaction mixture was extracted with EtOAc. The organic layer was washed with brine, dried over sodium sulfate, filtered, and concentrated down. The crude material was loaded onto a 25 g silica-gel cartridge and purified on a Biotage flash system eluting with DCM/MeOH, affording 0.086 g (89%) of SR-30014 as a light yellow solid. LC/MS (ESI, M+1): found 384.3

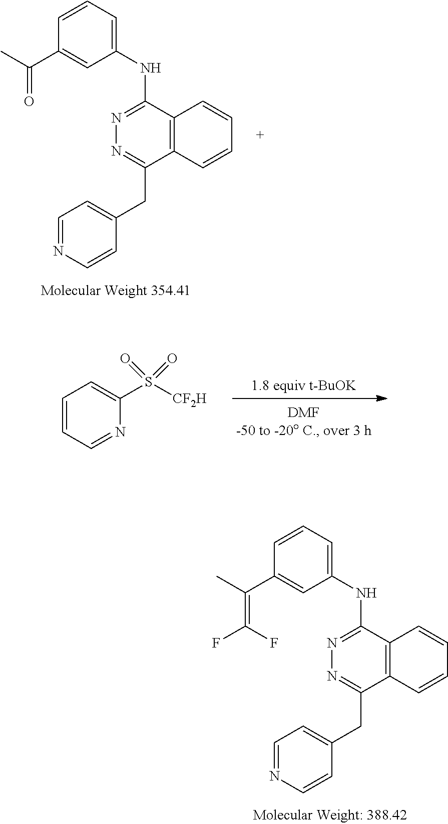 [SMILES: CC(=O)c1cccc(Nc2nnc(Cc3ccncc3)c3ccccc23)c1 ; O=S(=O)(c1ccccn1)C(F)F ; CC(=C(F)F)c1cccc(Nc2nnc(Cc3ccncc3)c3ccccc23)c1]

SR-31144: Potassium tert-butoxide was dissolved in 0.5 mL of DMF in a dry 2 mL vial under an argon atmosphere. A separate 5 mL vial was charged with SR1-34005 (0.282 mmol, 0.100 g) and 2-(difluoromethylsulfonyl)pyridine (0.235 mmol, 0.045 g) then subjected to three vacuum/purge cycles with argon. To the vial was injected 1 mL of DMF and the reaction mixture was cooled in a −50° C. bath. Once equilibrated to temperature the potassium tert-butoxide solution was injected and the reaction slowly warmed to −20° C. over 3 hours. The reaction mixture was quenched with the addition of 0.5 mL of sat. NH₄Cl(aq) then 0.5 mL of 3M HCl, and allowed to warm to r.t. The mixture was loaded onto a 25 g silica-gel cartridge and purified on a Biotage flash system eluting with DCM/MeOH, affording 0.052 g (47%) of SR-31144 as a red solid. ¹H NMR (400 MHz, d₆-DMSO) δ 12.67 (s, 1H), 8.77 (d, J=5.7 Hz, 2H), 8.67 (d, J=4.6 Hz, 1H), 8.29 (dd, J=7.8, 1.4 Hz, 1H), 8.11 (td, J=7.7, 1.7 Hz, 1H), 8.02-7.82 (m, 6H), 7.61 (dd, J=7.6, 4.7 Hz, 1H), 4.61 (s, 2H), 2.49 (s, 3H). LC/MS (ESI, M+1): found 389.3

### General Procedure B

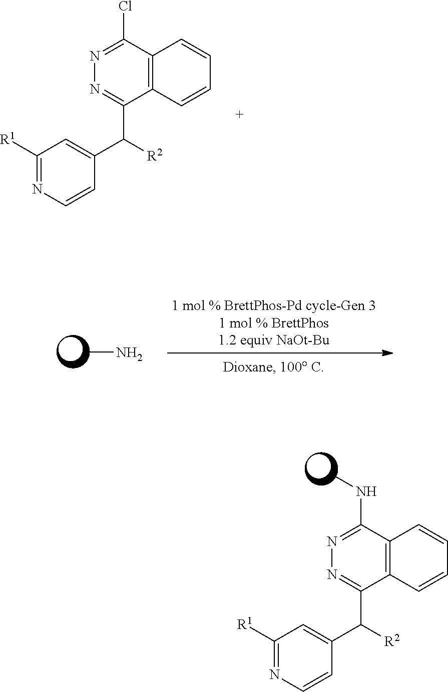

A 5 mL microwave vial was charged with 1-chloro-4-(pyridin-4-ylmethyl)phthalazine (0.25 mmol, 0.064 g), amine (0.3 mmol), BrettPhos-Pd-G3 (0.0025 mmol, 0.002 g), BrettPhos (0.0025 mmol, 0.001 g), and potassium phosphate tribasic (0.3 mmol, 0.064 g), sealed with a crimp cap, and subjected to three vacuum purge cycles with argon. While under a positive flow of argon 1,4-dioxane was injected in. The cap was rapped with a layer of parafilm and the vial was placed in a preheated reactor plate at 100° C. and stirred O.N. The cooled reaction mixture was directly loaded onto a 25 g silica-gel cartridge with the aid of some 99/1 DCM/MeOH and purified on a Biotage flash system eluting with DCM/MeOH. The fractions containing the product were concentrated down, then loaded onto a 60 g C18 silica-gel cartridge and purified on a Biotage flash system eluting with H₂O/MeOH+0.1% TFA.

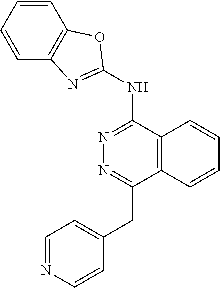 [SMILES: c1ccc2oc(Nc3nnc(Cc4ccncc4)c4ccccc34)nc2c1]

SR-30010: Following the general procedure B with 2-aminobenzoxazole (0.3 mmol, 0.067 g) afforded 0.063 g (72%) of SR-30010 as a light yellow solid. LC/MS (ESI, M+1): found 354.2

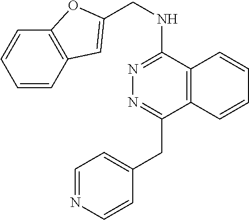 [SMILES: c1ccc2oc(CNc3nnc(Cc4ccncc4)c4ccccc34)cc2c1]

SR-30011: Following the general procedure B with benzofuranmethanamine (0.3 mmol, 0.038 mL) afforded 0.055 g (60%) of SR-30011 as a light yellow solid. LC/MS (ESI, M+1): found 367.3

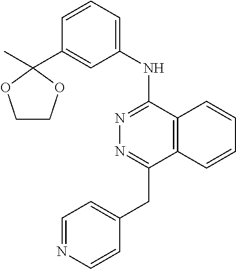 [SMILES: CC1(c2cccc(Nc3nnc(Cc4ccncc4)c4ccccc34)c2)OCCO1]

SR-30012: Following the general procedure B with 3-(2-methyl-1,3-dioxolan-2-yl)aniline (0.3 mmol, 0.054 5) afforded 0.049 g (49%) of SR-30012 as a cream solid. LC/MS (ESI, M+1): found 399.3

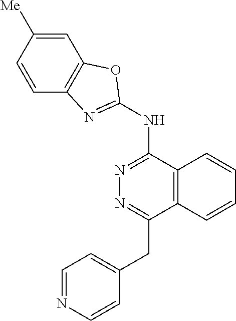 [SMILES: Cc1ccc2nc(Nc3nnc(Cc4ccncc4)c4ccccc34)oc2c1]

SR-32066: Following the general procedure B with 6-methylbenzoxazol-2-amine (0.3 mmol, 0.044 g) afforded 0.019 g (20%) of SR-32066 as a yellow solid. LC/MS (ESI, M+1): found 368.3

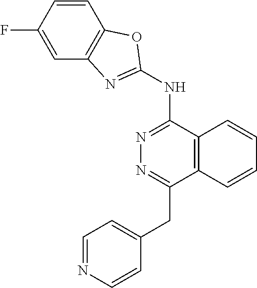 [SMILES: Fc1ccc2oc(Nc3nnc(Cc4ccncc4)c4ccccc34)nc2c1]

SR-32067: Following the general procedure B with 5-fluorobenzoxazol-2-amine (0.3 mmol, 0.046 g) afforded 0.030 g (32%) of SR-32067 as a burnt orange solid. ¹H NMR (400 MHz, d₆-DMSO) δ 14.34 (s, 1H), 8.49 (ddd, J=15.3, 4.4, 1.6 Hz, 2H), 8.34 (m, 1H), 8.14 (m, 1H), 8.02 (m, 1H), 7.41 (m, 1H), 7.30 (m, 2H), 7.02 (m, 1H), 6.75 (ddd, J=10.0, 8.6, 2.6 Hz, 1H), 4.77 (s, 1H), 4.54 (s, 1H). LC/MS (ESI, M+1): found 372.2

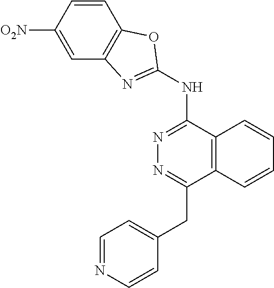 [SMILES: O=[N+]([O-])c1ccc2oc(Nc3nnc(Cc4ccncc4)c4ccccc34)nc2c1]

SR-32068: Following the general procedure B with 5-nitrobenzoxazol-2-amine (0.3 mmol, 0.054 g) afforded 0.016 g (16%) of SR— as a yellow solid. LC/MS (ESI, M+1): found 399.2

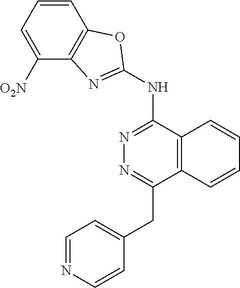 [SMILES: O=[N+]([O-])c1cccc2oc(Nc3nnc(Cc4ccncc4)c4ccccc34)nc12]

SR-32069: Following the general procedure B with 4-nitrobenzoxazol-2-amine (0.3 mmol, 0.054 g) afforded 0.035 g (35%) of SR-32069 as a yellow solid. LC/MS (ESI, M+1): found 399.2

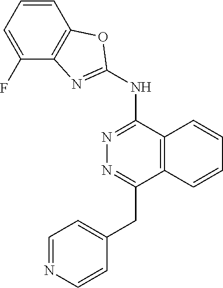 [SMILES: Fc1cccc2oc(Nc3nnc(Cc4ccncc4)c4ccccc34)nc12]

SR-32070: Following the general procedure B with 4-fluorobenzoxazol-2-amine (0.3 mmol, 0.046 g) afforded 0.042 g (45%) of SR-32070 as a light yellow solid. LC/MS (ESI, M+1): found 372.2

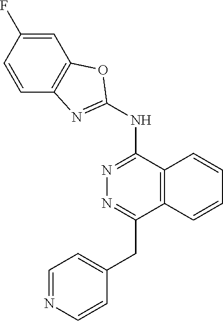 [SMILES: Fc1ccc2nc(Nc3nnc(Cc4ccncc4)c4ccccc34)oc2c1]

SR-32071: Following the general procedure B with 6-fluorobenzoxazol-2-amine (0.3 mmol, 0.046 g) afforded 0.007 g (8%) of SR-32071 as a dark red solid. LC/MS (ESI, M+1): found 372.2

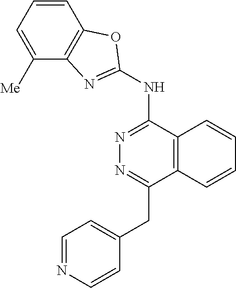 [SMILES: Cc1cccc2oc(Nc3nnc(Cc4ccncc4)c4ccccc34)nc12]

SR-32072: Following the general procedure B with 4-methylbenzoxazol-2-amine (0.3 mmol, 0.044 g) afforded 0.089 g (98%) of SR-32072 as a yellow solid. LC/MS (ESI, M+1): found 368.3

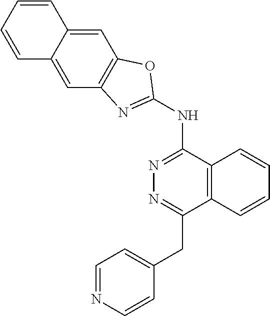 [SMILES: c1ccc2cc3oc(Nc4nnc(Cc5ccncc5)c5ccccc45)nc3cc2c1]

SR-32073: Following the general procedure B with naphtho[2,3-d]oxazol-2-amine (0.3 mmol, 0.055 g) afforded 0.022 g (21%) of SR-32073 as a yellow solid.

LC/MS (ESI, M+1): found 404.3

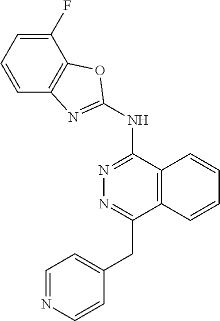 [SMILES: Fc1cccc2nc(Nc3nnc(Cc4ccncc4)c4ccccc34)oc12]

SR-32074: Following the general procedure B with 6-fluorobenzoxazol-2-amine (0.3 mmol, 0.046 g) afforded 0.026 g (28%) of SR-32074 as a tan solid. LC/MS (ESI, M+1): found 372.2

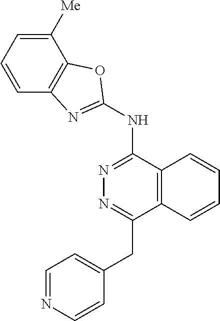 [SMILES: Cc1cccc2nc(Nc3nnc(Cc4ccncc4)c4ccccc34)oc12]

SR-32075: Following the general procedure B with 7-methylbenzoxazol-2-amine (0.3 mmol, 0.044 g) afforded 0.048 g (52%) of SR-32075 as a light brown solid. LC/MS (ESI, M+1): found 368.3

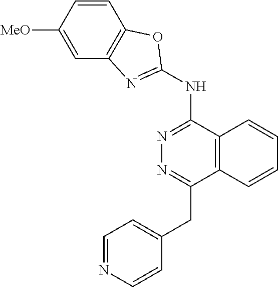 [SMILES: COc1ccc2oc(Nc3nnc(Cc4ccncc4)c4ccccc34)nc2c1]

SR-32076: Following the general procedure B with 5-methoxybenzoxazol-2-amine (0.3 mmol, 0.046 g) afforded 0.038 g (39%) of SR-32076 as a light brown solid. LC/MS (ESI, M+1): found 384.3

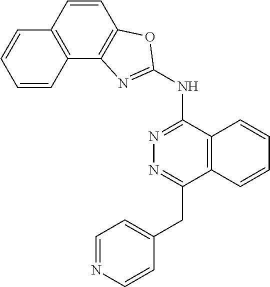 [SMILES: c1ccc2c(c1)ccc1oc(Nc3nnc(Cc4ccncc4)c4ccccc34)nc12]

SR-32077: Following the general procedure B with naphtho[1,2-d]oxazol-2-amine (0.3 mmol, 0.055 g) afforded 0.031 g (31%) of SR-32077 as a dark yello solid. LC/MS (ESI, M+1): found 404.3

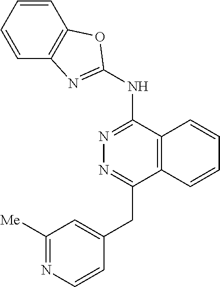 [SMILES: Cc1cc(Cc2nnc(Nc3nc4ccccc4o3)c3ccccc23)ccn1]

SR-33872: Following a modified version of general procedure B with 1-chloro-4-((2-methylpyridin-4-yl)methyl)phthalazine (0.25 mmol, 0.067 g) and 2-aminobenzoxazole (0.3 mmol, 0.040 g) afforded 0.018 g (20%) of SR-33872 as a yellow solid.

LC/MS (ESI, M+1): found 368.3

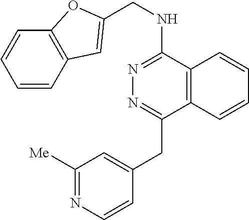 [SMILES: Cc1cc(Cc2nnc(NCc3cc4ccccc4o3)c3ccccc23)ccn1]

SR-33875: Following a modified version of general procedure B with 1-chloro-4-((2-methylpyridin-4-yl)methyl)phthalazine (0.25 mmol, 0.067 g) and benzofurnan-2-ylmethanamine (0.3 mmol, 0.044 g) afforded 0.042 g (44%) of SR-33875 as a yellow solid. LC/MS (ESI, M+1): found 381.3

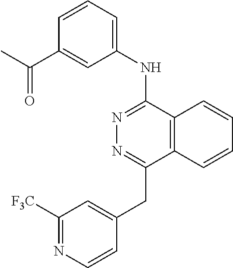 [SMILES: CC(=O)c1cccc(Nc2nnc(Cc3ccnc(C(F)(F)F)c3)c3ccccc23)c1]

SR-33873: Following a modified version of general procedure B with 1-chloro-4-((2-(trifluoromethyl)pyridin-4-yl)methyl)phthalazine (0.14 mmol, 0.046 g) and 3-aminoacetophenone (0.17 mmol, 0.023 g) afforded 0.042 g (70%) of SR-33873 as a yellow solid. LC/MS (ESI, M+1): found 423.3

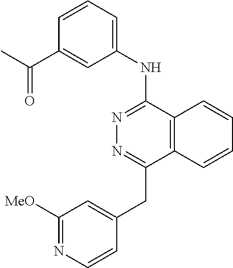 [SMILES: COc1cc(Cc2nnc(Nc3cccc(C(C)=O)c3)c3ccccc23)ccn1]

SR-33874: Following a modified version of general procedure B with 1-chloro-4-((2-methoxypyridin-4-yl)methyl)phthalazine (0.25 mmol, 0.071 g) and 3-aminoacetophenone (0.30 mmol, 0.041 g) afforded 0.037 g (38%) of SR-33874 as a light yellow solid. LC/MS (ESI, M+1): found 385.3

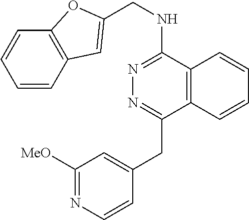 [SMILES: COc1cc(Cc2nnc(NCc3cc4ccccc4o3)c3ccccc23)ccn1]

SR-33875: Following a modified version of general procedure B with 1-chloro-4-((2-methoxypyridin-4-yl)methyl)phthalazine (0.25 mmol, 0.071 g) and benzofuran-2-ylmethanamine (0.30 mmol, 0.044 g) afforded 0.083 g (83%) of SR-33875 as a light yellow solid. LC/MS (ESI, M+1): found 397.3

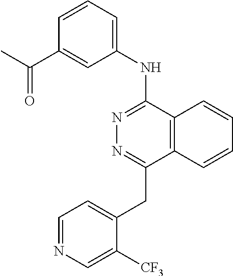 [SMILES: CC(=O)c1cccc(Nc2nnc(Cc3ccncc3C(F)(F)F)c3ccccc23)c1]

SR-33876: Following a modified version of general procedure B with 1-chloro-4-((3-(trifluoromethyl)pyridin-4-yl)methyl)phthalazine (0.25 mmol, 0.081 g) and 3-aminoacetophenone (0.30 mmol, 0.041 g) afforded 0.039 g (37%) of SR-33876 as a yellow solid. LC/MS (ESI, M+1): found 423.3

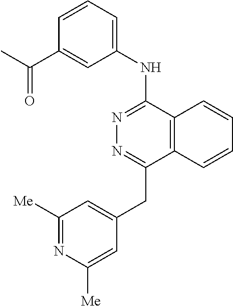 [SMILES: CC(=O)c1cccc(Nc2nnc(Cc3cc(C)nc(C)c3)c3ccccc23)c1]

SR-32064: Following a modified version of general procedure B with 1-chloro-4-((2,6-dimethylpyridin-4-yl)methyl)phthalazine (0.25 mmol, 0.071 g) and 3′-aminoacetophenone (0.3 mmol, 0.041 g) afforded 0.041 g (42%) of SR-32064 as a pale yellow solid. ¹H NMR (400 MHz, d₆-DMSO) δ 9.38 (s, 1H), 8.64 (m, 1H), 8.52 (t, J=2.0 Hz, 1H), 8.33 (ddd, J=8.2, 2.3, 1.0 Hz, 1H), 8.10 (dd, J=8.4, 1.2 Hz, 1H), 7.97 (m, 2H), 7.66 (dt, J=7.8, 1.2 Hz, 1H), 7.53 (t, J=7.9 Hz, 1H), 6.97 (s, 2H), 4.50 (s, 2H), 2.62 (s, 3H), 2.34 (s, 6H). LC/MS (ESI, M+1): found 383.3

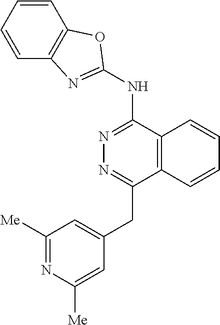 [SMILES: Cc1cc(Cc2nnc(Nc3nc4ccccc4o3)c3ccccc23)cc(C)n1]

SR-32065: Following a modified version of general procedure B with 1-chloro-4-((2,6-dimethylpyridin-4-yl)methyl)phthalazine (0.25 mmol, 0.071 g) and 2-aminobenzoxazole (0.3 mmol, 0.040 g) afforded 0.015 g (15%) of SR-32065 as a yellow solid. LC/MS (ESI, M+1): found 382.3

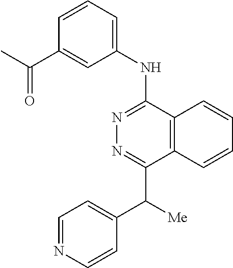 [SMILES: CC(=O)c1cccc(Nc2nnc(C(C)c3ccncc3)c3ccccc23)c1]

SR-34644: Following a modified version of general procedure B with 1-chloro-4-(1-(pyridin-4-yl)ethyl)phthalazine (0.25 mmol, 0.067 g) and 3-aminoacetophenone (0.30 mmol, 0.041 g) afforded 0.079 g (86%) of SR-34644 as a dark yellow solid. LC/MS (ESI, M+1): found 369.3

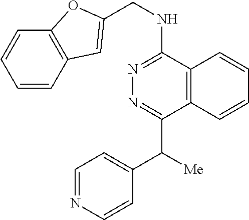 [SMILES: CC(c1ccncc1)c1nnc(NCc2cc3ccccc3o2)c2ccccc12]

SR-34645: Following a modified version of general procedure B with 1-chloro-4-(1-(pyridin-4-yl)ethyl)phthalazine (0.25 mmol, 0.067 g) and benzofuran-2-ylmethanamine (0.30 mmol, 0.044 g) afforded 0.077 g (81%) of SR-34645 as a dark orange solid. LC/MS (ESI, M+1): found 381.3

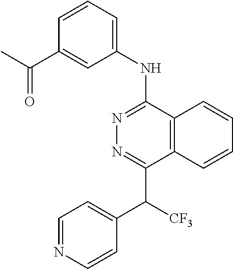 [SMILES: CC(=O)c1cccc(Nc2nnc(C(c3ccncc3)C(F)(F)F)c3ccccc23)c1]

SR-34646: Following a modified version of general procedure B with 1-chloro-4-(2,2,2-trifluoro-1-(pyridin-4-yl)ethyl)phthalazine (0.25 mmol, 0.081 g) and 3-aminoacetophenone (0.30 mmol, 0.041 g) afforded 0.027 g (25%) of SR-34646 as a dark yellow solid. LC/MS (ESI, M+1): found 423.3

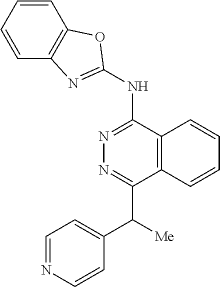 [SMILES: CC(c1ccncc1)c1nnc(Nc2nc3ccccc3o2)c2ccccc12]

SR-34779: Following a modified version of general procedure B with 1-chloro-4-(1-(pyridin-4-yl)ethyl)phthalazine (0.25 mmol, 0.067 g) and 2-aminobenzoxazole (0.30 mmol, 0.040 g) afforded 0.025 g (27%) of SR-34779 as a light yellow gel. ¹H NMR (400 MHz, d₆-DMSO) δ 8.75 (m, 3H), 8.47 (m, 1H), 8.36 (m, 1H), 8.28 (m, 1H), 8.17 (m, 2H), 7.99-7.81 (m, 5H), 5.59 (q, J=7.0 Hz, 1H), 1.84 (d, J=6.9 Hz, 3H). LC/MS (ESI, M+1): found 368.3

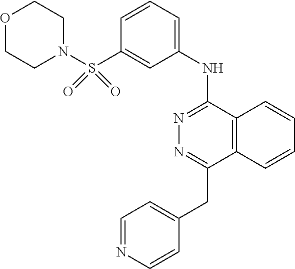 [SMILES: O=S(=O)(c1cccc(Nc2nnc(Cc3ccncc3)c3ccccc23)c1)N1CCOCC1]

SR-35424: Following the general procedure B with 3-(morpholinosulfonyl)aniline (0.3 mmol, 0.073 g) afforded 0.077 g (66%) of SR-35424 as a yellow solid. LC/MS (ESI, M+1): found 462.3

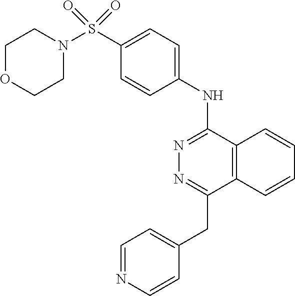 [SMILES: O=S(=O)(c1ccc(Nc2nnc(Cc3ccncc3)c3ccccc23)cc1)N1CCOCC1]

SR-35425: Following the general procedure B with 4-(morpholinosulfonyl)aniline (0.3 mmol, 0.073 g) afforded 0.081 g (70%) of SR-35425 as a yellow solid. LC/MS (ESI, M+1): found 462.2

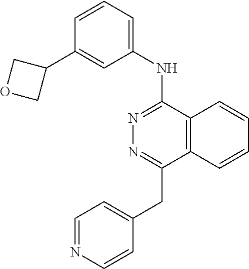 [SMILES: c1cc(Nc2nnc(Cc3ccncc3)c3ccccc23)cc(C2COC2)c1]

SR-35426: Following the general procedure B with 3-(oxetan-3-yl)aniline (0.3 mmol, 0.044 g) afforded 0.009 g (10%) of SR-35426 as a dark yellow solid. ¹H NMR (400 MHz, d₆-DMSO) δ 9.20 (s, 1H), 8.63 (d, J=8.2 Hz, 1H), 8.45 (d, J=5.0 Hz, 2H), 8.11 (m, 1H), 8.04-7.88 (m, 4H), 7.40-7.30 (m, 3H), 7.07 (d, J=7.6 Hz, 1H), 5.00 (dd, J=8.3, 5.8 Hz, 2H), 4.68 (dd, J=6.9, 5.8 Hz, 2H), 4.60 (s, 2H), 4.28 (quin, J=7.6, 7.2 Hz, 1H) LC/MS (ESI, M+1): found 369.3

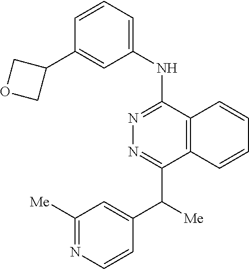 [SMILES: Cc1cc(C(C)c2nnc(Nc3cccc(C4COC4)c3)c3ccccc23)ccn1]

SR-35431: Following a modified version of general procedure B with 1-chloro-4-(1-(2-methylpyridin-4-yl)ethyl)phthalazine (0.25 mmol, 0.071 g) and 3-(oxetan-3-yl)aniline (0.3 mmol, 0.044 g) afforded 0.017 g (17%) of SR-35431 as a yellow solid. LC/MS (ESI, M+1): found 367.4

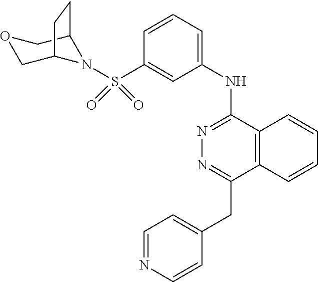 [SMILES: O=S(=O)(c1cccc(Nc2nnc(Cc3ccncc3)c3ccccc23)c1)N1C2CCC1COC2]

SR-35427: Following the general procedure B with 3-((3-oxa-8-azabicyclo[3.2.1]octan-8-yl)sulfonyl)aniline (0.3 mmol, 0.080 g) afforded 0.038 g (31%) of SR-35427 as a yellow solid. LC/MS (ESI, M+1): found 488.3

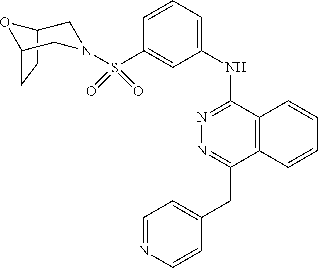 [SMILES: O=S(=O)(c1cccc(Nc2nnc(Cc3ccncc3)c3ccccc23)c1)N1CC2CCC(C1)O2]

SR-35428: Following the general procedure B with 3-((8-oxa-3-azabicyclo[3.2.1]octan-3-yl)sulfonyl)aniline (0.3 mmol, 0.080 g) afforded 0.049 g (40%) of SR-35428 as a yellow solid. LC/MS (ESI, M+1): found 488.3 General Procedure C

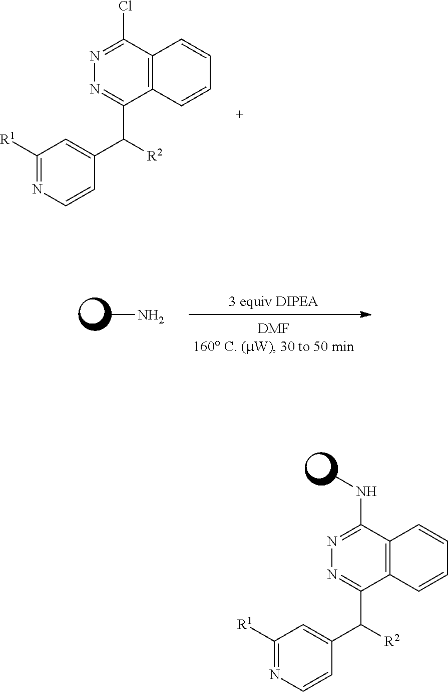

A 0.5-2 mL microwave vial was sequentially charged with 1-chloro-4-(1-(2-methylpyridin-4-yl)ethyl)phthalazine (0.25 mmol, 0.071 g), the amine (0.5 mmol), 0.5 mL of DMF, and DIPEA (0.75 mmol, 0.131 mL), and sealed with a crimp cap. The reaction was heated in a microwave reactor at 160° C. for 30 minutes. The cooled reaction mixture was directly loaded onto a 25 g silica-gel cartridge with the aid of DCM and purified on a Biotage flash system eluting with DCM/MeOH. The fractions containing the product were concentrated down, then loaded onto a 60 g C18 silica-gel cartridge and purified on a Biotage flash system eluting with H₂O/MeOH+0.1% TFA.

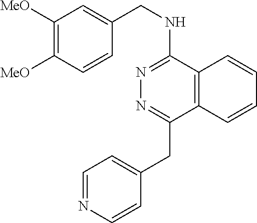 [SMILES: COc1ccc(CNc2nnc(Cc3ccncc3)c3ccccc23)cc1OC]

SR-27886: Following the general procedure C with 3,4-dimethoxybenzylamine (0.5 mmol, 0.075 mL) afforded 0.037 g (38%) of SR-27886 as a red oil. LC/MS (ESI, M+1): found 387.3

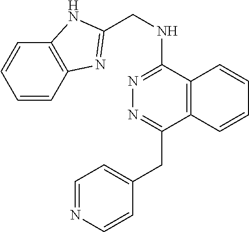 [SMILES: c1ccc2[nH]c(CNc3nnc(Cc4ccncc4)c4ccccc34)nc2c1]

SR-27887: Following the general procedure C with (1H-benzo[d]imidazole-2-yl)methanamine (0.375 mmol, 0.055 g) afforded 0.049 g (21%) of SR-27887 as a red-orange oil. ¹H NMR (400 MHz, d₆-DMSO) δ 9.94 (bs, 1H), 8.67 (bs, 1H), 8.55 (m, 1H), 8.50 (m, 2H), 8.15 (m, 1H), 8.00 (m, 2H), 7.57 (dd, J=6.0, 3.2 Hz, 2H), 7.36 (m, 2H), 7.24 (dd, J=6.0, 3.1 Hz, 2H), 5.09 (s, 2H), 4.58 (s, 2H) LC/MS (ESI, M+1): found 367.3

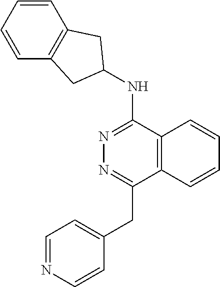 [SMILES: c1ccc2c(c1)CC(Nc1nnc(Cc3ccncc3)c3ccccc13)C2]

SR-27885: Following the general procedure C with 2-aminoindan (0.5 mmol, 0.066 g) afforded 0.046 g (19%) of SR-27885 as a yellow-orange oil. ¹H NMR (400 MHz, d₆-DMSO) δ 8.45 (m, 2H), 8.17 (m, 1H), 7.91 (m, 1H), 7.77 (m, 2H), 7.27 (m, 4H), 7.18 (m, 2H), 5.11 (m, 1H), 4.53 (s, 2H), 3.50 (dd, J=16.0, 7.5 Hz, 2H), 3.14 (m, 2H). LC/MS (ESI, M+1): found 353.3

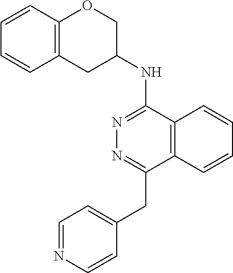 [SMILES: c1ccc2c(c1)CC(Nc1nnc(Cc3ccncc3)c3ccccc13)CO2]

SR-27889: Following the general procedure C with chroman-3-amine hydrochloride (0.5 mmol, 0.092 g) afforded 0.053 g (29%) of SR-27889 as a yellow-orange oil. ¹H NMR (400 MHz, d₆-DMSO) δ 8.44 (m, 2H), 8.37 (m, 1H), 7.91 (m, 1H), 7.78 (m, 2H), 7.26 (m, 2H), 7.13 (m, 2H), 6.87 (m, 2H), 6.66 (d, J=6.9 Hz, 1H), 4.87 (m, 1H), 4.54 (s, 2H), 4.45 (ddd, J=10.4, 3.2, 1.5 Hz, 1H), 4.18 (dtd, J=10.8, 8.3, 1.0 Hz, 1H). LC/MS (ESI, M+1): found 369.3

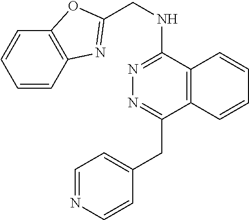 [SMILES: c1ccc2oc(CNc3nnc(Cc4ccncc4)c4ccccc34)nc2c1]

SR-27888: Following the general procedure C with 1,3-benzoxazol-2-ylmethylamine hydrochloride (0.5 mmol, 0.092 g) afforded 0.012 g (13%) of SR-27888 as a tan solid. LC/MS (ESI, M+1): found 368.3

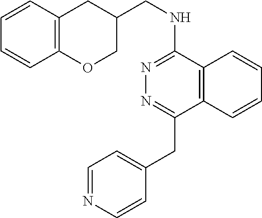 [SMILES: c1ccc2c(c1)CC(CNc1nnc(Cc3ccncc3)c3ccccc13)CO2]

SR-27890: Following the general procedure C with chroman-3-yl-methylamine (0.5 mmol, 0.076 mL) afforded 0.006 g (7%) of SR-27890 as a light yellow film. LC/MS (ESI, M+1): found 383.4

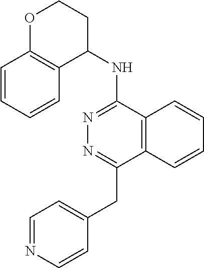 [SMILES: c1ccc2c(c1)OCCC2Nc1nnc(Cc2ccncc2)c2ccccc12]

SR-27891: Following the general procedure C with chroman-4-ylamine (0.5 mmol, 0.067 mL) afforded 0.013 g (14%) of SR-27891 as a light yellow film. LC/MS (ESI, M+1): found 369.3

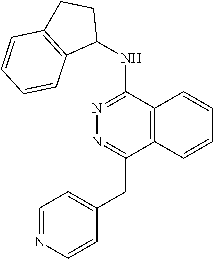 [SMILES: c1ccc2c(c1)CCC2Nc1nnc(Cc2ccncc2)c2ccccc12]

SR-27884: Following the general procedure C with 1-aminoindan (0.5 mmol, 0.063 mL) afforded 0.032 g (36%) of SR-27884 as a yellow solid. LC/MS (ESI, M+1): found 353.3

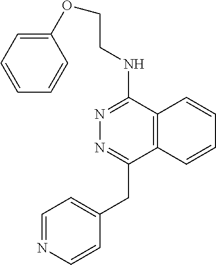 [SMILES: c1ccc(OCCNc2nnc(Cc3ccncc3)c3ccccc23)cc1]

SR-27892: Following the general procedure C with 2-phenoxyethanamine (0.5 mmol, 0.067 mL) afforded 0.039 g (43%) of SR-27892 as a yellow solid. LC/MS (ESI, M+1): found 357.3

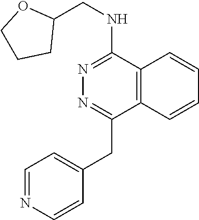 [SMILES: c1ccc2c(NCC3CCCO3)nnc(Cc3ccncc3)c2c1]

SR-30004: Following the general procedure C with tetrahydrofurfurylamine (0.5 mmol, 0.052 mL) afforded 0.071 g (88%) of SR-30004 as a yellow oil. LC/MS (ESI, M+1): found 321.3

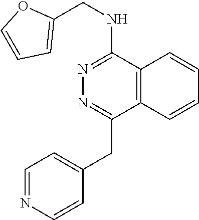 [SMILES: c1coc(CNc2nnc(Cc3ccncc3)c3ccccc23)c1]

SR-30005: Following the general procedure C with furfurylamine (0.5 mmol, 0.044 mL) afforded 0.035 g (44%) of SR-30005 as a yellow oil. LC/MS (ESI, M+1): found 317.2

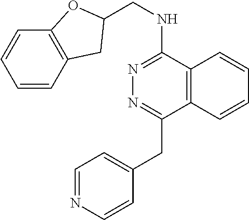 [SMILES: c1ccc2c(c1)CC(CNc1nnc(Cc3ccncc3)c3ccccc13)O2]

SR-30024: Following the general procedure C with (2,3-dihydrobenzofuran-2-yl)methanamine (0.5 mmol, 0.044 mL) afforded 0.054 g (59%) of SR-30024 as a pale orange solid. LC/MS (ESI, M+1): found 369.3

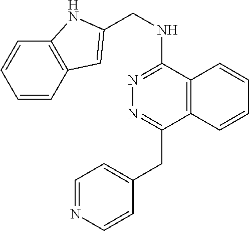 [SMILES: c1ccc2[nH]c(CNc3nnc(Cc4ccncc4)c4ccccc34)cc2c1]

SR-30006: Following the general procedure C with 1H-indole-2-methanamine (0.5 mmol, 0.073 mL) afforded 0.054 g (59%) of SR-30006 as a dark red solid. LC/MS (ESI, M+1): found 366.3

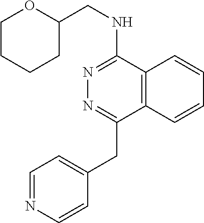 [SMILES: c1ccc2c(NCC3CCCCO3)nnc(Cc3ccncc3)c2c1]

SR-30007: Following the general procedure C with tetrahydropyran-2-ylmethylamine (0.5 mmol, 0.060 mL) afforded 0.041 g (49%) of SR-30007 as a yellow solid. LC/MS (ESI, M+1): found 335.3

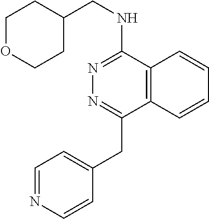 [SMILES: c1ccc2c(NCC3CCOCC3)nnc(Cc3ccncc3)c2c1]

SR-30008: Following the general procedure C with tetrahydropyran-4-ylmethylamine (0.5 mmol, 0.060 mL) afforded 0.043 g (51%) of SR-30008 as a pale yellow solid. LC/MS (ESI, M+1): found 335.3

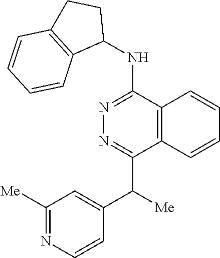 [SMILES: Cc1cc(C(C)c2nnc(NC3CCc4ccccc43)c3ccccc23)ccn1]

SR-35430: Following the general procedure C with 1-aminoindan (0.5 mmol, 0.063 g) afforded 0.046 g (48%) of SR-35430 as a light yellow solid. LC/MS (ESI, M+1): found 381.4

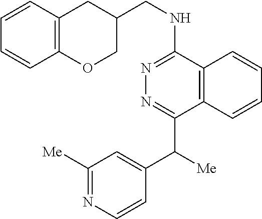 [SMILES: Cc1cc(C(C)c2nnc(NCC3COc4ccccc4C3)c3ccccc23)ccn1]

SR-35432: Following the general procedure C with chroman-3-yl-methylamine (0.5 mmol, 0.081 g) afforded 0.025 g (24%) of SR-35432 as a light yellow solid. LC/MS (ESI, M+1): found 411.3

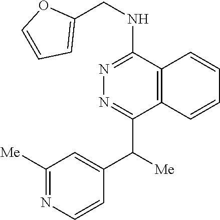 [SMILES: Cc1cc(C(C)c2nnc(NCc3ccco3)c3ccccc23)ccn1]

SR-35433: Following the general procedure C with furfurylamine (0.5 mmol, 0.044 g) afforded 0.019 g (22%) of SR-35433 as a dark yellow solid. LC/MS (ESI, M+1): found 345.3

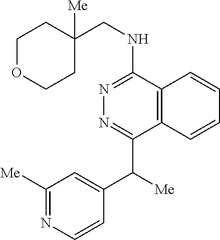 [SMILES: Cc1cc(C(C)c2nnc(NCC3(C)CCOCC3)c3ccccc23)ccn1]

SR-36584: Following the general procedure C with (4-methyltetrahydropyran-4-yl)methylamine (0.5 mmol, 0.070 mL) with 45 minutes of heating afforded 0.091 g (96%) of SR-36584 as a light yellow oil. LC/MS (ESI, M+1): found 377.4

### General Procedure D

A 0.5-2 mL microwave vial was sequentially charged with 1-chloro-4-(1-(2-methylpyridin-4-yl)ethyl)phthalazine (0.1 mmol, 0.028 g), the amine (0.2 mmol), 0.2 mL of DMF, and DIPEA (0.3 mmol, 0.052 mL), and sealed with a crimp cap. The reaction was heated in a microwave reactor at 160° C. for 45 to 50 minutes. The cooled reaction mixture was directly loaded onto a 10 g silica-gel cartridge with the aid of DCM and purified on a Biotage flash system eluting with DCM/MeOH. The fractions containing the product were concentrated down, then loaded onto a 30 g C18 silica-gel cartridge and purified on a Biotage flash system eluting with H₂O/MeOH+0.1% TFA.

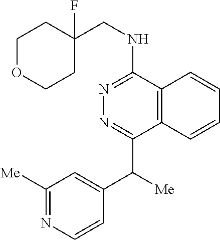 [SMILES: Cc1cc(C(C)c2nnc(NCC3(F)CCOCC3)c3ccccc23)ccn1]

SR-36585: Following the general procedure D with (4-fluorotetrahydropyran-4-yl)methylamine (0.2 mmol, 0.029 mL) afforded 0.018 g (49%) of SR-36585 as a clear film. LC/MS (ESI, M+1): found 381.3

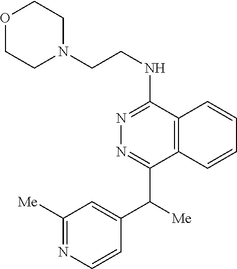 [SMILES: Cc1cc(C(C)c2nnc(NCCN3CCOCC3)c3ccccc23)ccn1]

SR-36586: Following the general procedure D with 4-(2-aminoethyl)morpholine (0.2 mmol, 0.026 mL) afforded 0.036 g (99%) of SR-36586 as a clear film. LC/MS (ESI, M+1): found 378.4

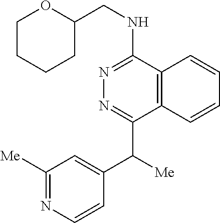 [SMILES: Cc1cc(C(C)c2nnc(NCC3CCCCO3)c3ccccc23)ccn1]

SR-36587: Following the general procedure D with tetrahydropyran-2-ylmethylamine (0.2 mmol, 0.023 g) afforded 0.024 g (68%) of SR-36587 as a clear film. LC/MS (ESI, M+1): found 363.4

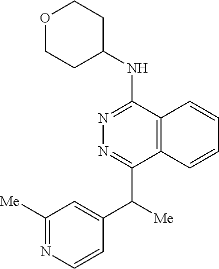 [SMILES: Cc1cc(C(C)c2nnc(NC3CCOCC3)c3ccccc23)ccn1]

SR-36588: Following the general procedure D with 4-aminotetrahydropyran (0.2 mmol, 0.020 mL) afforded 0.023 g (66%) of SR-36588 as a clear film. LC/MS (ESI, M+1): found 349.4

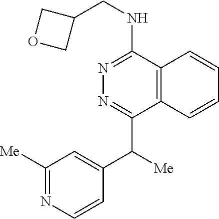 [SMILES: Cc1cc(C(C)c2nnc(NCC3COC3)c3ccccc23)ccn1]

SR-36589: Following the general procedure D with 3-oxetanemethanamine (0.2 mmol, 0.017 mL) afforded 0.028 g (83%) of SR-36589 as a clear film. LC/MS (ESI, M+1): found 335.3

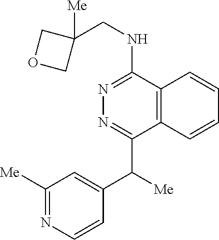 [SMILES: Cc1cc(C(C)c2nnc(NCC3(C)COC3)c3ccccc23)ccn1]

SR-36590: Following the general procedure D with (3-methyloxetan-3-yl)methanamine (0.2 mmol, 0.022 mL) afforded 0.030 g (88%) of SR-36590 as a clear film. LC/MS (ESI, M+1): found 349.4

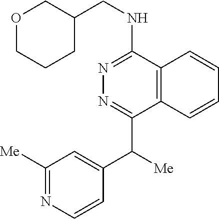 [SMILES: Cc1cc(C(C)c2nnc(NCC3CCCOC3)c3ccccc23)ccn1]

SR-36591: Following the general procedure D with tetrahydropyran-3-ylmethylamine hydrochloride (0.2 mmol, 0.030 g) afforded 0.029 g (82%) of SR-36591 as a clear film. LC/MS (ESI, M+1): found 363.4

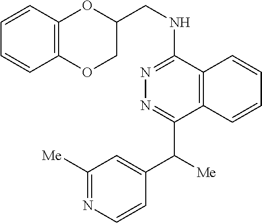 [SMILES: Cc1cc(C(C)c2nnc(NCC3COc4ccccc4O3)c3ccccc23)ccn1]

SR-36592: Following the general procedure D with (2,3-dihydrobenzo[1,4]dioxin-2yl)methylamine (0.2 mmol, 0.033 g) afforded 0.027 g (67%) of SR-36592 as a clear film. LC/MS (ESI, M+1): found 413.4

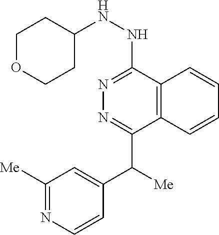 [SMILES: Cc1cc(C(C)c2nnc(NNC3CCOCC3)c3ccccc23)ccn1]

SR-36593: Following the general procedure D with (tetrahydropyran-4-yl)hydrazine (0.2 mmol, 0.023 g) afforded 0.036 g (99%) of SR-36593 as a red-orange solid. LC/MS (ESI, M+1): found 364.3

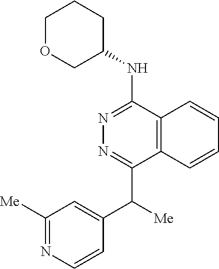

SR-36595-1: Following the general procedure D with (S)-tetrahydropyran-3-amine hydrochloride (0.2 mmol, 0.027 g) afforded 0.025 g (73%) of SR-36595-1 as a clear film. LC/MS (ESI, M+1): found 349.3

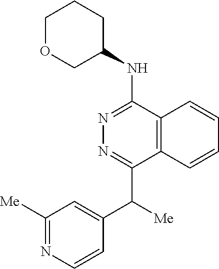

SR-36595-2: Following the general procedure D with (R)-tetrahydropyran-3-amine hydrochloride (0.2 mmol, 0.027 g) afforded 0.028 g (80%) of SR-36595-2 as a clear film. LC/MS (ESI, M+1): found 349.3

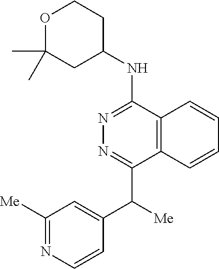 [SMILES: Cc1cc(C(C)c2nnc(NC3CCOC(C)(C)C3)c3ccccc23)ccn1]

SR-365%: Following the general procedure D with 2,2-dimethyltetrahydropyran-4-amine (0.2 mmol, 0.026 g) afforded 0.022 g (61%) of SR-36596 as a clear film. LC/MS (ESI, M+1): found 377.4

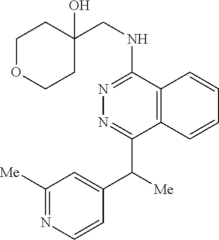 [SMILES: Cc1cc(C(C)c2nnc(NCC3(O)CCOCC3)c3ccccc23)ccn1]

SR-36597: Following the general procedure D with 4-(aminomethyl)tetrahydropyran-4-ol hydrochloride (0.2 mmol, 0.033 g) afforded 0.037 g (99%) of SR-36597 as a light yellow oil. This procedure was modified in that only a single purification was required with a 10 g silica-gel cartridge. LC/MS (ESI, M+1): found 379.4

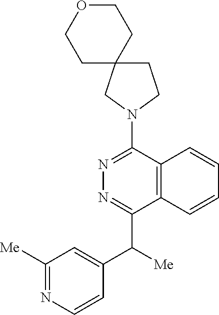 [SMILES: Cc1cc(C(C)c2nnc(N3CCC4(CCOCC4)C3)c3ccccc23)ccn1]

SR-36598: Following the general procedure D with 8-oxa-2-azaspiro[4.5]decane (0.2 mmol, 0.028 g) afforded 0.022 g (58%) of SR-36598 as a clear oil. LC/MS (ESI, M+1): found 389.4

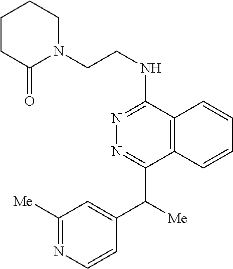 [SMILES: Cc1cc(C(C)c2nnc(NCCN3CCCCC3=O)c3ccccc23)ccn1]

SR-36599: Following the general procedure D with 1-(2-aminoethyl)piperidin-2-one (0.2 mmol, 0.028 g) afforded 0.022 g (56%) of SR-36599 as a clear oil. LC/MS (ESI, M+1): found 390.4

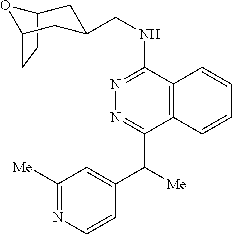 [SMILES: Cc1cc(C(C)c2nnc(NCC3CC4CCC(C3)O4)c3ccccc23)ccn1]

SR-36600: Following the general procedure D with (8-oxabicyclo[3.2.1]octan-3-yl)methanamine (0.2 mmol, 0.028 g) afforded 0.024 g (61%) of SR-36600 as a clear film. LC/MS (ESI, M+1): found 389.3

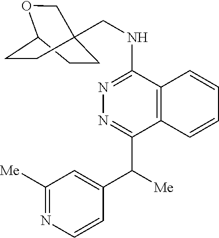 [SMILES: Cc1cc(C(C)c2nnc(NCC34CCC(CC3)OC4)c3ccccc23)ccn1]

SR-36601: Following the general procedure D with (2-oxabicyclo[2.2.2]octan-4-yl)methanamine (0.2 mmol, 0.028 g) afforded 0.037 g (99%) of SR-36601 as a clear oil. LC/MS (ESI, M+1): found 389.4

It is understood that the examples, schemes, and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### REFERENCES

1. Zhou M, Ottenberg G, Sferrazza G F, Lasmezas C I. Highly neurotoxic monomeric alpha-helical prion protein. Proc Natl Acad Sci USA. 2012; 109(8):3113-8. Epub 2012 Feb. 11. doi: 10.1073/pnas.1118090109. PubMed PMID: 22323583; PMCID: 3286986.
2. Zhou M, Ottenberg G, Sferrazza G F, Hubbs C, Fallahi M, Rumbaugh G, Brantley A F, Lasmdzas C I. Neuronal death induced by misfolded prion protein is due to NAD+ depletion and can be relieved in vitro and in vivo by NAD+ replenishment. Brain. 2015; 138(4):992-1008. doi: 10.1093/brain/awv002; PMCID: 25678560.
3. Schondorf D C, Ivanyuk D, Baden P, Sanchez-Martinez A, De Cicco S, Yu C, Giunta I, Schwarz L K, Di Napoli G, Panagiotakopoulou V, Nestel S, Keatinge M, Pruszak J, Bandmann O, Heimrich B, Gasser T, Whitworth A J, Deleidi M. The NAD+ Precursor Nicotinamide Riboside Rescues Mitochondrial Defects and Neuronal Loss in iPSC and Fly Models of Parkinson's Disease. Cell Rep. 2018; 23(10):2976-88. Epub 2018 Jun. 7. doi: 10.1016/j.celrep.2018.05.009. PubMed PMID: 29874584.
4. Shan C, Gong Y L, Zhuang Q Q, Hou Y F, Wang S M, Zhu Q, Huang G R, Tao B, Sun L H, Zhao H Y, Li S T, Liu J M. Protective effects of beta-nicotinamide adenine dinucleotide against motor deficits and dopaminergic neuronal damage in a mouse model of Parkinson's disease. Prog Neuropsychopharmacol Biol Psychiatry. 2019; 94:109670. Epub 2019 Jun. 21. doi: 10.1016/j.pnpbp.2019.109670. PubMed PMID: 31220519.
5. Sorrentino V, Romani M, Mouchiroud L, Beck J S, Zhang H, D'Amico D, Moullan N, Potenza F, Schmid A W, Rietsch S, Counts S E, Auwerx J. Enhancing mitochondrial proteostasis reduces amyloid-beta proteotoxicity. Nature. 2017; 552(7684):187-93. Epub 2017 Dec. 7. doi: 10.1038/nature25143. PubMed PMID: 29211722; PMCID: PMC5730497.
6. Hou Y, Lautrup S, Cordonnier S, Wang Y, Croteau D L, Zavala E, Zhang Y, Moritoh K, O'Connell J F, Baptiste B A, Stevnsner T V, Mattson M P, Bohr V A. NAD(+) supplementation normalizes key Alzheimer's features and DNA damage responses in a new A D mouse model with introduced DNA repair deficiency. Proc Natl Acad Sci USA. 2018; 115(8):E1876-E85. doi: 10.1073/pnas.1718819115. PubMed PMID: 29432159; PMCID: PMC5828618.
7. Hosseini L, Mahmoudi J, Pashazadeh F, Salehi-Pourmehr H, Sadigh-Eteghad S. Protective Effects of Nicotinamide Adenine Dinucleotide and Related Precursors in Alzheimer's Disease: A Systematic Review of Preclinical Studies. J Mol Neurosci. 2021; 71(7):1425-35. Epub 20210428. doi: 10.1007/s12031-021-01842-6. PubMed PMID: 33907963.
8. Wang X, He H J, Xiong X, Zhou S, Wang W W, Feng L, Han R, Xie C L. NAD(+) in Alzheimer's Disease: Molecular Mechanisms and Systematic Therapeutic Evidence Obtained in vivo. Front Cell Dev Biol. 2021; 9:668491. Epub 2021 Aug. 3. doi: 10.3389/fcell.2021.668491. PubMed PMID: 34414179; PMCID: PMC8369418.
9. Harlan B A, Killoy K M, Pehar M, Liu L, Auwerx J, Vargas M R. Evaluation of the NAD(+) biosynthetic pathway in ALS patients and effect of modulating NAD(+) levels in hSODI-linked ALS mouse models. Exp Neurol. 2020; 327:113219. Epub 2020 Feb. 6. doi: 10.1016/j.expneurol.2020.113219. PubMed PMID: 32014438; PMCID: PMC7089832.
10. Harlan B A, Pehar M, Sharma D R, Beeson G, Beeson C C, Vargas M R. Enhancing NAD+Salvage Pathway Reverts the Toxicity of Primary Astrocytes Expressing Amyotrophic Lateral Sclerosis-linked Mutant Superoxide Dismutase 1 (SOD1). J Biol Chem. 2016; 291(20):10836-46. doi: 10.1074/jbc.M 115.698779. PubMed PMID: 27002158; PMCID: PMC4865928.
11. Massudi H, Grant R, Braidy N, Guest J, Farnsworth B, Guillemin G J. Age-associated changes in oxidative stress and NAD+ metabolism in human tissue. PLoS One. 2012; 7(7):e42357. Epub 2012 Aug. 1. doi: 10.1371/journal.pone.0042357. PubMed PMID: 22848760; PMCID: 3407129.
12. Zhang H, Ryu D, Wu Y, Gariani K, Wang X, Luan P, D'Amico D, Ropelle E R, Lutolf M P, Aebersold R, Schoonjans K, Menzies K J, Auwerx J. NAD(+) repletion improves mitochondrial and stem cell function and enhances life span in mice. Science. 2016; 352(6292):1436-43. Epub 2016 Apr. 30. doi: 10.1126/science.aaf2693. PubMed PMID: 27127236.
13. Mouchiroud L, Houtkooper R H, Moullan N, Katsyuba E, Ryu D, Canto C, Mottis A, Jo Y S, Viswanathan M, Schoonjans K, Guarente L, Auwerx J. The NAD(+)/Sirtuin Pathway Modulates Longevity through Activation of Mitochondrial UPR and FOXO Signaling. Cell. 2013; 154(2):430-41. doi: 10.1016/j.cell.2013.06.016. PubMed PMID: 23870130; PMCID: PMC3753670.
14. Penberthy W T, Tsunoda I. The importance of NAD in multiple sclerosis. Curr Pharm Des. 2009; 15(1):64-99. Epub 2009 Jan. 20. PubMed PMID: 19149604; PMCID: 2651433.
15. Satchell M A, Zhang X, Kochanek P M, Dixon C E, Jenkins L W, Melick J, Szabo C, Clark R S. A dual role for poly-ADP-ribosylation in spatial memory acquisition after traumatic brain injury in mice involving NAD+ depletion and ribosylation of 14-3-3gamma. J Neurochem. 2003; 85(3):697-708. PubMed PMID: 12694396.
16. Brown K D, Maqsood S, Huang J Y, Pan Y, Harkcom W, Li W, Sauve A, Verdin E, Jaffrey S R. Activation of SIRT3 by the NAD(+) precursor nicotinamide riboside protects from noise-induced hearing loss. Cell Metab. 2014; 20(6):1059-68. Epub 2014 Dec. 4. doi: 10.1016/j.cmet.2014.11.003. PubMed PMID: 25470550; PMCID: PMC4940130.
17. Vaur P, Brugg B, Mericskay M, Li Z, Schmidt M S, Vivien D, Orset C, Jacotot E, Brenner C, Duplus E. Nicotinamide riboside, a form of vitamin B3, protects against excitotoxicity-induced axonal degeneration. FASEB J. 2017; 31(12):5440-52. doi: 10.1096/fj.201700221RR. PubMed PMID: 28842432.
18. Sasaki Y, Araki T, Milbrandt J. Stimulation of nicotinamide adenine dinucleotide biosynthetic pathways delays axonal degeneration after axotomy. J Neurosci. 2006; 26(33):8484-91. Epub 2006 Aug. 18. doi: 10.1523/JNEUROSCI.2320-06.2006. PubMed PMID: 16914673.
19. Ying W, Wei G, Wang D, Wang Q, Tang X, Shi J, Zhang P, Lu H. Intranasal administration with NAD+ profoundly decreases brain injury in a rat model of transient focal ischemia. Front Biosci. 2007; 12:2728-34. Epub 2006 Nov. 28. doi: 2267 [pii]. PubMed PMID: 17127275.
20. Hsu C P, Oka S, Shao D, Hariharan N, Sadoshima J. Nicotinamide phosphoribosyltransferase regulates cell survival through NAD+ synthesis in cardiac myocytes. Circ Res. 2009; 105(5):481-91. doi: 10.1161/CIRCRESAHA.109.203703. PubMed PMID: 19661458; PMCID: PMC2765790.
21. Yamamoto T, Byun J, Zhai P, Ikeda Y, Oka S, Sadoshima J. Nicotinamide mononucleotide, an intermediate of NAD+ synthesis, protects the heart from ischemia and reperfusion. PLoS One. 2014; 9(6):e98972. doi: 10.1371/journal.pone.0098972. PubMed PMID: 24905194; PMCID: PMC4048236.
22. Zhang M, Jiang N, Chu Y, Postnikova O, Varghese R, Horvath A, Cheema A K, Golestaneh N. Dysregulated metabolic pathways in age-related macular degeneration. Sci Rep. 2020; 10(1):2464. Epub 2020 Feb. 14. doi: 10.1038/s41598-020-59244-4. PubMed PMID: 32051464; PMCID: PMC7016007.
23. Lin J B, Kubota S, Ban N, Yoshida M, Santeford A, Sene A, Nakamura R, Zapata N, Kubota M, Tsubota K, Yoshino J, Imai S I, Apte R S. NAMPT-Mediated NAD(+) Biosynthesis Is Essential for Vision In Mice. Cell Rep. 2016; 17(1):69-85. doi: 10.1016/j.celrep.2016.08.073. PubMed PMID: 27681422; PMCID: PMC5104206.
24. Bai S, Sheline C T. NAD(+) maintenance attenuates light induced photoreceptor degeneration. Exp Eye Res. 2013; 108:76-83. Epub 2013 Jan. 1. doi: 10.1016/j.exer.2012.12.007. PubMed PMID: 23274583; PMCID: PMC3578030.
25. Zhang X, Henneman N F, Girardot P E, Sellers J T, Chrenek M A, Li Y, Wang J, Brenner C, Nickerson J M, Boatright J H. Systemic Treatment With Nicotinamide Riboside Is Protective in a Mouse Model of Light-Induced Retinal Degeneration. Invest Ophthalmol Vis Sci. 2020; 61(10):47. Epub 2020 Aug. 28. doi: 10.1167/iovs.61.10.47. PubMed PMID: 32852543; PMCID: PMC7452859.
26. Yoshino J, Mills K F, Yoon M J, Imai S. Nicotinamide mononucleotide, a key NAD(+) intermediate, treats the pathophysiology of diet- and age-induced diabetes in mice. Cell Metab. 2011; 14(4):528-36. doi: 10.1016/j.cmet.2011.08.014. PubMed PMID: 21982712; PMCID: PMC3204926.
27. Trammell S A, Weidemann B J, Chadda A, Yorek M S, Holmes A, Coppey L J, Obrosov A, Kardon R H, Yorek M A, Brenner C. Nicotinamide Riboside Opposes Type 2 Diabetes and Neuropathy in Mice. Sci Rep. 2016; 6:26933. Epub 2016 May 28. doi: 10.1038/srep26933. PubMed PMID: 27230286; PMCID: PMC4882590.
28. Costes S. Targeting protein misfolding to protect pancreatic beta-cells in type 2 diabetes. Curr Opin Pharmacol. 2018; 43:104-10. Epub 2018 Sep. 25. doi: 10.1016/j.coph.2018.08.016. PubMed PMID: 30245473.
29. Ueberberg S, Nauck M A, Uhl W, Montemurro C, Tannapfel A, Clark A, Meier J J. Islet amyloid in patients with diabetes due to exocrine pancreatic disorders, type 2 diabetes and non-diabetic patients. J Clin Endocrinol Metab. 2020. Epub 2020 Apr. 10. doi: 10.1210/clinem/dgaa176. PubMed PMID: 32271378.
30. Liu M, Hodish I, Haataja L, Lara-Lemus R, Rajpal G, Wright J, Arvan P. Proinsulin misfolding and diabetes: mutant INS gene-induced diabetes of youth. Trends Endocrinol Metab. 2010; 21(11):652-9. Epub 2010 Aug. 21. doi: 10.1016/j.tem.2010.07.001. PubMed PMID: 20724178; PMCID: PMC2967602.
31. Sun J, Cui J, He Q, Chen Z, Arvan P, Liu M. Proinsulin misfolding and endoplasmic reticulum stress during the development and progression of diabetes. Mol Aspects Med. 2015; 42:105-18. Epub 2015 Jan. 13. doi: 10.1016/j.mam.2015.01.001. PubMed PMID: 25579745; PMCID: PMC4404191.
32. Okabe K, Yaku K, Tobe K, Nakagawa T. Implications of altered NAD metabolism in metabolic disorders. J Biomed Sci. 2019; 26(1):34. Epub 2019 May 13. doi: 10.1186/s12929-019-0527-8. PubMed PMID: 31078136; PMCID: PMC6511662.
33. Romani M, Sorrentino V, Oh C M, Li H, de Lima T I, Zhang H, Shong M, Auwerx J. NAD(+) boosting reduces age-associated amyloidosis and restores mitochondrial homeostasis in muscle. Cell Rep. 2021; 34(3):108660. Epub 2021 Jan. 21. doi: 10.1016/j.celrep.2020.108660. PubMed PMID: 33472069; PMCID: PMC7816122.
34. Klimova N, Fearnow A, Long A, Kristian T. NAD(+) precursor modulates post-ischemic mitochondrial fragmentation and reactive oxygen species generation via SIRT3 dependent mechanisms. Exp Neurol. 2020; 325:113144. Epub 2019 Dec. 15. doi: 10.1016/j.expneurol.2019.113144. PubMed PMID: 31837320; PMCID: PMC8328278.
35. Wang X, Li H, Ding S. Pre-B-cell colony-enhancing factor protects against apoptotic neuronal death and mitochondrial damage in ischemia. Sci Rep. 2016; 6:32416. Epub 2016/09/01. doi: 10.1038/srep32416. PubMed PMID: 27576732; PMCID: PMC5006239.
36. Ralto K M, Rhee E P, Parikh S M. NAD(+) homeostasis in renal health and disease. Nat Rev Nephrol. 2020; 16(2):99-111. Epub 2019 Nov. 2. doi: 10.1038/s41581-019-0216-6. PubMed PMID: 31673160.
37. Dahlin J L, Nissink J W, Strasser J M, Francis S, Higgins L, Zhou H, Zhang Z, Walters M A. PAINS in the assay: chemical mechanisms of assay interference and promiscuous enzymatic inhibition observed during a sulfhydryl-scavenging HTS. J Med Chem. 2015; 58(5):2091-113. doi: 10.1021/jm5019093. PubMed PMID: 25634295; PMCID: PMC4360378.
38. Lipinski C A, Lombardo F, Dominy B W, Feeney P J. Experimental and computational approaches to estimate solubility and permeability in drug discovery and development settings. Adv Drug Deliv Rev. 2001; 46(1-3):3-26. Epub 2001 Mar. 22. PubMed PMID: 11259830.
39. Veber D F, Johnson S R, Cheng H Y, Smith B R, Ward K W, Kopple K D. Molecular properties that influence the oral bioavailability of drug candidates. J Med Chem. 2002; 45(12):2615-23. Epub 2002 May 31. PubMed PMID: 12036371.
40. Wang, S.; Qian, Q.; Gong, H.; “Nickel-Catalyzed Reductive Coupling of Aryl Halides with Secondary Alkyl Bromides and Allylic Acetate” Org. Lett. 2012, 14, 3352-3355.

What is claimed:

1. A compound having a structure of Formula (X), 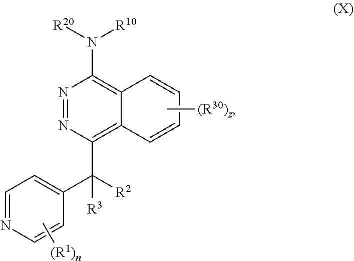
or a pharmaceutically acceptable salt thereof, or an isomer thereof;
wherein:
R¹⁰ is hydrogen, substituted or unsubstituted alkyl, substituted or unsubstituted heteroalkyl;
R²⁰ is -L¹-R²¹;
L¹ is a bond, substituted or unsubstituted alkylene, or substituted or unsubstituted heteroalkylene;
R²¹ is substituted or unsubstituted cycloalkyl, substituted or unsubstituted heterocycloalkyl, substituted or unsubstituted aryl, or substituted or unsubstituted heteroaryl;
R¹⁰ and R²⁰ are optionally joined to form a substituted or unsubstituted bicyclic heterocycloalkyl;
R¹ is independently halogen, —CX¹₃, —CHX¹₂, —CH₂X¹, —OCX¹₃, —OCH₂X¹, —OCHX¹₂, —CN, —OR^{1F}, —SR^{1F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, D, halogen, —CX²₃, —CHX²₂, —CH₂X², —OCX²₃, —OCH₂X², —OCHX²₂, —OR^{2F}, —SR^{2F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, D, halogen, —CX³₃, —CHX³₂, —CH₂X³, —OCX³₃, —OCH₂X³, —OCHX³₂, —OR^{3F}, —SR^{3F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³⁰ is independently halogen, —CX³⁰₃, —CHX³⁰₂, —CH₂X³⁰, —OCX³⁰₃, —OCH₂X³⁰, —OCHX³⁰₂, —CN, —OR^{30F}, —SR^{30F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is an integer of 0 to 4;
z is an integer of 0 to 4;
Each X¹, X², X³ and X³⁰ is independently —F, —Br, —Cl, or —I; and
Each R^{1F}, R^{2F}, R^{3F} and R^{30F} is independently hydrogen, substituted or unsubstituted alkyl, or substituted or unsubstituted heteroalkyl,
provided that when R¹⁰ is hydrogen and L¹ is a bond, then R²¹ is not unsubstituted phenyl nor phenyl substituted with halogen, —C(O)CH₃, —S(O)₂—NH₂, or substituted or unsubstituted alkyl; and when L¹ is a methylene, then R²¹ is not unsubstituted tetrahydro-pyranyl.

2. The compound of claim 1, wherein:
L¹ is a bond, substituted or unsubstituted C₁-C₄ alkylene, or substituted or unsubstituted 2 to 4 membered heteroalkylene.

3. The compound of an one of claims 1 to 2, wherein R¹⁰ and R²⁰ are joined to form a substituted or unsubstituted

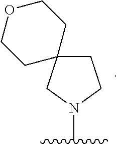 [SMILES: *N1CCC2(CCOCC2)C1]

4. The compound of any one of claims 1 to 2, wherein R²¹ is 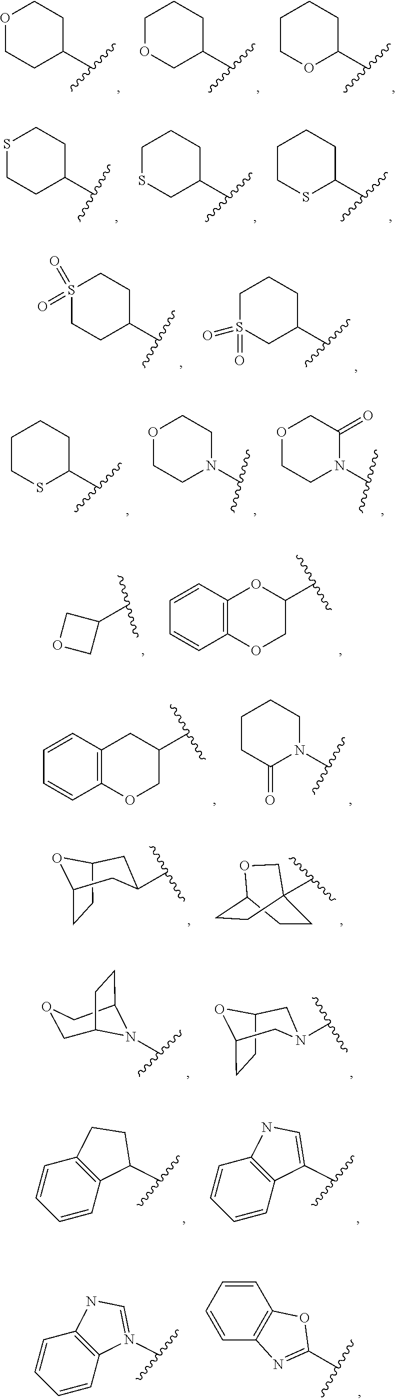 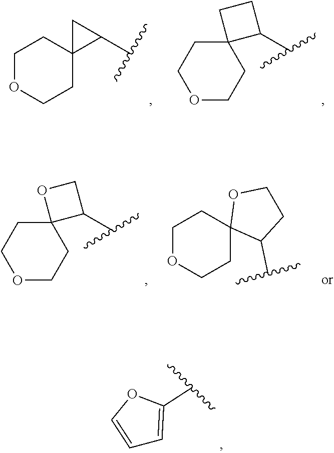 [SMILES: *C1CC12CCOCC2 ; *C1CCC12CCOCC2 ; *C1COC12CCOCC2 ; *C1CCOC12CCOCC2 ; *c1ccco1]
which is substituted or unsubstituted.

5. The compound of claim 1, wherein the compound has a structure of Formula (XI): 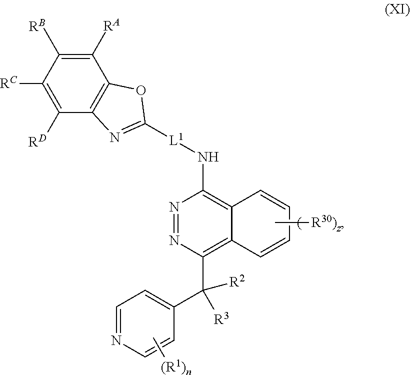
or a pharmaceutically acceptable salt thereof, or an isomer thereof;
wherein:
each R^{A}, R^{B}, R^{C}, and R^{D} is independently hydrogen, substituted or unsubstituted alkyl, substituted or unsubstituted heteroalkyl; or two of R^{A}, R^{B}, R^{C}, and R^{D} are joined to form a substituted or unsubstituted cycloalkyl, substituted or unsubstituted heterocycloalkyl, substituted or unsubstituted aryl, or substituted or unsubstituted heteroaryl.

6. The compound of claim 5, wherein:
R^{A} and R^{B} are joined to form a substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl;
R^{B} and R^{C} are joined to form a substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl; or
R^{C} and R^{D} are joined to form a substituted or unsubstituted C₅-C₆ cycloalkyl, substituted or unsubstituted 5 to 6 membered heterocycloalkyl, substituted or unsubstituted phenyl, or substituted or unsubstituted 5 to 6 membered heteroaryl.

7. The compound of any one of claims 5 and 6, wherein the compound has a structure of Formula (XI-a), (XI-b), or (XI-c), 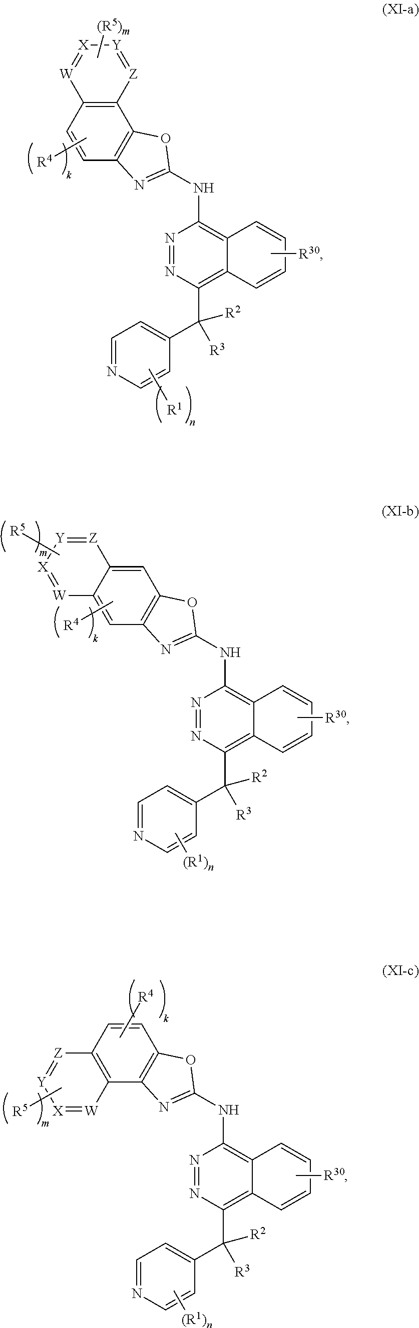
or a pharmaceutically acceptable salt thereof, or an isomer thereof;
wherein
Each W, X, Y and Z is independently ═N— or —CH═;
Each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)R^{4F}, —C(O)OR^{4F}, —S(O)₂RF, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Each R⁵ is independently halogen, —CX⁵₃, —CHX⁵₂, —CH₂X⁵, —OCX⁵₃, —OCH₂X⁵, —OCHX⁵₂, —CN, —OR^{5F}, —SR^{5F}, —C(O)OR^{5F}, —S(O)₂R^{5F}, —C(O)NHR^{5F}, —C(O)N(R^{5F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
k is an integer of 0 to 2;
m is an integer of 0 to 4;
Each X⁴ and X⁵ is independently —F, —Br, —Cl, or —I; and
Each R^{4F} and R^{5F} is independently hydrogen, substituted or unsubstituted alkyl, or substituted or unsubstituted heteroalkyl.

8. The compound of claim 7, the compound has the structure of (XI-a-1), (XI-b-a), or (XI-c-1), 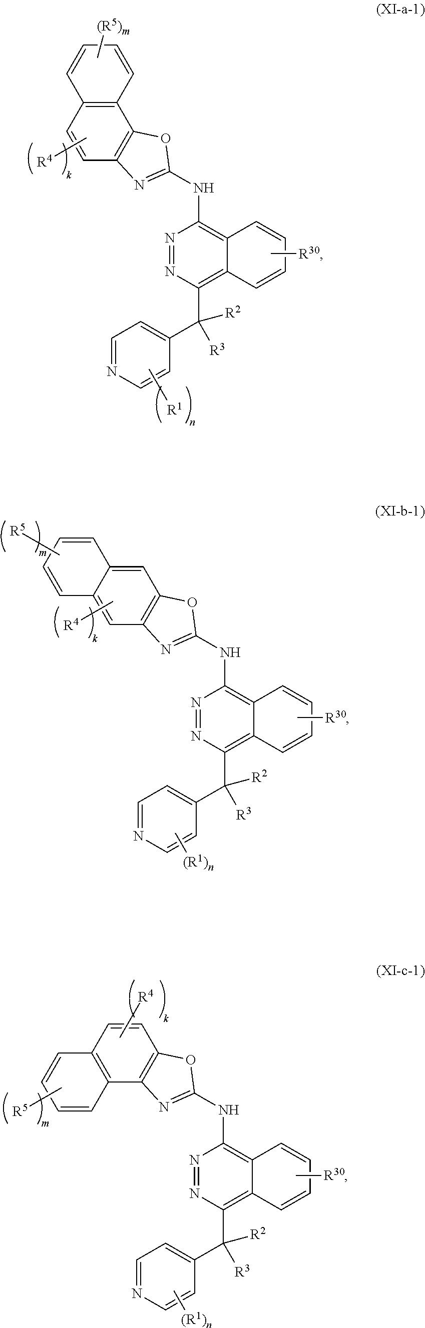
or a pharmaceutically acceptable salt thereof, or an isomer thereof.

9. The compound of any one of claims 5 and 6, wherein the compound has a structure of Formula (XI-d), (XI-e), (XI-f), (XI-d′), (XI-e′), or (XI-f′), 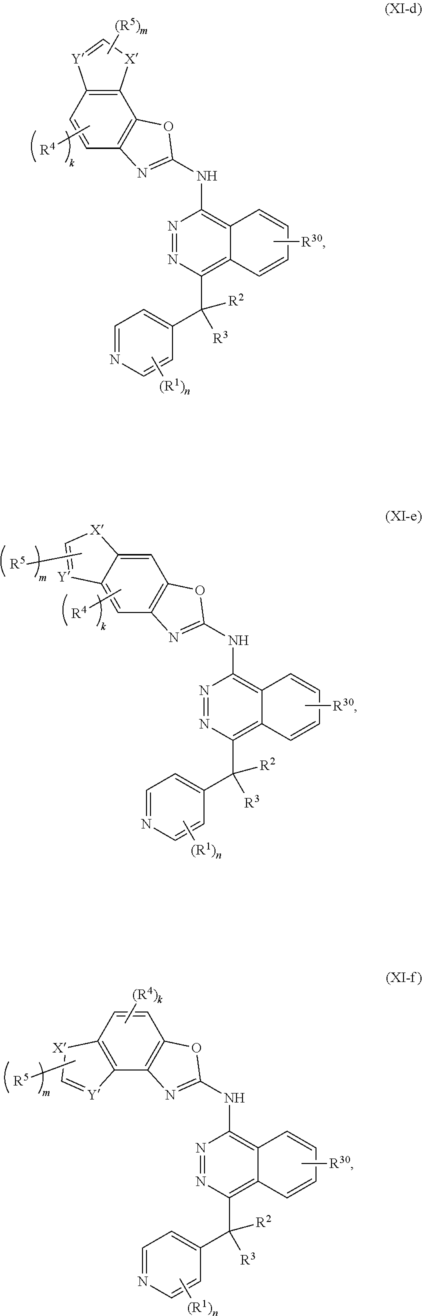 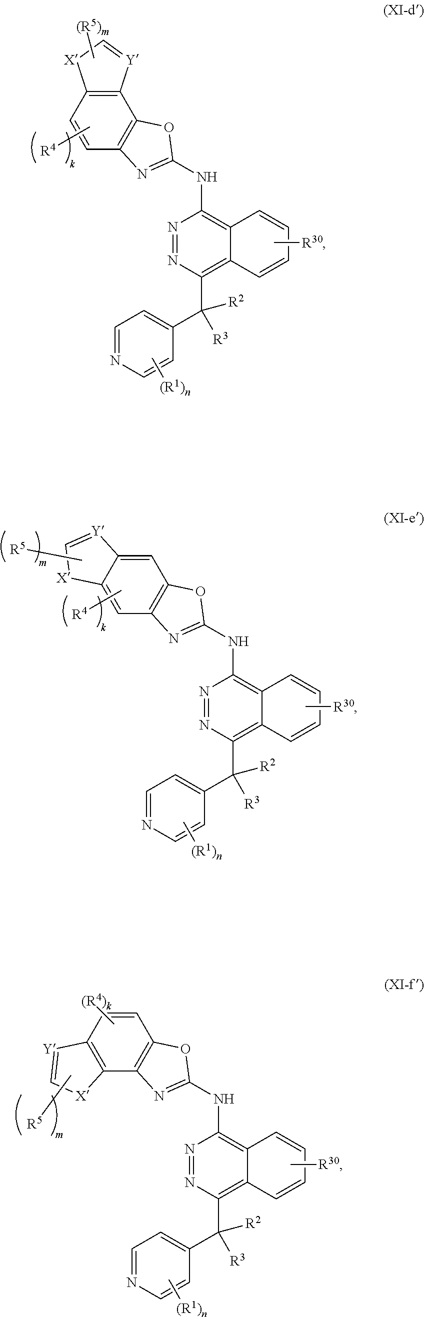
or a pharmaceutically acceptable salt thereof, or an isomer thereof;
wherein:
X′ is —O—, —NH—, or —CH₂—;
Y′ is —NH═, or —CH═;
k is an integer of 0 to 2;
m is an integer of 0 to 3;
Each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)R^{4F}, —C(O)OR^{4F}, —S(O)₂RF, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Each R⁵ is independently halogen, —CX⁵₃, —CHX⁵₂, —CH₂X⁵, —OCX⁵₃, —OCH₂X⁵, —OCHX⁵₂, —CN, —OR^{5F}, —SR^{5F}, —C(O)OR^{5F}, —S(O)₂R^{5F}, —C(O)NHR^{5F}, —C(O)N(R^{5F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Each X⁴ and X⁵ is independently —F, —Br, —Cl, or —I; and
Each R^{4F} and R^{5F} is independently hydrogen, substituted or unsubstituted alkyl, or substituted or unsubstituted heteroalkyl.

10. The compound of any one of claims 5 to 9, wherein:
R² is H, D, halogen, substituted or unsubstituted C₁-C₄ alkyl, —CX²₃, or OR^{2F};
R³ is H, D, halogen, substituted or unsubstituted C₁-C₄ alkyl, —CX³₃, or OR^{3F}; and
Each R^{2F} and R^{3F} is independently hydrogen, or unsubstituted C₁-C₄ alkyl.

11. The compound of any one of claims 7 to 10, wherein k is 1 or 2 and m is 1 or 2.

12. The compound of any one of claims 7 to 11, wherein:
Each R⁴ is independently halogen, —CX⁴₃, —OCX⁴₃, —CN, —OR^{4F}, —C(O)R^{4F}, —C(O)OR^{4F}, —S(O)₂R^{4F}, —C(O)NHR^{4F}, —C(O)N(R^{4F})₂, or substituted or unsubstituted C₁-C₄ alkyl;
Each R⁵ is independently halogen, —CX⁴⁵₃, —OCX⁵₃, —CN, —OR^{5F}, —C(O)OR^{5F}, —S(O)₂R^{5F}, —C(O)NHR^{5F}, —C(O)N(R^{5F})₂, or substituted or unsubstituted C₁-C₄ alkyl; and
Each R^{4F} and R^{5F} is independently a hydrogen, substituted or unsubstituted C₁-C₄ alkyl, substituted or unsubstituted phenyl, or substituted or unsubstituted 5 to 6 membered heteroaryl.

13. The compound of any one of claims 1 to 12, wherein:
R¹ is halogen, substituted or unsubstituted C₁-C₄ alkyl, —CN, —CX¹₃, or —OR^{1F}; and
R^{1F} is hydrogen or unsubstituted C₁-C₄ alkyl.

14. The compound of any one of claims 1 to 13, wherein:
R² is hydrogen, and R³ is hydrogen or substituted or unsubstituted C₁-C₄ alkyl.

15. The compound of any one of claims 1 to 14, wherein:
R³⁰ is halogen, substituted or unsubstituted C₁-C₄ alkyl, —CN, —CX¹₃, or —OR^{30F}; and
R^{30F} is hydrogen or unsubstituted C₁-C₄ alkyl.

16. A compound having a structure of Formula (XII), 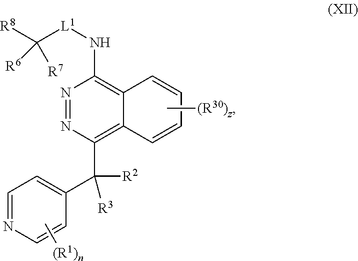
or a pharmaceutically acceptable salt thereof, or an isomer thereof;
wherein:
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is independently halogen, —CX¹₃, —CHX¹₂, —CH₂X¹, —OCX¹₃, —OCH₂X¹, —OCHX¹₂, —CN, —OR^{1F}, —SR^{1F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, D, halogen, —CX²₃, —CHX²₂, —CH₂X², —OCX²₃, —OCH₂X², —OCHX²₂, —OR^{2F}, —SR^{2F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is hydrogen, D, halogen, —CX³₃, —CHX³₂, —CH₂X³, —OCX³₃, —OCH₂X³, —OCHX³₂, —OR^{3F}, —SR^{3F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² and R³ are optionally joined together to form a substituted or unsubstituted cycloalkyl, or substituted or unsubstituted heterocycloalkyl;
Each R⁶ and R⁷ is independently hydrogen, substituted or unsubstituted alkyl, or substituted or unsubstituted heteroalkyl; or R⁶ and R⁷ are optionally joined together to form a substituted or unsubstituted cycloalkyl, or substituted or unsubstituted heterocycloalkyl;
R⁸ is a substituted or unsubstituted cycloalkyl, or substituted or unsubstituted heterocycloalkyl, substituted or unsubstituted aryl, or substituted or unsubstituted heteroaryl;
R³⁰ is independently halogen, —CX³⁰₃, —CHX³⁰₂, —CH₂X³⁰, —OCX³⁰₃, —OCH₂X³⁰, —OCHX³⁰₂, —CN, —OR^{30F}, —SR^{30F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is an integer of 0 to 4;
z is an integer of 0 to 4;
Each X¹, X², X³, and X³⁰ is independently —F, —Br, —Cl, or —I; and
Each R^{1F}, R^{2F}, R^{3F}, and R^{30F} is independently hydrogen, substituted or unsubstituted alkyl, or substituted or unsubstituted heteroalkyl,
provided that when n is 0, L¹ is a bond, R⁶ and R⁷ are hydrogen, then R⁸ is not unsubstituted tetrahydro-pyranyl.

17. The compound of claim 16, wherein R⁸ is 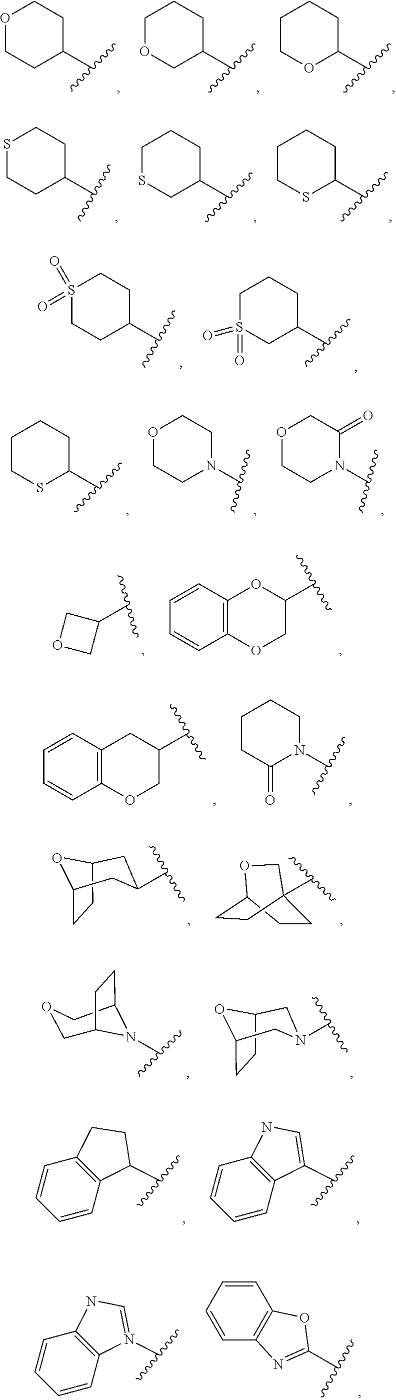 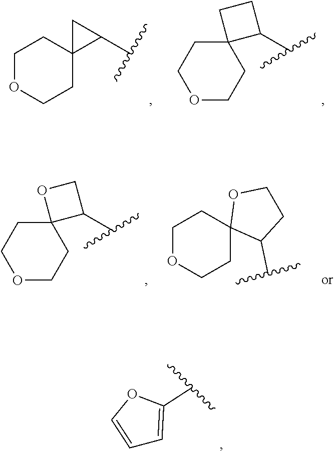 [SMILES: *C1CC12CCOCC2 ; *C1CCC12CCOCC2 ; *C1COC12CCOCC2 ; *C1CCOC12CCOCC2 ; *c1ccco1]
which is substituted or unsubstituted.

18. The compound of claim 16, wherein:
R⁸ is 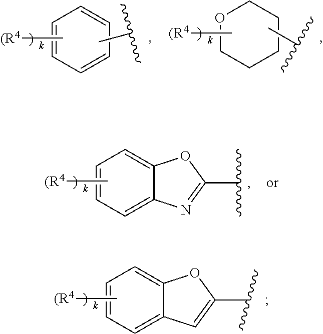
Each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)R^{4F}, —C(O)OR^{4F}, —S(O)₂R^{4F}, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
k is an integer of 0 to 5;
X⁴ is independently —F, —Br, —Cl, or —I; and
Each R^{4F} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

19. The compound of 16 to 18, wherein at least one of R² and R³ is not hydrogen.

20. The compound of any one of claims 16 to 19, wherein the compound has a structure of Formula (XII-a): 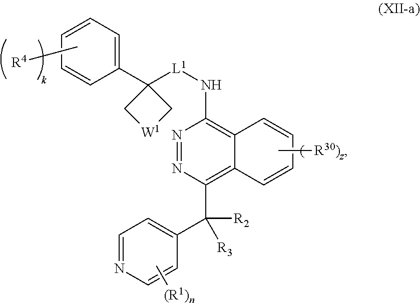
wherein:
W¹ is —O— or CH₂—;
L¹ is a bond or C₁-C₄ alkylene,
Each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)R^{4F}, —C(O)OR^{4F}, —S(O)₂RF, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
k is an integer of 0 to 5;
X⁴ is independently —F, —Br, —Cl, or —I; and
Each R^{4F} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

21. The compound of any one of claims 16 to 20, wherein:
Each R⁴ is independently halogen, —CX⁴₃, —OCX⁴₃, —CN, —OR^{4F}, —C(O)R^{4F}, —C(O)OR^{4F}, —S(O)₂R^{4F}, —C(O)NHR^{4F}, —C(O)N(R^{4F})₂, or substituted or unsubstituted C₁-C₄ alkyl; and
Each R^{4F} is independently a hydrogen, substituted or unsubstituted C₁-C₄ alkyl, substituted or unsubstituted 5 to 12 membered mono-cyclic or bi-cyclic heterocycloalkyl, substituted or unsubstituted phenyl, or substituted or unsubstituted 5 to 6 membered heteroaryl.

22. The compound of any one of claims 16 to 21, wherein:
R¹ is halogen, substituted or unsubstituted C₁-C₄ alkyl, —CN, —CX¹₃, or OR^{1F}; and
R^{1F} is hydrogen or unsubstituted C₁-C₄ alkyl.

23. The compound of any one of claims 16 to 22, wherein:
R² is hydrogen or substituted or unsubstituted C₁-C₄ alkyl, and R³ is substituted or unsubstituted C₁-C₄ alkyl.

24. The compound of any one of claims 16 to 23, wherein:
R³⁰ is halogen, substituted or unsubstituted C₁-C₄ alkyl, —CN, —CX¹₃, or —OR^{30F}; and
R^{30F} is hydrogen or unsubstituted C₁-C₄ alkyl.

25. A compound has a structure of Formula (XIII): 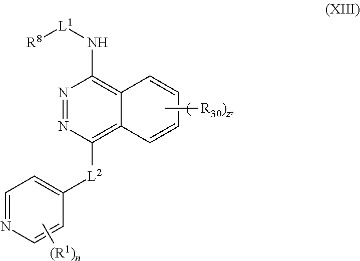
or a pharmaceutically acceptable salt thereof, or an isomer thereof;
wherein:
L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is —C(═O)—, —C(═CR^{9A}R^{9B})—, —CR^{10A}R^{10B}—, or —C(═NR¹¹)—;
R¹ is independently halogen, —CX¹₃, —CHX¹₂, —CH₂X¹, —OCX¹₃, —OCH₂X¹, —OCHX¹₂, —CN, —OR^{1F}, —SR^{1F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁸ is a substituted or unsubstituted cycloalkyl, substituted or unsubstituted heterocycloalkyl, substituted or unsubstituted aryl, or substituted or unsubstituted heteroaryl;
Each R^{9A} and R^{9B} is independently hydrogen, —CX⁹₃, —CHX⁹₂, —CH₂X⁹, —OCX⁹₃, —OCH₂X⁹, —OCHX⁹₂, —CN, —OR^{9F}, —SR^{9F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Each R^{10A} and R^{10B} is independently hydrogen, D, halogen, —CX¹⁰₃, —CHX¹⁰₂, —CH₂X¹⁰, —OCX¹⁰₃, —OCH₂X¹⁰, —OCHX¹⁰₂, —CN, —OR^{10F}, —SR^{10F}, —C(O)OR^{10F}, —C(O)NHR^{10F}, —C(O)N(R^{10F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10A} and R^{10B} are optionally joined together to form a substituted or unsubstituted cycloalkyl, or substituted or unsubstituted heterocycloalkyl;
R¹¹ is —R^{11F}, —OR^{11F}, —S(O₂)—R^{11F}, or —C(O)—R^{11F};
R³⁰ is independently halogen, —CX³⁰₃, —CHX³⁰₂, —CH₂X³⁰, —OCX³⁰₃, —OCH₂X³⁰, —OCHX³⁰₂, —CN, —OR^{30F}, —SR^{30F}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
n is an integer of 1 to 4;
z is an integer of 0 to 4;
Each X¹, X⁸, X⁹, X¹⁰, and X³⁰ is independently —F, —Br, —Cl, or —I;
Each R^{1F}, R^{8F}, R^{9F}, R^{10F} and R^{30F} is independently hydrogen, substituted or unsubstituted alkyl, or substituted or unsubstituted heteroalkyl; and
R^{11F} is a hydrogen, substituted or unsubstituted alkyl, substituted or unsubstituted aryl, or substituted or unsubstituted heteroaryl.

26. The compound of claim 25, wherein R⁸ is 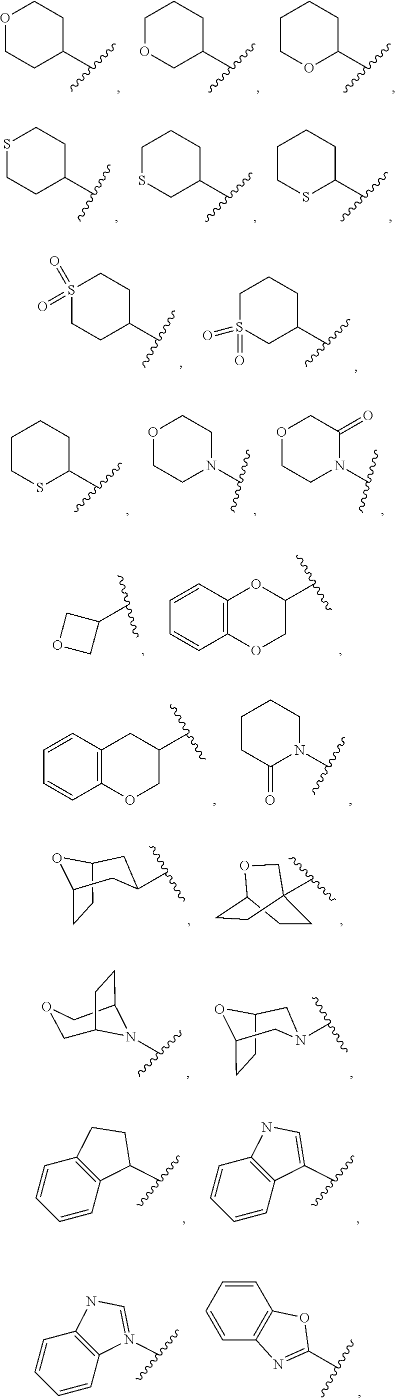 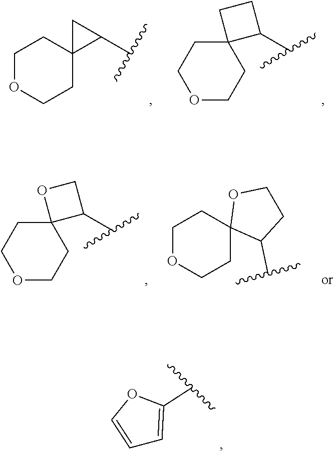 [SMILES: *C1CC12CCOCC2 ; *C1CCC12CCOCC2 ; *C1COC12CCOCC2 ; *C1CCOC12CCOCC2 ; *c1ccco1]
which is substituted or unsubstituted.

27. The compound of claim 25, wherein:
R⁸ is 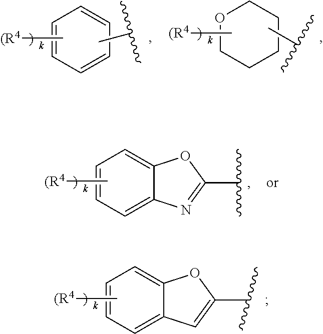
Each R⁴ is independently halogen, —CX⁴₃, —CHX⁴₂, —CH₂X⁴, —OCX⁴₃, —OCH₂X⁴, —OCHX⁴₂, —CN, —OR^{4F}, —SR^{4F}, —C(O)R^{4F}, —C(O)OR^{4F}, —S(O)₂RF, —C(O)NHR^{4F}, —C(O)N(R^{4F})₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
k is an integer of 0 to 5;
X⁴ is independently —F, —Br, —Cl, or —I; and
Each R^{4F} is independently a hydrogen, substituted or unsubstituted C₁-C₄ alkyl, substituted or unsubstituted 5 to 12 membered mono-cyclic or bi-cyclic heterocycloalkyl, substituted or unsubstituted phenyl, or substituted or unsubstituted 5 to 6 membered heteroaryl.

28. The compound of claim 27, wherein:
Each R⁴ is independently halogen, —CX⁴₃, —OCX⁴₃, —CN, —OR^{4F}, —C(O)R^{4F}, —C(O)OR^{4F}, —S(O)₂RF, —C(O)NHR^{4F}, —C(O)N(R^{4F})₂, or substituted or unsubstituted C₁-C₄ alkyl; and
Each R^{4F} is independently a hydrogen, substituted or unsubstituted C₁-C₄ alkyl, substituted or unsubstituted 5 to 12 membered mono-cyclic or bi-cyclic heterocycloalkyl, substituted or unsubstituted phenyl, or substituted or unsubstituted 5 to 6 membered heteroaryl.

29. The compound of 25 to 28, wherein at least one of R^{10A} and R^{10B} is not hydrogen.

30. The compound of any one of claims 25 and 29, wherein the compound has a structure of Formula (XIII-a), (XIII-b) or (XIII-c), 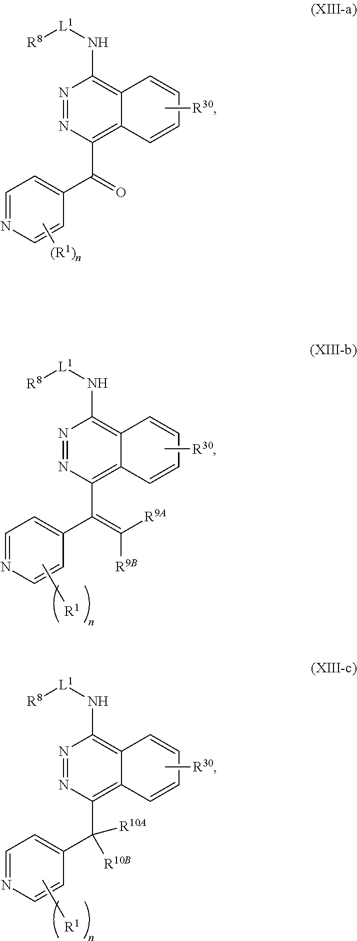
or a pharmaceutically acceptable salt thereof, or an isomer thereof;
wherein n is an integer of 1 to 4.

31. The compound of any one of claims 25 to 30, wherein L¹ is a bond or unsubstituted C₁-C₄ alkylene.

32. The compound of claim 30, wherein R^{10A} and R^{10B} are joined to form a substituted or unsubstituted C3-C₆ cycloalkyl, or substituted or unsubstituted 4 to 6 membered heterocycloalkyl.

33. The compound of claim 32, wherein the compound has a structure of Formula (XIII-c-1) or (XIII-c-2), 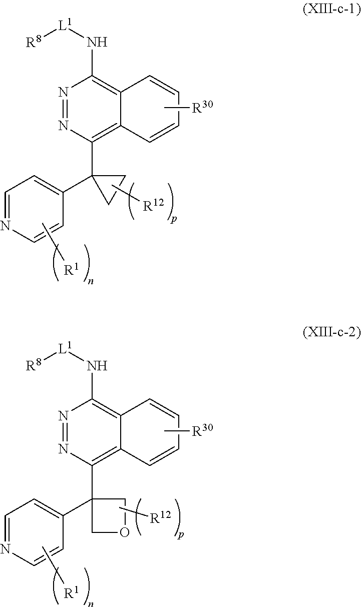
or a pharmaceutically acceptable salt thereof, or an isomer thereof;
wherein:
Each R¹² is independently halogen, —CX¹²₃, —CHX¹²₂, —CH₂X¹², —OCX¹²₃, —OCH₂X¹², —OCHX¹²₂, —CN, —OR^{12F}, —SR^{12F}, —C(O)OR^{12F}, —C(O)NHR^{12F}, —C(O)N(R^{12F})₂ substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X¹² is independently —F, —Br, —Cl, or —I;
Each R¹² is independently hydrogen, substituted or unsubstituted alkyl, or substituted or unsubstituted heteroalkyl; and
p is an integer from 0 to 4.

34. The compound of claim 30, wherein the compound has a structure of Formula (XIII-c-3), (XIII-c-4), or (XIII-c-5), 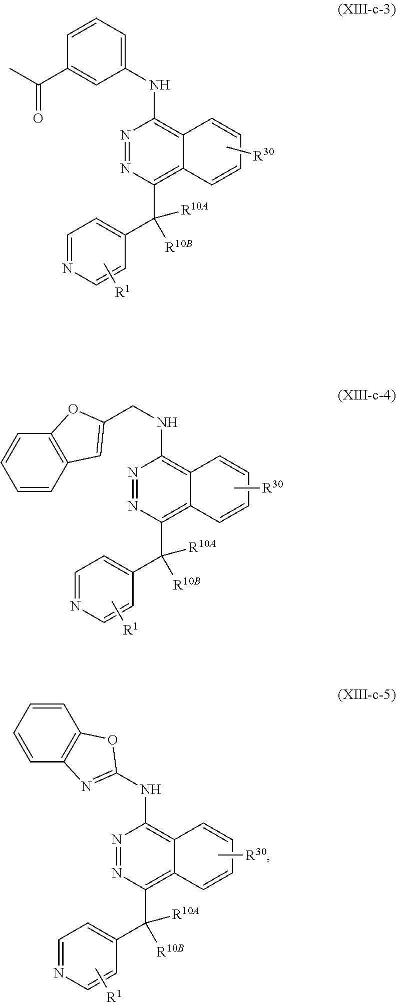
or a pharmaceutically acceptable salt thereof, or an isomer thereof.

35. The compound of claim 34, wherein:
R^{10A} is hydrogen or substituted or unsubstituted C₁-C₄ alkyl, and R^{10B} is substituted or unsubstituted C₁-C₄ alkyl.

36. The compound of any one of claims 25 to 31, wherein the compound has a structure of Formula (XIII-d-1), (XIII-d-2), (XIII-d-3), or (XIII-d-4), 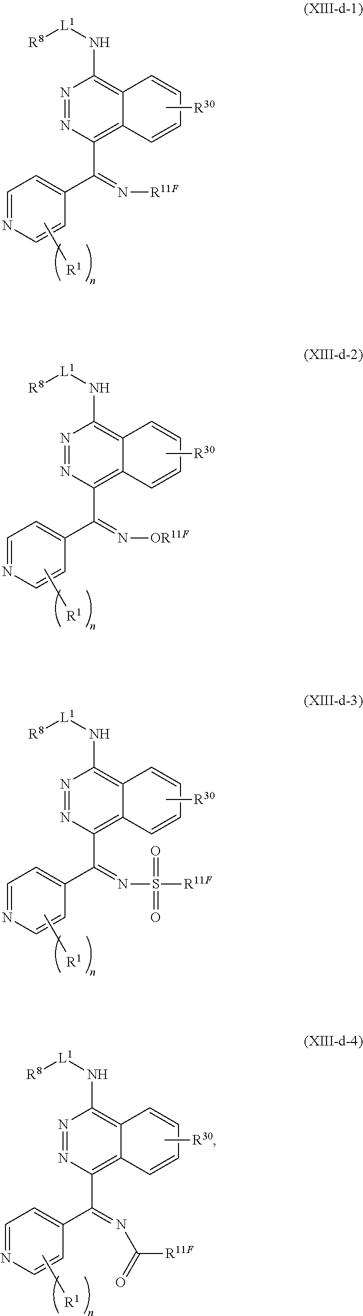
or a pharmaceutically acceptable salt thereof, or an isomer thereof.

37. The compound of claim 36, wherein R^{11F} is hydrogen or unsubstituted C₁-C₄ alkyl.

38. The compound of any one of claims 25 to 37, wherein:
R¹ is halogen, substituted or unsubstituted C₁-C₄ alkyl, —CN, —CX¹₃, or OR^{1F}; and
R^{1F} is hydrogen or unsubstituted C₁-C₄ alkyl.

39. The compound of any one of claims 25 to 35, wherein:
R³⁰ is halogen, substituted or unsubstituted C₁-C₄ alkyl, —CN, —CX¹₃, or —OR^{30F}; and
R^{30F} is hydrogen or unsubstituted C₁-C₄ alkyl.

40. The compound of any one of claims 1 to 40, wherein the compound is any compound of Tables 1-3.

41. A pharmaceutical composition comprising a compound of any one of claims 1 to 40, a pharmaceutically acceptable salt form thereof, an isomer thereof, or a crystal form thereof.

42. A pharmaceutical composition comprising a compound in any of compound in Tables 1-4, a pharmaceutically acceptable salt form thereof, an isomer thereof, or a crystal form thereof.

43. A method of inhibiting NAD consumption and/or increasing NAD synthesis in a subject, comprising administering to the subject an effective dose of a compound of any one of claims 1 to 40 or a pharmaceutical composition of any one of claims 41 to 42.

44. A method of preventing or inhibiting NAD depletion in a patient, or a method of improving a condition linked to alterations of NAD metabolism in a patient, comprising administering to the patient an effective dose of a compound of any one of claims 1 to 40 or a pharmaceutical composition of any one of claims 41 to 42.

45. A method of providing protection from toxicity of misfolded proteins in a patient, comprising administering to the patient an effective dose of a compound of any one of claims 1 to 40 or a pharmaceutical composition of any one of claims 41 to 42.

46. A method of preventing or treating a degenerative disease in a patient, comprising administering to the patient an effective dose of a compound of any one of claims 1 to 40 or a pharmaceutical composition of any one of claims 41 to 42.

47. The methods of claim 46, wherein the degenerative disease is a peripheral amyloidosis or a neurodegenerative disorder associated with misfolded protein-induced neurodegeneration and/or NAD depletion.

48. The methods of claim 46, wherein the degenerative disease is Creutzfeldt-Jakob Disease or other prion disease, Parkinson's disease, dementia with Lewy bodies, multiple system atrophy or other synucleinopathy, Alzheimer's disease, amyotrophic lateral sclerosis, fronto-temporal dementia or other tauopathy, multiple sclerosis, chronic traumatic encephalopathy, ATTR, brain ischemia or an axonopathy.

49. A method of preventing or treating a retinal disease in a patient, comprising administering to the patient an effective dose of a compound of any one of claims 1 to 40 or a pharmaceutical composition of any one of claims 41 to 42.

50. A method of preventing or treating a mitochondrial disease in a patient, comprising administering to the patient an effective dose of a compound of any one of claims 1 to 40 or a pharmaceutical composition of any one of claims 41 to 42.

51. A method of preventing or treating diabetes, non alcoholic fatty liver disease or other metabolic disease in a patient, comprising administering to the patient an effective dose of a compound of any one of claims 1 to 40 or a pharmaceutical composition of any one of claims 41 to 42.

52. A method of preventing or treating a kidney disease in a patient, comprising administering to the patient an effective dose of a compound of any one of claims 1 to 40 or a pharmaceutical composition of any one of claims 41 to 42.

53. A method of mitigating health effects of aging, comprising administering to the patient an effective dose of a compound of any one of claims 1 to 40 or a pharmaceutical composition of any one of claims 41 to 42.
